(19) United States
(12) Patent Application Publication
(10) Pub. No.: US 2024/0285751 A1
(43) Pub. Date: Aug. 29, 2024

(54) CORONAVIRUS VACCINES

(71) Applicants: DIOSynVax Ltd, Cambridge Cambridgeshire (GB); The Chancellor, Masters and Scholars of the University of Cambridge, Cambridge Cambridgeshire (GB)

(72) Inventors: Jonathan Luke HEENEY, Cambridge Cambridgeshire (GB); Sneha VISHWANATH, Cambridge Cambridgeshire (GB); George CARNELL, Cambridge Cambridgeshire (GB); David WELLS, Cambridge Cambridgeshire (GB); Matteo FERRARI, Cambridge Cambridgeshire (GB)

(21) Appl. No.: 17/916,369

(22) PCT Filed: Apr. 1, 2021
(86) PCT No.: PCT/GB2021/050830

§ 371 Date: Sep. 30, 2022

(30) Foreign Application Priority Data
Apr. 1, 2020 (GB) 2004826.0
Jul. 10, 2020 (GB) 2010672.0
Oct. 5, 2020 (GB) 2015775.6
Feb. 10, 2021 (GB) 2101824.7
Mar. 8, 2021 (GB) 2103214.9

(51) Int. Cl. A61K 39/215; A61K 39/00; A61P 31/14; C07K 14/005; C12N 7/00

(52) U.S. Cl. CPC A61K 39/215; A61P 31/14; C07K 14/005; C12N 7/00; A61K 2039/5256; C12N 2770/20034

(57) ABSTRACT

Designed coronavirus polypeptide sequences are described, and their use as vaccines against viruses of the coronavirus family. The designed sequences include designed coronavirus spike (S) proteins and fragments thereof, including designed S protein receptor binding domain (RBD) sequence SEQ ID NO:17, designed truncated S protein sequence SEQ ID NO:15, and designed full length S protein sequence SEQ ID NO:13. Designed coronavirus envelope (E), membrane (M), and nucleocapsid (N) protein sequences are also described, and their use as vaccines. Nucleic acid molecules encoding the polypeptides, vectors, fusion proteins, pharmaceutical compositions, cells, and their use as vaccines against viruses of the coronavirus family are also described.

This invention relates to nucleic acid molecules, polypeptides, vectors, cells, fusion proteins, pharmaceutical compositions, and their use as vaccines against viruses of the coronavirus family.

Coronaviruses (CoVs) cause a wide variety of animal and human disease. Notable human diseases caused by CoVs are zoonotic infections, such as severe acute respiratory syndrome (SARS) and Middle-East respiratory syndrome (MERS). Viruses within this family generally cause mild, self-limiting respiratory infections in immunocompetent humans, but can also cause severe, lethal disease characterised by onset of fever, extreme fatigue, breathing difficulties, anoxia, and pneumonia. CoVs transmit through close contact via respiratory droplets of infected subjects, with varying degrees of infectivity within each strain.

CoVs belong to the Coronaviridae family of viruses, all of which are enveloped. CoVs contain a single-stranded positive-sense RNA genome, with a length of between 25 and 31 kilobases (Siddell S. G. 1995, The Coronaviridae), the largest genome so far found in RNA viruses. The Coronaviridae family are subtyped into four genera: α, β, γ, and δ coronaviruses, based on phylogenetic clustering, with each genus subdivided again into clusters depending on the strain of the virus. For example, within the genus β-CoV (Group 2 CoV), four lineages (a, b, c, and d) are commonly recognized:
Lineage A (subgenus Embecovirus) includes HCoV-OC43 and HCoV-HKU1 (various species)
Lineage B (subgenus Sarbecovirus) includes SARSr-CoV (which includes all its strains such as SARS-CoV, SARS-CoV-2, and Bat SL-CoV-WIV1)
Lineage C (subgenus Merbecovirus) includes Tylonycteris bat coronavirus HKU4 (BtCoV-HKU4), Pipistrellus bat coronavirus HKU5 (BtCoV-HKU5), and MERS-CoV (various species)
Lineage D (subgenus Nobecovirus) includes Rousettus bat coronavirus HKU9 (BtCoV-HKU9)

CoV virions are spherical with characteristic club-shape spike projections emanating from the surface of the virion. The virions contain four main structural proteins: spike (S); membrane (M); envelope (E); and nucleocapsid (N) proteins, all of which are encoded by the viral genome. Some subsets of β-CoVs also comprise a fifth structural protein, hemagglutinin-esterase (HE), which enhances S protein-mediated cell entry and viral spread through the mucosa via its acetyl-esterase activity. Homo-trimers of the S glycoprotein make up the distinctive spike structure on the surface of the virus. These trimers are a class I fusion protein, mediating virus attachment to the host receptor by interaction of the S protein and its receptor. In most CoVs, S is cleaved by host cell protease into two separate polypeptides—S1 and S2. S1 contains the receptor-binding domain (RBD) of the S protein (the exact positioning of the RBD varies depending on the viral strain), while S2 forms the stem of the spike molecule.

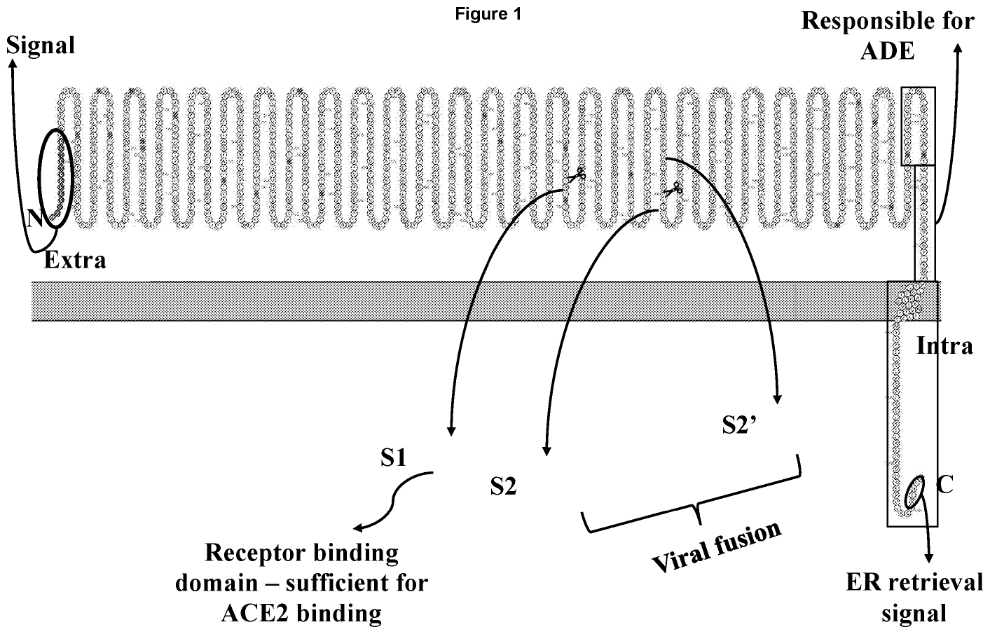

FIG. 1 shows SARS S-protein architecture. The N-terminal sequence is responsible for relaying extracellular signals intracellularly. Studies show that the N-terminal region of the S protein is much more diverse than the C-terminal region, which is highly conserved (Dong et al, Genomic and protein structure modelling analysis depicts the origin and infectivity of 2019-nCoV, a new coronavirus which caused a pneumonia outbreak in Wuhan, China. 2020). The figure shows the S domain, which comprises S1 and S2 domains, responsible for receptor binding and cell membrane fusion respectively.

RNA viruses generally have very high mutation rates compared to DNA viruses, because viral RNA polymerases lack the proofreading ability of DNA polymerases. This is one reason why the virus is able to transmit from its natural host reservoir to other species, and from human to human, and why it is difficult to make effective vaccines to prevent diseases caused by RNA viruses. In most cases, current vaccine candidates against RNA viruses are limited by the viral strain used as the vaccine insert, which is often chosen based on availability of a wild-type strain rather than by informed design. Technical challenges for developing vaccines for enveloped RNA viruses include: i) viral variation of wild-type field isolate glycoproteins (GPs) provide limited breadth of protection as vaccine antigens: ii) selection of vaccine antigens expressed by the vaccine inserts is highly empirical; immunogen selection is a slow, trial and error process; iii) in an evolving or unanticipated viral epidemic, developing new vaccine candidates is time-consuming and can delay vaccine deployment.

Before 2002, CoVs were only thought to cause mild respiratory problems, and were endemic in the human population, causing 15-30% of respiratory tract infections each year. Since their first discovery in the 1960's, the CoV family has expanded massively and has caused many outbreaks in both humans and animals. The SARS pandemic that occurred in 2002-2003 in the Guangdong Province of China was the most severe disease caused by any coronavirus known to that date. During that period, approximately 8098 cases occurred with 774 deaths (mortality rate ˜9.6% overall). The mortality rate was ˜50% in individuals over 90 years of age. The virus, identified as SARS-CoV, a group 2b β-CoV, originated in bats. Two novel virus isolates from bats show more similarity to the human SARS-CoV than any other virus identified to date, and bind to the same cellular receptor as human derived SARS-CoV—angiotensin converting enzyme 2 (ACE2).

While the SARS-CoV epidemic was controlled in 2003, a novel human CoV, a group 2c β-CoV, emerged in the Middle East in 2012. MERS is the causative agent of a series of highly pathogenic respiratory tract infections in the Middle East, with an initial mortality rate of 50%. An estimate of 2,494 cases and 858 deaths caused by MERS has been reported since its emergence, with a total estimated fatality rate by the World Health Organisation (WHO) of 34.4%. Along with SARS-CoV, this novel CoV originated from bats, likely with an intermediate host such as dromedary camels contributing to the spread of the outbreak. This virus utilises dipeptidyl peptidase (DPP4) as its receptor, another peptidase receptor. It is currently unclear why CoVs utilise host peptidases as their binding receptor, as entry occurs even in the absence of enzyme activity.

In the beginning of 2020, another novel CoV emerged; severe acute respiratory syndrome coronavirus 2 (SARS-CoV-2). The outbreak began in Wuhan, China in late 2019. By 30 Jan. 2020 the WHO declared a global health emergency as the virus had spread to over 25 countries within a month of its emergence. At the time of writing, the number of SARS-CoV-2 infections was increasing exponentially across many countries around the world, nearing 800,000 cases of infection, and causing over 40,000 total confirmed deaths.

Human cases or outbreaks of haemorrhagic fevers caused by coronaviruses occur sporadically and irregularly. The occurrence of outbreaks cannot be easily predicted. With a few exceptions, there is no cure or established drug treatment for CoV infections. Vaccines have only been approved for some CoVs, but these vaccines are not always used because they are either not very effective or in some cases have been reported to promote selection of novel pathogenic CoVs via recombination of circulating strains. By April 2020, several potential vaccines had been developed for SARS-CoV but none had been approved for use. A year later, several novel vaccines have had regulatory approval, and a mass vaccination programme is underway. The first mass vaccination programme started in early December 2020, and as of 15 Feb. 2021, the WHO estimates that 175.3 million vaccine doses have been administered. At least 7 different vaccines are being used worldwide. WHO issued an Emergency Use Listing (EUL) for the Pfizer-BioNTech COVID-19 vaccine (BNT162b2) on 31 Dec. 2020. On 15 Feb. 2021, WHO issued EULs for two versions of the AstraZeneca/Oxford COVID-19 vaccine (AZD1222). As of 18 Feb. 2021, the UK had administered 12 million people with their first dose of either of the Pfizer-BioNTech or the AstraZeneca/Oxford vaccine. Both the Pfizer and AstraZeneca vaccine use an mRNA platform encoding the S protein. Pfizer uses a nanoparticle vector for nucleic acid delivery, whereas AstraZeneca uses an adenoviral vector.

There are many hurdles to overcome in the development of an effective vaccine for CoVs. Firstly, immunity, whether it is natural or artificial, does not necessarily prevent subsequent infection (Fehr et al. Methods Mol Biol. 2015, 1282:1-23). Secondly, the propensity of the viruses to recombine may pose a problem by rendering the vaccine useless by increasing the genetic diversity of the virus. Additionally, vaccination with the viral S-protein has been shown to lead to enhanced disease in the case of FIPV (feline infectious peritonitis virus), a highly virulent strain of feline CoV. This enhanced pathogenicity of the disease is caused by non-neutralising antibodies that facilitate viral entry into host cells in a process called antibody-dependent enhancement (ADE). After primary infection of one strain of a virus, neutralising antibodies are produced against the same strain of the virus. However, if a different strain infects the host in a secondary infection, non-neutralising antibodies produced during the first infection, which do not neutralise the virus, instead, bind to the virus and then bind to the IgG Fc receptors on immune cells and mediate viral entry into these cells (Wan et al. Journal of Virology. 2020, 94(5):1-13).

When developing vaccines against viruses that are capable of ADE (or of triggering ADE-like pro-inflammatory responses), it is crucial that epitopes are identified that are responsible for eliciting non-neutralising antibodies, and that these epitopes are either masked by modification or are removed from the vaccine. These non-neutralising epitopes on the S-protein may also result in immune diversion wherein the non-neutralising epitopes outcompete neutralising epitopes for binding to antibodies. The neutralising epitopes are neglected by the immune system which fails to neutralise the antigen. In the case of recombinant RBD vaccines, previously buried surfaces containing non-neutralising immunodominant epitopes may become newly exposed which outcompete epitopes responsible for neutralisation by the immune system.

There is a need, therefore, to provide effective vaccines that induce a broadly neutralising immune response to protect against emerging and re-emerging diseases caused by CoVs, especially β-CoVs, such as SARS-CoV and the recent SARS-CoV-2. In particular, there is a need to provide vaccines lacking non-neutralising epitopes that may result in virus immune evasion and disease progression by ADE (or ADE-like pro-inflammatory responses).

### Designed Coronavirus Spike (S) Protein Sequences (Full-Length, Truncated, and Receptor Binding Domain, RBD)

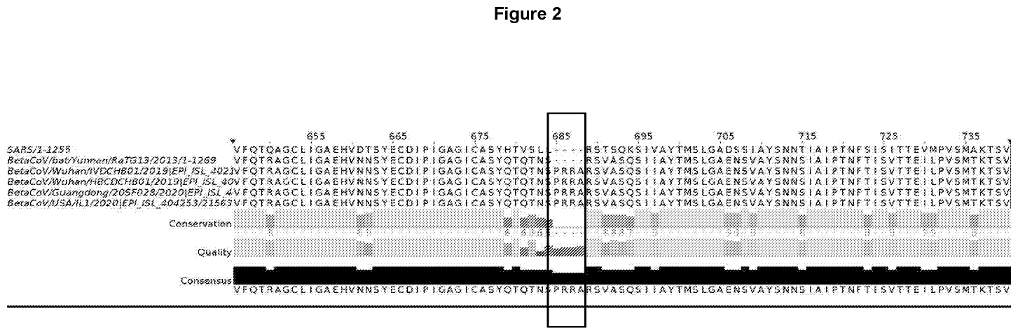

FIG. 2 shows a multiple sequence alignment of the S-protein (the region around the cleavage site 1) comparing SARS-CoV isolate (SARS-CoV-1), and closely related bat betacoronavirus (RaTG13) isolate, with four SARS-CoV-2 isolates. The SARS-CoV S-protein (1269 amino acid residues) shares a high sequence identity (˜73%) with the SARS-CoV-2 S-protein (1273 amino acid residues). Expansion of cleavage site one (shown as a boxed area in the figure) is observed in all SARS-CoV-2 strains so far. The majority of the insertions/substitutions are observed in the subunit 1, with minimal substitutions in the subunit S2, as compared to SARS-CoV-1. The C-terminus contains epitopes which elicit non-neutralising antibodies and are responsible for antibody dependent enhancement.

The applicant has generated a novel amino acid sequence for an S-protein, called CoV_T2_1 (also referred to below as Wuhan-Node-1), which has improved immunogenicity (which allows the protein and its derivatives to elicit a broadly neutralising immune response).

The amino acid sequences of the full length S-protein (SEQ ID NO:13) (CoV_T2_1; Wuhan-Node-1), truncated S-protein (tr, missing the C-terminal part of the S2 sequence) (SEQ ID NO:15) (CoV_T2_4; Wuhan_Node1_tr), and the receptor binding domain (RBD) (SEQ ID NO:17) (CoV_T2_7; Wuhan_Node1_RBD) (and their respective encoding nucleic acid sequences, SEQ ID NOs: 14, 16, 18) are provided in the examples below.

According to the invention there is provided an isolated polypeptide which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:17.

SEQ ID NO:17 is the amino acid sequence of a novel S-protein RBD designed by the applicant.

There is also provided according to the invention an isolated polypeptide comprising an amino acid sequence of SEQ ID NO: 15, or an amino acid sequence which has at least 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:15.

There is also provided according to the invention an isolated polypeptide comprising an amino acid sequence of SEQ ID NO: 13, or an amino acid sequence which has at least 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:13.

Examples 6 and 7 below provide amino acid sequence alignments of the novel S-protein RBD amino acid sequence (Wuhan_Node1_RBD (CoV_T2_7) (SEQ ID NO:17)) with the RBD amino acid sequences of SARS-TOR2 isolate AY274119 (AY274119_RBD (CoV_T2-5) (SEQ ID NO:5)), and SARS_CoV_2 isolate hCov-19/Wuhan/LVDC-HB-01/2019 (EPI_ISL_402119) (EPI_ISL_402119_RBD (CoV_T2_6) (SEQ ID NO:11)), respectively.

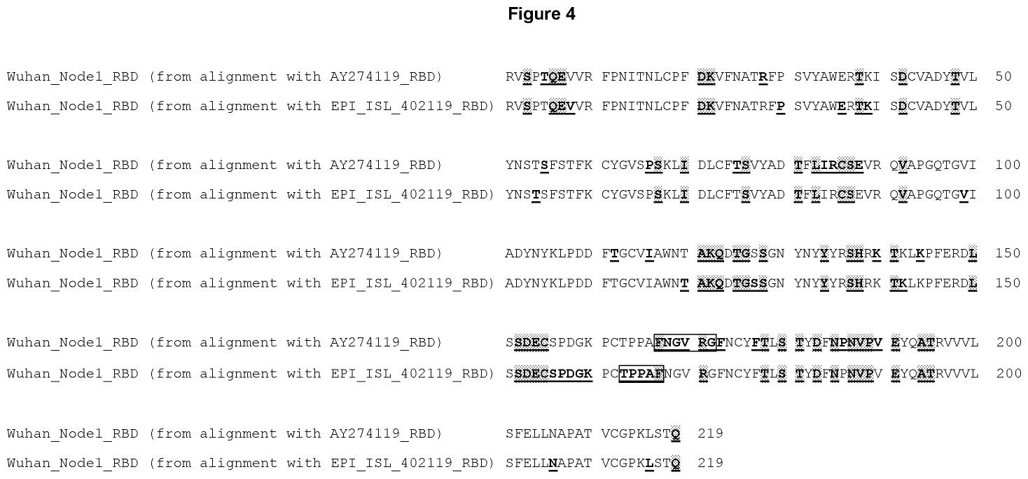

As explained in Example 9 below. FIG. 4 shows Wuhan_Node1_RBD (CoV_T2_7) amino acid sequence (SEQ ID NO:17) with amino acid residue differences highlighted in bold and underline from the respective alignments with AY274119_RBD (CoV_T2_5) (SEQ ID NO:5) and EPI_ISL_402119_RBD (CoV_T2_6) (SEQ ID NO:11) amino acid sequences (Examples 6 and 7, respectively). The amino acid residue differences from the two alignments are listed in the table below (the numbering of residue positions corresponds to positions of the Wuhan_Node1_RBD (CoV_T2_7) (SEQ ID NO:17) amino acid sequence. The common differences from the two alignments are at amino acid residues: 3, 6, 7, 21, 22, 38, 42, 48, 67, 70, 76, 81, 83, 86, 87, 92, 121, 122, 123, 125, 126, 128, 134, 137, 138, 141, 150, 152, 153, 154, 155, 167, 171, 178, 180, 181, 183, 185, 187, 188, 189, 191, 194, 195, 219 (shown with grey highlighting in FIG. 4, and in the table below):

Amino acid insertions are at positions 167-172 (compared to AY274119_RBD), and 163-167 (compared to EPI_ISL_402119_RBD) (shown boxed in FIG. 4).

Optionally an isolated polypeptide of the invention comprises at least one of the amino acid residues, at a position corresponding to the amino acid residue position of SEQ ID NO:17, as shown in Table 2 below:

TABLE 2

| Wuhan_Node1_RBD | |
|---|---|
| (CoV_T2_7) residue | |
| position | Amino acid residue |
| 3 | S |
| 6 | Q |
| 7 | E |
| 21 | D |
| 22 | K |
| 38 | T |
| 42 | D |
| 48 | T |
| 67 | S |
| 70 | I |
| 76 | S |
| 81 | T |
| 83 | L |
| 86 | C |
| 87 | S |
| 92 | V |
| 121 | A |
| 122 | K |
| 123 | Q |
| 125 | T |
| 126 | G |
| 128 | S |
| 134 | Y |
| 137 | S |
| 138 | H |
| 141 | T |
| 150 | L |
| 152 | S |
| 153 | D |
| 154 | E |
| 155 | C |
| 167 | F |
| 171 | R |
| 178 | T |
| 180 | S |
| 181 | T |
| 183 | D |
| 185 | N |
| 187 | N |
| 188 | V |
| 189 | P |
| 191 | E |
| 194 | A |
| 195 | T |
| 219 | Q |

Optionally an isolated polypeptide of the invention comprises at least five of the amino acid residues, at positions corresponding to the amino acid residue positions of SEQ ID NO:17, as shown in Table 2.

Optionally an isolated polypeptide of the invention comprises at least ten of the amino acid residues, at positions corresponding to the amino acid residue positions of SEQ ID NO:17, as shown in Table 2.

Optionally an isolated polypeptide of the invention comprises at least fifteen of the amino acid residues, at positions corresponding to the amino acid residue positions of SEQ ID NO:17, as shown in Table 2.

Optionally an isolated polypeptide of the invention comprises at least twenty of the amino acid residues, at positions corresponding to the amino acid residue positions of SEQ ID NO:17, as shown in Table 2.

Optionally an isolated polypeptide of the invention comprises at least twenty five of the amino acid residues, at positions corresponding to the amino acid residue positions of SEQ ID NO:17, as shown in Table 2.

Optionally an isolated polypeptide of the invention comprises at least thirty of the amino acid residues, at positions corresponding to the amino acid residue positions of SEQ ID NO:17, as shown in Table 2.

Optionally an isolated polypeptide of the invention comprises at least thirty five of the amino acid residues, at positions corresponding to the amino acid residue positions of SEQ ID NO:17, as shown in Table 2.

Optionally an isolated polypeptide of the invention comprises at least forty of the amino acid residues, at positions corresponding to the amino acid residue positions of SEQ ID NO:17, as shown in Table 2.

Optionally an isolated polypeptide of the invention comprises all of the amino acid residues, at positions corresponding to the amino acid residue positions of SEQ ID NO:17, as shown in Table 2.

Optionally an isolated polypeptide of the invention comprises at least one of the amino acid residues, at a position corresponding to the amino acid residue position of SEQ ID NO:17, as shown in Table 3 below:

TABLE 3

| Wuhan_Node1_RBD | |
|---|---|
| (CoV_T2_7) residue | |
| position | Amino acid residue |
| 3 | S |
| 6 | Q |
| 7 | E |
| 8 | V |
| 21 | D |
| 22 | K |
| 30 | P |
| 36 | E |
| 38 | T |
| 39 | K |
| 42 | D |
| 48 | T |
| 54 | T |
| 67 | S |
| 70 | I |
| 76 | S |
| 81 | T |
| 83 | L |
| 86 | C |
| 87 | S |
| 92 | V |
| 99 | V |
| 120 | T |
| 121 | A |
| 122 | K |
| 123 | Q |
| 125 | T |
| 126 | G |
| 127 | S |
| 128 | S |
| 134 | Y |
| 137 | S |
| 138 | H |
| 141 | T |
| 142 | K |
| 150 | L |
| 152 | S |
| 153 | D |
| 154 | E |
| 155 | C |
| 156 | S |
| 157 | P |
| 158 | D |
| 159 | G |
| 160 | K |
| 163 | T |
| 164 | P |
| 165 | P |
| 166 | A |
| 167 | F |
| 171 | R |
| 178 | T |
| 180 | S |
| 181 | T |
| 183 | D |
| 185 | N |
| 187 | N |
| 188 | V |
| 189 | P |
| 191 | E |
| 194 | A |
| 195 | T |
| 206 | N |
| 216 | L |
| 219 | Q |

Optionally an isolated polypeptide of the invention comprises at least five of the amino acid residues, at positions corresponding to the amino acid residue positions of SEQ ID NO:17, as shown in Table 3.

Optionally an isolated polypeptide of the invention comprises at least ten of the amino acid residues, at positions corresponding to the amino acid residue positions of SEQ ID NO:17, as shown in Table 3.

Optionally an isolated polypeptide of the invention comprises at least fifteen of the amino acid residues, at positions corresponding to the amino acid residue positions of SEQ ID NO:17, as shown in Table 3.

Optionally an isolated polypeptide of the invention comprises at least twenty of the amino acid residues, at positions corresponding to the amino acid residue positions of SEQ ID NO:17, as shown in Table 3.

Optionally an isolated polypeptide of the invention comprises at least twenty five of the amino acid residues, at positions corresponding to the amino acid residue positions of SEQ ID NO:17, as shown in Table 3.

Optionally an isolated polypeptide of the invention comprises at least thirty of the amino acid residues, at positions corresponding to the amino acid residue positions of SEQ ID NO:17, as shown in Table 3.

Optionally an isolated polypeptide of the invention comprises at least thirty five of the amino acid residues, at positions corresponding to the amino acid residue positions of SEQ ID NO:17, as shown in Table 3.

Optionally an isolated polypeptide of the invention comprises at least forty of the amino acid residues, at positions corresponding to the amino acid residue positions of SEQ ID NO:17, as shown in Table 3.

Optionally an isolated polypeptide of the invention comprises at least forty five of the amino acid residues, at positions corresponding to the amino acid residue positions of SEQ ID NO:17, as shown in Table 3.

Optionally an isolated polypeptide of the invention comprises at least fifty of the amino acid residues, at positions corresponding to the amino acid residue positions of SEQ ID NO:17, as shown in Table 3.

Optionally an isolated polypeptide of the invention comprises at least fifty five of the amino acid residues, at positions corresponding to the amino acid residue positions of SEQ ID NO:17, as shown in Table 3.

Optionally an isolated polypeptide of the invention comprises at least sixty of the amino acid residues, at positions corresponding to the amino acid residue positions of SEQ ID NO:17, as shown in Table 3.

Optionally an isolated polypeptide of the invention comprises all of the amino acid residues, at positions corresponding to the amino acid residue positions of SEQ ID NO:17, as shown in Table 3.

Optionally an isolated polypeptide of the invention comprises at least one of the amino acid residues, at a position corresponding to the amino acid residue position of SEQ ID NO:17, as shown in Table 4 below:

TABLE 4

| Wuhan_Node1_RBD | |
|---|---|
| (CoV_T2_7) residue | |
| position | Amino acid residue |
| 3 | S |
| 5 | T |
| 6 | Q |
| 7 | E |
| 2 | D |
| 22 | K |
| 28 | R |
| 38 | T |
| 42 | D |
| 48 | T |
| 55 | S |
| 66 | P |
| 67 | S |
| 70 | I |
| 75 | T |
| 76 | S |
| 81 | T |
| 83 | L |
| 84 | I |
| 85 | R |
| 86 | C |
| 87 | S |
| 88 | E |
| 92 | V |
| 112 | T |
| 116 | I |
| 121 | A |
| 122 | K |
| 123 | Q |
| 125 | T |
| 126 | G |
| 128 | S |
| 134 | Y |
| 137 | S |
| 138 | H |
| 140 | K |
| 141 | T |
| 144 | K |
| 150 | L |
| 152 | S |
| 153 | D |
| 154 | E |
| 155 | C |
| 167 | F |
| 168 | N |
| 169 | G |
| 170 | V |
| 171 | R |
| 172 | G |
| 173 | F |
| 177 | F |
| 178 | T |
| 180 | S |
| 181 | T |
| 183 | D |
| 185 | N |
| 186 | P |
| 187 | N |
| 188 | V |
| 189 | P |
| 190 | V |
| 191 | E |
| 194 | A |
| 195 | T |
| 219 | Q |

Optionally an isolated polypeptide of the invention comprises at least five of the amino acid residues, at positions corresponding to the amino acid residue positions of SEQ ID NO: 17, as shown in Table 4.

Optionally an isolated polypeptide of the invention comprises at least ten of the amino acid residues, at positions corresponding to the amino acid residue positions of SEQ ID NO: 17, as shown in Table 4.

Optionally an isolated polypeptide of the invention comprises at least fifteen of the amino acid residues, at positions corresponding to the amino acid residue positions of SEQ ID NO:17, as shown in Table 4.

Optionally an isolated polypeptide of the invention comprises at least twenty of the amino acid residues, at positions corresponding to the amino acid residue positions of SEQ ID NO:17, as shown in Table 4.

Optionally an isolated polypeptide of the invention comprises at least twenty five of the amino acid residues, at positions corresponding to the amino acid residue positions of SEQ ID NO:17, as shown in Table 4.

Optionally an isolated polypeptide of the invention comprises at least thirty of the amino acid residues, at positions corresponding to the amino acid residue positions of SEQ ID NO:17, as shown in Table 4.

Optionally an isolated polypeptide of the invention comprises at least thirty five of the amino acid residues, at positions corresponding to the amino acid residue positions of SEQ ID NO:17, as shown in Table 4.

Optionally an isolated polypeptide of the invention comprises at least forty of the amino acid residues, at positions corresponding to the amino acid residue positions of SEQ ID NO:17, as shown in Table 4.

Optionally an isolated polypeptide of the invention comprises at least forty five of the amino acid residues, at positions corresponding to the amino acid residue positions of SEQ ID NO:17, as shown in Table 4.

Optionally an isolated polypeptide of the invention comprises at least fifty of the amino acid residues, at positions corresponding to the amino acid residue positions of SEQ ID NO:17, as shown in Table 4.

Optionally an isolated polypeptide of the invention comprises at least fifty five of the amino acid residues, at positions corresponding to the amino acid residue positions of SEQ ID NO:17, as shown in Table 4.

Optionally an isolated polypeptide of the invention comprises at least sixty of the amino acid residues, at positions corresponding to the amino acid residue positions of SEQ ID NO:17, as shown in Table 4.

Optionally an isolated polypeptide of the invention comprises all of the amino acid residues, at positions corresponding to the amino acid residue positions of SEQ ID NO:17, as shown in Table 4.

According to the invention there is also provided an isolated polypeptide, which comprises a coronavirus S protein RBD domain with any, or all of the amino acid residues at positions corresponding to the amino acid residue positions as shown in Table 5 below:

TABLE 5

| S protein RBD | |
|---|---|
| residue position | Amino acid residue |
| 3 | S |
| 6 | Q |
| 7 | E |
| 21 | D |
| 22 | K |
| 38 | T |
| 42 | D |
| 48 | T |
| 67 | S |
| 70 | I |
| 76 | S |
| 81 | T |
| 83 | L |
| 86 | C |
| 87 | S |
| 92 | V |
| 121 | A |
| 122 | K |
| 123 | Q |
| 125 | T |
| 126 | G |
| 128 | S |
| 134 | Y |
| 137 | S |
| 138 | H |
| 141 | T |
| 150 | L |
| 152 | S |
| 153 | D |
| 154 | E |
| 155 | C |
| 167 | F |
| 171 | R |
| 178 | T |
| 180 | S |
| 181 | T |
| 183 | D |
| 185 | N |
| 187 | N |
| 188 | V |
| 189 | P |
| 191 | E |
| 194 | A |
| 195 | T |
| 219 | Q |

According to the invention there is also provided an isolated polypeptide, which comprises a coronavirus S protein RBD domain with any, or all of the amino acid residues at positions corresponding to the amino acid residue positions as shown in Table 6 below:

TABLE 6

| S protein RBD | |
|---|---|
| residue position | Amino acid residue |
| 3 | S |
| 6 | Q |
| 7 | E |
| 8 | V |
| 21 | D |
| 22 | K |
| 30 | P |
| 36 | E |
| 38 | T |
| 39 | K |
| 42 | D |
| 48 | T |
| 54 | T |
| 67 | S |
| 70 | 1 |
| 76 | S |
| 81 | T |
| 83 | L |
| 86 | C |
| 87 | S |
| 92 | V |
| 99 | V |
| 120 | T |
| 121 | A |
| 122 | K |
| 123 | Q |
| 125 | T |
| 126 | G |
| 127 | S |
| 128 | S |
| 134 | Y |
| 137 | S |
| 138 | H |
| 141 | T |
| 142 | K |
| 150 | L |
| 152 | S |
| 153 | D |
| 154 | E |
| 155 | C |
| 156 | S |
| 157 | P |
| 158 | D |
| 159 | G |
| 160 | K |
| 163 | T |
| 164 | P |
| 165 | P |
| 166 | A |
| 167 | F |
| 171 | R |
| 178 | T |
| 180 | S |
| 181 | T |
| 183 | D |
| 185 | N |
| 187 | N |
| 188 | V |
| 189 | P |
| 191 | E |
| 194 | A |
| 195 | T |
| 206 | N |
| 216 | L |
| 219 | Q |

There is also provided according to the invention an isolated polypeptide, which comprises a coronavirus S protein RBD domain with any, or all of the amino acid residues at positions corresponding to the amino acid residue positions as shown in Table 7 below:

TABLE 7

| S protein RBD | |
|---|---|
| residue position | Amino acid residue |
| 3 | S |
| 5 | T |
| 6 | Q |
| 7 | E |
| 21 | D |
| 22 | K |
| 28 | R |
| 38 | T |
| 42 | D |
| 48 | T |
| 55 | S |
| 66 | P |
| 67 | S |
| 70 | I |
| 75 | T |
| 76 | S |
| 81 | T |
| 83 | L |
| 84 | I |
| 85 | R |
| 86 | C |
| 87 | S |
| 88 | E |
| 92 | V |
| 112 | T |
| 116 | I |
| 121 | A |
| 122 | K |
| 123 | Q |
| 125 | T |
| 126 | G |
| 128 | S |
| 134 | Y |
| 137 | S |
| 138 | H |
| 140 | K |
| 141 | T |
| 144 | K |
| 150 | L |
| 152 | S |
| 153 | D |
| 154 | E |
| 155 | C |
| 167 | F |
| 168 | N |
| 169 | G |
| 170 | V |
| 171 | R |
| 172 | G |
| 173 | F |
| 177 | F |
| 178 | T |
| 180 | S |
| 181 | T |
| 183 | D |
| 185 | N |
| 186 | P |
| 187 | N |
| 188 | V |
| 189 | P |
| 190 | V |
| 191 | E |
| 194 | A |
| 195 | T |
| 219 | Q |

Optionally an isolated polypeptide of the invention which comprises a coronavirus S protein RBD domain comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:5.

Optionally an isolated polypeptide of the invention which comprises a coronavirus S protein RBD domain comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:11.

Further novel S protein RBD sequences are referred to herein as CoV_S_T2_13-CoV_S_T2_18 (SEQ ID NOs: 27-32, respectively). CoV_S_T2_13 is the direct output of our design algorithm, and CoV_S_T2_14-CoV_S_T2_18 are epitope-enriched versions of CoV_S_T2_13. The amino acid sequences of these designed sequences are provided below, and in Example 12:

>COV_S_T2_13
(SEQ ID NO: 27)
RVAPTKEVVR FPNITNLCPF GEVFNATRFP SVYAWERKRI
SNCVADYSVL YNSTSFSTFK CYGVSPTKLN DLCFTNVYAD
SFVIRGDEVR QIAPGQTGVI ADYNYKLPDD FTGCVIAWNT
NNLDSTTGGN YNYLYRSLRK SKLKPFERDI SSDIYSPGGK
PCSGVEGFNC YYPLRSYGFF PTNGVGYQPY RVVVLSFELL
NAPATVCGPK LSTD
>COV_S_T2_14
(SEQ ID NO: 28)
RVAPTKEVVR FPNITNLCPF GEVFNATKFP SVYAWERKKI
SNCVADYSVL YNSTSFSTFK CYGVSPTKLN DLCFTNVYAD
SFVIRGDEVR QIAPGQTGVI ADYNYKLPDD FTGCVIAWNT
NNIDSTTGGN YNYLYRSLRK SKLKPFERDI SSDIYSPGGK
PCSGVEGFNC YYPLRSYGFF PTNGVGYQPY RVVVLSFELL
NAPATVCGPK LSTD
>COV_S_T2_15
(SEQ ID NO: 29)
RVAPTKEVVR FPNITNLCPF GEVFNATRFP SVYAWERKRI
SNCVADYSVL YNSTFFSTFK CYGVSPTKLN DLCFSNVYAD
SFVIRGDEVR QIAPGQTGVI ADYNYKLPDD FMGCVIAWNT
NNLDSTTGGN YNYLYRSLRK SKLKPFERDI SSDIYSPGGK
PCSGVEGFNC YYPLRSYGFF PTNGVGYQPY RVVVLSFELL
NAPATVCGPK LSTD
>COV_S_T2_16
(SEQ ID NO: 30)
RVAPTKEVVR FPNITNLCPF GEVFNATRFP SVYAWERKRI
SNCVADYSVL YNSTSFSTFK CYGVSPTKLN DLCFTNVYAD
SFVIRGDEVR QIAPGQTGKI ADYNYKLPDD FTGCVIAWNT
NNLDSTTGGN YNYLYRLFRK SNLKPFERDI SSDIYQAGST
PCSGVEGFNC YFPLQSYGFQ PTNGVGYQPY RVVVLSFELL
NAPATVCGPK LSTD
>COV_S_T2_17
(SEQ ID NO: 31)
RVAPTKEVVR FPNITNLCPF GEVFNATKFP SVYAWERKKI
SNCVADYSVL YNSTSFSTFK CYGVSPTKLN DLCFTNVYAD
SFVIRGDEVR QIAPGQTGVI ADYNYKLPDD FTGCVIAWNT
NNIDSTTGGN YNYLYRSLRK SKLKPFERDI SSDIYSPGGK
PCSGVEGFNC YYPLRSYGFF PTNGTGYQPY RVVVLSFELL
NAPATVCGPK LSTD
>COV_S_T2_18
(SEQ ID NO: 32)
RVAPTKEVVR FPNITNLCPF GEVFNATRFP SVYAWERKRI
SNCVADYSVL YNSTFFSTEK CYGVSPTKLN DLCFSNVYAD
SFVIRGDEVR QIAPGQTGVI ADYNYKLPDD FMGCVIAWNT
NNLDSTTGGN YNYLYRSLRK SKLKPFERDI SSDIYSPGGK
PCSGVEGFNC YYPLRSYGFF PTNGTGYQPY RVVVLSFELL
NAPATVCGPK LSTD

Alignment of these sequences with SARS2 Reference sequence (EPI_ISL_402119_RBD (CoV_T2_6) (SEQ ID NO:11)) is shown in Example 12 below.

The amino acid differences of the designed sequences from the SARS2 reference sequence are shown in Table 8.1 below (with differences from the reference sequence highlighted in bold, and differences that are common to all the designed sequences underlined):

TABLE 8.1

| SARS2 RBD | | T2_13 | T2_14 | T2_15 | T2_16 | T2_17 | T2_18 |
|---|---|---|---|---|---|---|---|
| (CoV_T2_6; SEQ | | residue | residue | residue | residue | residue | residue |
| ID NO: 11) | Reference | (SEQ ID | (SEQ ID | (SEQ ID | (SEQ ID | (SEQ ID | (SEQ ID |
| residue position | residue | NO: 27) | NO: 28) | NO: 29) | NO: 30) | NO: 31) | NO: 32) |
| 3 | Q | A | A | A | A | A | A |
| 6 | E | K | K | K | K | K | K |
| 7 | S | E | E | E | E | E | E |
| 8 | I | V | V | V | V | V | V |
| 28 | R | R | K | R | R | K | R |
| 30 | A | P | P | P | P | P | P |
| 36 | N | E | E | E | E | E | E |
| 39 | R | R | K | R | R | K | R |
| 54 | A | T | T | T | T | T | T |
| 55 | S | S | S | F | S | S | F |
| 75 | T | T | T | S | T | T | S |
| 99 | K | V | V | V | K | V | V |
| 112 | T | T | T | M | T | T | M |
| 120 | S | T | T | T | T | T | T |
| 123 | L | L | I | L | L | I | L |
| 126 | K | T | T | T | T | T | T |
| 127 | V | T | T | T | T | T | T |
| 137 | L | S | S | S | L | S | S |
| 138 | F | L | L | L | F | L | L |
| 142 | N | K | K | K | N | K | K |
| 152 | T | S | S | S | S | S | S |
| 153 | F | D | D | D | D | D | D |
| 156 | Q | S | S | S | Q | S | S |
| 157 | A | P | P | P | A | P | P |
| 159 | S | G | G | G | S | G | G |
| 160 | T | K | K | K | T | K | K |
| 163 | N | S | S | S | S | S | S |
| 172 | F | Y | Y | Y | F | Y | Y |
| 175 | Q | R | R | R | Q | R | R |
| 180 | Q | F | F | F | Q | F | F |
| 185 | V | V | V | V | V | T | T |
| 201 | H | N | N | N | N | N | N |
| 211 | K | L | L | L | L | L | L |
| 214 | N | D | D | D | D | D | D |
| Total no of | — | 27 | 30 | 30 | 16 | 31 | 31 |
| differences from | | | | | | | |
| reference | | | | | | | |
| Percentage | — | 87.38 | 85.98 | 85.98 | 92.52 | 85.51 | 85.51 |
| identity with | | | | | | | |
| reference | | | | | | | |

The amino acid changes common to all of the designed sequences are summarised in Table 8.2 below:

TABLE 8.2

| SARS2 RBD | | |
|---|---|---|
| (CoV_T2_6; SEQ | | |
| ID NO: 11) | Reference | Design |
| residue position | residue | residue |
| 3 | Q | A |
| 6 | E | K |
| 7 | S | E |
| 8 | I | V |
| 30 | A | P |
| 36 | N | E |
| 54 | A | T |
| 120 | S | T |
| 126 | K | T |
| 127 | V | T |
| 152 | T | S |
| 153 | E | D |
| 163 | N | S |
| 201 | H | N |
| 211 | K | L |
| 214 | N | D |

Optional additional changes are summarised in Table 8.3 below:

TABLE 8.3

| SARS2 RBD | | |
|---|---|---|
| (CoV_T2_6; SEQ | | |
| ID NO: 11) | Reference | Design |
| residue position | residue | residue |
| 99 | K | V |
| 137 | L | S |
| 138 | F | |
| 142 | N | K |
| 156 | Q | S |
| 157 | A | |
| 159 | S | G |
| 160 | T | K |
| 172 | F | Y |
| 175 | Q | R |
| 180 | Q | F |

The additional changes listed in Table 8.3 are found in SEQ ID NOs:27-29, 31, and 32.

Further optional additional changes are summarised in Tables 8.4-8.6 below:

TABLE 8.4

| SARS2 RBD | | | |
|---|---|---|---|
| (CoV_T2_6; SEQ | | | Found |
| ID NO: 11) | Reference | Design | in SEQ |
| residue position | residue | residue | ID NO: |
| 28 | R | K | 28, 31 |
| 39 | R | K | 28, 31 |
| 123 | L | I | 28, 31 |

TABLE 8.5

| SARS2 RBD | | | |
|---|---|---|---|
| (CoV_T2_6; SEQ | | | Found |
| ID NO:11) | Reference | Design | in SEQ |
| residue position | residue | residue | ID NO: |
| 55 | S | F | 29, 32 |
| 75 | T | S | 29, 32 |
| 112 | T | M | 29, 32 |

TABLE 8.6

| SARS2 RBD | | | |
|---|---|---|---|
| (CoV_T2_6; SEQ | | | Found |
| ID NO: 11) | Reference | Design | in SEQ |
| residue position | residue | residue | ID NO: |
| 185 | V | T | 31, 32 |

According to the invention there is provided an isolated polypeptide, which comprises an amino acid sequence according to any of SEQ ID NOs: 27-32.

According to the invention there is also provided an isolated polypeptide comprising an amino acid sequence of SEQ ID NO: 27 (COV_S_T2_13), or an amino acid sequence which has at least 88%, 89%, 90%, 91%, 92%, 93%, 94%, 95%, 96%, 97%, 98%, or 99% amino acid identity over its entire length with the amino acid sequence of SEQ ID NO:27.

According to the invention there is also provided an isolated polypeptide comprising an amino acid sequence of SEQ ID NO: 28 (COV_S_T2_14), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28.

According to the invention there is also provided an isolated polypeptide comprising an amino acid sequence of SEQ ID NO: 29 (COV_S_T2_15), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29.

According to the invention there is also provided an isolated polypeptide comprising an amino acid sequence of SEQ ID NO: 30 (COV_S_T2_16), or an amino acid sequence which has at least 93%, 94%, 95%, 96%, 97%, 98%, or 99% amino acid identity over its entire length with the amino acid sequence of SEQ ID NO:30.

According to the invention there is also provided an isolated polypeptide comprising an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31.

According to the invention there is also provided an isolated polypeptide comprising an amino acid sequence of SEQ ID NO: 32 (COV_S_T2_18), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32.

Optionally a polypeptide of the invention comprising an amino acid sequence of SEQ ID NO:27 (COV_S_T2_13), or an amino acid sequence which has at least 88%, 89%, 90%, 91%, 92%, 93%, 94%, 95%, 96%, 97%, 98%, or 99% amino acid identity over its entire length with the amino acid sequence of SEQ ID NO:27, comprises at least one, or all of the amino acid residues at positions corresponding to the amino acid residue positions of SEQ ID NO: 11 as shown in Table 8.2 above.

Optionally a polypeptide of the invention comprising an amino acid sequence of SEQ ID NO: 28 (COV_S_T2_14), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28, comprises at least one, or all of the amino acid residues at positions corresponding to the amino acid residue positions as shown in Table 8.2 above.

Optionally a polypeptide of the invention comprising an isolated polypeptide comprising an amino acid sequence of SEQ ID NO: 29 (COV_S_T2_15), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29, comprises at least one, or all of the amino acid residues at positions corresponding to the amino acid residue positions as shown in Table 8.2 above.

Optionally a polypeptide of the invention comprising an amino acid sequence of SEQ ID NO: 30 (COV_S_T2_16), or an amino acid sequence which has at least 93%, 94%, 95%, 96%, 97%, 98%, or 99% amino acid identity over its entire length with the amino acid sequence of SEQ ID NO:30, comprises at least one, or all of the amino acid residues at positions corresponding to the amino acid residue positions as shown in Table 8.2 above.

Optionally a polypeptide of the invention comprising an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31, comprises at least one, or all of the amino acid residues at positions corresponding to the amino acid residue positions as shown in Table 8.2 above.

Optionally a polypeptide of the invention comprising an amino acid sequence of SEQ ID NO: 32 (COV_S_T2_18), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, comprises at least one, or all of the amino acid residues at positions corresponding to the amino acid residue positions as shown in Table 8.2 above.

Optionally a polypeptide of the invention comprising an amino acid sequence of SEQ ID NO:27 (COV_S_T2_13), or an amino acid sequence which has at least 88%, 89%, 90%, 91%, 92%, 93%, 94%, 95%, 96%, 97%, 98%, or 99% amino acid identity over its entire length with the amino acid sequence of SEQ ID NO:27, further comprises at least one, or all of the amino acid residues at positions corresponding to the amino acid residue positions as shown in Table 8.3 above.

Optionally a polypeptide of the invention comprising an amino acid sequence of SEQ ID NO: 28 (COV_S_T2_14), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28, further comprises at least one, or all of the amino acid residues at positions corresponding to the amino acid residue positions as shown in Table 8.3 above.

Optionally a polypeptide of the invention comprising an amino acid sequence of SEQ ID NO: 29 (COV_S_T2_15), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29, further comprises at least one, or all of the amino acid residues at positions corresponding to the amino acid residue positions as shown in Table 8.3 above.

Optionally a polypeptide of the invention comprising an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31, further comprises at least one, or all of the amino acid residues at positions corresponding to the amino acid residue positions as shown in Table 8.3 above.

Optionally a polypeptide of the invention comprising an amino acid sequence of SEQ ID NO: 32 (COV_S_T2_18), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, further comprises at least one, or all of the amino acid residues at positions corresponding to the amino acid residue positions as shown in Table 8.3 above. Optionally a polypeptide of the invention comprising an amino acid sequence of SEQ ID NO: 28 (COV_S_T2_14), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28, further comprises at least one, or all of the amino acid residues at positions corresponding to the amino acid residue positions as shown in Table 8.4 above.

Optionally a polypeptide of the invention comprising an isolated polypeptide comprising an amino acid sequence of SEQ ID NO: 29 (COV_S_T2_15), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29, further comprises at least one, or all of the amino acid residues at positions corresponding to the amino acid residue positions as shown in Table 8.5 above.

Optionally a polypeptide of the invention comprising an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31, further comprises at least one, or all of the amino acid residues at positions corresponding to the amino acid residue positions as shown in Table 8.4 above.

Optionally a polypeptide of the invention comprising an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31, further comprises at least one, or all of the amino acid residues at positions corresponding to the amino acid residue positions as shown in Table 8.6 above.

Optionally a polypeptide of the invention comprising an amino acid sequence of SEQ ID NO: 32 (COV_S_T2_18), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, further comprises at least one, or all of the amino acid residues at positions corresponding to the amino acid residue positions as shown in Table 8.5 above.

Optionally a polypeptide of the invention comprising an amino acid sequence of SEQ ID NO: 32 (COV_S_T2_18), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, further comprises at least one, or all of the amino acid residues at positions corresponding to the amino acid residue positions as shown in Table 8.6 above.

According to the invention there is also provided an isolated polypeptide which comprises a coronavirus S protein RBD domain with at least one of the amino acid residues at positions corresponding to the amino acid residue positions of SEQ ID NO:11, as shown in Table 8.2 above.

Optionally an isolated polypeptide of the invention which comprises a coronavirus S protein RBD domain with at least one of the amino acid residues at positions corresponding to the amino acid residue positions of SEQ ID NO:11, as shown in Table 8.2 above, comprises at least five amino acid residues at positions corresponding to the amino acid residue positions of SEQ ID NO:11, as shown in Table 8.2 above.

Optionally an isolated polypeptide of the invention which comprises a coronavirus S protein RBD domain with at least one of the amino acid residues at positions corresponding to the amino acid residue positions of SEQ ID NO:11, as shown in Table 8.2 above, comprises at least ten amino acid residues at positions corresponding to the amino acid residue positions of SEQ ID NO:11, as shown in Table 8.2 above.

Optionally an isolated polypeptide of the invention which comprises a coronavirus S protein RBD domain with at least one of the amino acid residues at positions corresponding to the amino acid residue positions of SEQ ID NO:11, as shown in Table 8.2 above, comprises at least fifteen amino acid residues at positions corresponding to the amino acid residue positions of SEQ ID NO:11, as shown in Table 8.2 above.

Optionally an isolated polypeptide of the invention which comprises a coronavirus S protein RBD domain with at least one of the amino acid residues at positions corresponding to the amino acid residue positions of SEQ ID NO:11, as shown in Table 8.2 above, comprises all of the amino acid residues at positions corresponding to the amino acid residue positions of SEQ ID NO:11, as shown in Table 8.2 above.

Optionally an isolated polypeptide of the invention which comprises a coronavirus S protein RBD domain with at least one, five, ten, fifteen, or all, of the amino acid residues at positions corresponding to the amino acid residue positions of SEQ ID NO: 11, as shown in Table 8.2 above further comprises at least one, or all of the amino acid residues at positions corresponding to the amino acid residue positions of SEQ ID NO:11, as shown in Table 8.3 above.

Optionally an isolated polypeptide of the invention which comprises a coronavirus S protein RBD domain with at least one, five, ten, fifteen, or all, of the amino acid residues at positions corresponding to the amino acid residue positions of SEQ ID NO:11, as shown in Table 8.2 above and at least one, or all of the amino acid residues at positions corresponding to the amino acid residue positions of SEQ ID NO:11, as shown in Table 8.3 above, further comprises at least one, or all of the amino acid residues at positions corresponding to the amino acid residue positions of SEQ ID NO:11, as shown in any of Tables 8.4 to 8.6 above.

Optionally an isolated polypeptide of the invention which comprises a coronavirus S protein RBD domain comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:5.

Optionally an isolated polypeptide of the invention which comprises a coronavirus S protein RBD domain comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:11.

### Discontinuous Epitope Sequences of Designed S Protein RBD Sequences COV_S_T2_14-18 (SEQ ID NOs: 28-32)

The sequence alignment below shows the designed S protein RBD sequences COV_S_T2_13-18 aligned. The coloured boxes show the residues of discontinuous epitopes present in sequences COV_S_T2_14-18 shown in different colour. The changes made relative to the COV_S_T2_13 sequence to provide discontinuous epitopes that elicit a broader or more potent immune response are shown by the boxed regions:

| | | |
|---|---|---|
| COV_S_T2_13 | 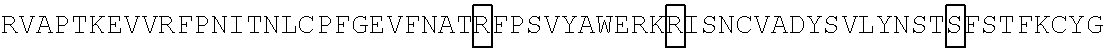 | 63 |
| COV_S_T2_14 | 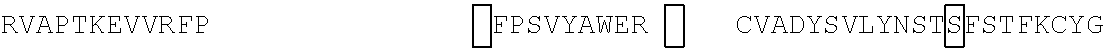 | 63 |
| COV_S_T2_15 | 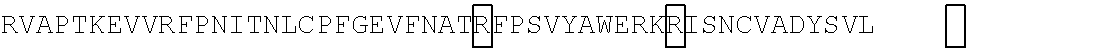 | 63 |
| COV_S_T2_16 | 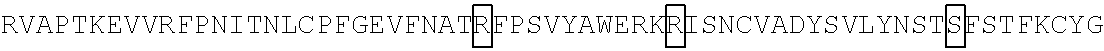 | 63 |
| COV_S_T2_17 | 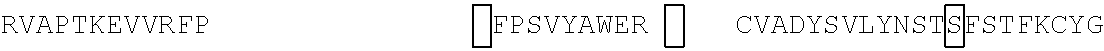 | 63 |
| COV_S_T2_18 | 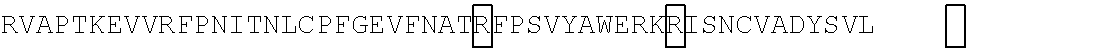 | 63 |
| COV_S_T2_13 | 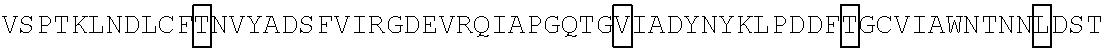 | 126 |
| COV_S_T2_14 | 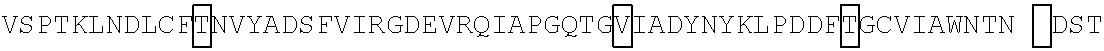 | 126 |
| COV_S_T2_15 | 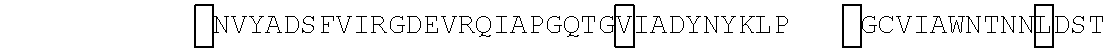 | 126 |
| COV_S_T2_16 | 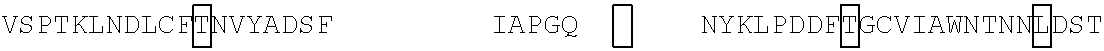 | 126 |
| COV_S_T2_17 | 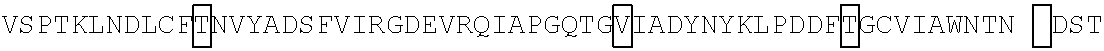 | 126 |
| COV_S_T2_18 | 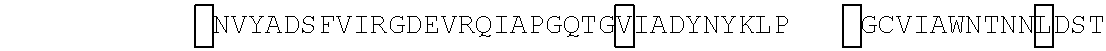 | 126 |
| COV_S_T2_13 | 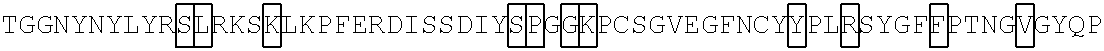 | 189 |
| COV_S_T2_14 | 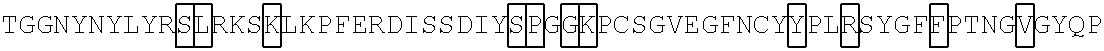 | 189 |
| COV_S_T2_15 | 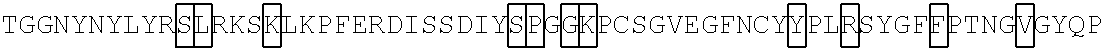 | 189 |
| COV_S_T2_16 | 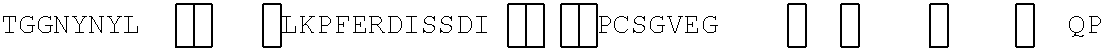 | 189 |
| COV_S_T2_17 | 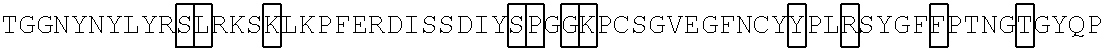 | 189 |
| COV_S_T2_18 | 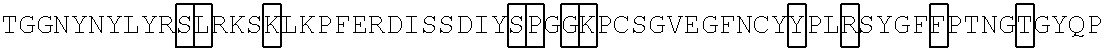 | 189 |
| COV_S_T2_13 | YRVVVLSFELLNAPATVCGPKLSTD | 214 |
| COV_S_T2_14 | YRVVVLSFELLNAPATVCGPKLSTD | 214 |
| COV_S_T2_15 | 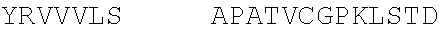 | 214 |
| COV_S_T2_16 | YRVVVLSFELLNAPATVCGPKLSTD | 214 |
| COV_S_T2_17 | YRVVVLSFELLNAPATVCGPKLSTD | 214 |
| COV_S_T2_18 | 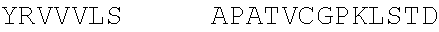 | 214 |

The residues of the discontinuous epitope present in COV_S_T2_14 and COV_S_T2_17 (marked in black) are as follows:

| | i) |
|---|---|
| | residues 13-28; |
| (SEQ ID NO: 57) | |
| | NITNLCPFGEVENATK |
| | ii) |
| | residues 38-42; |
| (SEQ ID NO: 58) | |
| | KKISN |
| | iii) |
| | residues 122-123 |
| (SEQ ID NO: 59) | |
| | NI |

The residues of the discontinuous epitope present in COV_S_T2_15 and COV_S_T2_18 (marked in purple) are as follows:

| | i) |
|---|---|
| | residues 51-75; |
| (SEQ ID NO: 60) | |
| | YNSTFFSTFKCYGVSPTKLNDLCFS |
| | ii) |
| | residues 109-112 |
| (SEQ ID NO: 61) | |
| | DDFM |
| | iii) |
| | residues 197-201 |
| (SEQ ID NO: 62) | |
| | FELLN |

The residues of the discontinuous epitope present in COV_S_T2_16 (marked in orange) are as follows:

| | i) |
|---|---|
| | residues 85-91; |
| (SEQ ID NO: 63) | |
| | RGDEVRQ |
| | ii) |
| | residues 97-103; |
| (SEQ ID NO: 64) | |
| | TGKIADY |
| | iii) |
| | residues 135-142; |
| (SEQ ID NO: 65) | |
| | YRLFRKSN |
| | iv) |
| | residues 155-160 |
| (SEQ ID NO: 66) | |
| | YQAGST |
| | v) |
| | residues 168-187 |
| (SEQ ID NO: 67) | |
| | FNCYFPLQSYGFQPTNGVGY |

The residues of the discontinuous epitope present in COV_S_T2_13, COV_S_T2_15, COV_S_T2_16, and COV_S_T2_18 (vertically adjacent the epitope marked in black) are as follows;

| | (i) |
|---|---|
| | residues 13-28; |
| (SEQ ID NO: 68) | |
| | NITNLCPFGEVENATR |
| | (ii) |
| | residues 38-42; |
| (SEQ ID NO: 69) | |
| | KRISN |
| | (iii) |
| | residues 122-123 |
| (SEQ ID NO: 70) | |
| | NL |

The residues of the discontinuous epitope present in COV_S_T2_13, COV_S_T2_15, COV_S_T2_16, and COV_S_T2_17 (vertically adjacent the epitope marked in black) are as follows;

| | (i) |
|---|---|
| | residues 51-75; |
| (SEQ ID NO: 71) | |
| | YNSTSFSTFKCYGVSPTKLNDLCFT |
| | (ii) |
| | residues 109-112 |
| (SEQ ID NO: 72) | |
| | DDFT |
| | (iii) |
| | residues 197-201 |
| (SEQ ID NO: 62) | |
| | FELLN |

The residues of the discontinuous epitope present in COV_S_T2_13, COV_S_T2_14, and COV_S_T2_15 (vertically adjacent the epitope marked in orange) are as follows:

| | (i) |
|---|---|
| | residues 85-91; |
| (SEQ ID NO: 63) | |
| | RGDEVRQ |
| | (ii) |
| | residues 97-103; |
| (SEQ ID NO: 73) | |
| | TGVIADY |
| | (iii) |
| | residues 135-142; |
| (SEQ ID NO: 74) | |
| | YRSLRKSK |
| | (iv) |
| | residues 155-160 |
| (SEQ ID NO: 75) | |
| | YSPGGK |
| | (v) |
| | residues 168-187 |
| (SEQ ID NO: 76) | |
| | FNCYYPLRSYGFFPTNGVGY |

The residues of the discontinuous epitope present in COV_S_T2_17 and COV_S_T2_18 (vertically adjacent the epitope marked in orange) are as follows:

| | (i) |
|---|---|
| | residues 85-91; |
| (SEQ ID NO: 63) | |
| | RGDEVRQ |
| | (ii) |
| | residues 97-103; |
| (SEQ ID NO: 73) | |
| | TGVIADY |
| | (iii) |
| | residues 135-142; |
| (SEQ ID NO: 74) | |
| | YRSLRKSK |
| | (iv) |
| | residues 155-160 |
| (SEQ ID NO: 75) | |
| | YSPGGK |
| | (v) |
| | residues 168-187 |
| (SEQ ID NO: 77) | |
| | FNCYYPLRSYGFFPTNGTGY |

According to the invention there is provided an isolated polypeptide comprising an amino acid sequence with the following discontinuous amino acid sequences:

| | i) |
|---|---|
| (SEQ ID NO: 57) | |
| | NITNLCPFGEVFNATK; |
| | ii) |
| (SEQ ID NO: 58) | |
| | KKISN; |
| | iii) |
| (SEQ ID NO: 59) | |
| | NI. |

According to the invention there is provided an isolated polypeptide comprising an amino acid sequence with the following discontinuous amino acid sequences:

| | i) |
|---|---|
| (SEQ ID NO: 60) | |
| | YNSTFFSTFKCYGVSPTKLNDLCFS; |
| | ii) |
| (SEQ ID NO: 61) | |
| | DDFM; |
| | iii) |
| (SEQ ID NO: 62) | |
| | FELLN. |

According to the invention there is provided an isolated polypeptide comprising an amino acid sequence with the following discontinuous amino acid sequences:

| | i) |
|---|---|
| (SEQ ID NO: 63) | |
| | RGDEVRQ; |
| | ii) |
| (SEQ ID NO: 64) | |
| | TGKIADY; |
| | iii) |
| (SEQ ID NO: 65) | |
| | YRLFRKSN; |
| | iv) |
| (SEQ ID NO: 66) | |
| | YQAGST; |
| | v) |
| (SEQ ID NO: 67) | |
| | FNCYFPLQSYGFQPTNGVGY. |

Optionally one or more residues of the amino acid residues of SEQ ID NOs:63-67 in a polypeptide of the invention comprising discontinuous amino acid sequences of SEQ ID NOs:63-67 may be changed (for example, by substitution or deletion) to provide a glycosylation site.

According to the invention there is also provided an isolated polypeptide comprising an amino acid sequence with the following discontinuous amino acid sequences:

| | (i) |
|---|---|
| (SEQ ID NO: 68) | |
| | NITNLCPFGEVFNATR; |
| | (ii) |
| (SEQ ID NO: 69) | |
| | KRISN; |
| | (iii) |
| (SEQ ID NO: 70) | |
| | NL |

According to the invention there is provided an isolated polypeptide comprising an amino acid sequence with the following discontinuous amino acid sequences:

| | (i) |
|---|---|
| (SEQ ID NO: 71) | |
| | YNSTSFSTFKCYGVSPTKLNDLCFT; |
| | (ii) |
| (SEQ ID NO: 72) | |
| | DDFT |
| | (iii) |
| (SEQ ID NO: 62) | |
| | FELLN |

According to the invention there is provided an isolated polypeptide comprising an amino acid sequence with the following discontinuous amino acid sequences:

| | (i) |
|---|---|
| (SEQ ID NO: 63) | |
| | RGDEVRQ; |
| | (ii) |
| (SEQ ID NO: 73) | |
| | TGVIADY; |
| | (iii) |
| (SEQ ID NO: 74) | |
| | YRSLRKSK; |
| | (iv) |
| (SEQ ID NO: 75) | |
| | YSPGGK |
| | (v) |
| (SEQ ID NO: 76) | |
| | FNCYYPLRSYGFFPTNGVGY |

According to the invention there is provided an isolated polypeptide comprising an amino acid sequence with the following discontinuous amino acid sequences:

| | (i) |
|---|---|
| (SEQ ID NO: 63) | |
| | RGDEVRQ; |
| | (ii) |
| (SEQ ID NO: 73) | |
| | TGVIADY; |
| | (iii) |
| (SEQ ID NO: 74) | |
| | YRSLRKSK; |
| | (iv) |
| (SEQ ID NO: 75) | |
| | YSPGGK |
| | (v) |
| (SEQ ID NO: 77) | |
| | FNCYYPLRSYGFFPTNGTGY |

Optionally the discontinuous amino acid sequences of each polypeptide of the invention are present in the order recited.

Optionally each discontinuous amino acid sequence is separated by at least 3 amino acid residues from an adjacent discontinuous amino acid sequence.

Optionally each discontinuous amino acid sequence is separated by up to 100 amino acid residues from an adjacent discontinuous amino acid sequence.

Optionally a polypeptide of the invention comprising the recited discontinuous amino acid sequences is up to 250, 500, 750, 1,000, 1,250, or 1,500 amino acid residues in length.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28, comprises the following discontinuous amino acid sequences:

| | i) |
|---|---|
| (SEQ ID NO: 57) | |
| | NITNLCPFGEVFNATK; |
| | ii) |
| (SEQ ID NO: 58) | |
| | KKISN; |
| | iii) |
| (SEQ ID NO: 59) | |
| | NI |

Optionally the discontinuous amino acid sequences (i), (ii), and (iii) are at amino acid residue positions corresponding to (i) residues 13-28; (ii) residues 38-42; and (iii) residues 122-123 of SEQ ID NO:28, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29, comprises the following discontinuous amino acid sequences:

| | i) |
|---|---|
| (SEQ ID NO: 60) | |
| | YNSTFFSTFKCYGVSPTKLNDLCFS; |
| (SEQ ID NO: 61) | |
| | DDFM; |
| (SEQ ID NO: 62) | |
| | FELLN. |

Optionally the discontinuous amino acid sequences (i), (ii), and (iii) are at amino acid residue positions corresponding to (i) residues 51-75; (ii) residues 109-112; and (iii) residues 197-201 of SEQ ID NO:29, respectively.

Optionally an isolated polypeptide of the invention comprising an amino acid sequence which has at least 93%, 94%, 95%, 96%, 97%, 98%, or 99% amino acid identity over its entire length with the amino acid sequence of SEQ ID NO:30, comprises the following discontinuous amino acid sequences:

| | i) |
|---|---|
| (SEQ ID NO: 63) | |
| | RGDEVRQ; |
| | ii) |
| (SEQ ID NO: 64) | |
| | TGKIADY; |
| | iii) |
| (SEQ ID NO: 65) | |
| | YRLFRKSN; |
| | iv) |
| (SEQ ID NO: 66) | |
| | YQAGST; |
| | v) |
| (SEQ ID NO: 67) | |
| | FNCYFPLQSYGFQPTNGVGY. |

Optionally the discontinuous amino acid sequences (i), (ii), (ii), (iv), and (v) are at amino acid residue positions corresponding to (i) residues 85-91, (ii) residues 97-103, (iii) residues 135-142, (iv) residues 155-160, and (v) residues 168-187 of SEQ ID NO:30, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31, comprises the following discontinuous amino acid sequences:

| | i) |
|---|---|
| (SEQ ID NO: 57) | |
| | NITNLCPFGEVENATK; |
| | ii) |
| (SEQ ID NO: 58) | |
| | KKISN; |
| | iii) |
| (SEQ ID NO: 59) | |
| | NI. |

Optionally the discontinuous amino acid sequences (i), (ii), and (iii) are at amino acid residue positions corresponding to (i) residues 13-28; (ii) residues 38-42; and (iii) residues 122-123 of SEQ ID NO:31, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, comprises the following discontinuous amino acid sequences:

| | i) |
|---|---|
| (SEQ ID NO: 60) | |
| | YNSTFFSTFKCYGVSPTKLNDLCFS; |
| | ii) |
| (SEQ ID NO: 61) | |
| | DDFM; |
| | iii) |
| (SEQ ID NO: 62) | |
| | FELLN. |

Optionally the discontinuous amino acid sequences (i), (ii), and (iii) are at amino acid residue positions corresponding to (i) residues 51-75; (ii) residues 109-112; and (iii) residues 197-201 of SEQ ID NO:32, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29, comprises the following discontinuous amino acid sequences:

| | (i) |
|---|---|
| (SEQ ID NO: 68) | |
| | NITNLCPFGEVFNATR; |
| | (ii) |
| (SEQ ID NO: 69) | |
| | KRISN; |
| | (iii) |
| (SEQ ID NO: 70) | |
| | NL |

Optionally the discontinuous amino acid sequences (i), (ii), and (iii) are at amino acid residue positions corresponding to (i) residues 13-28; (ii) residues 38-42; and (iii) residues 122-123 of SEQ ID NO:29, respectively.

Optionally an isolated polypeptide of the invention comprising an amino acid sequence which has at least 93%, 94%, 95%, 96%, 97%, 98%, or 99% amino acid identity over its entire length with the amino acid sequence of SEQ ID NO:30, comprises the following discontinuous amino acid sequences:

| | (i) |
|---|---|
| (SEQ ID NO: 68) | |
| | NITNLCPFGEVFNATR; |
| | (ii) |
| (SEQ ID NO: 69) | |
| | KRISN; |
| | (iii) |
| (SEQ ID NO: 70) | |
| | NL |

Optionally the discontinuous amino acid sequences (i), (ii), and (iii) are at amino acid residue positions corresponding to (i) residues 13-28; (ii) residues 38-42; and (iii) residues 122-123 of SEQ ID NO:30, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, comprises the following discontinuous amino acid sequences:

| | (i) |
|---|---|
| (SEQ ID NO: 68) | |
| | NITNLCPFGEVFNATR; |
| | (ii) |
| (SEQ ID NO: 69) | |
| | KRISN; |
| | (iii) |
| (SEQ ID NO: 70) | |
| | NL |

Optionally the discontinuous amino acid sequences (i), (ii), and (iii) are at amino acid residue positions corresponding to (i) residues 13-28; (ii) residues 38-42; and (iii) residues 122-123 of SEQ ID NO:32, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28, comprises the following discontinuous amino acid sequences:

| | (i) |
|---|---|
| (SEQ ID NO: 71) | |
| | YNSTSFSTFKCYGVSPTKLNDLCFT; |
| | (ii) |
| (SEQ ID NO: 72) | |
| | DDFT |
| | (iii) |
| (SEQ ID NO: 62) | |
| | FELLN |

Optionally the discontinuous amino acid sequences (i), (ii), and (iii) are at amino acid residue positions corresponding to (i) residues 51-75; (ii) residues 109-112; and (iii) residues 197-201 of SEQ ID NO:28, respectively.

Optionally an isolated polypeptide of the invention comprising an amino acid sequence which has at least 93%, 94%, 95%, 96%, 97%, 98%, or 99% amino acid identity over its entire length with the amino acid sequence of SEQ ID NO:30, comprises the following discontinuous amino acid sequences:

| | (i) |
|---|---|
| (SEQ ID NO: 71) | |
| | YNSTSFSTFKCYGVSPTKLNDLCFT; |
| | (ii) |
| (SEQ ID NO: 72) | |
| | DDFT |
| | (iii) |
| (SEQ ID NO: 62) | |
| | FELLN |

Optionally the discontinuous amino acid sequences (i), (ii), and (iii) are at amino acid residue positions corresponding to (i) residues 51-75; (ii) residues 109-112; and (iii) residues 197-201 of SEQ ID NO:30, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31, comprises the following discontinuous amino acid sequences:

| | (i) |
|---|---|
| (SEQ ID NO: 71) | |
| | YNSTSFSTFKCYGVSPTKLNDLCFT; |
| | (ii) |
| (SEQ ID NO: 72) | |
| | DDFT |
| | (iii) |
| (SEQ ID NO: 62) | |
| | FELLN |

Optionally the discontinuous amino acid sequences (i), (ii), and (iii) are at amino acid residue positions corresponding to (i) residues 51-75; (ii) residues 109-112; and (iii) residues 197-201 of SEQ ID NO:31, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28, comprises the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
v)
(SEQ ID NO: 76)
FNCYYPLRSYGFFPTNGVGY

Optionally the discontinuous amino acid sequences (i), (ii), and (iii) are at amino acid residue positions corresponding to (i) residues 51-75; (ii) residues 109-112; and (iii) residues 197-201 of SEQ ID NO:28, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29, comprises the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
v)
(SEQ ID NO: 76)
FNCYYPLRSYGFFPTNGVGY

Optionally the discontinuous amino acid sequences (i), (ii), and (iii) are at amino acid residue positions corresponding to (i) residues 51-75; (ii) residues 109-112; and (iii) residues 197-201 of SEQ ID NO:29, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31, comprises the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
v)
(SEQ ID NO: 76)
FNCYYPLRSYGFFPTNGTGY

Optionally the discontinuous amino acid sequences (i), (ii), and (iii) are at amino acid residue positions corresponding to (i) residues 51-75; (ii) residues 109-112; and (iii) residues 197-201 of SEQ ID NO:31, respectively.

Optionally an isolated polypeptide of the invention comprising an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32, comprises the following discontinuous amino acid sequences:

(i)
(SEQ ID NO: 63)
RGDEVRQ;
(ii)
(SEQ ID NO: 73)
TGVIADY;
(iii)
(SEQ ID NO: 74)
YRSLRKSK;
(iv)
(SEQ ID NO: 75)
YSPGGK
v)
(SEQ ID NO: 76)
FNCYYPLRSYGFFPTNGTGY

Optionally the discontinuous amino acid sequences (i), (ii), and (iii) are at amino acid residue positions corresponding to (i) residues 51-75; (ii) residues 109-112; and (iii) residues 197-201 of SEQ ID NO:32, respectively.

Designed Coronavirus S Protein RBD Sequences with Altered Glycosylation Sites

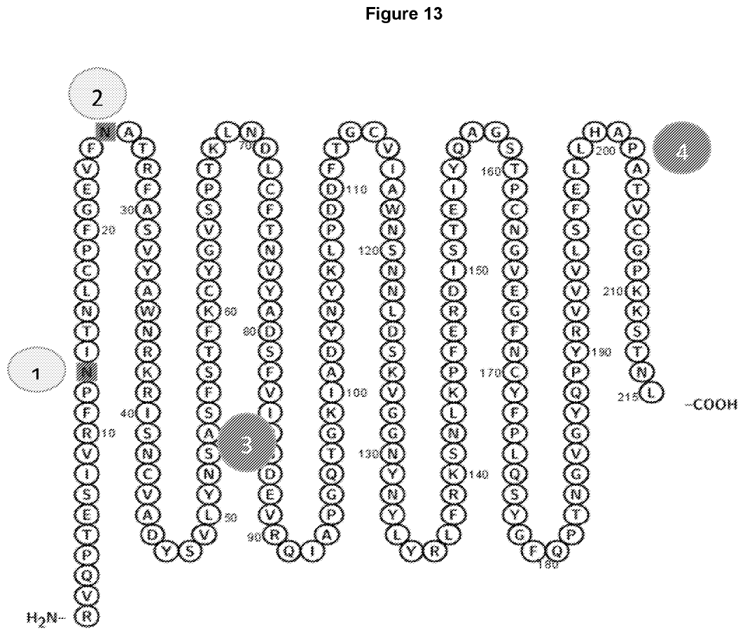

Masking/de-masking of epitopes has been shown to alter the immune response by masking non-neutralising epitopes, or by de-masking important epitopes in MERS (Du L et. al., Nat. Comm, volume 7, Article number: 13473 (2016)). We have prepared additional designed S protein RBD sequences (SARS2 RBD designs M7, M8, M9, and M10) in which we have deleted a glycosylation site of SARS2 RBD sequence, or introduced a glycosylation site to SARS2 RBD sequence. The changes made are illustrated in FIG. 13, and discussed in Example 14 below. Designs M7 and M9 include a glycosylation site introduced at the position indicated by circled number 4 (residue position 203) in FIG. 13. Designs M8 and M10 include a deleted glycosylation site at each of the positions indicated by circled numbers 1 and 2 (residue positions 13 and 25, respectively) in FIG. 13. The M8 design also includes an introduced glycosylation site at the position indicated by circled number 3 (residue position 54).

The amino acid sequences of SARS2 RBD designs M7, M8, M9, and M10 are shown below, and in Example 14:

>M7
(SEQ ID NO: 33)
RVQPTESIVR FPNITNLCPF GEVFNATRFA SVYAWNRKRI SNCVADYSVL YNSASFSTFK CYGVSPTKLN
DLCFTNVYAD SFVIRGDEVR QIAPGQTGKI ADYNYKLPDD FTGCVIAWNS NNLDSKVGGN YNYLYRLFRK
SNLKPFERDI STEIYQAGST PCNGVEGFNC YFPLQSYGFQ PTNGVGYQPY RVVVLSFELL HANATVCGPK
KSTN
>M8
(SEQ ID NO: 34)
RVQPTESIVR FPQITNLCPF GEVFQATRFA SVYAWNRKRI SNCVADYSVL YNSTSFSTFK CYGVSPTKLN
DLCFTNVYAD SFVIRGDEVR QIAPGQTGKI ADYNYKLPDD FTGCVIAWNS NNLDSKVGGN YNYLYRLFRK
SNLKPFERDI STEIYQAGST PCNGVEGFNC YFPLQSYGFQ PTNGVGYQPY RVVVLSFELL HAPATVCGPK
KSTN
>M9
(SEQ ID NO: 35)
RVSPTQEVVR FPNITNLCPF DKVFNATRFP SVYAWERTKI SDCVADYTVL YNSTSFSTFK CYGVSPSKLI
DLCFTSVYAD TFLIRCSEVR QVAPGQTGVI ADYNYKLPDD FTGCVIAWNT AKQDTGSSGN YNYYYRSHRK
TKLKPFERDL SSDECSPDGK PCTPPAFNGV RGFNCYFTLS TYDFNPNVPV EYQATRVVVL SFELLNANAT
VCGPKLSTQ
>M10
(SEQ ID NO: 36)
RVSPTQEVVR FPQITNLCPF DKVFQATRFP SVYAWERTKI SDCVADYTVL YNSTSFSTFK CYGVSPSKLI
DLCFTSVYAD TFLIRCSEVR QVAPGQTGVI ADYNYKLPDD FTGCVIAWNT AKQDTGSSGN YNYYYRSHRK
TKLKPFERDL SSDECSPDGK PCTPPAFNGV RGFNCYFTLS TYDFNPNVPV EYQATRVVVL SFELLNAPAT
VCGPKLSTQ

Alignment of these sequences with the SARS2 Reference sequence (EPI_ISL_402119_RBD (CoV_T2_6) (SEQ ID NO:11)) is shown in Example 14 below.

The amino acid differences of the designed sequences from the SARS2 reference sequence are shown in Table 9 below (with differences from the reference sequence highlighted in bold):

TABLE 9

| | SARS2 RBD | | | | | |
|---|---|---|---|---|---|---|
| | (SEQ ID | | | | | |
| Circled | NO: 11) | | M7 residue | M8 residue | M9 residue | M10 residue |
| number of | residue | Reference | (SEQ ID | (SEQ ID | (SEQ ID | (SEQ ID |
| FIG. 13 | position | residue | NO: 33) | NO: 34) | NO: 35) | NO: 36) |
| | 3 | Q | | | S | S |
| | 6 | E | | | Q | Q |
| | 7 | S | | | E | E |
| | 8 | I | | | V | V |
| 1 | 13 | N | | Q | | Q |
| | 21 | G | | | D | K |
| | 22 | E | | | D | K |
| 2 | 25 | N | | Q | | Q |
| | 30 | A | | | P | P |
| | 36 | N | | | E | E |
| | 38 | K | | | T | K |
| | 39 | R | | | T | K |
| | 42 | N | | | D | D |
| | 48 | S | | | T | T |
| 3 | 54 | A | | T | T | T |
| | 67 | T | | | S | S |
| | 70 | N | | | I | I |
| | 76 | N | | | S | S |
| | 81 | S | | | T | T |
| | 83 | V | | | L | L |
| | 86 | G | | | C | C |
| | 87 | D | | | S | S |
| | 92 | I | | | V | V |
| | 99 | K | | | V | V |
| | 120 | S | | | T | T |
| | 121 | N | | | A | A |
| | 122 | N | | | K | K |
| | 123 | L | | | Q | Q |
| | 125 | S | | | T | T |
| | 126 | K | | | G | G |
| | 127 | V | | | S | S |
| | 128 | G | | | S | S |
| | 134 | L | | | Y | Y |
| | 137 | L | | | S | S |
| | 138 | F | | | H | H |
| | 141 | S | | | T | T |
| | 142 | N | | | K | K |
| | 150 | I | | | L | L |
| | 152 | T | | | S | S |
| | 153 | E | | | D | D |
| | 154 | I | | | E | E |
| | 155 | Y | | | C | C |
| | 156 | Q | | | S | S |
| | 157 | A | | | P | P |
| | 158 | G | | | D | D |
| | 159 | S | | | G | G |
| | 160 | T | | | K | K |
| | * | — | | | T | T |
| | * | — | | | P | P |
| | * | — | | | P | P |
| | * | — | | | A | A |
| | * | — | | | F | F |
| | 166 | E | | | R | R |
| | 173 | P | | | T | T |
| | 175 | Q | | | S | S |
| | 176 | S | | | T | T |
| | 178 | G | | | D | D |
| | 180 | Q | | | N | N |
| | 182 | T | | | N | N |
| | 183 | N | | | V | V |
| | 184 | G | | | P | P |
| | 186 | E | | | E | E |
| | 189 | P | | | A | A |
| | 190 | Y | | | T | T |
| | 201 | H | | | N | N |
| 4 | 203 | P | N | | N | — |
| | 211 | K | | | L | L |
| | 214 | Q | | | D | Q |
| Total no of | | | 1 | 3 | 66 | 67 |
| differences | | | | | | |
| from | | | | | | |
| reference | | | | | | |
| Percentage | | | 99.53% | 98.60% | 69.12% | 68.69% |
| identity | | | | | | |
| with | | | | | | |
| reference | | | | | | |

Residues inserted between amino acid residue positions 162 and 163 of SEQ ID NO:11.

According to the invention there is provided an isolated polypeptide, which comprises an amino acid sequence according to SEQ ID NO:33, 34, 35, or 38.

According to the invention there is also provided an isolated polypeptide comprising an amino acid sequence of SEQ ID NO: 34 (M8), or an amino acid sequence which has at least 99% amino acid identity over its entire length with the amino acid sequence of SEQ ID NO:34.

Optionally a polypeptide of the invention comprising an amino acid sequence of SEQ ID NO:34 (M8), or an amino acid sequence which has at least 99% amino acid identity over its entire length with the amino acid sequence of SEQ ID NO:34, comprises at least one, or all of the following amino acid residues at positions corresponding to the amino acid residue positions of SEQ ID NO:11: 13Q, 25Q, 54T.

According to the invention there is also provided an isolated polypeptide which comprises a coronavirus S protein RBD domain with at least one of the following amino acid residues at positions corresponding to the amino acid residue positions of SEQ ID NO:11: 13Q, 25Q, 54T, 203N.

According to the invention there is also provided an isolated polypeptide comprising an amino acid sequence of SEQ ID NO: 35 (M9), or an amino acid sequence which has at least 70% amino acid identity over its entire length with the amino acid sequence of SEQ ID NO:35.

Optionally an isolated polypeptide of the invention which comprises an amino acid sequence of SEQ ID NO: 35 (M9), or an amino acid sequence which has at least 70% amino acid identity over its entire length with the amino acid sequence of SEQ ID NO:35, comprises at least one, or all of the amino acid residues at positions corresponding to the amino acid residue positions as shown in Table 9.1 below.

TABLE 9.1

| SARS2 RBD | |
|---|---|
| (SEQ ID | M9 residue |
| NO: 11) residue | (SEQ ID |
| position | NO: 35) |
| 3 | S |
| 6 | Q |
| 7 | E |
| 8 | V |
| 21 | D |
| 22 | D |
| 30 | P |
| 36 | E |
| 38 | T |
| 39 | T |
| 42 | D |
| 48 | T |
| 54 | T |
| 67 | S |
| 70 | I |
| 76 | S |
| 81 | T |
| 83 | L |
| 86 | C |
| 87 | S |
| 92 | V |
| 99 | V |
| 120 | T |
| 121 | A |
| 122 | K |
| 123 | Q |
| 125 | T |
| 126 | G |
| 127 | S |
| 128 | S |
| 134 | Y |
| 137 | S |
| 138 | H |
| 141 | T |
| 142 | K |
| 150 | L |
| 152 | S |
| 153 | D |
| 154 | E |
| 155 | C |
| 156 | S |
| 157 | P |
| 158 | D |
| 159 | G |
| 160 | K |
| * | T |
| * | P |
| * | P |
| * | A |
| * | F |
| 166 | R |
| 173 | T |
| 175 | S |
| 176 | T |
| 178 | D |
| 180 | N |
| 182 | N |
| 183 | V |
| 184 | P |
| 186 | E |
| 189 | A |
| 190 | T |
| 201 | N |
| 203 | N |
| 211 | L |
| 214 | Q |

Residues for insertion between amino acid residue positions 162 and 163 of SEQ ID NO:11.

Optionally an isolated polypeptide of the invention which comprises an amino acid sequence of SEQ ID NO: 35 (M9), or an amino acid sequence which has at least 70% amino acid identity over its entire length with the amino acid sequence of SEQ ID NO:35, comprises at least one, or both of the following amino acid residues at positions corresponding to the amino acid residue positions of SEQ ID NO:11: 54T, 203N.

According to the invention there is also provided an isolated polypeptide comprising an amino acid sequence of SEQ ID NO: 36 (M10), or an amino acid sequence which has at least 69% amino acid identity over its entire length with the amino acid sequence of SEQ ID NO:36.

Optionally an isolated polypeptide of the invention which comprises an amino acid sequence of SEQ ID NO: 36 (M10), or an amino acid sequence which has at least 69% amino acid identity over its entire length with the amino acid sequence of SEQ ID NO:36, comprises at least one, or all of the amino acid residues at positions corresponding to the amino acid residue positions as shown in Table 9.2 below.

TABLE 9.2

| SARS2 RBD | |
|---|---|
| (SEQ ID | M10 residue |
| NO: 11) residue | (SEQ ID |
| position | NO: 36) |
| 3 | S |
| 6 | Q |
| 7 | E |
| 8 | V |
| 13 | Q |
| 21 | K |
| 22 | K |
| 25 | Q |
| 30 | P |
| 36 | E |
| 38 | K |
| 39 | K |
| 42 | D |
| 48 | T |
| 54 | T |
| 67 | S |
| 70 | I |
| 76 | S |
| 81 | T |
| 83 | L |
| 86 | C |
| 87 | S |
| 92 | V |
| 99 | V |
| 120 | T |
| 121 | A |
| 122 | K |
| 123 | Q |
| 125 | T |
| 126 | G |
| 127 | S |
| 128 | S |
| 134 | Y |
| 137 | S |
| 138 | H |
| 141 | T |
| 142 | K |
| 150 | L |
| 152 | S |
| 153 | D |
| 154 | E |
| 155 | C |
| 156 | S |
| 157 | P |
| 158 | D |
| 159 | G |
| 160 | K |
| * | T |
| * | P |
| * | P |
| * | A |
| * | F |
| 166 | R |
| 173 | T |
| 175 | S |
| 176 | T |
| 178 | D |
| 180 | N |
| 182 | N |
| 183 | V |
| 184 | P |
| 186 | E |
| 189 | A |
| 190 | T |
| 201 | N |
| 211 | L |
| 214 | Q |

* Residues from insertion between amino acid residue positions 162 and 163 of SEQ ID NO: 11.

Optionally an isolated polypeptide of the invention which comprises an amino acid sequence of SEQ ID NO: 36 (M10), or an amino acid sequence which has at least 69% amino acid identity over its entire length with the amino acid sequence of SEQ ID NO:36, comprises at least one, or all of the following amino acid residues at positions corresponding to the amino acid residue positions of SEQ ID NO:11: 13Q, 25Q, 54T.

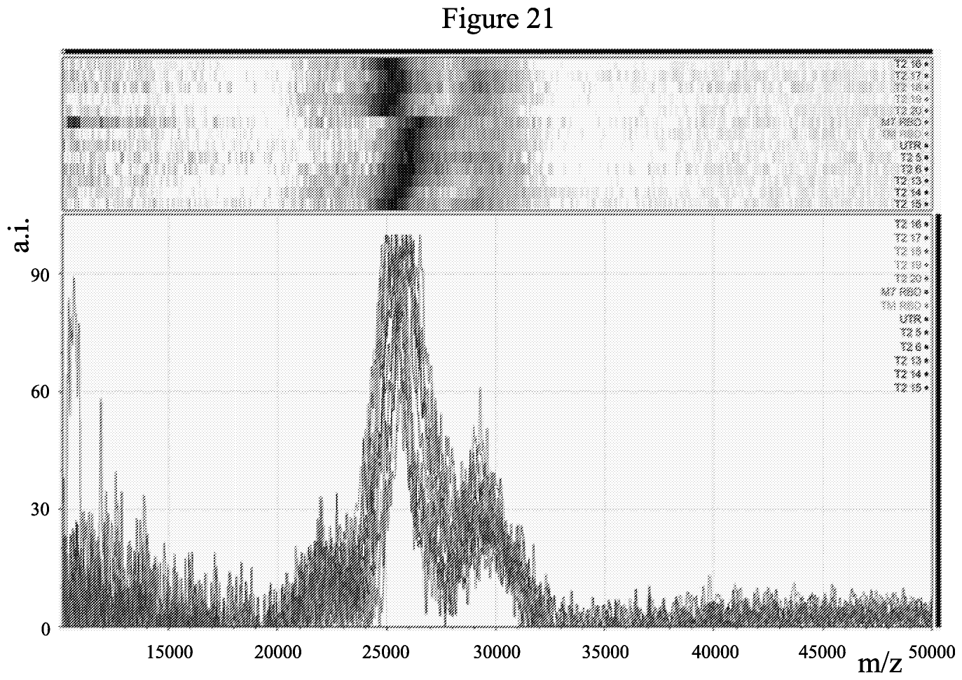

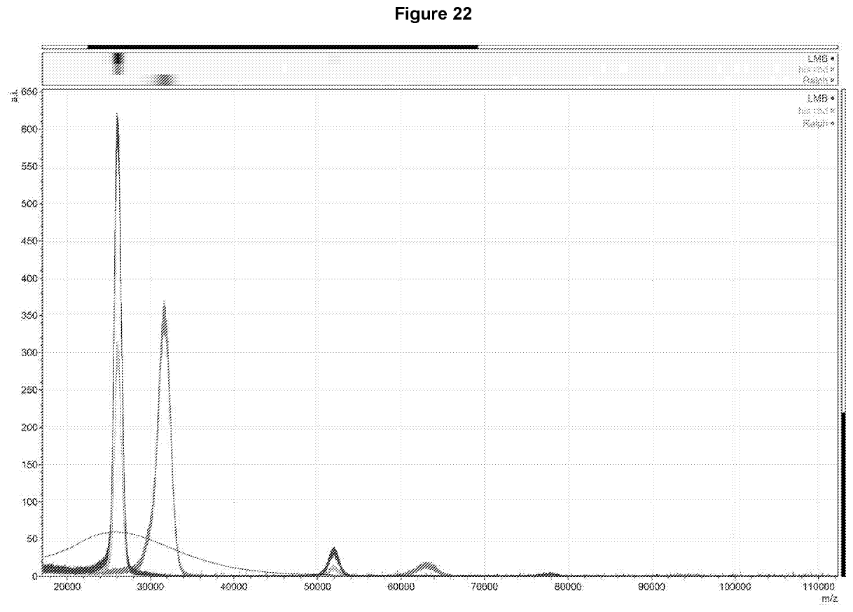

The effect of glycosylation of the RBD protein is believed to be important. We have found that M7 and wild-type SARS2 RBD DNA (believed to result in expression of glycosylated RBD protein) is superior to recombinant SARS2 RBD protein (non-glycosylated, or sparsely glycosylated) in inducing neutralising responses to SARS2. Example 28 below describes Mass spectroscopy data obtained to study glycosylation of SARS-CoV-2 (SARS2) RBD proteins in supernatants derived from HEK cells transfected with pEVAC plasmid encoding SARS-CoV-2 RBD sequences, compared with recombinant SARS-CoV-2 RBD proteins (see FIGS. 21 and 22). It was concluded from the results that there are two main glycosylated forms of the proteins obtained from the supernatants, in comparison to purified (recombinant) protein. The purified protein is non-glycosylated or sparsely glycosylated. This difference in glycosylation is believed to be important, as the glycosylation sites surround the epitope region and are conserved in most sarbecoviruses. These glycosylation sites are also important for interaction with some of the antibodies.

Optionally a polypeptide of the invention comprising an amino acid sequence of a designed coronavirus spike (S) protein (full-length, truncated, or RBD) comprises at least one glycosylation site in the RBD sequence.

Optionally a polypeptide of the invention comprising an amino acid sequence of a designed coronavirus spike (S) protein (full-length, truncated, or RBD) comprises at least two glycosylation sites in the RBD sequence.

Optionally a polypeptide of the invention comprising an amino acid sequence of a designed coronavirus spike (S) protein (full-length, truncated, or RBD) comprises at least three glycosylation sites in the RBD sequence.

Optionally a polypeptide of the invention comprising an amino acid sequence of a designed coronavirus spike (S) protein (full-length, truncated, or RBD) comprises a glycosylation site located within the last 10 amino acids of the RBD sequence, preferably at a residue position corresponding to residue position 203 of the RBD sequence.

According to the invention there is also provided an isolated polypeptide, which comprises an amino acid sequence of a SARS2 RBD with a glycosylation site located within the last 10 amino acids of the SARS2 RBD sequence, preferably at a residue position corresponding to residue position 203 of the RBD sequence.

We have also found that immunisation of mice with a wild-type SARS1 S protein, or RBD protein, or a wild-type SARS2 S protein, or RBD protein, induced antibodies that bind SARS2 RBD.

There is also provided according to the invention an isolated polypeptide comprising an amino acid sequence of SEQ ID NO:5.

There is also provided according to the invention an isolated polypeptide comprising an amino acid sequence of SEQ ID NO:11.

A conventional way to produce cross-reactive antigens is to generate a consensus sequence based on natural diversity. Antigenic sequences encoded by nucleic acid sequences of the invention described herein account for sampling bias and coevolution between sites. The result is a realistic molecule which induces an immune response to a range of viruses. As a further refinement, we enrich the antigenic sequences for known and predicted epitopes. We have developed an algorithm to select the combination of epitopes that maximise population protection against a range of target viruses. This algorithm identifies conserved epitopes whilst penalising redundancy and ensuring that the selected epitopes are bound by a range of common MHC alleles.

To avoid disease enhancement we modify the antigens, deleting regions associated with immunopathology, often referred to as antibody dependent enhancement (ADE) and/or complement triggered, or virus triggered proinflammatory responses. In order to validate these modifications, we have developed assays to screen against such ADE-like effects. Using assays modified from Yip et al. (Yip et al. “Antibody-dependent infection of human macrophages by severe acute respiratory syndrome coronavirus”, Virol J. 2014; 11: 82; Jaume et al. “Anti-Severe Acute Respiratory Syndrome Coronavirus Spike Antibodies Trigger Infection of Human Immune Cells via a pH- and Cysteine Protease-Independent FcγR Pathway” Journal Of Virology, October 2011, p. 10582-10597), non-neutralising antibodies to the non-RBD site of the S protein that allow SARS-CoV-1 to enter non-ACE2 expressing immune cells, which bear Fc-γ-RII, can be identified.

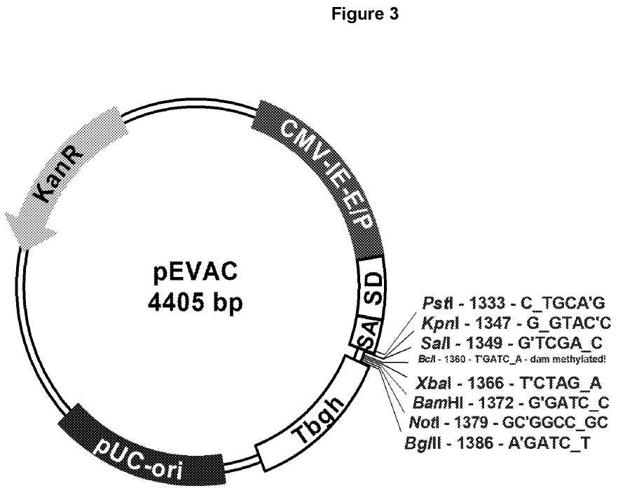

After designing antigens, DNA sequences encoding them are optimised for expression in mammalian cells. In this DNA form, multiple synthetic genes of the target antigens are inserted into a DNA plasmid vector (for example, pEVAC—see FIG. 3), which is used for both in vitro and in vivo immune screening.

### Designed Coronavirus Full-Length S Protein Sequence to Protect Against COVID-19 Variants

Multiple SARS-CoV-2 variants are circulating globally. Several new variants emerged in the fall of 2020, most notably:

In the United Kingdom (UK), a new variant of SARS-CoV-2 (known as 20I/501Y.V1. VOC 202012/01, or B.1.1.7) emerged with a large number of mutations. This variant has since been detected in numerous countries around the world, including the United States (US). In January 2021, scientists from UK reported evidence that suggests the B.1.1.7 variant may be associated with an increased risk of death compared with other variants, although more studies are needed to confirm this finding. This variant was reported in the US at the end of December 2020.

In South Africa, another variant of SARS-CoV-2 (known as 20H/501Y.V2 or B.1.351) emerged independently of B.1.1.7. This variant shares some mutations with B.1.1.7. Cases attributed to this variant have been detected in multiple countries outside of South Africa. This variant was reported in the US at the end of January 2021.

In Brazil, a variant of SARS-CoV-2 (known as P.1) emerged that was first was identified in four travelers from Brazil, who were tested during routine screening at Haneda airport outside Tokyo, Japan. This variant has 17 unique mutations, including three in the receptor binding domain of the spike protein. This variant was detected in the US at the end of January 2021.

Scientists are working to learn more about these variants to better understand how easily they might be transmitted and the effectiveness of currently authorized vaccines against them. New information about the virologic, epidemiologic, and clinical characteristics of these variants is rapidly emerging.

As described in more detail in Example 30 below, we have designed a new full-length S protein sequence (referred to as “VOC Chimera”, or COV_S_T2_29) for use as a COVID-19 vaccine insert to protect against variants B.1.1.7, P.1, and B.1.351. The amino acid sequence of the designed full-length S protein sequence is given below, and in Example 30:

| >COV_S_T2_29 (VOC chimera) | |
|---|---|
| (SEQ ID NO: 53) | |
| MFVFLVLLPL VSSQCVNFTN RTQLPSAYTN SFTRGVYYPD KVFRSSVLHS TQDLFLPFFS | 60 |
| NVTWFHAISG TNGTKRFDNP VLPFNDGVYF ASTEKSNIIR GWIFGTTLDS KTQSLLIVNN | 120 |
| ATNVVIKVCE FQFCNDPFLG VYHKNNKSWM ESEFRVYSSA NNCTFEYVSQ PFLMDLEGKQ | 180 |
| GNFKNLREFV FKNIDGYFKI YSKHTPINLV RDLPQGFSAL EPLVDLPIGI NITRFQTLLA | 240 |
| LHRSYLTPGD SSSGWTAGAA AYYVGYLQPR TFLLKYNENG TITDAVDCAL DPLSETKCTL | 300 |
| KSFTVEKGIY QTSNFRVQPT ESIVRFPNIT NLCPFGEVFN ATRFASVYAW NRKRISNCVA | 360 |
| DYSVLYNSAS FSTFKCYGVS PTKLNDLCFT NVYADSFVIR GDEVRQIAPG QTGNIADYNY | 420 |
| KLPDDFTGCV IAWNSNNLDS KVGGNYNYLY RLFRKSNLKP FERDISTEIY QAGSTPCNGV | 480 |
| KGFNCYFPLQ SYGFQPTYGV GYQPYRVVVL SFELLHAPAT VCGPKKSTNL VKNKCVNFNF | 540 |
| NGLTGTGVLT ESNKKFLPFQ QFGRDIADTT DAVRDPQTLE ILDITPCSFG GVSVITPGTN | 600 |
| TSNQVAVLYQ GVNCTEVPVA IHADQLTPTW RVYSTGSNVF QTRAGCLIGA EHVNNSYECD | 660 |
| IPIGAGICAS YQTQTNSHRR ARSVASQSII AYTMSLGAEN SVAYSNNSIA IPTNFTISVT | 720 |
| TEILPVSMTK TSVDCTMYIC GDSTECSNLL LQYGSFCTQL NRALTGIAVE QDKNTQEVFA | 780 |
| QVKQIYKTPP IKDFGGFNFS QILPDPSKPS KRSFIEDLLF NKVTLADAGF IKQYGDCLGD | 840 |
| IAARDLICAQ KFNGLTVLPP LLTDEMIAQY TSALLAGTIT SGWTFGAGAA LQIPFAMQMA | 900 |
| YRFNGIGVTQ NVLYENQKLI ANQFNSAIGK IQDSLSSTAS ALGKLQDVVN QNAQALNTLV | 960 |
| KQLSSNFGAI SSVLNDILSR LDPPEAEVQI DRLITGRLQS LQTYVTQQLI RAAEIRASAN | 1020 |
| LAATKMSECV LGQSKRVDFC GKGYHLMSFP QSAPHGVVFL HVTYVPAQEK NFTTAPAICH | 1080 |
| DGKAHFPREG VFVSNGTHWF VTQRNFYEPQ IITTDNTFVS GNCDVVIGIV NNTVYDPLQP | 1140 |
| ELDSFKEELD KYFKNHTSPD VDLGDISGIN ASVVNIQKEI DRLNEVAKNL NESLIDLQEL | 1200 |
| GKYEQYIKWP WYIWLGFIAG LIAIVMVTIM LCCMTSCCSC LKGCCSCGSC CKFDEDDSEP | 1260 |
| VLKGVKLHYT | 1270 |

Alignment of this sequence with SARS2 Reference sequence (EPI_ISL_402130 (Wuhan strain) (SEQ ID NO:52)) is shown in Example 30 below.

The amino acid differences of the designed sequence COV_S_T2_29 (SEQ ID NO:53) from the SARS2 reference sequence (SEQ ID NO:52) are shown in Table 9.3 below:

TABLE 9.3

| SARS2 S | | |
|---|---|---|
| protein | | |
| residue | | |
| position | SARS2 Reference | COV_S_T2_29 |
| (SEQ ID | amino acid residue | amino acid residue |
| NO: 52) | (SEQ ID NO: 52) | (SEQ ID NO: 53) |
| 18 | L | F |
| 20 | T | N |
| 26 | P | S |
| 69 | H | — (deletion) |
| 70 | V | — (deletion) |
| 144 | Y | — (deletion) |
| 417 | K | N |
| 484 | E | K |
| 501 | N | Y |
| 614 | D | G |
| 681 | P | H |
| 986 | K | P |
| 987 | V | P |

According to the invention there is provided an isolated polypeptide, which comprises an amino acid sequence of SEQ ID NO:53.

According to the invention there is also provided an isolated polypeptide comprising an amino acid sequence of SEQ ID NO:53, or an amino acid sequence which has at least 99% amino acid identity over its entire length with the amino acid sequence of SEQ ID NO:53.

Optionally a polypeptide of the invention comprising an amino acid sequence of SEQ ID NO:53, or an amino acid sequence which has at least 99% amino acid identity over its entire length with the amino acid sequence of SEQ ID NO:53, comprises at least one, or all of the amino acid residues or deletions, at positions corresponding to the amino acid residue positions of SEQ ID NO:52, as shown in Table 9.4 below:

TABLE 9.4

| SARS2 S | |
|---|---|
| protein | |
| residue | |
| position | COV_S_T2_29 |
| (SEQ ID | amino acid residue |
| NO: 52 | (SEQ ID NO: 53) |
| 18 | F |
| 20 | N |
| 26 | S |
| 69 | — (deletion) |
| 70 | — (deletion |
| 144 | — (deletion) |
| 417 | N |
| 484 | K |
| 501 | Y |
| 614 | G |
| 681 | H |
| 986 | P |
| 987 | P |

Optionally a polypeptide of the invention comprising an amino acid sequence of SEQ ID NO:53, or an amino acid sequence which has at least 99% amino acid identity over its entire length with the amino acid sequence of SEQ ID NO:53, comprises at least five of the amino acid residues or deletions, at positions corresponding to the amino acid residue positions of SEQ ID NO:52, as shown in Table 9.4.

Optionally a polypeptide of the invention comprising an amino acid sequence of SEQ ID NO:53, or an amino acid sequence which has at least 99% amino acid identity over its entire length with the amino acid sequence of SEQ ID NO:53, comprises at least ten of the amino acid residues or deletions, at positions corresponding to the amino acid residue positions of SEQ ID NO:52, as shown in Table 9.4.

Optionally a polypeptide of the invention comprising an amino acid sequence of SEQ ID NO:53, or an amino acid sequence which has at least 99% amino acid identity over its entire length with the amino acid sequence of SEQ ID NO:53, comprises amino acid residue P at position 986, and amino acid residue P at position 987, corresponding to the amino acid residue positions of SEQ ID NO:52, and at least one, or all of the amino acid residues or deletions, at positions corresponding to the amino acid residue positions of SEQ ID NO:52, as shown in Table 9.5 below:

TABLE 9.5

| SARS2 S | |
|---|---|
| protein | |
| residue | |
| position | COV_S_T2_29 |
| SEQ ID | amino acid residue |
| NO: 52 | (SEQ ID NO: 53) |
| 18 | F |
| 20 | N |
| 26 | S |
| 69 | — (deletion) |
| 70 | — (deletion) |
| 144 | — (deletion) |
| 417 | N |
| 484 | K |
| 501 | Y |
| 614 | G |
| 681 | H |

According to the invention there is also provided an isolated polypeptide which comprises a coronavirus S protein with at least one, or all of the amino acid residues or deletions, at positions corresponding to the amino acid residue positions of SEQ ID NO:52, as shown in Table 9.4 above.

Optionally an isolated polypeptide of the invention which comprises at least one of the amino acid residues or deletions, at positions corresponding to the amino acid residue positions of SEQ ID NO:52, as shown in Table 9.4 above, comprises at least five of the amino acid residues or deletions, at positions corresponding to the amino acid residue positions of SEQ ID NO:52, as shown in Table 9.4 above.

Optionally an isolated polypeptide of the invention which comprises at least one of the amino acid residues or deletions, at positions corresponding to the amino acid residue positions of SEQ ID NO:52, as shown in Table 9.4 above, comprises at least ten of the amino acid residues or deletions, at positions corresponding to the amino acid residue positions of SEQ ID NO:52, as shown in Table 9.4 above.

Optionally the coronavirus S protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:52.

Optionally an isolated polypeptide of the invention which comprises at least one of the amino acid residues or deletions, at positions corresponding to the amino acid residue positions of SEQ ID NO:52, as shown in Table 9.4 above, comprises amino acid residue P at position 986, and amino acid residue P at position 987, corresponding to the amino acid residue positions of SEQ ID NO:52, and at least one, or all of the amino acid residues or deletions, at positions corresponding to the amino acid residue positions of SEQ ID NO:52, as shown in Table 9.5 above.

### Designed Coronavirus S Protein Sequence in Closed State to Protect Against COVID-19 Variants, and Predicted Future Variants

The majority of SARS-CoV-2 vaccines in use or in advanced clinical development are based on the viral spike protein (S) as their immunogen. S is present on virions as pre-fusion trimers in which the receptor binding domain (RBD) is stochastically open or closed. Neutralizing antibodies have been described that act against both open and closed conformations. The long-term success of vaccination strategies will depend upon inducing antibodies that provide long-lasting broad immunity against evolving, circulating SARS-CoV-2 strains, while avoiding the risk of antibody dependent enhancement as observed with other Coronavirus vaccines.

Carnell et al. (“SARS-CoV-2 spike protein arrested in the closed state induces potent neutralizing responses”; https://doi.org/10.1101/2021.01.14426695, posted 14 Jan. 2021) have assessed the results of immunization in a mouse model using an S protein trimer that is arrested in the closed state to prevent exposure of the receptor binding site and therefore interaction with the receptor. The authors compared this with a range of other modified S protein constructs, including representatives used in current vaccines. They found that all trimeric S proteins induce a long-lived, strongly neutralizing antibody response as well as T-cell responses. Notably, the protein binding properties of sera induced by the closed spike differed from those induced by standard S protein constructs. Closed S proteins induced more potent neutralising responses than expected based on the degree to which they inhibit interactions between the RBD and ACE2. The authors conclude that these observations suggest that closed spikes recruit different, but equally potent, virus-inhibiting immune responses than open spikes, and that this is likely to include neutralizing antibodies against conformational epitopes present in the closed conformation.

We have appreciated that the amino acid changes of the designed S protein sequences disclosed herein (and especially of SEQ ID NO:53 as described in Example 30) may optionally be present in a designed S protein that is arrested in the closed state, and thereby further improve the antibody response of the designed sequences. In particular, use of such structural constraints may reduce immunodominance to key regions, and spread the antibody response to focus on other, or less immunodominant sites.

Example 31 below describes optional additional amino acid changes that may be made to a designed S protein sequence to allow it to form a closed structure.

Optionally a designed S protein sequence of the invention may comprise cysteine residues at positions corresponding to positions 413 and 987 of the full length S protein sequence. For example, G413C and V987C.

For example, a designed S protein sequence of the invention may comprise the following amino acid sequence (SEQ ID NO:54) (with cysteine residues at positions 410 and 984, which correspond to positions 413 and 987, respectively, of SEQ ID NO:52):

| | |
|---|---|
| MFVFLVLLPL VSSQCVNFTN RTQLPSAYTN SFTRGVYYPD KVFRSSVLHS TQDLFLPFFS | 60 |
| NVTWFHAISG TNGTKRFDNP VLPFNDGVYF ASTEKSNIIR GWIFGTTLDS KTQSLLIVNN | 120 |
| ATNVVIKVCE FQFCNDPFLG VYHKNNKSWM ESEFRVYSSA NNCTFEYVSQ PFLMDLEGKQ | 180 |
| GNFKNLREFV FKNIDGYFKI YSKHTPINLV RDLPQGFSAL EPLVDLPIGI NITRFQTLLA | 240 |
| LHRSYLTPGD SSSGWTAGAA AYYVGYLQPR TFLLKYNENG TITDAVDCAL DPLSETKCTL | 300 |
| KSFTVEKGIY QTSNFRVQPT ESIVRFPNIT NLCPFGEVFN ATRFASVYAW NRKRISNCVA | 360 |
| DYSVLYNSAS FSTFKCYGVS PTKLNDLCFT NVYADSFVIR GDEVRQIAPC QTGNIADYNY | 420 |
| KLPDDFTGCV IAWNSNNLDS KVGGNYNYLY RLFRKSNLKP FERDISTEIY QAGSTPCNGV | 480 |
| KGFNCYFPLQ SYGFQPTYGV GYQPYRVVVL SFELLHAPAT VCGPKKSTNL VKNKCVNFNF | 540 |
| NGLTGTGVLT ESNKKFLPFQ QFGRDIADTT DAVRDPQTLE ILDITPCSFG GVSVITPGTN | 600 |
| TSNQVAVLYQ GVNCTEVPVA IHADQLTPTW RVYSTGSNVF QTRAGCLIGA EHVNNSYECD | 660 |
| IPIGAGICAS YQTQTNSHRR ARSVASQSII AYTMSLGAEN SVAYSNNSIA IPTNFTISVT | 720 |
| TEILPVSMTK TSVDCTMYIC GDSTECSNLL LQYGSFCTQL NRALTGIAVE QDKNTQEVFA | 780 |
| QVKQIYKTPP IKDFGGFNFS QILPDPSKPS KRSFIEDLLF NKVTLADAGF IKQYGDCLGD | 840 |
| IAARDLICAQ KFNGLTVLPP LLTDEMIAQY TSALLAGTIT SGWTFGAGAA LQIPFAMQMA | 900 |
| YRFNGIGVTQ NVLYENQKLI ANQFNSAIGK IQDSLSSTAS ALGKLQDVVN QNAQALNTLV | 960 |
| KQLSSNFGAI SSVLNDILSR LDPCEAEVQI DRLITGRLQS LQTYVTQQLI RAAEIRASAN | 1020 |
| LAATKMSECV LGQSKRVDFC GKGYHLMSFP QSAPHGVVFL HVTYVPAQEK NFTTAPAICH | 1080 |
| DGKAHFPREG VFVSNGTHWF VTQRNFYEPQ IITTDNTFVS GNCDVVIGIV NNTVYDPLQP | 1140 |
| ELDSFKEELD KYFKNHTSPD VDLGDISGIN ASVVNIQKEI DRLNEVAKNL NESLIDLQEL | 1200 |
| GKYEQYIKWP WYIWLGFIAG LIAIVMVTIM LCCMTSCCSC LKGCCSCGSC CKFDEDDSEP | 1260 |
| VLKGVKLHYT | 1270 |

According to the invention there is provided an isolated polypeptide, which comprises an amino acid sequence of SEQ ID NO:54.

According to the invention there is also provided an isolated polypeptide comprising an amino acid sequence of SEQ ID NO:54, or an amino acid sequence which has at least 99% amino acid identity over its entire length with the amino acid sequence of SEQ ID NO:54.

Optionally a polypeptide of the invention comprising an amino acid sequence of SEQ ID NO:54, or an amino acid sequence which has at least 99% amino acid identity over its entire length with the amino acid sequence of SEQ ID NO:54, comprises at least one, or all of the amino acid residues or deletions, at positions corresponding to the amino acid residue positions of SEQ ID NO:52, as shown in Table 9.4 below:

TABLE 9.4

| SARS2 S | |
|---|---|
| protein | |
| residue | |
| position | COV_S_T2_29 |
| (SEQ ID | amino acid residue |
| NO: 52) | (SEQ ID NO: 53) |
| 18 | F |
| 20 | N |
| 26 | S |
| 69 | — (deletion) |
| 70 | — (deletion) |
| 144 | — (deletion |
| 417 | N |
| 484 | K |
| 501 | Y |
| 614 | G |
| 681 | H |
| 986 | P |
| 987 | P |

Optionally a polypeptide of the invention comprising an amino acid sequence of SEQ ID NO:54, or an amino acid sequence which has at least 99% amino acid identity over its entire length with the amino acid sequence of SEQ ID NO:54, comprises at least five of the amino acid residues or deletions, at positions corresponding to the amino acid residue positions of SEQ ID NO:52, as shown in Table 9.4.

Optionally a polypeptide of the invention comprising an amino acid sequence of SEQ ID NO:54, or an amino acid sequence which has at least 99% amino acid identity over its entire length with the amino acid sequence of SEQ ID NO:54, comprises at least ten of the amino acid residues or deletions, at positions corresponding to the amino acid residue positions of SEQ ID NO:52, as shown in Table 9.4.

Optionally a polypeptide of the invention comprising an amino acid sequence of SEQ ID NO:54, or an amino acid sequence which has at least 99% amino acid identity over its entire length with the amino acid sequence of SEQ ID NO:54, comprises at least one, or all of the amino acid residues or deletions, at positions corresponding to the amino acid residue positions of SEQ ID NO:52, as shown in Table 9.5 below:

Optionally a polypeptide of the invention comprising an amino acid sequence of SEQ ID NO:54, or an amino acid sequence which has at least 99% amino acid identity over its entire length with the amino acid sequence of SEQ ID NO:54, comprises amino acid residue P at position 986 corresponding to the amino acid residue positions of SEQ ID NO:52, and at least one, or all of the amino acid residues or deletions, at positions corresponding to the amino acid residue positions of SEQ ID NO:52, as shown in Table 9.5 below:

TABLE 9.5

| SARS2 S | |
|---|---|
| protein | |
| residue | |
| position | COV_S_T2_29 |
| (SEQ ID | amino acid residue |
| NO: 52 | (SEQ ID NO: 53) |
| 18 | F |
| 20 | N |
| 26 | S |
| 69 | — (deletion) |
| 70 | — (deletion) |
| 144 | — (deletion) |
| 417 | N |
| 484 | K |
| 501 | Y |
| 614 | G |
| 681 | H |

According to the invention there is also provided an isolated polypeptide which comprises a coronavirus S protein comprising cysteine amino acid residues at positions corresponding to positions 413 and 987 of SEQ ID NO:52, and at least one, or all of the amino acid residues or deletions, at positions corresponding to the amino acid residue positions of SEQ ID NO:52, as shown in Table 9.5 above.

Optionally an isolated polypeptide of the invention which comprises cysteine amino acid residues at positions corresponding to positions 413 and 987 of SEQ ID NO:52, and at least one of the amino acid residues or deletions, at positions corresponding to the amino acid residue positions of SEQ ID NO:52, as shown in Table 9.5 above, comprises at least five of the amino acid residues or deletions, at positions corresponding to the amino acid residue positions of SEQ ID NO:52, as shown in Table 9.5 above.

Optionally an isolated polypeptide of the invention which comprises cysteine amino acid residues at positions corresponding to positions 413 and 987 of SEQ ID NO:52, and at least one of the amino acid residues or deletions, at positions corresponding to the amino acid residue positions of SEQ ID NO:52, as shown in Table 9.5 above, comprises at least ten of the amino acid residues or deletions, at positions corresponding to the amino acid residue positions of SEQ ID NO:52, as shown in Table 9.5 above.

Optionally an isolated polypeptide of the invention which comprises cysteine amino acid residues at positions corresponding to positions 413 and 987 of SEQ ID NO:52, and at least one of the amino acid residues or deletions, at positions corresponding to the amino acid residue positions of SEQ ID NO:52, as shown in Table 9.5 above, comprises amino acid residue P at position 986.

We have also appreciated that any SARS-CoV-2 spike protein may be modified to include cysteine residues at positions corresponding to positions 413 and 987 of SEQ ID NO:52 to allow it to form a spike protein arrested in the closed state, in accordance with Carnell et al. (supra), and thereby elicit more potent neutralising responses compared with the corresponding unmodified protein. For example, Jeong et al. (https://virological.org/t/assemblies-of-putative-sars-cov2-spike-encoding-mma-sequences-for-vaccines-bnt-162b2-and-mma-1273/663—version 0.2Beta Mar. 30, 2021) have recently reported experimental sequence information for the RNA components of the initial Moderna (https://pubmed.ncbi.nlm.nih.gov/32756549/) and Pfizer/BioNTech (https://pubmed.ncbi.nlm.nih.gov/33301246/) COVID-19 vaccines, allowing a working assembly of the former and a confirmation of previously reported sequence information for the latter RNA (see the sequences provided in FIGS. 1 and 2 of the document). Spike protein encoded by such sequence may be modified to include cysteine residues at positions corresponding to positions 413 and 987 of SEQ ID NO:52.

According to the invention there is also provided an isolated polypeptide which comprises a coronavirus S protein comprising cysteine amino acid residues at positions corresponding to positions 413 and 987 of SEQ ID NO:52.

Optionally the coronavirus S protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:52.

SARS-CoV-2 is continually evolving, with more contagious mutations spreading rapidly. Zahradnik et al., 2021 (“SARS-CoV-2 RBD in vitro evolution follows contagious mutation spread, yet generates an able infection inhibitor”; doi: https://doi.org/10.1101/2021.01.06.425392, posted 29 Jan. 2021) recently reported using in vitro evolution to affinity maturate the receptor-binding domain (RBD) of the spike protein towards ACE2 resulting in the more contagious mutations, S477N, E484K, and N501Y, to be among the first selected, explaining the convergent evolution of the “European” (20E-EU1), “British” (501.V1), “South African” (501.V2), and “Brazilian” variants (501.V3). The authors report that further in vitro evolution enhancing binding by 600-fold provides guidelines towards potentially new evolving mutations with even higher infectivity. For example, Q498R epistatic to N501Y.

We have also appreciated that the designed S protein sequences (RBD, truncated, or full-length) disclosed herein (and especially in the sections entitled “Designed Coronavirus full-length S protein sequence to protect against COVID-19 variants”, and “Designed Coronavirus S protein sequence in closed state to protect against COVID-19 variants, and predicted future variants” above, and in Examples 30 and 31 below) may optionally also include amino acid substitutions at one or more residue positions predicted to be mutated in future COVID-19 variants with a vaccine escape response, for example at one or more (or all) of positions 446, 452, 477, and 498 (for example, G446R, S477N, Q498R, especially Q498R).

Optionally an isolated polypeptide of the invention includes amino acid changes at one or more (or all) of the following positions (corresponding to amino acid residue positions of SEQ ID NO:52): 446, 452, 477, and 498 (for example, G446R, S477N, Q498R, especially Q498R).

Optionally an isolated polypeptide of the invention includes amino acid changes at positions (corresponding to amino acid residue positions of SEQ ID NO:52): Q498R and N501Y.

### Designed Coronavirus Envelope (E) Protein Sequences

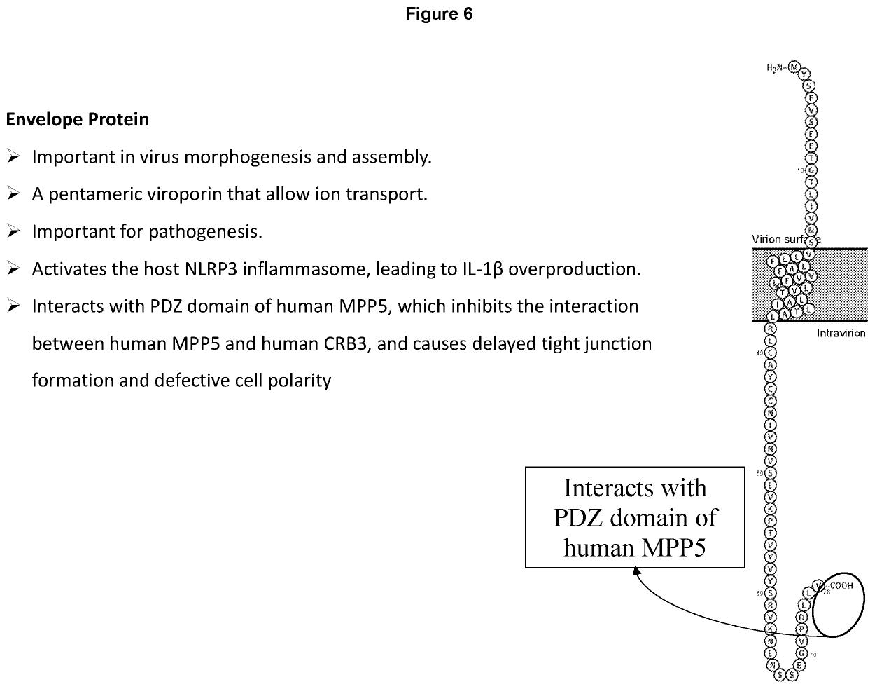

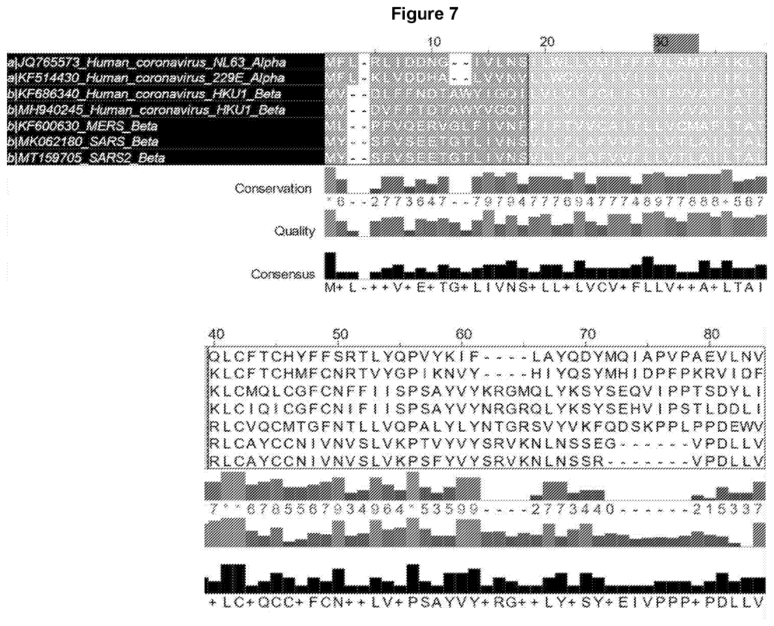

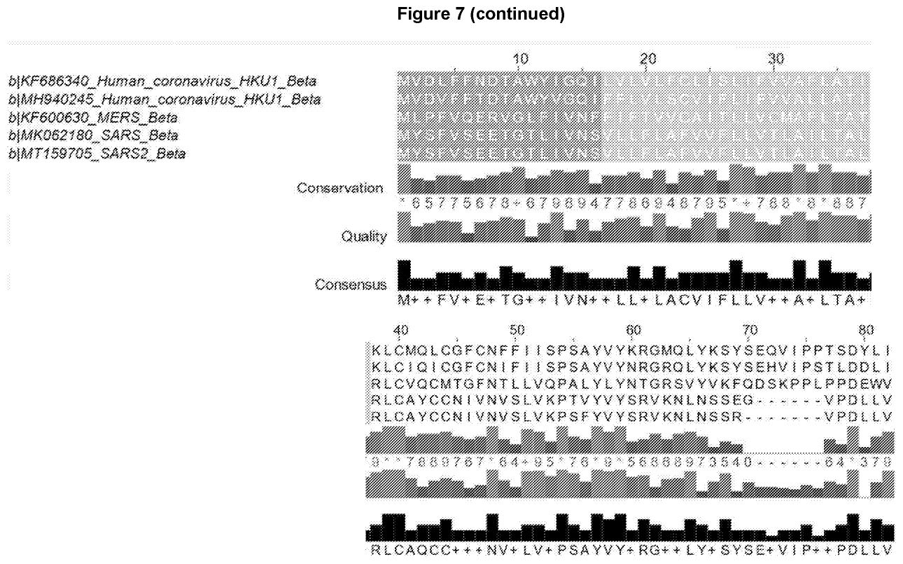

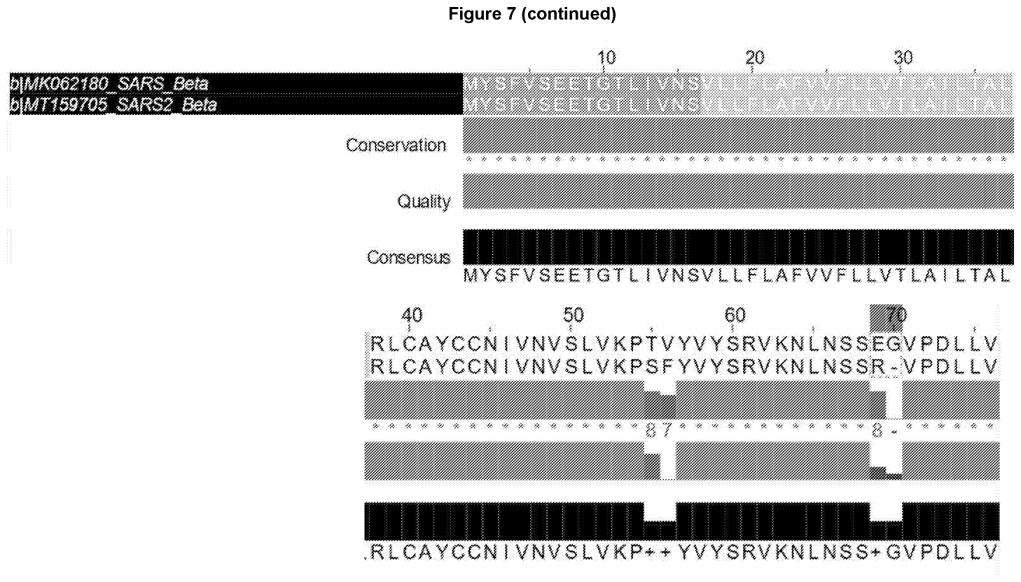

We have also generated novel amino acid sequences for coronavirus Envelope (E) protein. FIG. 6 shows an amino acid sequence of the SARS Envelope (E) protein (SEQ ID NO:21), and illustrates key features of the sequence. As described in Example 10 below, FIG. 7 shows a multiple sequence alignment of coronavirus E protein sequences, comparing sequences for isolates of NL63 and 229E (alpha-coronaviruses), and HKU1, MERS, SARS, and SARS2 (beta-coronaviruses). The alignment shows that the C-terminal end of the E protein for the SARS2 and SARS sequences (beta-coronaviruses of subgenus Sarbeco) includes a deletion, compared with the other sequences, and that the SARS2 E protein sequence includes a deletion, and an Arginine (positively charged) amino acid residue, compared with the SARS sequence.

The novel amino acid sequences for coronavirus E protein are called COV_E_T2_1 (a designed Sarbecovirus sequence) (SEQ ID NO:22) and COV_E_T2_2 (a designed SARS2 sequence) (SEQ ID NO:23):

>COV_E_T2_1
(SEQ ID NO: 22)
MYSFVSEETG TLIVNSVLLF LAFVVFLLVT LAILTALRLC
AYCCNIVNVS LVKPTFYVYS RVKNLNSSQG VPDLLV
>COV_E_T2_2
(SEQ ID NO: 23)
MYSFVSEETG TLIVNSVLLF LAFVVFLLVT LAILTALRLC
AYCCNIVNVS LVKPTFYVYS RVKNLNSSR- VPDLLV

Alignment of the SARS2 reference E protein sequence in FIG. 7 with these designed sequences highlights that there are four amino acid differences between the SARS2 reference E protein sequence and the COV_E_T2_1 designed sequence (SEQ ID NO:22), and two amino acid differences between the SARS2 reference E protein sequence and the COV_E_T2_2 designed sequence (SEQ ID NO:23):

| | | |
|---|---|---|
| SARS2 | 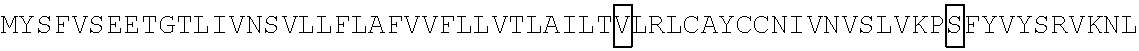 | 65 |
| COV_E_T2_1 | 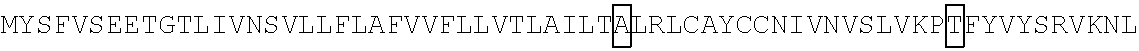 | 65 |
| COV_E_T2_2 | 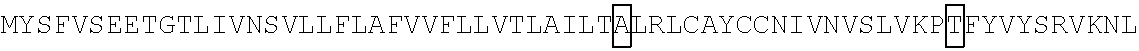 | 65 |
| SARS2 | 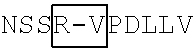 | 75 |
| COV_E_T2_1 | 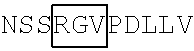 | 76 |
| COV_E_T2_2 | 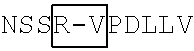 | 75 |

The C-terminal sequence of the COV_E_T2_2 sequence is identical to the SARS2 reference sequence. The C-terminal of the E protein is one of the identified epitopes for E-protein, so the amino acid deletion and the substitution with an Arginine residue present in the SARS2 reference sequence (compared with the SARS reference sequence in FIG. 6) have been retained in the COV_E_T2_2 designed sequence. The amino acid differences at the other positions are optimised to maximise induction of an immune response that recognises all Sarbeco viruses.

The amino acid differences are summarised in the table below:

TABLE 10.1

| SARS2 E | SARS2 | | |
|---|---|---|---|
| protein | Reference | COV_E_T2_1 | COV_E_T2_2 |
| residue | Amino acid | Amino | Amino |
| position | residue | acid residue | acid residue |
| 36 | V | A | A |
| 55 | S | T | T |
| 69 | R | Q | R |
| 70 | — | G | — |

There is also provided according to the invention an isolated polypeptide which comprises an amino acid sequence of SEQ ID NO:22, or an amino acid sequence which has at least 95%, 96%, 97%, 98%, or 99% amino acid identity over its entire length with the amino acid sequence of SEQ ID NO:22.

Optionally an isolated polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, comprises one or both amino acid residues, at positions corresponding to the amino acid residue positions of SEQ ID NO:22, as shown in the table below:

TABLE 10.2

| SARS2 E protein | COV_E_T2_1 Amino |
|---|---|
| residue position | acid residue |
| 36 | A |
| 55 | T |

Optionally an isolated polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, comprises any, at least two, at least three, or all, of the amino acid residues, at positions corresponding to the amino acid residue positions of SEQ ID NO:22, as shown in the table below:

TABLE 10.3

| SARS2 E protein | COV_E_T2_1 Amino |
|---|---|
| residue position | acid residue |
| 36 | A |
| 55 | T |
| 69 | Q |
| 70 | G |

There is also provided according to the invention an isolated polypeptide, which comprises an amino acid sequence of SEQ ID NO:23, or an amino acid sequence which has at least 98%, or 99% amino acid identity over its entire length with the amino acid sequence of SEQ ID NO:23.

Optionally an isolated polypeptide of the invention which comprises an amino acid sequence of SEQ ID NO:23, or an amino acid sequence which has at least 98%, or 99% amino acid identity over its entire length with the amino acid sequence of SEQ ID NO:23, comprises one or both amino acid residues, at positions corresponding to the amino acid residue positions of SEQ ID NO:23, as shown in the table below:

TABLE 10.4

| SARS2 E protein | COV_E_T2_2 Amino |
|---|---|
| residue position | acid residue |
| 36 | A |
| 55 | T |

According to the invention there is also provided an isolated polypeptide, which comprises a coronavirus E protein with one or both of the amino acid residues at positions corresponding to the amino acid residue positions as shown in the table below:

TABLE 10.5

| E protein residue | |
|---|---|
| position | Amino acid residue |
| 36 | A |
| 55 | T |

According to the invention there is also provided an isolated polypeptide, which comprises a coronavirus E protein with any, at least two, at least three, or all, of the amino acid residues at positions corresponding to the amino acid residue positions as shown in the table below:

TABLE 10.6

| E protein residue | |
|---|---|
| position | Amino acid residue |
| 36 | A |
| 55 | T |
| 69 | Q |
| 70 | G |

Optionally an isolated polypeptide of the invention which comprises a coronavirus E protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:21.

In the alignment above residue 36 of the SARS2 reference sequence is shown as V, but is actually A (as correctly shown in FIG. 7 and SEQ ID NO:21). Alignment of SEQ ID NO:21 with the designed sequences highlights that there are three amino acid differences between the alternative SARS2 reference E protein sequence and the COV_E_T2_1 designed sequence (SEQ ID NO:22), and one amino acid difference between the SARS2 reference E protein sequence and the COV_E_T2_2 designed sequence (SEQ ID NO:23):

| | | |
|---|---|---|
| SARS2 | 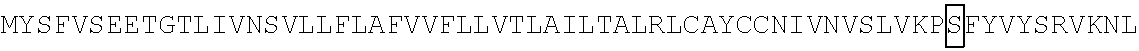 | 65 |
| COV_E_T2_1 | 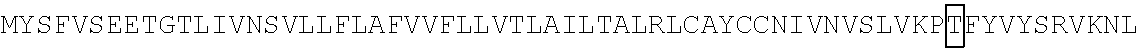 | 65 |
| COV_E_T2_2 | 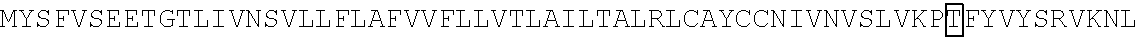 | 65 |
| SARS2 COV_E_T2_1 COV_E_T2_2 | 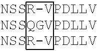 | 75 76 75 |

The amino acid differences are summarised in the table below:

TABLE 10.7

| SARS2 E | SARS2 | | |
|---|---|---|---|
| protein | Reference | COV_E_T2_1 | COV_E_T2_2 |
| residue | Amino acid | Amino | Amino |
| position | residue | acid residue | acid residue |
| 55 | S | T | T |
| 69 | R | Q | R |
| 70 | — | G | — |

There is also provided according to the invention an isolated polypeptide which comprises an amino acid sequence of SEQ ID NO:22 (COV_E_T2_1), or an amino acid sequence which has at least 97%, 98%, or 99% amino acid identity over its entire length with the amino acid sequence of SEQ ID NO:22.

Optionally an isolated polypeptide of the invention which comprises an amino acid sequence of SEQ ID NO:22, or an amino acid sequence which has at least 97%, 98%, or 99% amino acid identity over its entire length with the amino acid sequence of SEQ ID NO:22, comprises the amino acid residue, at a position corresponding to the amino acid residue position of SEQ ID NO:22, as shown in the table below:

TABLE 10.8

| SARS2 E protein | COV_E_T2_1 Amino |
|---|---|
| residue position | acid residue |
| 55 | T |

Optionally an isolated polypeptide of the invention which comprises an amino acid sequence of SEQ ID NO:22, or an amino acid sequence which has at least 97%, 98%, or 99% amino acid identity over its entire length with the amino acid sequence of SEQ ID NO:22, comprises any, at least two, or all, of the amino acid residues, at positions corresponding to the amino acid residue positions of SEQ ID NO:22, as shown in the table below:

TABLE 10.9

| SARS2 E protein | COV_E_T2_1 Amino |
|---|---|
| residue position | acid residue |
| 55 | T |
| 69 | Q |
| 70 | G |

There is also provided according to the invention an isolated polypeptide, which comprises an amino acid sequence of SEQ ID NO:23, or an amino acid sequence which has at least 99% amino acid identity over its entire length with the amino acid sequence of SEQ ID NO:23.

Optionally an isolated polypeptide of the invention which comprises an amino acid sequence of SEQ ID NO:23, or an amino acid sequence which has at least 99% amino acid identity over its entire length with the amino acid sequence of SEQ ID NO:23, comprises an amino acid residues, at a position corresponding to the amino acid residue positions of SEQ ID NO:23, as shown in the table below:

TABLE 10.10

| SARS2 E protein | COV_E_T2_2 Amino |
|---|---|
| residue position | acid residue |
| 55 | T |

According to the invention there is also provided an isolated polypeptide, which comprises a coronavirus E protein with the amino acid residue at a position corresponding to the amino acid residue position as shown in the table below:

TABLE 10.11

| E protein residue | Amino |
|---|---|
| position | acid residue |
| 55 | T |

According to the invention there is also provided an isolated polypeptide, which comprises a coronavirus E protein with any, at least two, or all, of the amino acid residues at positions corresponding to the amino acid residue positions as shown in the table below:

TABLE 10.12

| E protein residue | Amino |
|---|---|
| position | acid residue |
| 55 | T |
| 69 | Q |
| 70 | G |

Optionally an isolated polypeptide of the invention which comprises a coronavirus E protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:21.

SARS-CoV envelope (E) gene encodes a 76-amino acid transmembrane protein with ion channel (IC) activity, an important function in virus-host interaction. Infection of mice with viruses lacking or displaying E protein IC activity revealed that activation of the inflammasome pathway, and the exacerbated inflammatory response induced by SARS-CoV, was decreased in infections by ion channel-deficient viruses (Nieto-Torres et al., 2014, Severe Acute Respiratory Syndrome Coronavirus Envelope Protein Ion Channel Activity Promotes Virus Fitness and Pathogenesis. PLoS Pathog 10(5): e1004077).

We have made new E protein designs Cov_E_T2_3, CoV_E_T2_4 and CoV_E_T2_5, which correspond to new designs of SARS2 reference (SEQ ID NO:41), CoV_E_T2_1 (SEQ ID NO:22), and CoV_E_T2_2 (SEQ ID NO:23) (see Example 10), respectively. These new designs have a point mutation. N15A, which abrogates the ion channel activity, but does not influence the stability of the structure. Nieto-Torres et al., supra, discusses this mutation as well as the toxicity and inflammatory action of SARS E on the host cell.

The amino acid sequence of SARS2 envelope protein reference (SEQ ID NO:41) is:

(SEQ ID NO: 41)
MYSFVSEETG TLIVNSVLLF LAFVVFLLVT LAILTALRLC
AYCCNIVNVS LVKPSFYVYS RVKNLNSSRV PDLLV

The amino acid sequences of the new E protein designs are shown below, and in Example 25:

>COV_E_T2_3 (SARS2_mutant)
(SEQ ID NO: 42)
MYSFVSEETG TLIVASVLLF LAFVVFLLVT LAILTALRLC
AYCCNIVNVS LVKPSFYVYS RVKNLNSSR-VPDLLV
>COV_E_T2_4 (Env1_mutant)
(SEQ ID NO: 43)
MYSFVSEETG TLIVASVLLF LAFVVFLLVT LAILTALRLC
AYCCNIVNVS LVKPTFYVYS RVKNLNSSQG VPDLLV
>COV_E_T2_5 (Env2_mutant)
(SEQ ID NO: 44)
MYSFVSEETG TLIVASVLLF LAFVVFLLVT LAILTALRLC
AYCCNIVNVS LVKPTFYVYS RVKNLNSSR-VPDLLV

Alignment of the E protein designs with SARS2 E protein reference sequence is shown below:

| | | | |
|---|---|---|---|
| SARS2 | 1 | MYSFVSEETGTLIVNSVLLFLAFVVFLLVTLAILTALRLCAYCCNIVNVSLVKPSFYVYSRV | 62 |
| COV_E_T2_1 | 1 | MYSFVSEETGTLIVNSVLLFLAFVVFLLVTLAILTALRLCAYCCNIVNVSLVKPTFYVYSRV | 62 |
| COV_E_T2_2 | 1 | MYSFVSEETGTLIVNSVLLFLAFVVFLLVTLAILTALALCAYCCNIVNVSLVKPTFYVYSRV | 62 |
| COV_E_T2_3 | 1 | MYSFVSEETGTLIVASVLLFLAFVVFLLVTLAILTALRLCAYCCNIVNVSLVKPSFYVYSRV | 62 |
| COV_E_T2_4 | 1 | MYSFVSEETGTLIVASVLLFLAFVVFLLVTLAILTALRLCAYCCNIVNVSLVKPTFYVYSRV | 62 |
| COV_E_T2_5 | 1 | MYSFVSEETGTLIVASVLLFLAFVVFLLVTLAILTALRLCAYCCNIVNVSLVKPTFYVYSRV | 62 |
| SARS2 | 63 | KNLNSSR-VPDLLV | 75 |
| COV_E_T2_1 | 63 | KNLNSSQGVPDLLV | 76 |
| COV_E_T2_2 | 63 | KNLNSSR-VPDLLV | 75 |
| COV_E_T2_3 | 63 | KNLNSSR-VPDLLV | 75 |
| COV_E_T2_4 | 63 | KNLNSSQGVPDLLV | 76 |
| COV_E_T2_5 | 63 | KNLNSSR-VPDLLV | 75 |

The amino acid differences of the designed sequences from the SARS2 reference sequence (SEQ ID NO:41) are shown in the table below (with differences from the reference sequence highlighted in bold):

According to the invention there is provided an isolated polypeptide, which comprises an amino acid sequence according to any of SEQ ID NOs:36-38.

According to the invention there is also provided an isolated polypeptide comprising an amino acid sequence of SEQ ID NO:42 (COV_E_T2_3), or an amino acid sequence which has at least 99% amino acid identity over its entire length with the amino acid sequence of SEQ ID NO:42.

Optionally a polypeptide of the invention comprising an amino acid sequence of SEQ ID NO:42 (COV_E_T2_3), or an amino acid sequence which has at least 99% amino acid identity over its entire length with the amino acid sequence of SEQ ID NO:42, comprises amino acid residue A at a position corresponding to amino acid residue position 15 of SEQ ID NO:41.

According to the invention there is also provided an isolated polypeptide comprising an amino acid sequence of SEQ ID NO:43 (COV_E_T2_4), or an amino acid sequence which has at least 95%, 96%, 97%, 98%, or 99% amino acid identity over its entire length with the amino acid sequence of SEQ ID NO:43.

Optionally a polypeptide of the invention comprising an amino acid sequence of SEQ ID NO:43 (COV_E_T2_4), or an amino acid sequence which has at least 95%, 96%, 97%, 98%, or 99% amino acid identity over its entire length with the amino acid sequence of SEQ ID NO:43, comprises at least one, or all of the following amino acid residues at positions corresponding to the amino acid residue positions of SEQ ID NO:41: 15A, 55T, 69Q, 70G.

According to the invention there is also provided an isolated polypeptide comprising an amino acid sequence of SEQ ID NO:44 (COV_E_T2_5), or an amino acid sequence which has at least 98%, or 99% amino acid identity over its entire length with the amino acid sequence of SEQ ID NO:44.

Optionally a polypeptide of the invention comprising an amino acid sequence of SEQ ID NO:44 (COV_E_T2_5), or an amino acid sequence which has at least 98%, or 99% amino acid identity over its entire length with the amino acid sequence of SEQ ID NO:44, comprises at least one, or all of the following amino acid residues at positions corresponding to the amino acid residue positions of SEQ ID NO:41: 15A, 55T.

According to the invention there is also provided an isolated polypeptide which comprises a coronavirus E protein with at least one of the following amino acid residues at positions corresponding to the amino acid residue positions of SEQ ID NO:41: 15A, 55T, 69Q, 70G.

Optionally an isolated polypeptide of the invention which comprises a coronavirus E protein, comprises the following amino acid residues at positions corresponding to the amino acid residue positions of SEQ ID NO:41: 15A, 55T.

Optionally an isolated polypeptide of the invention which comprises a coronavirus E protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:21.

### Designed Coronavirus Membrane (M) Protein Sequences

The applicant has also generated novel amino acid sequences for coronavirus Membrane (M) protein:
COV_M_T2_1 Sarbecovirus root ancestor (SEQ ID NO:24);
COV_M_T2_2 Epitope optimised version of SARS2 clade ancestor Node88b (D4 removed), SARS2 equivalent of B cell epitope from start and end added, and then T cell epitopes added whilst observing coevolving site constraints (SEQ ID NO:25).

The amino acid sequences of these designed sequences are:
>COV_M_T2_1/1-221 Sarbeco_M_root:

(SEQ ID NO: 24)
MADNGTITVE ELKQLLEQWN LVIGFLFLAW IMLLQFAYSN
RNRFLYIIKL VFLWLLWPVT LACFVLAAVY RINWVTGGIA
IAMACIVGLM WLSYFVASFR LFARTRSMWS FNPETNILLN
VPLRGTILTR PLMESELVIG AVIIRGHLRM AGHSLGRCDI
KDLPKEITVA TSRTLSYYKL GASQRVGTDS GFAAYNRYRI
GNYKLNTDHA GSNDNIALLV Q

>COV_M_T2_2/1-222 Sarbeco_M_Node88b_epitope_optimised:

(SEQ ID NO: 25)
MADSNGTITV EELKKLLEQW NLVIGFLFLT WICLLQFAYS
NRNRFLYIIK LIFLWLLWPV TLACFVLAAV YRINWVTGGI
AIAMACIVGL MWLSYFVASF RLFARTRSMW SFNPETNILL
NVPLRGSIIT RPLMESELVI GAVILRGHLR MAGHSLGRCD
IKDLPKEITV ATSRTLSYYK LGASQRVASD SGFAVYNRYR
IGNYKLNTDH SSSSDNIALL VQ

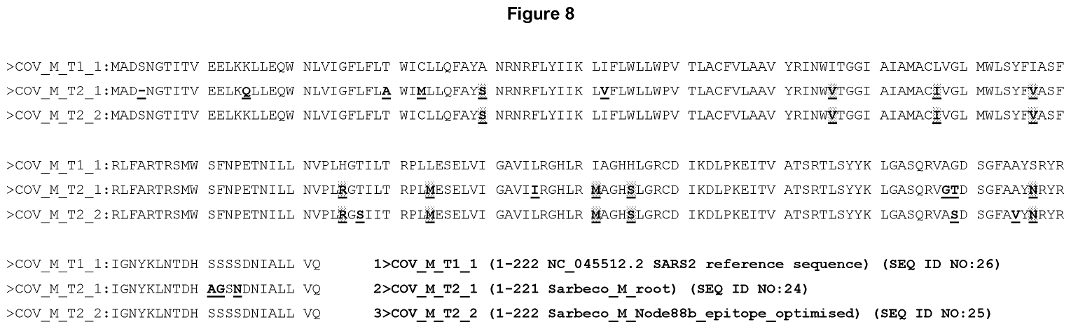

As described in Example 11 below, FIG. 8 shows alignment of a SARS2 reference M protein sequence (SEQ ID NO:26) with the designed sequences. The alignment shown in FIG. 8 highlights the amino acid differences between the SARS2 reference M protein sequence and the COV_M_T2_1 and COV_M_T2_2 designed sequences, as shown in the table below:

TABLE 11.1

| SARS2 M | SARS2 | COV_M_ | COV_M_ |
|---|---|---|---|
| reference | Reference | T2_1 | T2_2 |
| protein | Amino | Amino | Amino |
| residue | acid | acid | acid |
| position | residue | residue | residue |
| (SEQ ID | (SQ ID | (SEQ ID | (SEQ ID |
| NO: 26) | NO: 26) | NO: 24) | NO: 25) |
| 4 | S | — | S |
| 15 | K | Q | K |
| 30 | T | A | T |
| 33 | C | M | C |
| 40 | A | [symbol] | [symbol] |
| 52 | I | V | I |
| 76 | I | [symbol] | [symbol] |
| 87 | L | [symbol] | [symbol] |
| 97 | I | [symbol] | [symbol] |
| 125 | H | [symbol] | [symbol] |
| 127 | T | T | S |
| 134 | L | [symbol] | [symbol] |
| 145 | L | I | L |
| 151 | I | [symbol] | [symbol] |
| 155 | H | [symbol] | [symbol] |
| 188 | A | G | A |
| 189 | G | T | S |
| 195 | A | A | V |
| 197 | S | [symbol] | [symbol] |
| 211 | S | A | S |
| 212 | S | G | S |
| 214 | S | N | S |

According to the invention there is also provided an isolated polypeptide which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24.

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises at least one of the amino acid residues, at a position corresponding to the amino acid residue position of SEQ ID NO:26, as shown in the table below:

TABLE 11.2

| SARS2 M protein | COV_M_T2_1 Amino |
|---|---|
| residue position | acid residue |
| 40 | S |
| 76 | V |
| 87 | I |
| 97 | V |
| 125 | R |
| 134 | M |
| 151 | M |
| 155 | S |
| 197 | N |

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises at least five of the amino acid residues, at positions corresponding to the amino acid residue positions of SEQ ID NO:26, as shown in Table 11.2.

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises all of the amino acid residues, at positions corresponding to the amino acid residue positions of SEQ ID NO:26, as shown in Table 11.2.

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises at least one of the amino acid residues, at a position corresponding to the amino acid residue position of SEQ ID NO:26, as shown in the table below:

TABLE 11.3

| SARS2 M protein | COV_M_T2_1 Amino |
|---|---|
| residue position | acid residue |
| 4 | — (deletion) |
| 15 | Q |
| 30 | A |
| 33 | M |
| 40 | S |
| 52 | V |
| 76 | V |
| 87 | I |
| 97 | V |
| 125 | R |
| 134 | M |
| 145 | I |
| 151 | M |
| 155 | S |
| 188 | G |
| 189 | T |
| 197 | N |
| 211 | A |
| 212 | G |
| 214 | N |

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises at least five of the amino acid residues, at positions corresponding to the amino acid residue positions of SEQ ID NO:26, as shown in Table 11.3.

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises at least ten of the amino acid residues, at positions corresponding to the amino acid residue positions of SEQ ID NO:26, as shown in Table 11.3.

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises at least fifteen of the amino acid residues, at positions corresponding to the amino acid residue positions of SEQ ID NO:26, as shown in Table 11.3.

Optionally an isolated polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, comprises all of the amino acid residues, at positions corresponding to the amino acid residue positions of SEQ ID NO:26, as shown in Table 11.3.

There is also provided according to the invention an isolated polypeptide which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises at least one of the amino acid residues, at a position corresponding to the amino acid residue positions of SEQ ID NO:25, as shown in the table below:

TABLE 11.4

| SARS2 M protein | COV_M_T2_2 Amino |
|---|---|
| residue position | acid residue |
| 40 | S |
| 76 | V |
| 87 | I |
| 97 | V |
| 125 | R |
| 134 | M |
| 151 | M |
| 155 | S |
| 197 | N |

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises at least five of the amino acid residues, at positions corresponding to the amino acid residue positions of SEQ ID NO:25, as shown in Table 11.4.

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises all of the amino acid residues, at positions corresponding to the amino acid residue positions of SEQ ID NO:25, as shown in Table 11.4.

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises at least one of the amino acid residues, at a position corresponding to the amino acid residue position of SEQ ID NO:25, as shown in the table below:

TABLE 11.5

| SARS2 M protein | COV_M_T2_2 Amino |
|---|---|
| residue position | acid residue |
| 40 | S |
| 76 | V |
| 87 | I |
| 97 | V |
| 125 | R |
| 127 | S |
| 134 | M |
| 151 | M |
| 155 | S |
| 189 | S |
| 195 | V |
| 197 | N |

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises at least five of the amino acid residues, at positions corresponding to the amino acid residue positions of SEQ ID NO:25, as shown in Table 11.5.

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises at least ten of the amino acid residues, at positions corresponding to the amino acid residue positions of SEQ ID NO:25, as shown in Table 11.5.

Optionally an isolated polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25, comprises all of the amino acid residues, at positions corresponding to the amino acid residue positions of SEQ ID NO:25, as shown in Table 11.5.

According to the invention there is also provided an isolated polypeptide, which comprises a coronavirus M protein with any, or all of the amino acid residues at positions corresponding to the amino acid residue positions as shown in the table below:

TABLE 11.6

| M protein residue | Amino acid |
|---|---|
| position | residue |
| 40 | S |
| 76 | V |
| 87 | I |
| 97 | V |
| 125 | R |
| 134 | M |
| 151 | M |
| 155 | S |
| 197 | N |

According to the invention there is also provided an isolated polypeptide, which comprises a coronavirus M protein with any, or all of the amino acid residues at positions corresponding to the amino acid residue positions as shown in the table below:

TABLE 11.7

| M protein residue | Amino acid |
|---|---|
| position | residue |
| 4 | — (deletion) |
| 15 | Q |
| 30 | A |
| 33 | M |
| 40 | S |
| 52 | V |
| 76 | V |
| 87 | I |
| 97 | V |
| 125 | R |
| 134 | M |
| 145 | I |
| 151 | M |
| 155 | S |
| 188 | G |
| 189 | T |
| 197 | N |
| 211 | A |
| 212 | G |
| 214 | N |

According to the invention there is also provided an isolated polypeptide, which comprises a coronavirus M protein with any, or all of the amino acid residues at positions corresponding to the amino acid residue positions as shown in the table below:

TABLE 11.8

| M protein residue | Amino acid |
|---|---|
| position | residue |
| 40 | S |
| 76 | V |
| 87 | I |
| 97 | V |
| 125 | R |
| 134 | M |
| 151 | M |
| 155 | S |
| 197 | N |

According to the invention there is also provided an isolated polypeptide, which comprises a coronavirus M protein with any, or all of the amino acid residues at positions corresponding to the amino acid residue positions as shown in the table below:

TABLE 11.9

| M protein residue | Amino acid |
|---|---|
| position | residue |
| 40 | S |
| 76 | V |
| 87 | I |
| 97 | V |
| 125 | R |
| 127 | S |
| 134 | M |
| 151 | M |
| 155 | S |
| 189 | S |
| 195 | V |
| 197 | N |

Optionally an isolated polypeptide of the invention which comprises a coronavirus M protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:26.

We have made further new M protein designs (COV_M_T2_3, COV_M_T2_4, COV_M_T2_5)). In these designs, we have deleted the first and the second transmembrane region of the membrane protein to abrogate its interaction with the S protein:
The string construct with S, M and E was showing higher order aggregates.
Abrogation of interaction between S and M—can reduce aggregation.
M-del constructs (Cov_M_T2_(3-5)) designed to abrogate the interaction with S.

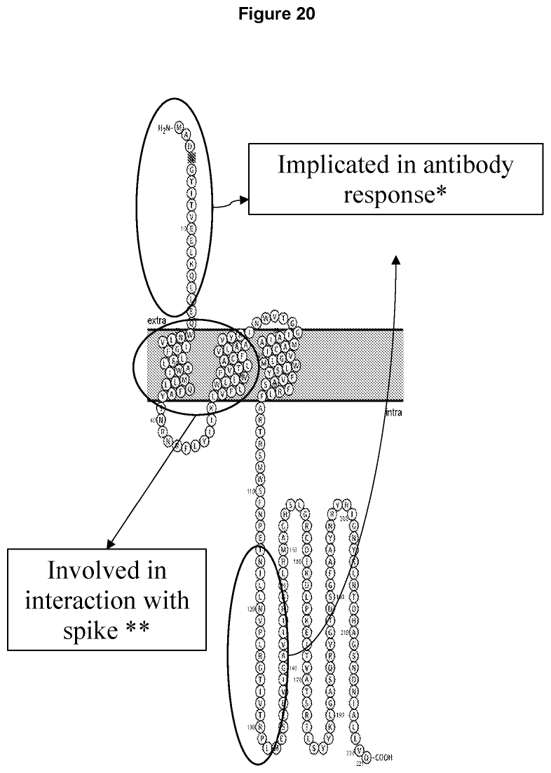

FIG. 20 shows an illustration of the M protein. Interaction between the M, E and N proteins is important for viral assembly. The M protein also binds to the nucleocapsid, and this interaction promotes the completion of virion assembly. These interactions have been mapped to the C-terminus of the endo-domain of the M protein, and the C-terminal domain of the N-protein. In FIG. 20, * denotes identification of immunodominant epitopes on the membrane protein of the Severe Acute Respiratory Syndrome-Associated Coronavirus, and ** denotes mapping of the Coronavirus membrane protein domains involved in interaction with the Spike protein.

The amino acid sequences of the new M protein designs are given below:

>COV_M_T2_3
(SEQ ID NO: 48)
MADSNGTITV EELKKLLEQI TGGIAIAMAC LVGLMWLSYF
IASFRLFART RSMWSFNPET NILLNVPLHG TILTRPLLES
ELVIGAVILR GHLRIAGHHL GRCDIKDLPK EITVATSRTL
SYYKLGASQR VAGDSGFAAY SRYRIGNGKL NTDHSSSSDN
IALLVQ
>COV_M_T2_4
(SEQ ID NO: 49)
MADNGTITVE ELKQLLEQVT GGIAIAMACI VGLMWLSYFV
ASFRLFARTR SMWSFNPETN ILLNVPLRGT ILTRPLMESE
LVIGAVIIRG HLRMAGHSLG RCDIKDLPKE ITVATSRTLS
YYKLGASQRV GTDSGFAAYN RYRIGNGKLN TDHAGSNDNI
ALLVQ
>COV_M_T2_5
(SEQ ID NO: 50)
MADSNGTITV EELKKLLEQV TGGIAIAMAC IVGLMWLSYF
VASFRLFART RSMWSFNPET NILLNVPLRG SIITRPLMES
SYYKLGASQR VASDSGFAVY ELVIGAVILR GHLRMAGHSL
GRCDIKDLPK EITVATSRTL NRYRIGNGKL NTDHSSSSDN
IALLVQ

Sequence alignment of the new M protein designs (COV_M_T2_3, COV_M_T2_4, COV_M_T2_5) with the previous M protein designs (COV_M_T1_1, COV_M_T2_1, COV_M_T2_2) is shown below:

| | | |
|---|---|---|
| COV_M_T1_1 | MADSNGTITVEELKKELEQWNLVIGFLFLTWICLLQFAYANRNRFLYIIKLIFLWLLWPVTLA | 63 |
| COV_M_T2_3 | MADSNGTITVEELKKELEQ-------------------------------------------- | 19 |
| COV_M_T2_1 | MAD-NGTITVEELKQLLEQWNLVIGFLFLAWIMLLQFAYSNRNRFLYIIKLVFLWLLWPVTLA | 62 |
| COV_M_T2_4 | MAD-NGTITVEELKQLLEQ-------------------------------------------- | 18 |
| COV_M_T2_2 | MADSNGTITVEELKKLLEQWNLVIGFLFLTWICLLQFAYSNRNRFLYIIKLIFLWLLWPVTLA | 63 |
| COV_M_T2_5 | MADSNGTITVEELKKLLEQ-------------------------------------------- | 19 |
| COV_M_T1_1 | CFVLAAVYRINWITGGIAIAMACLVGLMWLSYFIASFRLFARTRSMWSFNPETNILLNVPLHG | 126 |
| COV_M_T2_3 | ------------ITGGIAIAMACLVGLMWLSYFIASFRLFARTRSMWSFNPETNILLNVPLHG | 70 |
| COV_M_T2_1 | CFVLAAVYRINWVTGGIAIAMACIVGLMWLSYFVASFRLFARTRSMWSFNPETNILLNVPLRG | 125 |
| COV_M_T2_4 | ------------VTGGIATAMACIVGLMWLSYFVASFRLFARTRSMWSFNPETNILLNVPLRG | 69 |
| COV_M_T2_2 | CFVLAAVYRINWVTGGIAIAMACIVGLMWLSYFVASFRLFARTRSMWSFNPETNILLNVPLRG | 126 |
| COV_M_T2_5 | ------------VTGGIAIAMACIVGLMWLSYFVASFRLFARTRSMWSFNPETNILLNVPLRG | 70 |
| COV_M_T1_1 | TILTRPLLESELVIGAVILRGHLRIAGHHLGRCDLKDLPKEITVATSRTLSYYKLGASQRVAG | 189 |
| COV_M_T2_3 | TILTRPLLESELVIGAVILRGHLRIAGHHLGRCDLKDLPKEITVATSRTLSYYKLGASQRVAG | 133 |
| COV_M_T2_1 | TILTRPLMESELVIGAVIIRGHLRMAGHSLGRCDLKDLPKEITVATSRTLSYYKLGASQRVGT | 188 |
| COV_M_T2_4 | TILTRPLMESELVIGAVIIRGHLRMAGHSLGRCDIKDLPKEITVATSRTLSYYKLGASQRVGT | 132 |
| COV_M_T2_2 | SIITRPLMESELVIGAVILRGHLRMAGHSLGRCDIKDLPKEITVATSRTLSYYKLGASQRVAS | 189 |
| COV_M_T2_5 | SIITRPLMESELVIGAVILRGHLRMAGHSLGRCDIKDLPKEITVATSRTLSYYKLGASQRVAS | 133 |
| COV_M_T1_1 | DSGFAAYSRYRIGNYKLNTDHSSSSDNIALLVQ | 222 |
| COV_M_T2_3 | DSGFAAYSRYRIGNGKLNTDHSSSSDNIALLVQ | 166 |
| COV_M_T2_1 | DSGFAAYNRYRIGNYKLNTDHAGSNDNIALLVQ | 221 |
| COV_M_T2_4 | DSGFAAYNRYRIGNGKLNTDHAGSNDNIALLVQ | 165 |
| COV_M_T2_2 | DSGFAVYNRYRIGNYKLNTDNSSSSDNIALLVQ | 222 |
| COV_M_T2_5 | DSGFAVYNRYRIGNGKLNTDHSSSSDNIALLVQ | 166 |

The amino acid differences of the designed sequences from the SARS2 M protein reference sequence are shown in the table below (with differences from the reference sequence highlighted in bold):

According to the invention there is also provided an isolated polypeptide which comprises an amino acid sequence of SEQ ID NO:48, or an amino acid sequence which has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:48.

Optionally an isolated polypeptide of the invention which comprises an amino acid sequence of SEQ ID NO:48, or an amino acid sequence which has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:48, comprises a deletion of amino acid residues at positions corresponding to positions 20-75 of SEQ ID NO:26.

Optionally an isolated polypeptide of the invention which comprises an amino acid sequence of SEQ ID NO:48, or an amino acid sequence which has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:48, comprises amino acid residue G at a position corresponding to amino acid residue position 204 of SEQ ID NO:26.

According to the invention there is also provided an isolated polypeptide which comprises an amino acid sequence of SEQ ID NO:49, or an amino acid sequence which has at least 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:49.

Optionally an isolated polypeptide of the invention which comprises an amino acid sequence of SEQ ID NO:49, or an amino acid sequence which has at least 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:49, comprises a deletion of amino acid residues at positions corresponding to positions 20-75 of SEQ ID NO:26.

Optionally an isolated polypeptide of the invention which comprises an amino acid sequence of SEQ ID NO:49, or an amino acid sequence which has at least 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:49, comprises at least one, or all, of the amino acid residues, at a position corresponding to the amino acid residue position of SEQ ID NO:26, as shown in the table below:

TABLE 11.11

| SARS2 M protein | Amino |
|---|---|
| residue position | acid |
| (SEQ ID NO: 26) | residue |
| 20-75 | Deleted |
| 76 | V |
| 87 | I |
| 97 | V |
| 125 | R |
| 134 | M |
| 151 | M |
| 155 | S |
| 189 | T |
| 197 | N |
| 204 | G |

Optionally an isolated polypeptide of the invention which comprises an amino acid sequence of SEQ ID NO:49, or an amino acid sequence which has at least 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:49, comprises at least one, or all, of the amino acid residues, at a position corresponding to the amino acid residue position of SEQ ID NO:26, as shown in the table below:

TABLE 11.12

| SARS2 M protein | COV_M_T2_4 |
|---|---|
| residue position | Amino acid |
| (SEQ ID | residue (SEQ |
| NO: 26) | ID NO: 49) |
| 4 | Deleted |
| 15 | Q |
| 20-75 | Deleted |
| 76 | V |
| 87 | I |
| 97 | V |
| 125 | R |
| 134 | M |
| 145 | I |
| 151 | M |
| 155 | S |
| 188 | G |
| 189 | T |
| 197 | N |
| 204 | G |
| 211 | A |
| 212 | G |
| 214 | N |

According to the invention there is also provided an isolated polypeptide which comprises an amino acid sequence of SEQ ID NO:50, or an amino acid sequence which has at least 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:50.

Optionally an isolated polypeptide of the invention which comprises an amino acid sequence of SEQ ID NO:50, or an amino acid sequence which has at least 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:50, comprises a deletion of amino acid residues at positions corresponding to positions 20-75 of SEQ ID NO:26.

Optionally an isolated polypeptide of the invention which comprises an amino acid sequence of SEQ ID NO:50, or an amino acid sequence which has at least 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:50, comprises at least one, or all, of the amino acid residues, at a position corresponding to the amino acid residue position of SEQ ID NO:26, as shown in the table below:

TABLE 11.11

| SARS2 M protein | Amino |
|---|---|
| residue position | acid |
| (SEQ ID NO: 26) | residue |
| 20-75 | Deleted |
| 76 | V |
| 87 | I |
| 97 | V |
| 125 | R |
| 134 | M |
| 151 | M |
| 155 | S |
| 189 | T |
| 197 | N |
| 204 | G |

Optionally an isolated polypeptide of the invention which comprises an amino acid sequence of SEQ ID NO:50, or an amino acid sequence which has at least 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:50, comprises at least one, or all, of the amino acid residues, at a position corresponding to the amino acid residue position of SEQ ID NO:26, as shown in the table below:

TABLE 11.13

| SARS2 M protein | COV_M_T2_5 |
|---|---|
| residue position | Amino acid |
| (SEQ ID | residue (SEQ |
| NO: 26) | ID NO: 50) |
| 20-75 | Deleted |
| 76 | V |
| 87 | I |
| 97 | V |
| 125 | R |
| 127 | S |
| 129 | I |
| 134 | M |
| 151 | M |
| 155 | S |
| 189 | S |
| 195 | V |
| 197 | N |
| 204 | G |

According to the invention there is also provided an isolated polypeptide, which comprises a coronavirus M protein with any, or all of the amino acid residues at positions corresponding to the amino acid residue positions as shown in the table below:

TABLE 11.11

| SARS2 M protein | Amino |
|---|---|
| residue position | acid |
| (SEQ ID NO: 26) | residue |
| 20-75 | Deleted |
| 76 | V |
| 87 | I |
| 97 | V |
| 125 | R |
| 134 | M |
| 151 | M |
| 155 | S |
| 189 | T |
| 197 | N |
| 204 | G |

According to the invention there is also provided an isolated polypeptide, which comprises a coronavirus M protein with any, or all of the amino acid residues at positions corresponding to the amino acid residue positions as shown in the table below:

TABLE 11.12

| | COV_M_T2_4 |
|---|---|
| SARS2 M protein | Amino acid |
| residue position | residue (SEQ |
| (SEQ ID NO: 26) | ID NO: 49) |
| 4 | Deleted |
| 15 | Q |
| 20-75 | Deleted |
| 76 | V |
| 87 | I |
| 97 | V |
| 125 | R |
| 134 | M |
| 145 | I |
| 151 | M |
| 155 | S |
| 188 | G |
| 189 | T |
| 197 | N |
| 204 | G |
| 211 | A |
| 212 | G |
| 214 | N |

According to the invention there is also provided an isolated polypeptide, which comprises a coronavirus M protein with any, or all of the amino acid residues at positions corresponding to the amino acid residue positions as shown in the table below:

TABLE 11.13

| SARS2 M protein | COV_M_T2_5 |
|---|---|
| residue position | Amino acid |
| (SEQ ID | residue (SEQ |
| NO: 26) | ID NO: 50) |
| 20-75 | Deleted |
| 76 | V |
| 87 | I |
| 97 | V |
| 125 | R |
| 127 | S |
| 129 | I |
| 134 | M |
| 151 | M |
| 155 | S |
| 189 | S |
| 195 | V |
| 197 | N |
| 204 | G |

Optionally an isolated polypeptide of the invention which comprises a coronavirus M protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:26.

### Designed Coronavirus Nucleoprotein (N) Sequences

We have made new N protein designs, COV_N_T2_1 (SEQ ID NO:46) and COV_N_T2_2 (SEQ ID NO:47). The amino acid sequences of these designs is shown below, and in Example 15. Sequence COV_N_T2_2 was designed using a methodology and algorithm which selected predicted epitopes to include based on their conservation across the sarbecoviruses (whilst minimising redundancy), the frequency and number of MHC alleles the epitope is restricted by the predicted epitope quality, and a handful of user specified weightings.

>YP_009724397.2/1-419 nucleocapsid phosphoprotein
(SARS-COV-2](reference sequence)
(SEQ ID NO: 45)
MSDNGPQ-NQ RNAPRITFGG PSDSTGSNQN GERSGARSKQ
RRPQGLPNNT ASWFTALTQH GKEDLKFPRG QGVPINTNSS
PDDQIGYYRR ATRRIRGGDG KMKDLSPRWY FYYLGTGPEA
GLPYGANKDG IIWVATEGAL NTPKDHIGTR NPANNAAIVL
QLPQGTTLPK GFYAEGSRGG SQASSRSSSR SRNSSRNSTP
GSSRGTSPAR MAGNGGDAAL ALLLLDRLNQ LESKMSGKGQ
QQQGQTVTKK SAAEASKKPR QKRTATKAYN VTQAFGRRGP
EQTQGNFGDQ ELIRQGTDYK HWPQIAQFAP SASAFFGMSR
IGMEVTPSGT WLTYTGAIKL DDKDPNFKDQ VILLNKHIDA
YKTFPPTEPK KDKKKKADET QALPQRQKKQ QTVTLLPAAD
LDDFSKQLQQ SMSSA--DST QA
>COV_N_T2 1/1-418 Node1b 321-323 deleted
(SEQ ID NO: 46)
MSDNGPQ-NQ RSAPRITFGG PSDSTDNNQN GERSGARPKQ
RRPQGLPNNT ASWFTALTQH GKEDLRFPRG QGVPINTNSG
KDDQIGYYRR ATRRVRGGDG KMKELSPRWY FYYLGTGPEA
ALPYGANKEG IVWVATEGAL NTPKDHIGTR NPNNNAAIVL
QLPQGTTLPK GFYAEGSRGG SQASSRSSSR SRGNSRNSTP
GSSRGTSPAR MASGGGDTAL ALLLLDRLNQ LESKVSGKGQ
QQQGQTVTKK SAAEASKKPR QKRTATKQYN VTQAFGRRGP
EQTQGNFGDQ ELIRQGTDYK HWPQIAQFAP SASAFFGMSR
---EVTPSGT WLTYHGAIKL DDKDPQFKDN VILLNKHIDA
YKTFPPTEPK KDKKKKADEA QPLPQRQKKQ PTVILLPAAD
LDDFSKQLQN SMSGASADST QA
>COV_N_T2 2/1-417 epitope optimised 321-323
deleted
(SEQ ID NO: 47)
MTDNGQQ-GP RNAPRITF-G VSDNFDNNQD GGRSGARPKQ
RRPQGLPNNT ASWFTALTQH GKEDLRFPRG QGVPINTNSS
PDDQIGYYRR ATRRIRGGDG KMKDLSPRWY FYYLGTGPEA
ALPYGANKEG IVWVATEGAL NTPKDHIGTR NPNNNAAIVL
QLPQGTTLPK GFYAEGSRGG SQASSRSSSR SRNSSRNSTP
GSSRGTSPAR NLQAGGDTAL ALLLLDRLNQ LESKMSGKGQ
QQQGQTVTKK SAAEASKKPR QKRTATKQYN VTQAFGRRGP
EQTQGNEGDQ ELIRQGTDYK QWPQIAQFAP SASAFFGMSR
---EVTPSGT WLTYTGAIKL DDKDPQFKDN VILLNKHIDA
YKTFPPTEPK KDKKKKADEA QPLPQRQKKQ QTVTLLPAAD
LDDFSRQLQN SMSGASADST QA

Alignment of the N protein designs with SARS2 N protein reference sequence is shown below:

| | | |
|---|---|---|
| YP_009724397 | MSDNGPQNQRNAPRITFGGPSDSTGSNQNGERSGARSKQRRPGGLPNNTASWFTALTQHGKEDLKFPRGQGVPINTNSSPDDGIGYYRRATRRIPGGDGKMKDLSPPW | 108 |
| COV_N_T2_1 | MSDNGPQNQRSAPRITFGGPSDSTDNNQNGERSGARPKQRRPGGLPNNTASWFTALTQHGKEDLRFPRGQGVPINTNSGKDDGIGYYRRATRRVRGGDGKMKELSPRW | 108 |
| COV_N_T2_2 | MTDNGQQGPRNAPRITF-GVSDNFDNNQDGGRSGARPKQRRPQGLPNNTASWFTALTQHGKEDLRFPRGQGVPINTNSSPDDGIGYYRRATRRIRGGDGKMKDLSPPW | 107 |
| YP_009724397 | YFYYLGTGPEAGLPYGANKDGIIWVATEGALNTPKDHIGTRNPANNAAIVLQLPQGTTLPK-GFYAEGSRGGGQASSRGGSRSPNSSRNSTPGSSRGTSPARMAGNGG | 215 |
| COV_N_T2_1 | YFYYLGTGPEAALPYGANKEGIVWVATEGALNTPKDHIGTRNPNNNAAIVLQLPQGTTLPK-GFYAEGSRGGGQASSRSSSRSRGNSRNSTPGSSRGTSPARMASGGG | 215 |
| COV_N_T2_2 | YFYYLGTGPEAALPYGANKEGIVWVATEGALNTPKDHIGTRNPNNNAAIVLQLPQGTTLPK-GFYAEGSRGGGQASSRSSSRSRNSSRNSTPGSSRGTSPARNLQAGG | 214 |
| YP_009724397 | DAALALLLLDRLNGLESKMSGKGQQQQGQTVTKKSAAEASKKPRQKRTATKAYNVTQAFGRRGPEGTQGNFGDQELIRQGTDYKHWPQIAQFAPSASAFFGMSRIGME | 320 |
| COV_N_T2_1 | DTALALLLLDRLNGLESKVSGKGQQQQGQTVTKKSAAEASKKPRQKRTATKQYNVTQAFGRRGPEGTQGNFGDQELIRQGTDYKHWPQIAQFAPSALAFFGMSR---E | 320 |
| COV_N_T2_2 | DTALALLLLDRLNGLESKMSGKGQQQQGQTYTKFSAAEASKKPRQKRTATKQYNVTQAFGRRGPEGTQGNFGDQELIRQGTDYKQWPQIAQFAPSASAFFGMSR---E | 319 |
| YP_009724397 | VTPSGTWLTYTGAIKLDDKDPNFKDQVILLNKHIDAYKTFPPTEPKKDKKKKADETQALPQRQKKGGTVTLLPAAD-LDDFSKGLQQSMSGA--DSTQA | 419 |
| COV_N_T2_1 | VTPSGTWLTYHGAIKLDDKDPQFKDNVILLNKHIDAYKTFPPTEPKKDKKKKADEAQPLPQRQKKGPTVTLLPAAD-LDDFSKGLQNSMSGASADSTQA | 418 |
| COV_N_T2_2 | VTPSGTWLTYTGAIKLDDKDPQFKDNVILLNKHIDAYKTFPPTEPKKDKKKKADEAQPLPQRQKKGGTVTLLPAAD-LDDFSRGLQNSMSGASADSTQA | 417 |

The amino acid differences of the designed sequences from the SARS2 reference sequence are shown in the Table 12.1 below (with differences from the reference sequence highlighted in bold, and differences that are common to all the designed sequences underlined):

Positions 415 and 416 of the SARS2 N protein reference residue position column are italicized as they are not residues of the reference sequences, but include insertions in the N_T2_1 and N_T2_2 sequences.

The amino acid changes common to both of the designed sequences are summarised in the table below:

TABLE 12.2

| | Amino acid |
|---|---|
| | residue of |
| SARS2 N | designed |
| (SEQ ID | sequences |
| NO: 45) | (SEQ ID |
| residue | Nos: |
| position | 46, 47) |
| 26 | N |
| 37 | P |
| 65 | R |
| 120 | A |
| 128 | E |
| 131 | V |
| 152 | N |
| 217 | T |
| 267 | Q |
| 345 | Q |
| 349 | N |
| 379 | A |
| 409 | N |
| 413 | G |
| 415 | S (insertion) |
| 416 | A (insertion) |

Optional additional changes are summarised in the table below:

TABLE 12.3

| SARS2 N | Amino acid |
|---|---|
| protein | residue of |
| (SEQ ID | designed |
| NO: 45) | sequence |
| residue | (SEQ ID |
| position | NO: 46) |
| 11 | S |
| 79 | G |
| 80 | K |
| 94 | V |
| 103 | E |
| 192 | G |
| 193 | N |
| 212 | S |
| 213 | G |
| 234 | V |
| 320 | — |
| 321 | — |
| 322 | — |
| 334 | H |
| 390 | P |

Alternative optional additional changes are summarised in the table below:

TABLE 12.4

| SARS2 N | |
|---|---|
| protein | Amino |
| (SEQ ID | acid |
| NO: 45) | residue |
| residue | (SEQ ID |
| position | NO: 47) |
| 2 | T |
| 6 | Q |
| 8 | G |
| 9 | P |
| 18 | — |
| 20 | V |
| 23 | N |
| 24 | F |
| 25 | D |
| 29 | D |
| 31 | G |
| 211 | L |
| 212 | Q |
| 213 | A |
| 300 | Q |
| 320 | — |
| 321 | — |
| 322 | — |
| 406 | R |

According to the invention there is provided an isolated polypeptide which comprises an amino acid sequence of SEQ ID NO:46 (COV_N_T2_1), or an amino acid sequence which has at least 93%, 94%, 95%, 96%, 97%, 98%, or 99% amino acid identity over its entire length with the amino acid sequence of SEQ ID NO:46.

Optionally a polypeptide of the invention comprising an isolated polypeptide comprising an amino acid sequence of SEQ ID NO:46, or an amino acid sequence which has at least 93%, 94%, 95%, 96%, 97%, 98%, or 99% amino acid identity over its entire length with the amino acid sequence of SEQ ID NO:46, further comprises at least one, or all of the amino acid residues at positions corresponding to the amino acid residue positions as shown in Table 12.2 above.

Optionally a polypeptide of the invention comprising an isolated polypeptide comprising an amino acid sequence of SEQ ID NO:46, or an amino acid sequence which has at least 93%, 94%, 95%, 96%, 97%, 98%, or 99% amino acid identity over its entire length with the amino acid sequence of SEQ ID NO:46, further comprises at least one, or all of the amino acid residues at positions corresponding to the amino acid residue positions as shown in Table 12.3 above.

According to the invention there is also provided an isolated polypeptide which comprises an amino acid sequence of SEQ ID NO:47 (COV_N_T2_2), or an amino acid sequence which has at least 92%, 93%, 94%, 95%, 96%, 97%, 98%, or 99% amino acid identity over its entire length with the amino acid sequence of SEQ ID NO:47.

Optionally a polypeptide of the invention comprising an isolated polypeptide comprising an amino acid sequence of SEQ ID NO:47, or an amino acid sequence which has at least 92%, 93%, 94%, 95%, 96%, 97%, 98%, or 99% amino acid identity over its entire length with the amino acid sequence of SEQ ID NO:47, further comprises at least one, or all of the amino acid residues at positions corresponding to the amino acid residue positions as shown in Table 12.2 above.

Optionally a polypeptide of the invention comprising an isolated polypeptide comprising an amino acid sequence of SEQ ID NO:47, or an amino acid sequence which has at least 92%, 93%, 94%, 95%, 96%, 97%, 98%, or 99% amino acid identity over its entire length with the amino acid sequence of SEQ ID NO:47, further comprises at least one, or all of the amino acid residues at positions corresponding to the amino acid residue positions as shown in Table 12.4 above.

According to the invention there is also provided an isolated polypeptide, which comprises a coronavirus N protein with at least one, or all of the amino acid residues at positions corresponding to the amino acid residue positions of SEQ ID NO:45 as shown in Table 12.2 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least five amino acid residues at positions corresponding to the amino acid residue positions of SEQ ID NO:45, as shown in Table 12.2 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least ten amino acid residues at positions corresponding to the amino acid residue positions of SEQ ID NO:45, as shown in Table 12.2 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least fifteen amino acid residues at positions corresponding to the amino acid residue positions of SEQ ID NO:45, as shown in Table 12.2 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least one, or all of the amino acid residues at positions corresponding to the amino acid residue positions of SEQ ID NO:45, as shown in Table 12.3 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least five of the amino acid residues at positions corresponding to the amino acid residue positions of SEQ ID NO:45, as shown in Table 12.3 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least ten of the amino acid residues at positions corresponding to the amino acid residue positions of SEQ ID NO:45, as shown in Table 12.3 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least one, or all of the amino acid residues at positions corresponding to the amino acid residue positions of SEQ ID NO:45, as shown in Table 12.4 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least five of the amino acid residues at positions corresponding to the amino acid residue positions of SEQ ID NO:45, as shown in Table 12.4 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least ten of the amino acid residues at positions corresponding to the amino acid residue positions of SEQ ID NO:45, as shown in Table 12.4 above.

Optionally an isolated polypeptide of the invention which comprises a coronavirus N protein with at least one, or all of the amino acid residues at positions corresponding to the amino acid residue positions of SEQ ID NO:45, as shown in Table 12.2 above, comprises at least fifteen of the amino acid residues at positions corresponding to the amino acid residue positions of SEQ ID NO:45, as shown in Table 12.4 above.

Optionally an isolated polypeptide of the invention which comprises a coronavirus N protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:45.

Polypeptides of the invention are particularly advantageous because they can elicit a broadly neutralising immune response to several different types of coronavirus, in particular several different types of β-coronavirus. Polypeptides of the invention comprising an amino acid sequence of SEQ ID NO:15 (or an amino acid sequence which has at least 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:15), or SEQ ID NO:17 (or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:17) are also advantageous because they lack non-neutralising epitopes that may result in virus immune evasion and disease progression by ADE (or ADE-like pro-inflammatory responses).

Similarly, polypeptides of the invention comprising a novel designed coronavirus E protein amino acid sequence (for example, an amino acid sequence of SEQ ID NO:22, or an amino acid sequence which has at least 95%, 96%, 97%, 98%, or 99% amino acid identity over its entire length with the amino acid sequence of SEQ ID NO:22, or an amino acid sequence of SEQ ID NO:23, or an amino acid sequence which has at least 98%, or 99% amino acid identity over its entire length with the amino acid sequence of SEQ ID NO:23), or a coronavirus M protein amino acid sequence (for example, an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or an amino acid sequence of SEQ ID NO:25, or an amino acid sequence which has at least 95%, 96%, 97%, 98%, or 99% amino acid identity over its entire length with the amino acid sequence of SEQ ID NO:25) are advantageous because they lack non-neutralising epitopes that may result in virus immune evasion and disease progression by ADE (or ADE-like pro-inflammatory responses).

A polypeptide of the invention may include one or more conservative amino acid substitutions. Conservative amino acid substitutions are those substitutions that, when made, least interfere with the properties of the original polypeptide, that is, the structure and especially the function of the protein is conserved and not significantly changed by such substitutions. Examples of conservative substitutions are shown below:

| Original Residue | Conservative Substitutions |
|---|---|
| Ala | Ser |
| Arg | Lys |
| Asn | Gln, His |
| Asp | Glu |
| Cys | Ser |
| Gin | Asn |
| Glu | Asp |
| His | Asn; Gln |
| Ile | Leu, Val |
| Leu | Ile; Val |
| Lys | Arg; Gln; |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, serine or threonine, is substituted for (or by) a hydrophobic residue, for example, leucine, isoleucine, phenylalanine, valine or alanine; (b) a cysteine or proline is substituted for (or by) any other residue; (c) a residue having an electropositive side chain, for example, lysine, arginine, or histidine, is substituted for (or by) an electronegative residue, for example, glutamate or aspartate; or (d) a residue having a bulky side chain, for example, phenylalanine, is substituted for (or by) one not having a side chain, for example, glycine.

The term “broadly neutralising immune response” is used herein to mean an immune response elicited in a subject that is sufficient to inhibit (i.e. reduce), neutralise or prevent infection, and/or progress of infection, of a virus within the coronavirus family. Optionally a broadly neutralising immune response is sufficient to inhibit, neutralise or prevent infection, and/or progress of infection, of more than one type of β-coronavirus (for example, SARS-CoV, and SARS-CoV-2). Optionally a broadly neutralising immune response is sufficient to inhibit, neutralise or prevent infection, and/or progress of infection, of more than one type of β-coronavirus within the same β-coronavirus lineage (for example, more than one type of β-coronavirus within the subgenus Sarbecovirus, such as SARS-CoV, SARS-CoV-2, and Bat SL-CoV-WIV1). Optionally a broadly neutralising immune response is sufficient to inhibit, neutralise or prevent infection, and/or progress of infection, of coronaviruses of different β-coronavirus lineages, such as lineage B (for example, SARS-CoV, and SARS-CoV-2) and lineage C (for example, MERS-CoV). Optionally a broadly neutralising immune response is sufficient to inhibit, neutralise or prevent infection, and/or progress of infection, of most or all different β-coronaviruses. Optionally a broadly neutralising immune response is sufficient to inhibit, neutralise or prevent infection, and/or progress of infection, of most or all different viruses of the coronavirus family.

The immune response may be humoral and/or a cellular immune response. A cellular immune response is a response of a cell of the immune system, such as a B-cell, T-cell, macrophage or polymorphonucleocyte, to a stimulus such as an antigen or vaccine. An immune response can include any cell of the body involved in a host defence response, including for example, an epithelial cell that secretes an interferon or a cytokine. An immune response includes, but is not limited to, an innate immune response or inflammation.

Optionally a polypeptide of the invention induces a protective immune response. A protective immune response refers to an immune response that protects a subject from infection or disease (i.e. prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, or antibody production.

Optionally a polypeptide of the invention is able to induce the production of antibodies and/or a T-cell response in a human or non-human animal to which the polypeptide has been administered (either as a polypeptide or, for example, expressed from an administered nucleic acid expression vector).

Optionally a polypeptide of the invention is a glycosylated polypeptide.

### Nucleic Acid Molecules

According to the invention there is also provided an isolated nucleic acid molecule encoding a polypeptide of the invention, or the complement thereof.

There is also provided according to the invention an isolated nucleic acid molecule comprising a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length to a nucleic acid molecule of the invention encoding a polypeptide of the invention, or the complement thereof.

Optionally an isolated nucleic acid molecule of the invention comprises a nucleotide sequence of SEQ ID NO:18, 16, or 14, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with a nucleotide sequence of SEQ ID NO: 18, 16, or 14 over its entire length, or the complement thereof.

According to the invention there is also provided an isolated nucleic acid molecule which comprises a nucleotide sequence encoding a polypeptide of the invention comprising an amino acid sequence of SEQ ID NO:33, 34, 35, or 36.

Optionally the nucleotide sequence encoding a polypeptide comprising an amino acid sequence of SEQ ID NO:33, 34, 35, or 36 comprises a nucleotide sequence of SEQ ID NO:37, 38, 39, or 40, respectively.

According to the invention there is also provided an isolated nucleic acid molecule which comprises a nucleotide sequence encoding an isolated polypeptide of the invention comprising an amino acid sequence of SEQ ID NO: 34 (M8), or an amino acid sequence which has at least 99% amino acid identity over its entire length with the amino acid sequence of SEQ ID NO:34.

According to the invention there is also provided an isolated nucleic acid molecule which comprises a nucleotide sequence encoding an isolated polypeptide which comprises a coronavirus S protein RBD domain with at least one of the following amino acid residues at positions corresponding to the amino acid residue positions of SEQ ID NO:11: 13Q, 25Q, 54T, 203N.

According to the invention there is also provided an isolated nucleic acid molecule which comprises a nucleotide sequence encoding an isolated polypeptide comprising an amino acid sequence of SEQ ID NO: 35 (M9), or an amino acid sequence which has at least 70% amino acid identity over its entire length with the amino acid sequence of SEQ ID NO:35.

According to the invention there is also provided an isolated nucleic acid molecule which comprises a nucleotide sequence encoding an isolated polypeptide comprising an amino acid sequence of SEQ ID NO: 36 (M10), or an amino acid sequence which has at least 69% amino acid identity over its entire length with the amino acid sequence of SEQ ID NO:36.

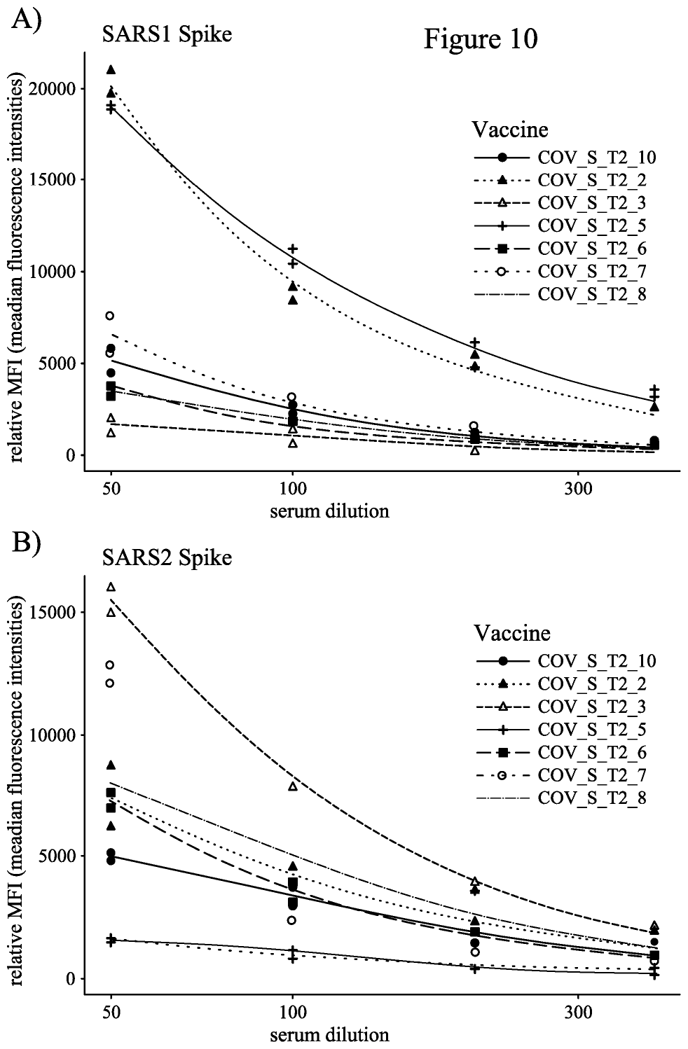

We have found that immunisation of mice with nucleic acid (in particular. DNA) encoding SARS2 truncated S protein induces production of antibodies that are able to bind SARS2 spike protein (see Example 17, FIG. 10).

According to the invention there is provided an isolated nucleic acid molecule encoding a SARS2 truncated S protein of amino acid sequence SEQ ID NO:9 (CoV_T2_3).

Optionally the isolated nucleic acid molecule encoding a SARS2 truncated S protein of amino acid sequence SEQ ID NO:9 (CoV_T2_3) comprises a nucleotide sequence of SEQ ID NO:10.

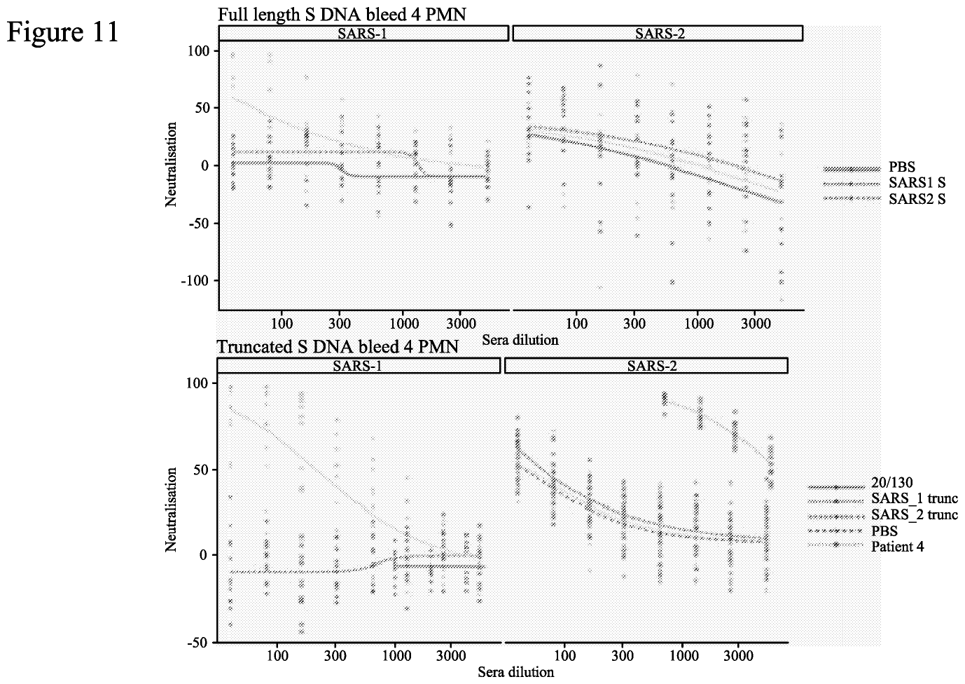

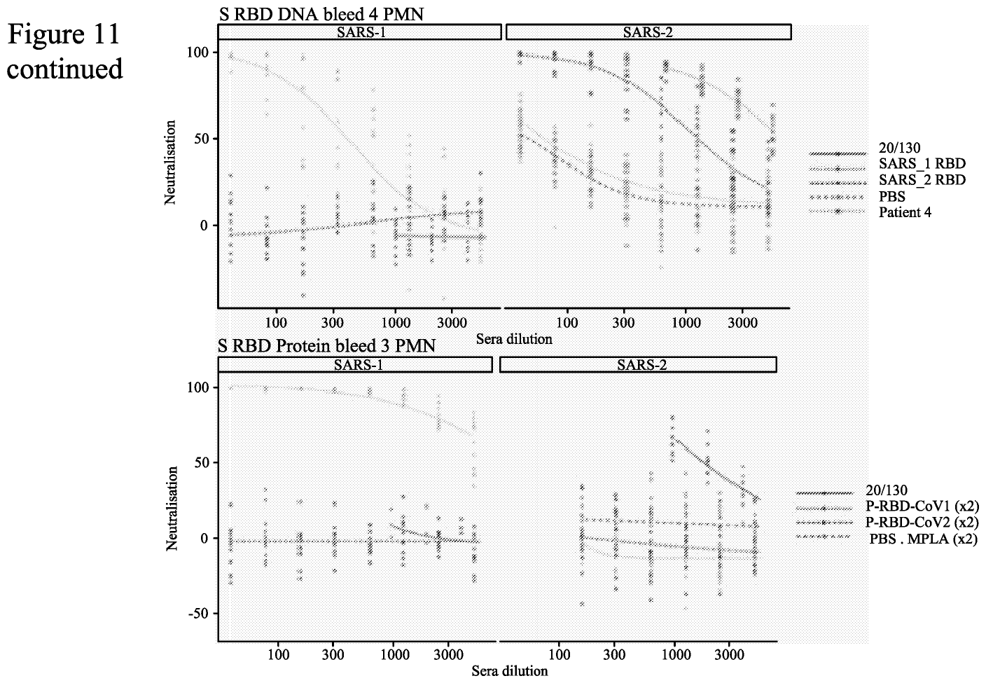

We have also found that immunisation of mice with nucleic acid (in particular, DNA) encoding SARS2 S protein RBD induces production of antibodies that are able to neutralise SARS2 pseudotype virus (see Example 18, FIG. 11).

We have also found that M7 and wild-type SARS2 RBD DNA (believed to result in expression of glycosylated RBD protein) is superior to recombinant SARS2 RBD protein (non-glycosylated, or sparsely glycosylated) in inducing neutralising responses to SARS2.

According to the invention there is provided an isolated nucleic acid molecule encoding a SARS2 S protein RBD of amino acid sequence SEQ ID NO: 11 (CoV_T2_6).

Optionally the isolated nucleic acid molecule encoding a SARS2 S protein RBD of amino acid sequence SEQ ID NO:11 (CoV_T2_6) comprises a nucleotide sequence of SEQ ID NO:12.

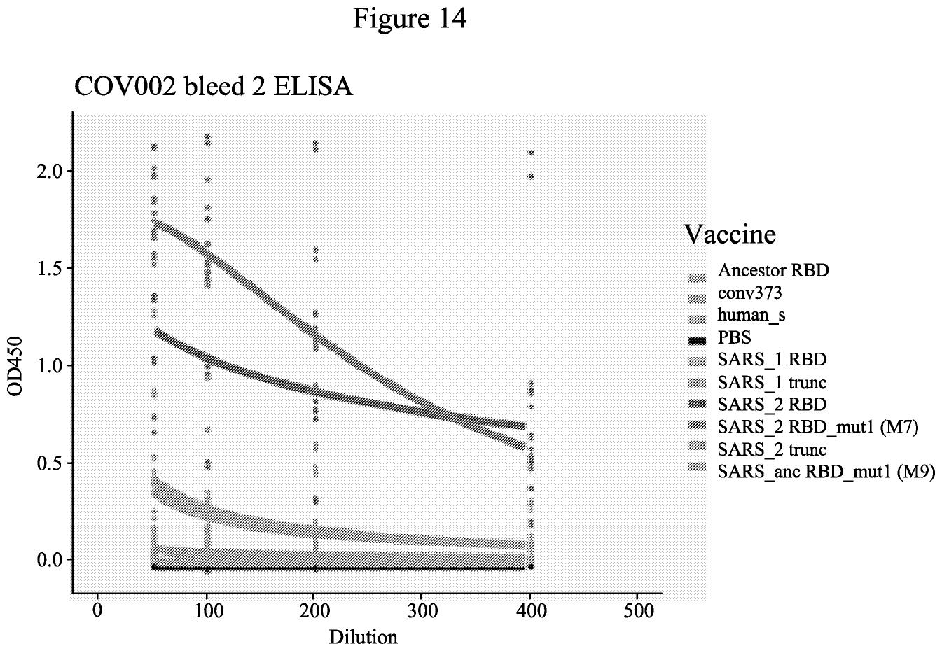

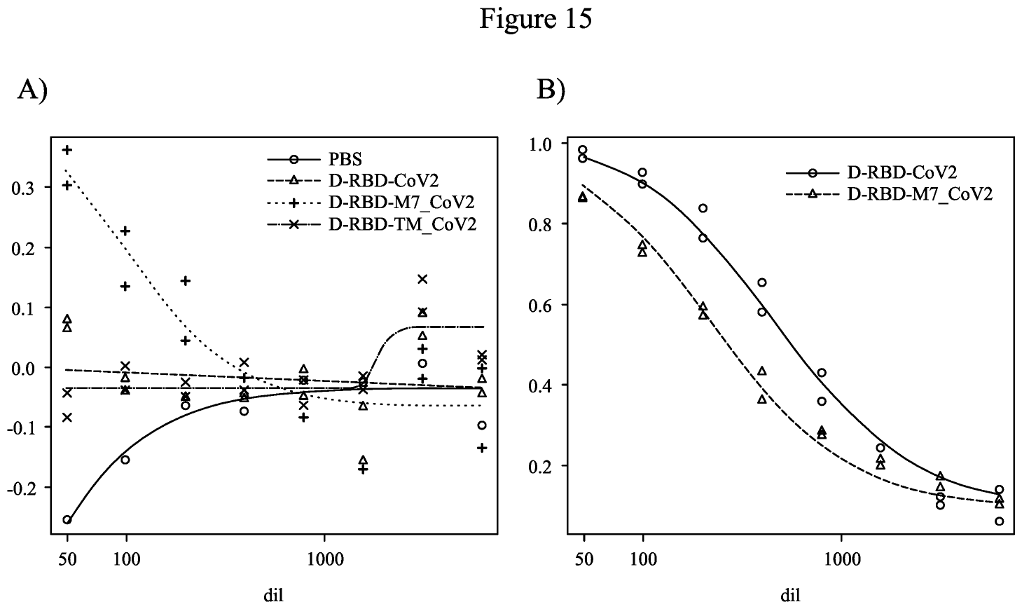

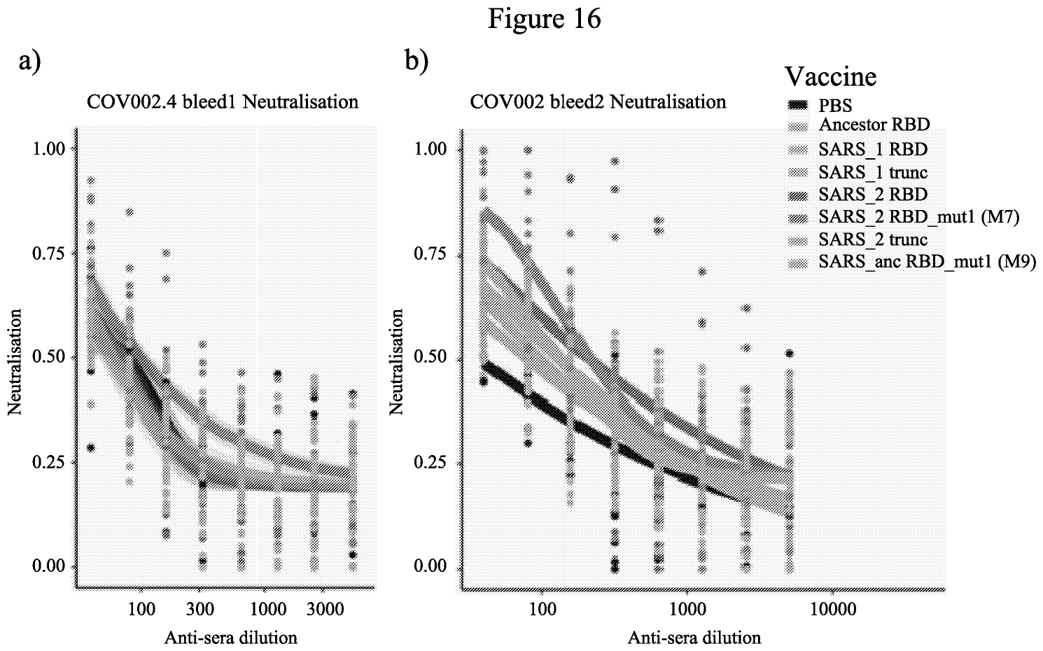

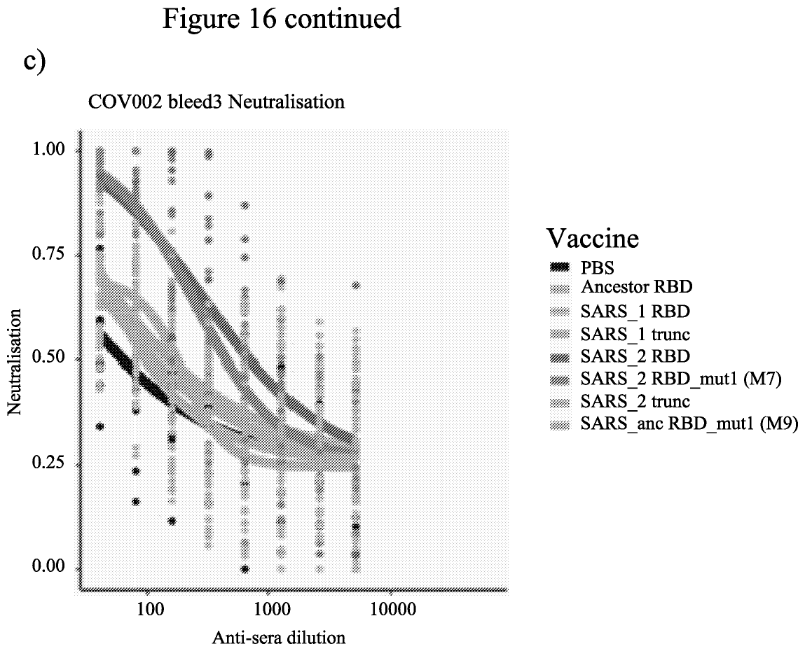

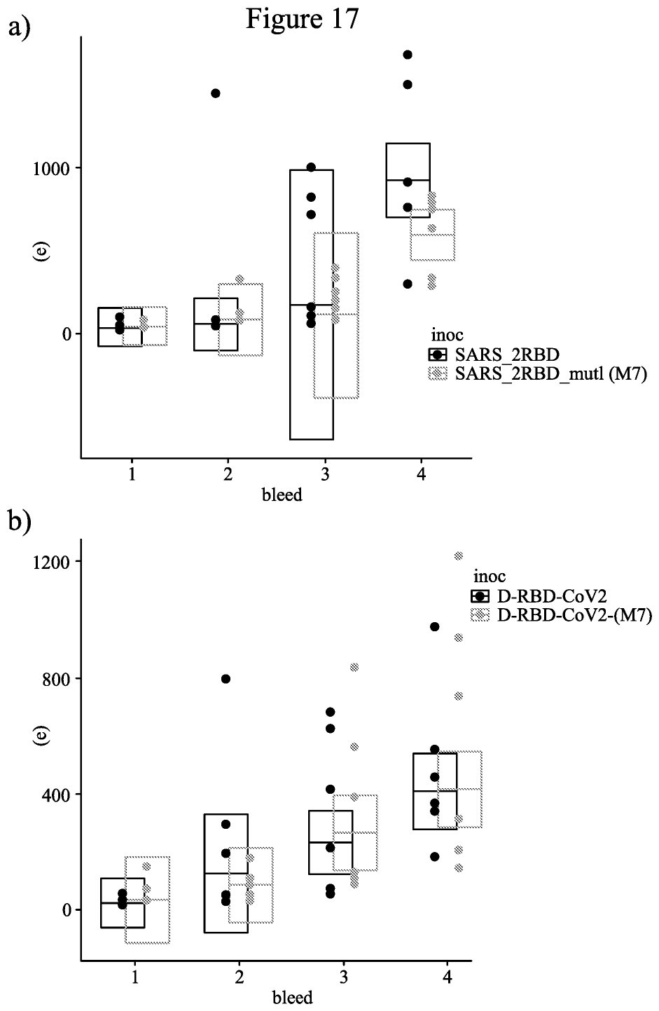

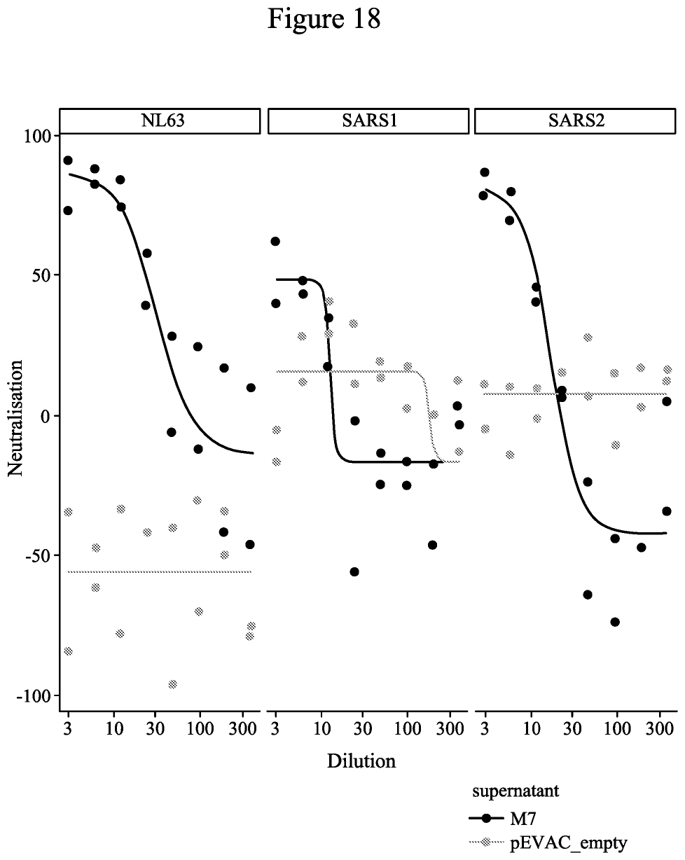

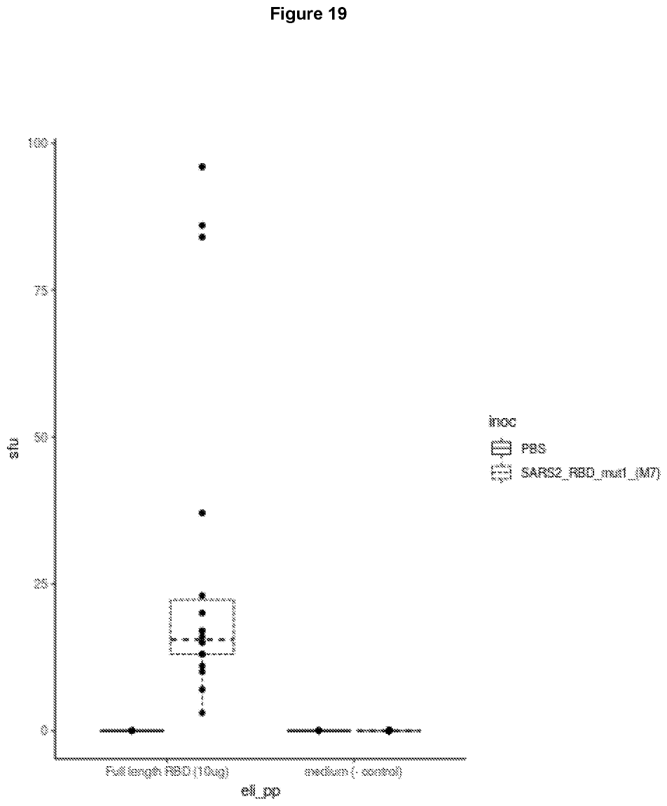

We have also found that nucleic acid (in particular, DNA) encoding the designed M7 SARS2 S protein RBD has especially advantageous effects. In particular, we have found that:
immunisation of mice with a DNA vaccine comprising nucleic acid encoding M7 SARS2 RBD (SEQ ID NO:33) induced an immune response with stronger binding to SARS2 RBD than wild-type SARS2 RBD (see Example 20, and FIG. 14);
immunisation of mice with a DNA vaccine encoding M7 SARS2 RBD (SEQ ID NO:33) elicits a neutralising immune response more rapidly than a DNA vaccine encoding wild-type SARS2 RBD (see Example 21, and FIG. 15);
immunisation of mice with a DNA vaccine encoding M7 SARS2 RBD (SEQ ID NO:33) induces a more neutralising response than a DNA vaccine encoding wild-type SARS2 RBD in sera collected from bleeds at weeks 1 and 2 (see Example 22, and FIGS. 16, 17);
supernatant comprising M7 SARS2 RBD competes effectively with three ACE2 binding viruses for ACE2 cell entry (see Example 23, and FIG. 18); and
T cell responses were induced by a DNA vaccine encoding M7 SARS2 RBD (SEQ ID NO:33) that were reactive against peptides of an RBD peptide pool, but not against full length RBD or medium (see Example 24, and FIG. 19).

There is also provided according to the invention an isolated nucleic acid molecule comprising a nucleotide sequence of SEQ ID NO:37.

### Sequence Identity

The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a given gene or protein will possess a relatively high degree of sequence identity when aligned using standard methods. Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970: Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989: Corpet et al., Nucleic Acids' Research 16:10881-10890, 1988; and Pearson and Lipman, Proc. Natl. Acad. Sci. U.S.A. 85:2444, 1988. Altschul et al., Nature Genet. 6:119-129, 1994. The NCBI Basic Local Alignment Search Tool (BLAST™) (Altschul et al., J. Mol. Biol. 215:403-410, 1990) is available from several sources, including the National Center for Biotechnology Information (NCBI, Bethesda. MD) and on the Internet, for use in connection with the sequence analysis programs blastp, blastn, blastx, tblastn and tblastx.

Sequence identity between nucleic acid sequences, or between amino acid sequences, can be determined by comparing an alignment of the sequences. When an equivalent position in the compared sequences is occupied by the same nucleotide, or amino acid, then the molecules are identical at that position. Scoring an alignment as a percentage of identity is a function of the number of identical nucleotides or amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4: 29; program available from http://bitincka.com/ledion/matgat), Gap (Needleman & Wunsch, 1970, J. Mol. Biol. 48: 443-453), FASTA (Altschul et al., 1990, J. Mol. Biol. 215: 403-410; program available from http://www.ebi.ac.uk/fasta), Clustal W 2.0 and X 2.0 (Larkin et al., 2007, Bioinformatics 23: 2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence.

### Corresponding Positions

Sequences described herein include reference to an amino acid sequence comprising an amino acid residue “at a position corresponding to an amino acid residue position” of another sequence. Such corresponding positions may be identified, for example, from an alignment of the sequences using a sequence alignment method described herein, or another sequence alignment method known to the person of ordinary skill in the art.

### Vectors

There is also provided according to the invention a vector comprising a nucleic acid molecule of the invention.

Optionally a vector of the invention comprises a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:17.

Optionally a vector of the invention comprises a nucleic acid molecule encoding a polypeptide of the invention which comprises an amino acid sequence of SEQ ID NO: 15, or an amino acid sequence which has at least 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:15.

Optionally a vector of the invention comprises a nucleic acid molecule encoding a polypeptide of the invention which comprises an amino acid sequence of SEQ ID NO: 13, or an amino acid sequence which has at least 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:13.

Optionally a vector of the invention comprises a nucleic acid molecule encoding a polypeptide of the invention which comprises an amino acid sequence of SEQ ID NO: 27 (COV_S_T2_13), or an amino acid sequence which has at least 88%, 89%, 90%, 91%, 92%, 93%, 94%, 95%, 96%, 97%, 98%, or 99% amino acid identity over its entire length with the amino acid sequence of SEQ ID NO:27.

Optionally a vector of the invention comprises a nucleic acid molecule encoding a polypeptide of the invention which comprises an amino acid sequence of SEQ ID NO: 28 (COV_S_T2_14), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:28.

Optionally a vector of the invention comprises a nucleic acid molecule encoding a polypeptide of the invention which comprises an amino acid sequence of SEQ ID NO: 29 (COV_S_T2_15), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:29.

Optionally a vector of the invention comprises a nucleic acid molecule encoding a polypeptide of the invention which comprises an amino acid sequence of SEQ ID NO: 30 (COV_S_T2_16), or an amino acid sequence which has at least 93%, 94%, 95%, 96%, 97%, 98%, or 99% amino acid identity over its entire length with the amino acid sequence of SEQ ID NO:30.

Optionally a vector of the invention comprises a nucleic acid molecule encoding a polypeptide of the invention which comprises an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31.

Optionally a vector of the invention comprises a nucleic acid molecule encoding a polypeptide of the invention which comprises an amino acid sequence of SEQ ID NO: 32 (COV_S_T2_18), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:32.

Optionally a vector of the invention comprises a nucleic acid molecule encoding a polypeptide of the invention which comprises an amino acid sequence of SEQ ID NO: 33.

Optionally a vector of the invention comprises a nucleic acid molecule encoding a polypeptide of the invention which comprises an amino acid sequence of SEQ ID NO: 34, or an amino acid sequence which has at least 99% amino acid identity over its entire length with the amino acid sequence of SEQ ID NO:34.

Optionally a vector of the invention comprises a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22.

Optionally a vector of the invention comprises a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23.

Optionally a vector of the invention comprises a nucleic acid molecule encoding a polypeptide of the invention which comprises an amino acid sequence of SEQ ID NO:42 (COV_E_T2_3), or an amino acid sequence which has at least 99% amino acid identity over its entire length with the amino acid sequence of SEQ ID NO:42.

Optionally a vector of the invention comprises a nucleic acid molecule encoding a polypeptide of the invention which comprises an amino acid sequence of SEQ ID NO:43 (COV_E_T2_4), or an amino acid sequence which has at least 95%, 96%, 97%, 98%, or 99% amino acid identity over its entire length with the amino acid sequence of SEQ ID NO:43.

Optionally a vector of the invention comprises a nucleic acid molecule encoding a polypeptide of the invention which comprises an amino acid sequence of SEQ ID NO:44 (COV_E_T2_5), or an amino acid sequence which has at least 98%, or 99% amino acid identity over its entire length with the amino acid sequence of SEQ ID NO:44.

Optionally a vector of the invention comprises a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24.

Optionally a vector of the invention comprises a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a vector of the invention comprises a nucleic acid molecule encoding a polypeptide of the invention which comprises an amino acid sequence of SEQ ID NO:46 (COV_N_T2_1), or an amino acid sequence which has at least 93%, 94%, 95%, 96%, 97%, 98%, or 99% amino acid identity over its entire length with the amino acid sequence of SEQ ID NO:46.

Optionally a vector of the invention comprises a nucleic acid molecule encoding a polypeptide of the invention which comprises an amino acid sequence of SEQ ID NO:47 (COV_N_T2_2), or an amino acid sequence which has at least 92%, 93%, 94%, 95%, 96%, 97%, 98%, or 99% amino acid identity over its entire length with the amino acid sequence of SEQ ID NO:47.

Optionally a vector of the invention further comprises a promoter operably linked to the nucleic acid.

Optionally the promoter is for expression of a polypeptide encoded by the nucleic acid in mammalian cells.

Optionally the promoter is for expression of a polypeptide encoded by the nucleic acid in yeast or insect cells.

Optionally a vector of the invention comprises more than one nucleic acid molecule encoding a different polypeptide of the invention. Advantageously, a vector of the invention comprises a nucleic acid molecule of the invention encoding a designed coronavirus S protein (full length, truncated, or RBD) of the invention and/or a nucleic acid molecule of the invention encoding a designed coronavirus E protein of the invention and/or a nucleic acid molecule of the invention encoding a designed coronavirus M protein of the invention.

Optionally a vector of the invention comprises more than one nucleic acid molecule encoding a different polypeptide of the invention. Advantageously, a vector of the invention comprises a nucleic acid molecule of the invention encoding a designed coronavirus S protein (full length, truncated, or RBD) of the invention and/or a nucleic acid molecule of the invention encoding a designed coronavirus E protein of the invention and/or a nucleic acid molecule of the invention encoding a designed coronavirus M protein of the invention and/or a nucleic acid molecule of the invention encoding a designed coronavirus N protein of the invention Optionally a vector of the invention comprises a nucleic acid molecule of the invention encoding a designed coronavirus S protein (full length, truncated, or RBD) of the invention and a nucleic acid molecule of the invention encoding a designed coronavirus E protein of the invention.

Optionally a vector of the invention comprises a nucleic acid molecule of the invention encoding a designed coronavirus S protein (full length, truncated, or RBD) of the invention and a nucleic acid molecule of the invention encoding a designed coronavirus M protein of the invention.

Optionally a vector of the invention comprises a nucleic acid molecule of the invention encoding a designed coronavirus S protein (full length, truncated, or RBD) of the invention and a nucleic acid molecule of the invention encoding a designed coronavirus N protein of the invention.

Optionally a vector of the invention comprises a nucleic acid molecule of the invention encoding a designed coronavirus E protein of the invention and a nucleic acid molecule of the invention encoding a designed coronavirus M protein of the invention.

Optionally a vector of the invention comprises a nucleic acid molecule of the invention encoding a designed coronavirus E protein of the invention and a nucleic acid molecule of the invention encoding a designed coronavirus N protein of the invention.

Optionally a vector of the invention comprises a nucleic acid molecule of the invention encoding a designed coronavirus S protein (full length, truncated, or RBD) of the invention and a nucleic acid molecule of the invention encoding a designed coronavirus E protein of the invention and a nucleic acid molecule of the invention encoding a designed coronavirus M protein of the invention.

Optionally a vector of the invention comprises a nucleic acid molecule of the invention encoding a designed coronavirus S protein (full length, truncated, or RBD) of the invention and a nucleic acid molecule of the invention encoding a designed coronavirus E protein of the invention and a nucleic acid molecule of the invention encoding a designed coronavirus N protein of the invention.

Optionally a vector of the invention comprises a nucleic acid molecule of the invention encoding a designed coronavirus E protein of the invention and a nucleic acid molecule of the invention encoding a designed coronavirus M protein of the invention and a nucleic acid molecule of the invention encoding a designed coronavirus N protein of the invention.

Optionally a vector of the invention comprises a nucleic acid molecule of the invention encoding a designed coronavirus S protein (full length, truncated, or RBD) of the invention and a nucleic acid molecule of the invention encoding a designed coronavirus E protein of the invention and a nucleic acid molecule of the invention encoding a designed coronavirus M protein of the invention and a nucleic acid molecule of the invention encoding a designed coronavirus N protein of the invention.

Optionally a vector of the invention comprises:
a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:17; and
a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23.

Optionally a vector of the invention comprises:
a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:17; and
a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a vector of the invention comprises:
a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23; and
a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a vector of the invention comprises:
a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:17; and
a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23; and
a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a vector of the invention which further comprises, for each nucleic acid molecule of the vector encoding a polypeptide, a separate promoter operably linked to that nucleic acid molecule.

Optionally the, or each promoter is for expression of a polypeptide encoded by the nucleic acid molecule in mammalian cells.

Optionally the, or each promoter is for expression of a polypeptide encoded by the nucleic acid molecule in yeast or insect cells.

Optionally the vector is a vaccine vector.

Optionally the vector is a viral vaccine vector, a bacterial vaccine vector, an RNA vaccine vector, or a DNA vaccine vector.

A nucleic acid molecule of the invention may comprise a DNA or an RNA molecule. For embodiments in which the nucleic acid molecule comprises an RNA molecule, it will be appreciated that the molecule may comprise an RNA sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with, or identical with, any of SEQ ID NOs: 18, 16, or 14, in which each ‘T’ nucleotide is replaced by ‘U’, or the complement thereof.

For example, it will be appreciated that where an RNA vaccine vector comprising a nucleic acid of the invention is provided, the nucleic acid sequence of the nucleic acid of the invention will be an RNA sequence, so may comprise for example an RNA nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with, or identical with, any of SEQ ID NOs: 18, 16, or 14 in which each ‘T’ nucleotide is replaced by ‘U’, or the complement thereof.

Viral vaccine vectors use live viruses to deliver nucleic acid (for example, DNA or RNA) into human or non-human animal cells. The nucleic acid contained in the virus encodes one or more antigens that, once expressed in the infected human or non-human animal cells, elicit an immune response. Both humoral and cell-mediated immune responses can be induced by viral vaccine vectors. Viral vaccine vectors combine many of the positive qualities of nucleic acid vaccines with those of live attenuated vaccines. Like nucleic acid vaccines, viral vaccine vectors carry nucleic acid into a host cell for production of antigenic proteins that can be tailored to stimulate a range of immune responses, including antibody, T helper cell (CD4+ T cell), and cytotoxic T lymphocyte (CTL, CD8+ T cell) mediated immunity. Viral vaccine vectors, unlike nucleic acid vaccines, also have the potential to actively invade host cells and replicate, much like a live attenuated vaccine, further activating the immune system like an adjuvant. A viral vaccine vector therefore generally comprises a live attenuated virus that is genetically engineered to carry nucleic acid (for example, DNA or RNA) encoding protein antigens from an unrelated organism. Although viral vaccine vectors are generally able to produce stronger immune responses than nucleic acid vaccines, for some diseases viral vectors are used in combination with other vaccine technologies in a strategy called heterologous prime-boost. In this system, one vaccine is given as a priming step, followed by vaccination using an alternative vaccine as a booster. The heterologous prime-boost strategy aims to provide a stronger overall immune response. Viral vaccine vectors may be used as both prime and boost vaccines as part of this strategy. Viral vaccine vectors are reviewed by Ura et al., 2014 (Vaccines 2014, 2, 624-641) and Choi and Chang, 2013 (Clinical and Experimental Vaccine Research 2013; 2:97-105).

Optionally the viral vaccine vector is based on a viral delivery vector, such as a Poxvirus (for example, Modified Vaccinia Ankara (MVA), NYVAC, AVIPOX), herpesvirus (e.g. HSV, CMV, Adenovirus of any host species), Morbillivirus (e.g. measles). Alphavirus (e.g. SFV. Sendai), Flavivirus (e.g. Yellow Fever), or Rhabdovirus (e.g. VSV)-based viral delivery vector, a bacterial delivery vector (for example, Salmonella, E. coli), an RNA expression vector, or a DNA expression vector.

Optionally the nucleic acid expression vector is a nucleic acid expression vector, and a viral pseudotype vector.

Optionally the nucleic acid expression vector is a vaccine vector.

Optionally the nucleic acid expression vector comprises, from a 5′ to 3′ direction: a promoter; a splice donor site (SD); a splice acceptor site (SA); and a terminator signal, wherein the multiple cloning site is located between the splice acceptor site and the terminator signal.

Optionally the promoter comprises a CMV immediate early 1 enhancer/promoter (CMV-IE-E/P) and/or the terminator signal comprises a terminator signal of a bovine growth hormone gene (Tbgh) that lacks a KpnI restriction endonuclease site.

Optionally the nucleic acid expression vector further comprises an origin of replication, and nucleic acid encoding resistance to an antibiotic. Optionally the origin of replication comprises a pUC-plasmid origin of replication and/or the nucleic acid encodes resistance to kanamycin.

Optionally the vector is a pEVAC-based expression vector.

Optionally the nucleic acid expression vector comprises a nucleic acid sequence of SEQ ID NO:20 (pEVAC). The pEVAC vector has proven to be a highly versatile expression vector for generating viral pseudotypes as well as direct DNA vaccination of animals and humans. The pEVAC expression vector is described in more detail in Example 8 below. FIG. 3 shows a plasmid map for pEVAC.

There is also provided according to the invention an isolated cell comprising or transfected with a vector of the invention.

There is also provided according to the invention a fusion protein comprising a polypeptide of the invention.

### Pharmaceutical Compositions

According to the invention there is also provided a pharmaceutical composition comprising a polypeptide of the invention, and a pharmaceutically acceptable carrier, excipient, or diluent.

Optionally a pharmaceutical composition of the invention comprises more than one different polypeptide of the invention.

Advantageously, a pharmaceutical composition of the invention comprises a designed coronavirus S protein (full length, truncated, or RBD) of the invention and/or a designed coronavirus E protein of the invention and/or a designed coronavirus M protein of the invention.

Advantageously, a pharmaceutical composition of the invention comprises a designed coronavirus S protein (full length, truncated, or RBD) of the invention and/or a designed coronavirus E protein of the invention and/or a designed coronavirus M protein of the invention and/or a designed coronavirus N protein of the invention.

Optionally a pharmaceutical composition of the invention comprises a designed coronavirus S protein (full length, truncated, or RBD) of the invention and a designed coronavirus E protein of the invention.

Optionally a pharmaceutical composition of the invention comprises a designed coronavirus S protein (full length, truncated, or RBD) of the invention and a designed coronavirus M protein of the invention.

Optionally a pharmaceutical composition of the invention comprises a designed coronavirus S protein (full length, truncated, or RBD) of the invention and a designed coronavirus N protein of the invention.

Optionally a pharmaceutical composition of the invention comprises a designed coronavirus E protein of the invention and a designed coronavirus M protein of the invention.

Optionally a pharmaceutical composition of the invention comprises a designed coronavirus E protein of the invention and a designed coronavirus N protein of the invention.

Optionally a pharmaceutical composition of the invention comprises a designed coronavirus S protein (full length, truncated, or RBD) of the invention and a designed coronavirus E protein of the invention and a designed coronavirus M protein of the invention.

Optionally a pharmaceutical composition of the invention comprises a designed coronavirus S protein (full length, truncated, or RBD) of the invention and a designed coronavirus E protein of the invention and a designed coronavirus N protein of the invention.

Optionally a pharmaceutical composition of the invention comprises a designed coronavirus E protein of the invention and a designed coronavirus M protein of the invention and a designed coronavirus N protein of the invention.

Optionally a pharmaceutical composition of the invention comprises:
a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:17; and
a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a polypeptide of the invention which comprises an amino acid sequence of SEQ ID NO:23, or an amino acid sequence which has at least 98%, or 99% amino acid identity over its entire length with the amino acid sequence of SEQ ID NO:23.

Optionally a pharmaceutical composition of the invention comprises:
a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:17; and
a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a pharmaceutical composition of the invention comprises:
a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a polypeptide of the invention which comprises an amino acid sequence of SEQ ID NO:23, or an amino acid sequence which has at least 98%, or 99% amino acid identity over its entire length with the amino acid sequence of SEQ ID NO:23; and
a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a pharmaceutical composition of the invention comprises:
a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:17; and
a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a polypeptide of the invention which comprises an amino acid sequence of SEQ ID NO:23, or an amino acid sequence which has at least 98%, or 99% amino acid identity over its entire length with the amino acid sequence of SEQ ID NO:23; and
a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

According to the invention there is also provided a pharmaceutical composition comprising a nucleic acid of the invention, and a pharmaceutically acceptable carrier, excipient, or diluent.

Optionally a pharmaceutical composition of the invention comprises more than one nucleic acid molecule of the invention encoding a different polypeptide of the invention.

Advantageously, a pharmaceutical composition of the invention comprises a nucleic acid molecule of the invention encoding a designed coronavirus S protein (full length, truncated, or RBD) of the invention and/or a nucleic acid molecule of the invention encoding a designed coronavirus E protein of the invention and/or a nucleic acid molecule of the invention encoding a designed coronavirus M protein of the invention.

Advantageously, a pharmaceutical composition of the invention comprises a nucleic acid molecule of the invention encoding a designed coronavirus S protein (full length, truncated, or RBD) of the invention and/or a nucleic acid molecule of the invention encoding a designed coronavirus E protein of the invention and/or a nucleic acid molecule of the invention encoding a designed coronavirus M protein of the invention and/or a nucleic acid molecule of the invention encoding a designed coronavirus N protein of the invention.

Optionally a pharmaceutical composition of the invention comprises a nucleic acid molecule of the invention encoding a designed coronavirus S protein (full length, truncated, or RBD) of the invention and a nucleic acid molecule of the invention encoding a designed coronavirus E protein of the invention.

Optionally a pharmaceutical composition of the invention comprises a nucleic acid molecule of the invention encoding a designed coronavirus S protein (full length, truncated, or RBD) of the invention and a nucleic acid molecule of the invention encoding a designed coronavirus M protein of the invention.

Optionally a pharmaceutical composition of the invention comprises a nucleic acid molecule of the invention encoding a designed coronavirus S protein (full length, truncated, or RBD) of the invention and a nucleic acid molecule of the invention encoding a designed coronavirus N protein of the invention.

Optionally a pharmaceutical composition of the invention comprises a nucleic acid molecule of the invention encoding a designed coronavirus E protein of the invention and a nucleic acid molecule of the invention encoding a designed coronavirus M protein of the invention.

Optionally a pharmaceutical composition of the invention comprises a nucleic acid molecule of the invention encoding a designed coronavirus E protein of the invention and a nucleic acid molecule of the invention encoding a designed coronavirus N protein of the invention.

Optionally a pharmaceutical composition of the invention comprises a nucleic acid molecule of the invention encoding a designed coronavirus S protein (full length, truncated, or RBD) of the invention and a nucleic acid molecule of the invention encoding a designed coronavirus E protein of the invention and a nucleic acid molecule of the invention encoding a designed coronavirus M protein of the invention.

Optionally a pharmaceutical composition of the invention comprises a nucleic acid molecule of the invention encoding a designed coronavirus S protein (full length, truncated, or RBD) of the invention and a nucleic acid molecule of the invention encoding a designed coronavirus E protein of the invention and a nucleic acid molecule of the invention encoding a designed coronavirus N protein of the invention.

Optionally a pharmaceutical composition of the invention comprises a nucleic acid molecule of the invention encoding a designed coronavirus E protein of the invention and a nucleic acid molecule of the invention encoding a designed coronavirus M protein of the invention and a nucleic acid molecule of the invention encoding a designed coronavirus N protein of the invention.

Optionally a pharmaceutical composition of the invention comprises:
a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:17; and
a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23.

Optionally a pharmaceutical composition of the invention comprises:
a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:17; and
a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a pharmaceutical composition of the invention comprises:
a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23; and
a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

Optionally a pharmaceutical composition of the invention comprises:
a nucleic acid molecule encoding a polypeptide of the invention which comprises an amino acid sequence of SEQ ID NO: 17, or an amino acid sequence which has at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over its entire length with the amino acid sequence of SEQ ID NO:17; and
a nucleic acid molecule encoding a polypeptide of the invention which comprises an amino acid sequence of SEQ ID NO:22, or an amino acid sequence which has at least 95%, 96%, 97%, 98%, or 99% amino acid identity over its entire length with the amino acid sequence of SEQ ID NO:22, or a nucleic acid molecule encoding a polypeptide of the invention which comprises an amino acid sequence of SEQ ID NO:23, or an amino acid sequence which has at least 98%, or 99% amino acid identity over its entire length with the amino acid sequence of SEQ ID NO:23; and
a nucleic acid molecule encoding a polypeptide of the invention which comprises an amino acid sequence of SEQ ID NO:24, or an amino acid sequence which has at least 91%, 92%, 93%, 94%, 95%, 96%, 97%, 98%, or 99% amino acid identity over its entire length with the amino acid sequence of SEQ ID NO:24, or a nucleic acid molecule encoding a polypeptide of the invention which comprises an amino acid sequence of SEQ ID NO:25, or an amino acid sequence which has at least 95%, 96%, 97%, 98%, or 99% amino acid identity over its entire length with the amino acid sequence of SEQ ID NO:25.

According to the invention there is also provided a pharmaceutical composition comprising a vector of the invention, and a pharmaceutically acceptable carrier, excipient, or diluent.

Optionally a pharmaceutical composition of the invention further comprises an adjuvant for enhancing an immune response in a subject to the polypeptide, or to a polypeptide encoded by the nucleic acid, of the composition.

Optionally a pharmaceutical composition of the invention further comprises an adjuvant for enhancing an immune response in a subject to the polypeptides, or to polypeptides encoded by the nucleic acids, of the composition.

There is also provided according to the invention a pseudotyped virus comprising a polypeptide of the invention.

### Methods of Treatment and Uses

There is also provided according to the invention a method of inducing an immune response to a coronavirus in a subject, which comprises administering to the subject an effective amount of a polypeptide of the invention, a nucleic acid of the invention, a vector of the invention, or a pharmaceutical composition of the invention.

There is also provided according to the invention a method of immunising a subject against a coronavirus, which comprises administering to the subject an effective amount of a polypeptide of the invention, a nucleic acid of the invention, a vector of the invention, or a pharmaceutical composition of the invention.

There is further provided according to the invention a polypeptide of the invention, a nucleic acid of the invention, a vector of the invention, or a pharmaceutical composition of the invention, for use as a medicament.

There is further provided according to the invention a polypeptide of the invention, a nucleic acid of the invention, a vector of the invention, or a pharmaceutical composition of the invention, for use in the prevention, treatment, or amelioration of a coronavirus infection.

There is also provided according to the invention use of a polypeptide of the invention, a nucleic acid of the invention, a vector of the invention, or a pharmaceutical composition of the invention, in the manufacture of a medicament for the prevention, treatment, or amelioration of a coronavirus infection.

Optionally the coronavirus is a β-coronavirus.

Optionally the β-coronavirus is a lineage B or C β-coronavirus.

Optionally the β-coronavirus is a lineage B β-coronavirus.

Optionally the lineage B β-coronavirus is SARS-CoV or SARS-CoV-2.

Optionally the lineage C β-coronavirus is MERS-CoV.

### Administration

Any suitable route of administration may be used. Methods of administration include, but are not limited to, intradermal, intramuscular, intraperitoneal, parenteral, intravenous, subcutaneous, vaginal, rectal, intranasal, inhalation or oral. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local.

Compositions may be administered in any suitable manner, such as with pharmaceutically acceptable carriers. Pharmaceutically acceptable carriers are determined in part by the particular composition being administered, as well as by the particular method used to administer the composition. Preparations for parenteral administration include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Administration can be accomplished by single or multiple doses. The dose administered to a subject in the context of the present disclosure should be sufficient to induce a beneficial therapeutic response in a subject overtime, or to inhibit or prevent infection. The dose required will vary from subject to subject depending on the species, age, weight and general condition of the subject, the severity of the infection being treated, the particular composition being used and its mode of administration. An appropriate dose can be determined by one of ordinary skill in the art using only routine experimentation.

### Pharmaceutically Acceptable Carriers

Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, and magnesium carbonate. Any of the common pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used with the compositions and methods provided herein are normal saline and sesame oil.

In some embodiments, the compositions comprise a pharmaceutically acceptable carrier and/or an adjuvant. For example, the adjuvant can be alum. Freund's complete adjuvant, a biological adjuvant or immunostimulatory oligonucleotides (such as CpG oligonucleotides).

The pharmaceutically acceptable carriers (vehicles) useful in this disclosure are conventional. Remington's Pharmaceutical Sciences, by E. W. Martin, Mack Publishing Co., Easton, ^{PA}, 15th Edition (1975), describes compositions and formulations suitable for pharmaceutical delivery of one or more therapeutic compositions, such as one or more influenza vaccines, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Optionally a polypeptide, nucleic acid, or composition of the invention is administered intramuscularly.

Optionally a polypeptide, nucleic acid, or composition of the invention is administered intramuscularly, intradermally, subcutaneously by needle or by gene gun, or electroporation.

Embodiments of the invention are now described, by way of example only, with reference to the accompanying drawings, in which:

FIG. 1 shows SARS S-protein architecture;

FIG. 2 shows a multiple sequence alignment of the S-protein (region around the S1 cleavage site) comparing SARS-CoV-1 isolate (SEQ ID NO:88) and closely related bat betacoronavirus Isolate (SEQ ID NO:87) with four SARS-CoV-2 Isolates (SEQ ID NO:88-91), and a consensus sequence (SEQ ID NO:92);

FIG. 3 shows a plasmid map for pEVAC DNA vector:

FIG. 4 shows Wuhan_Node1_RBD (CoV_T2_7) amino acid sequence (SEQ ID NO:17) with amino acid residue differences highlighted in bold and underline from the respective alignments with AY274119_RBD (CoV_T2_5) (SEQ ID NO:5) and EPI_ISL_402119_RBD (CoV_T2_6) (SEQ ID NO:11) amino acid sequences. Common differences from the two alignments are shown highlighted in grey. Amino acid Insertions are shown boxed;

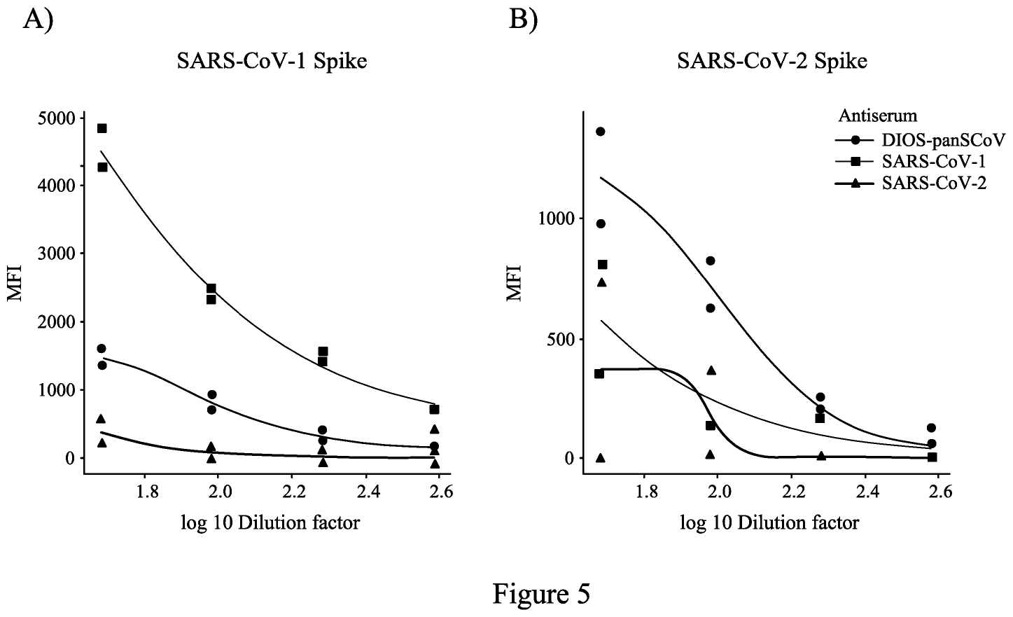

FIG. 5 shows dose response curves of antibody binding to full length Spike protein of SARS-CoV-1, or SARS-CoV-2 expressed on HEK293T cells. Flow cytometry based cell display assay reported in MFI (Median Fluorescent Intensity). In the left hand figure, the upper to lower curves are SARS-CoV-1, DIOS-panSCoV, SARS-CoV2; in the right hand figure, the upper to lower curves are DIOS-panSCoV, SARS-CoV-1. SARS-CoV2;

FIG. 6 shows coronavirus SARS Envelope protein sequence (SEQ ID NO:21), and its significant elements;

FIG. 7 shows a multiple sequence alignment of coronavirus Envelope protein sequences, comparing sequences for Isolates of NL63 (SEQ ID NO:93), 229E (SEQ ID NO:94), HKU1 (SEQ ID NOs:95-96), MERS (SEQ ID NO:97), SARS (SEQ ID NO:21), and SARS2 (SEQ ID NO:41), and consensus E protein sequences (SEQ ID NOs: 98-100);

FIG. 8 shows a multiple sequence alignment of coronavirus Membrane (M) protein sequences, comparing sequences for a SARS2 reference sequence (isolate NC_045512.2) against CoV_M_T2_1 (Sarbeco_M_root) and CoV_M_T2_2 (Sarbeco_M_Node88b_epktope_optimised);

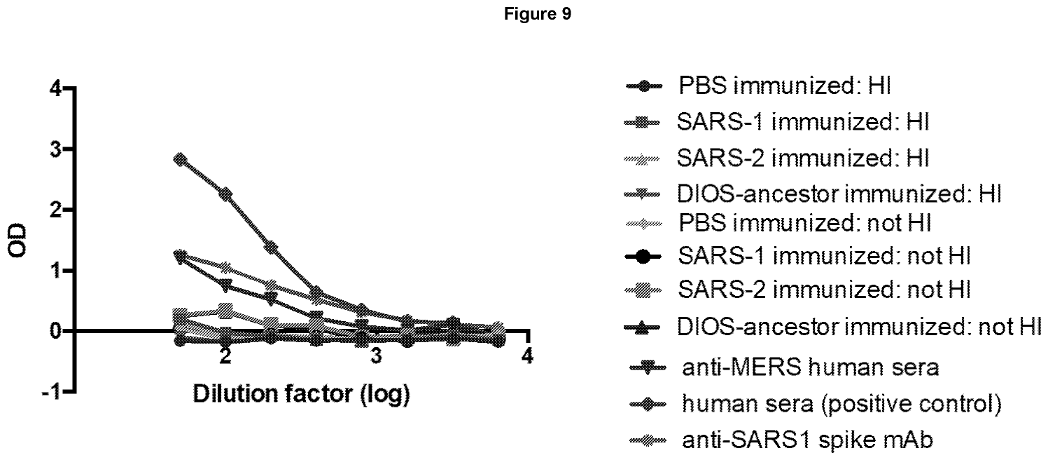

FIG. 9 shows binding (by ELISA) of mouse sera, collected following immunisation of mice with different full-length S protein genes, to SARS2 RBD:

FIG. 10 shows binding by FACS of mouse sera, collected following immunisation of mice with different DNA vaccines, to SARS1 spike protein and SARS2 spike protein;

FIG. 11 shows the ability of DNA vaccines encoding wild-type SARS1 or SARS2 spike protein (full-length, truncated, or RBD) to induce a neutralisation response to SARS1 and SARS2 pseudotypes—the only SARS2 immunogen which induces SARS2 pseudotype neutralising antibodies is the DNA encoding SARS2 RBD;

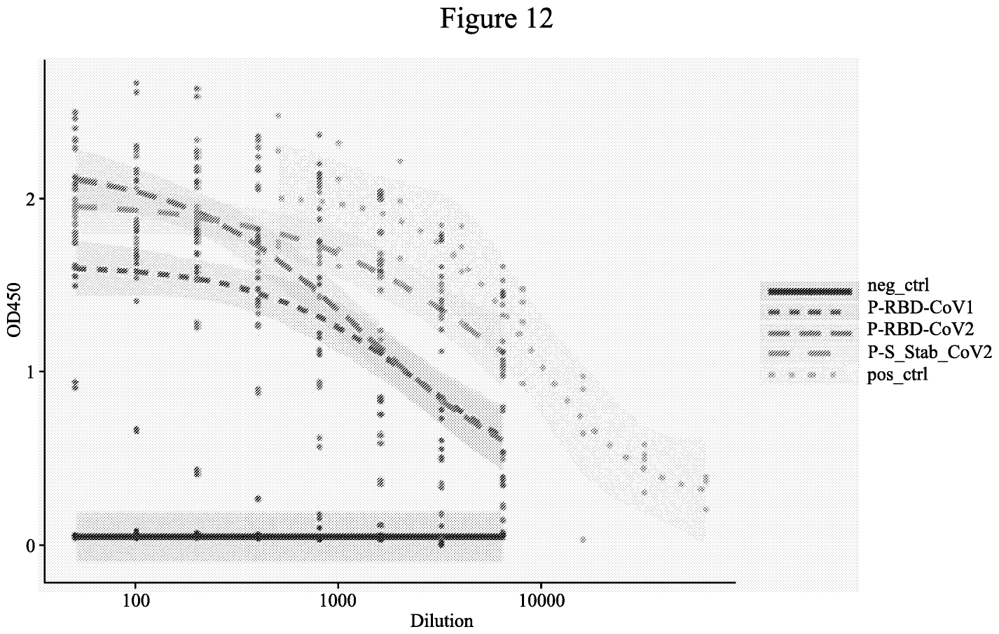

FIG. 12 shows the ability of SARS1 and SARS2 RBD protein vaccines to induce antibodies to SARS2 RBD;

FIG. 13 illustrates new RBD antigen designs based on the amino acid sequence of the RBD region (SEQ ID NO:106);

FIG. 14 shows the ability of different S protein RBD DNA vaccines to induce antibodies to SARS2 RBD—M7 DNA vaccine induces a stronger binding response (by ELISA) to SARS2 RBD than wild-type SARS2 RBD DNA vaccine (the uppermost curve, from the left hand end of the figure, is for SARS_2 RBD_mut1 (M7), the next curve down is for SARS_2 RBD);

FIG. 15 shows the results of a competition assay for inhibition of RBD-ACE2 interaction by sera collected following immunisation with M7 and wild-type SARS2 RBD DNA vaccines—the results show that M7 RBD DNA vaccine elicits a faster neutralisation response than wild-type RBD DNA vaccine;

FIG. 16 shows a SARS2 pseudotype neutralisation response induced by M7 and wild-type SARS2 RBD DNA vaccines: FIG. 16(a) bleed at week 2 from the immunised mice, FIG. 16(b) bleed at week 3 from the immunised mice, and FIG. 16(c) bleed at week 4 from the immunised mice—M7 is more neutralising in the early stages (the uppermost curve, from the left hand end of FIGS. 16 (a), (b), (c), is for SARS2 RBD_mut1 (M7), the next curve down is for SARS_2 RBD);

FIG. 17 shows SARS2 pseudotype neutralisation IC₅₀ values for sera collected from the mice immunised with wild-type SARS2 RBD DNA vaccine, and M7 SARS2 RBD DNA vaccine. The dots in FIG. 17 show IC₅₀ values for individual mice, and the horizontal cross bars show the estimate based on all mice with 95% confidence intervals;

FIG. 18 shows that the supernatant of cells expressing M7 competes with other ACE2 binding viruses for ACE2 cell entry;

FIG. 19 shows the results of an ELISPOT assay showing T cell response to M7 SARS2 RBD DNA vaccine;

FIG. 20 shows an illustration of the M protein (SEQ ID NO:101), and its significant elements;

FIG. 21 shows the spectra overlap (MALDI MS) of supernatants derived from HEK cells transfected with pEVAC plasmid encoding S protein RBD sequences;

FIG. 22 shows spectra for recombinant RBD proteins;

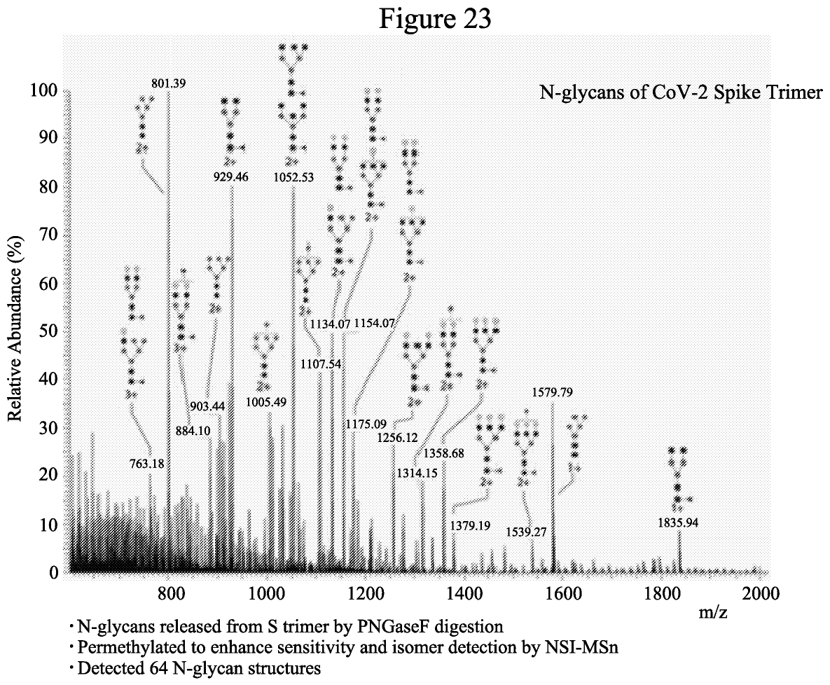

FIG. 23 provides a reference for glycosylation of the S protein;

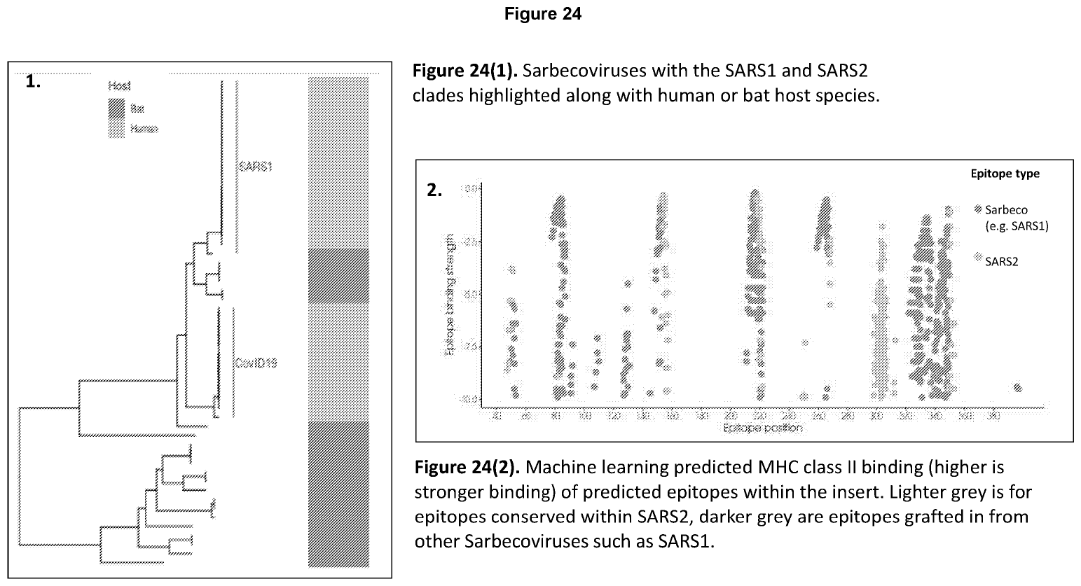

FIG. 24 shows coronavirus vaccine pan-Sarbecovirus vaccine coverage. Pan-Sarbecovirus protection: Beta-Coronaviruses including SARS-CoV-2 (SARS2), -1 (SARS1) & the many ACE2 receptor using Bat SARSr-CoV that threaten to spillover into humans. Antigenic coverage achieved by universal Sarbecovirus B-cell and T-cell antigen targets: Part 1. Sarbecoviruses with the SARS1 and SARS2 clades highlighted along with human or bat host species. Part 2. Machine learning predicted MHC class II binding (higher is stronger binding) of predicted epitopes within the insert. Lighter grey is for epitopes conserved within SARS2, darker grey are epitopes grafted in from other Sarbecoviruses such as SARS1;

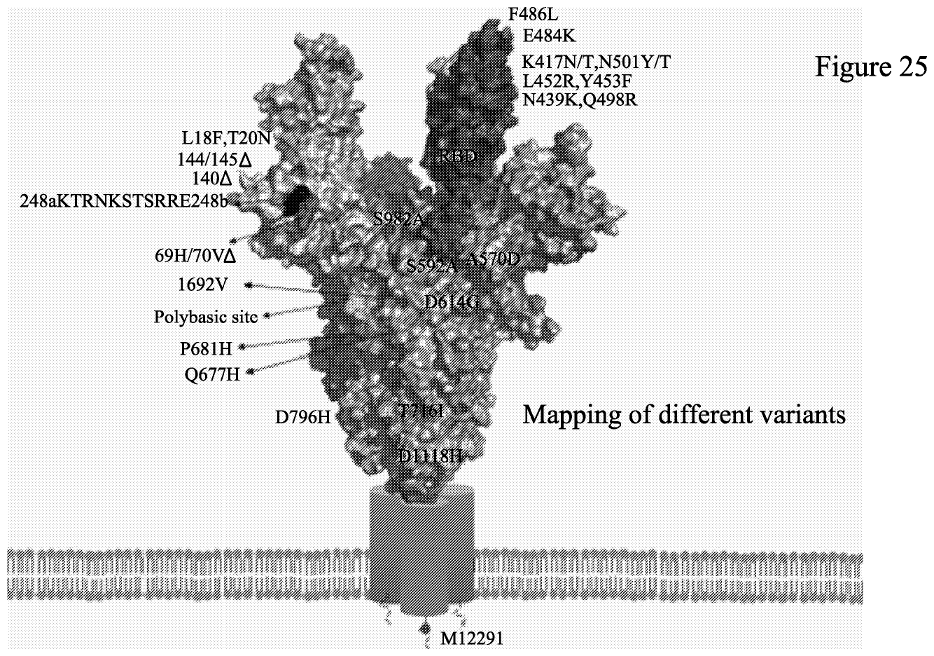

FIG. 25 illustrates mapping of different SARS-CoV-2 variants:

Inclusive list of all the important variants: Pink=exposed mutation; Black=insertion; Yellow=partially buried or fully buried; Purple=in the cytoplasmic tail; Blue colour=RBD; Wheat colour=NTD;

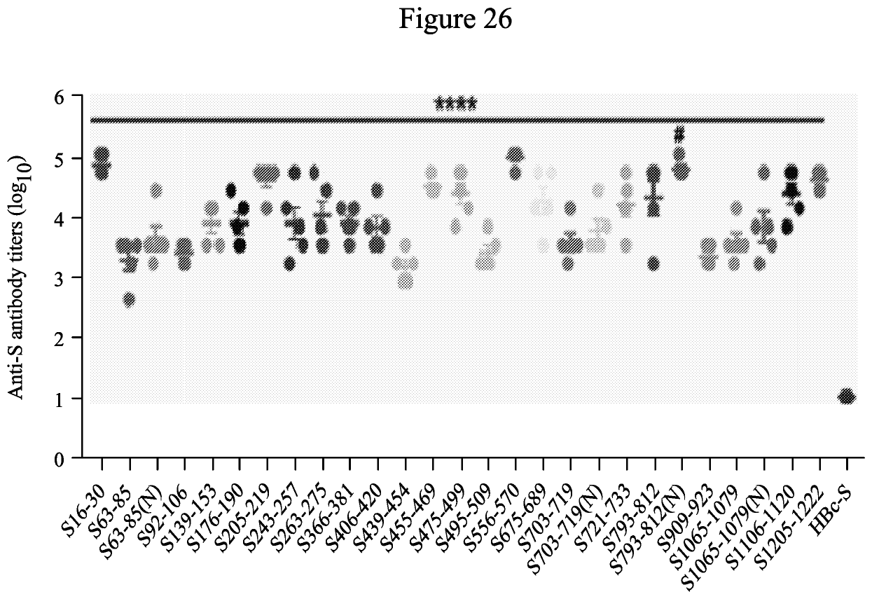

FIG. 26 shows the immunodominant and neutralization linear epitopes for SARS-CoV-2:

| EpitopesEpitopes | Variant | Immuno-dominant* |
|---|---|---|
| 16-30 | Japan | Yes |
| 92-106 | | |
| 139-153 | UK, Japan | |
| 243-257 | | |
| 406-420 | Japan, South Africa | |
| 439-454 | | |
| 455-499 | Japan, South Africa | Yes |
| 556-570 | UK | Yes |
| 675-689 | UK | |
| 721-733 | | Yes |

Study limited to Chinese population. Expressed peptides as VSV.
*Against G614 variant

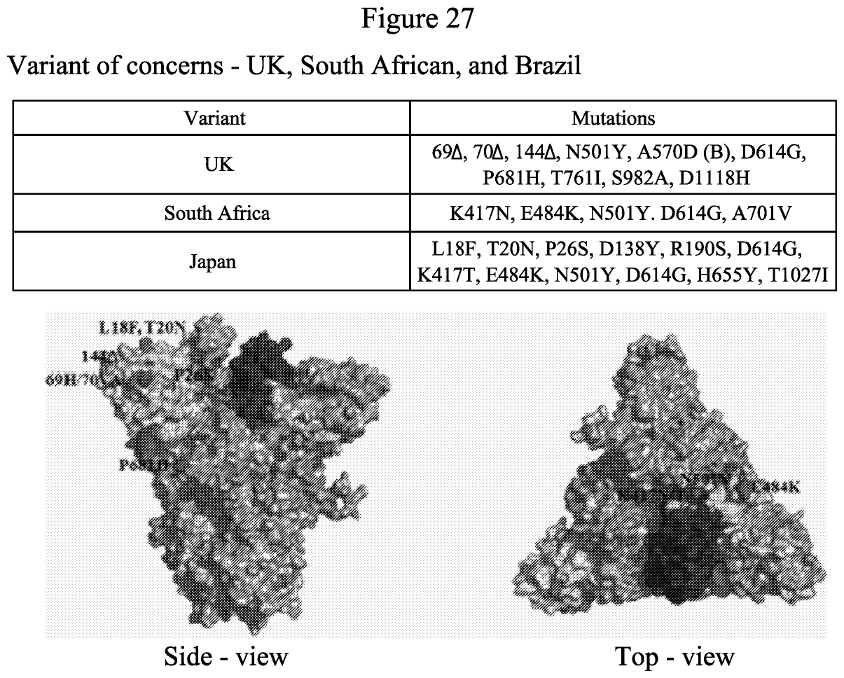

FIG. 27 contains a table describing the mutations in the variants of concern (UK, South African, and Brazil), and structural figures with immunodominant epitope coloured teal and mutations shown in red. RBD—Blue; NTD—wheat;

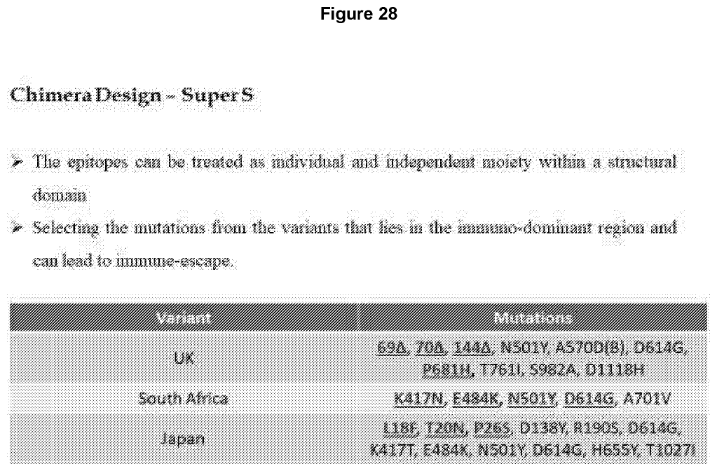

FIG. 28 explains the chimeric design of a super spike protein according to an embodiment of the invention;

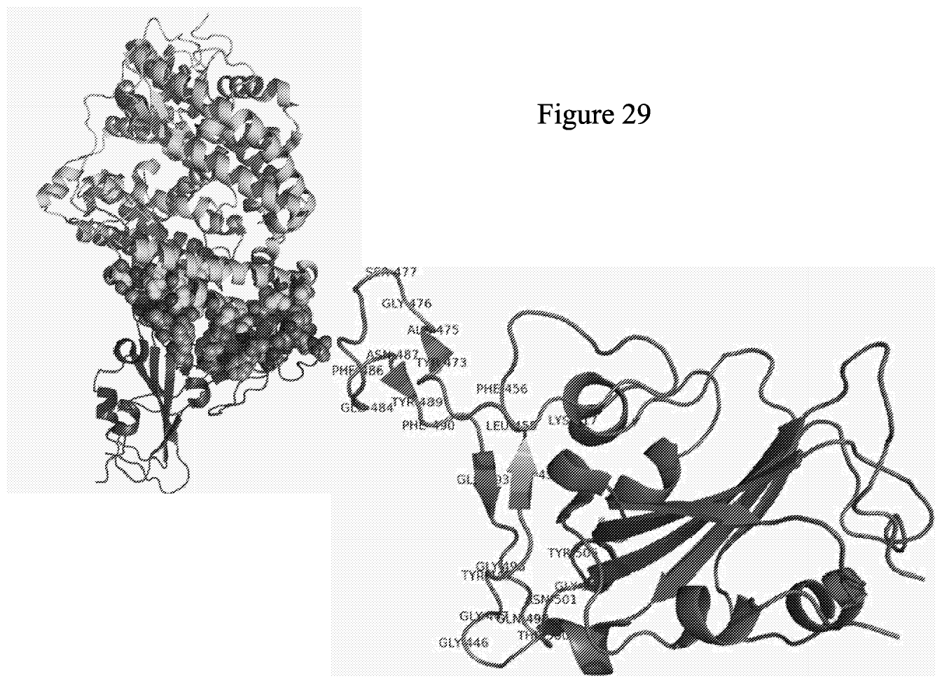

FIG. 29 illustrates the positions of the mutations on a structural image of the spike protein:

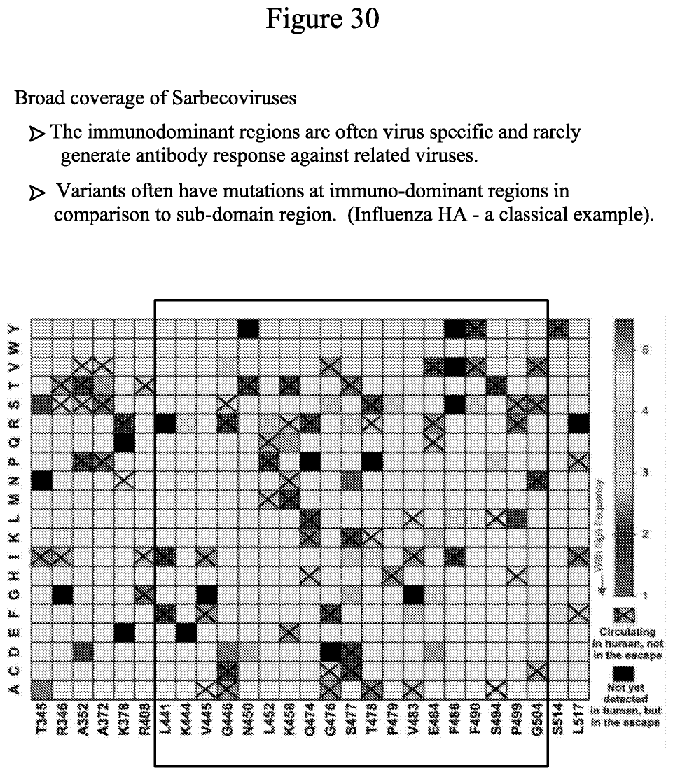

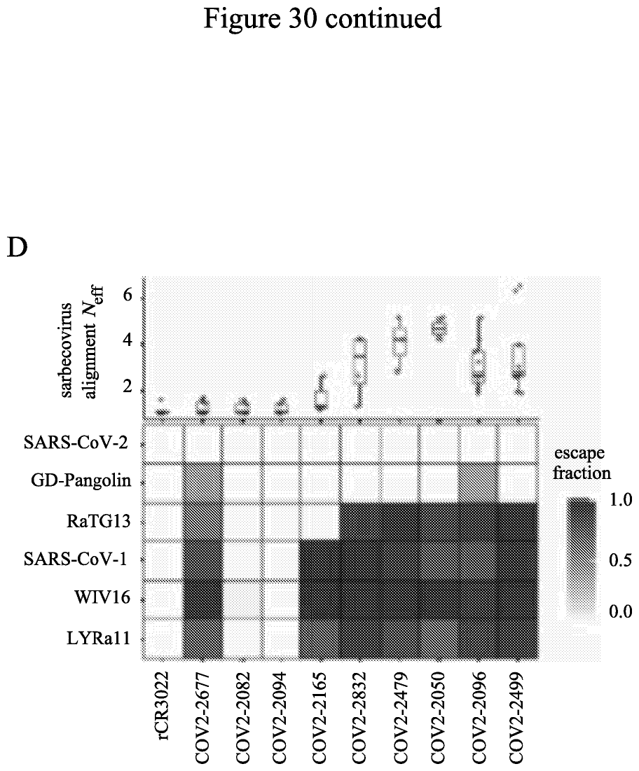

FIG. 30 shows data taken from the literature, showing maximum of current variants have mutation in RBM region and the other epitopes in RBD are conserved and the antibodies against them cross-react; Boxed is the RBM. Figure D—top is the distribution of entropy. Lower the spread, better conserved in the represented sarbecoviruses. All the antibodies targeting this region show cross-neutralisation (white boxes). Black or grey boxes indicate no neutralisation;

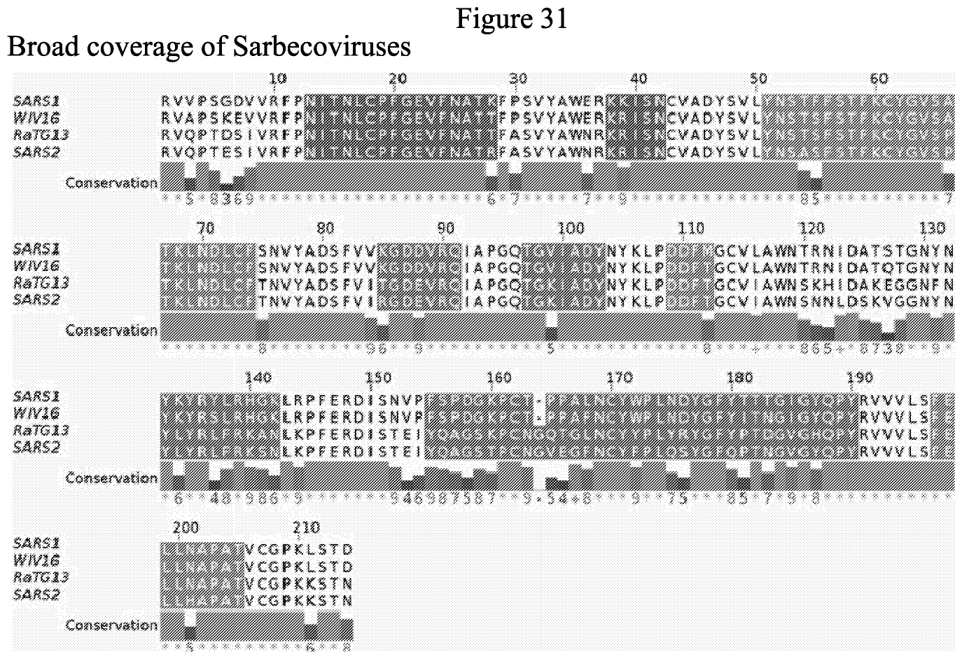

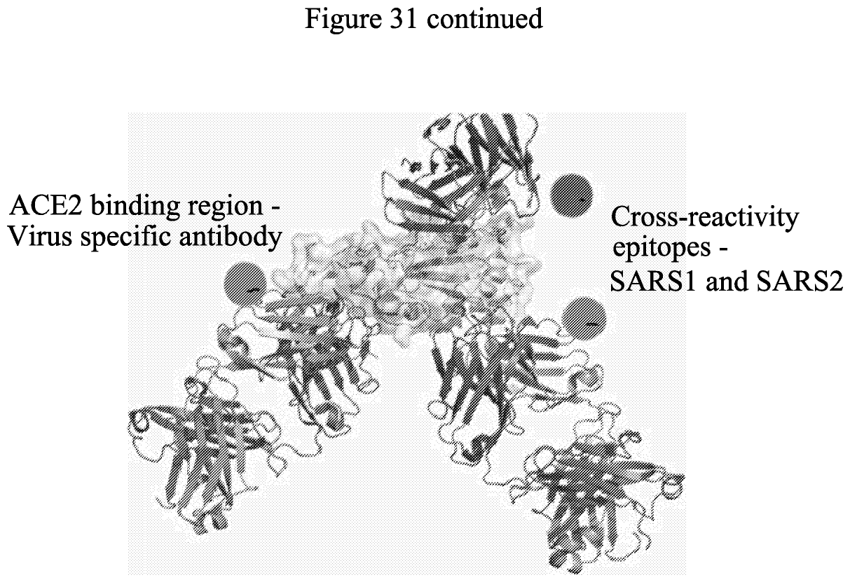

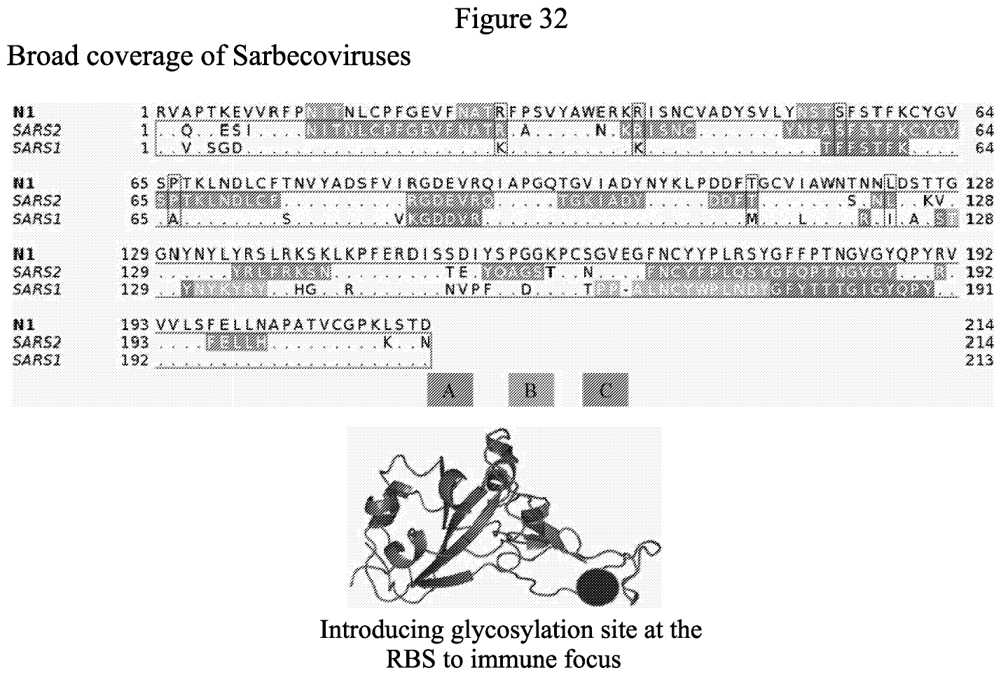

FIGS. 31 and 32 illustrate use of the structural information to identify epitopes, and to include this in the design of S proteins of the invention, and diverting the immune response by glycosylation. FIG. 31 shows RBD sequences of SARS1 (SEQ ID NO:5), WIV16 (SEQ ID NO:102), RaTG13 (SEQ ID NO:103), and SARS2 (SEQ ID NO:11). In FIG. 32, N1—Phylogenetically optimised design (CoV_S_T2_13) (SEQ ID NO:27), SARS2 N1 (SEQ ID NO:104), and SARS1 N1 (SEQ ID NO:105);

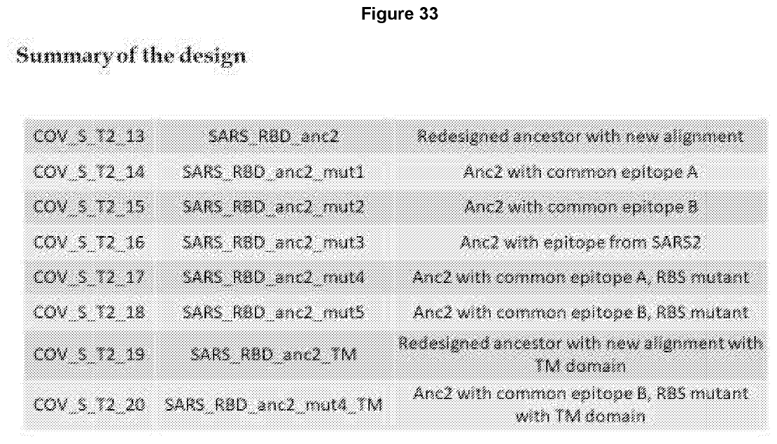

FIG. 33 summarises designs according to embodiments of the invention;

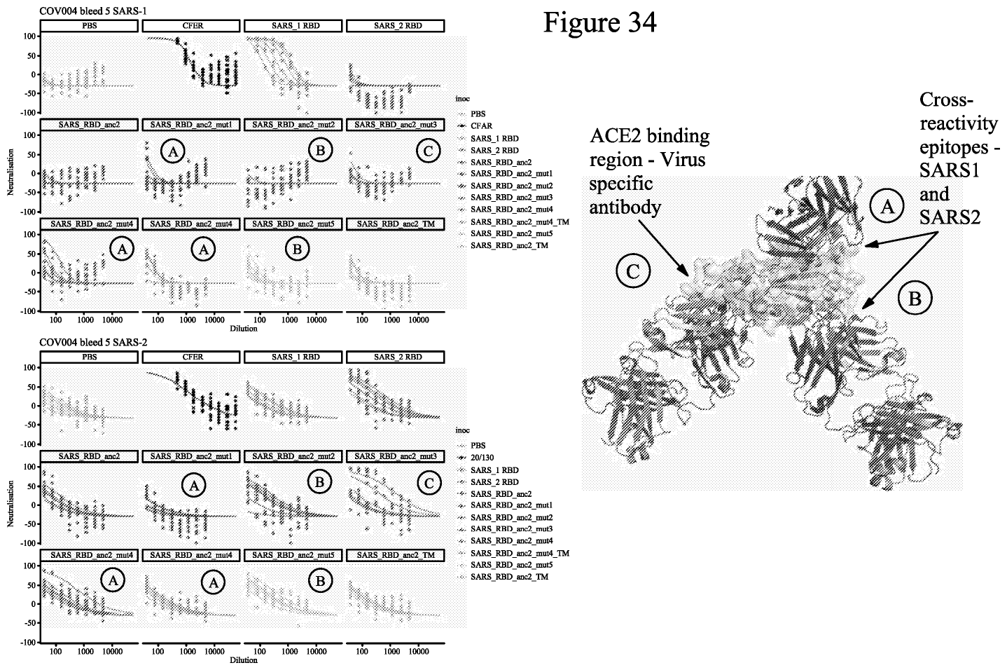

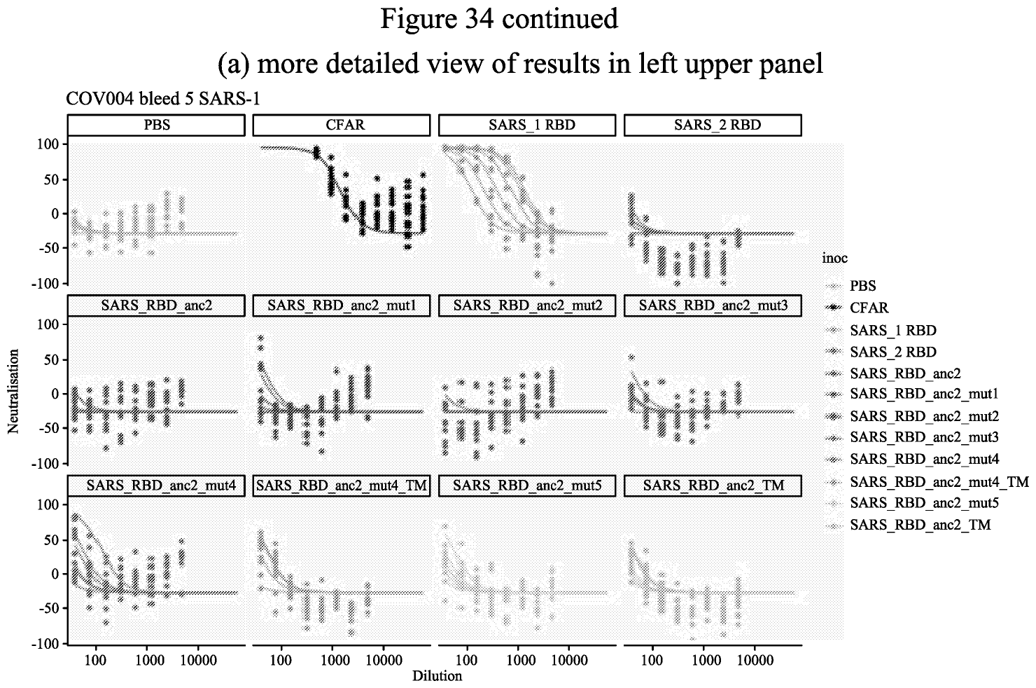

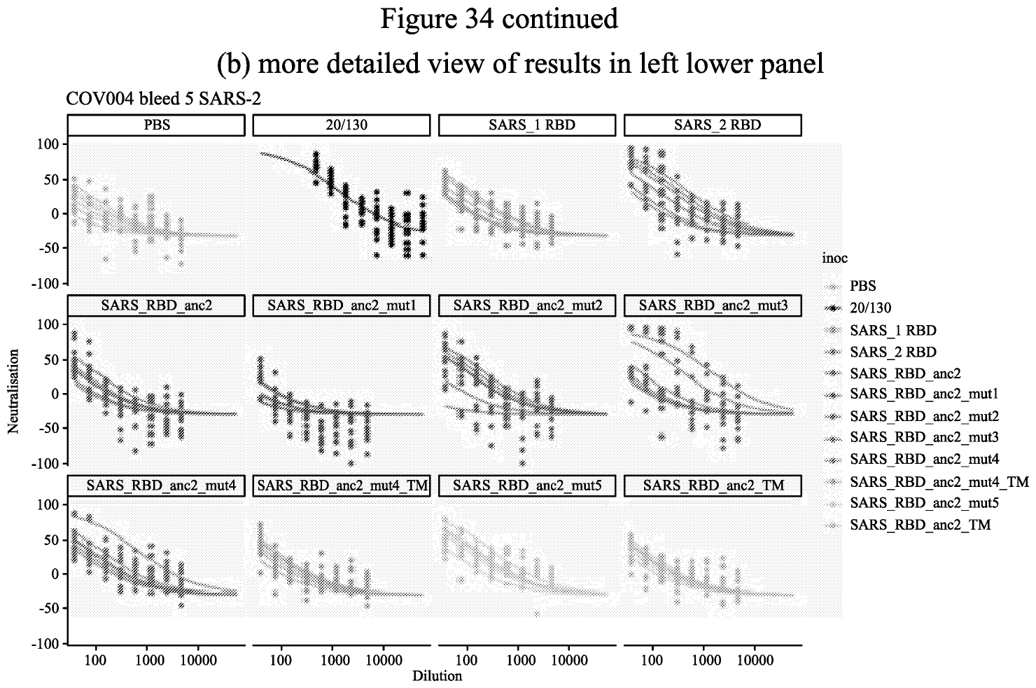

FIG. 34 summarises data obtained for designs according to embodiments of the invention;

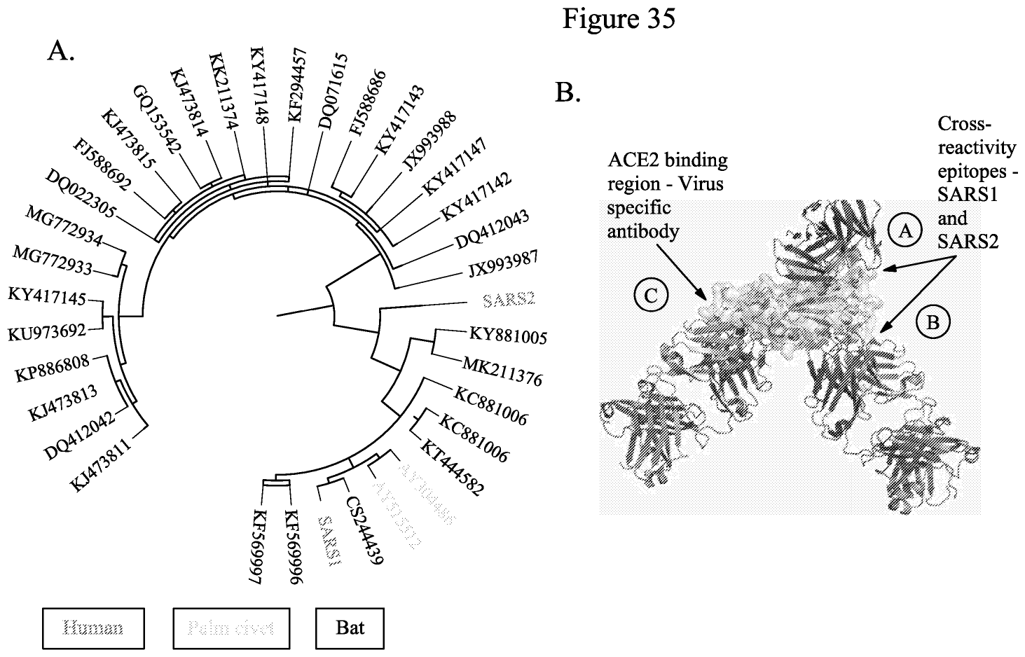

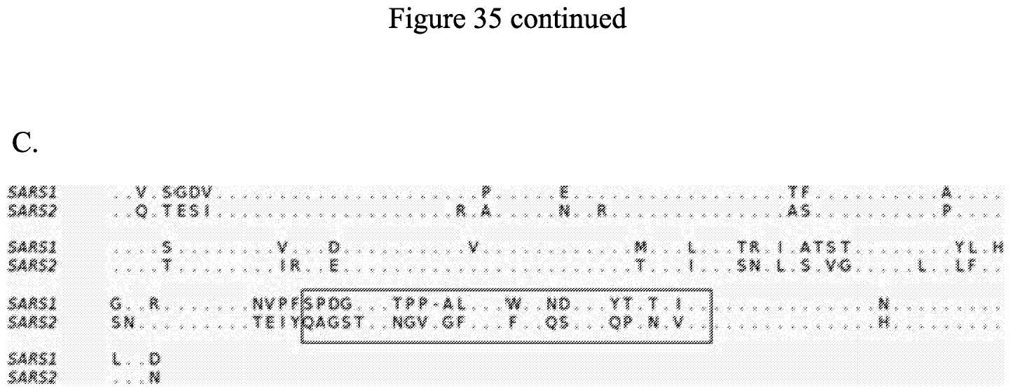

FIG. 35 In-silico design of a vaccine according to an embodiment of the invention:
A. Phylogenetic tree generated for sarbecoviruses using protein sequence of receptor binding domain (RBD) of the spike protein. The tree was generated using IQ-Tree. Human viruses are represented in green, palm civet viruses in pink and bat viruses in dark grey.
B. Structural model of the antibody-RBD complex. The antibodies are represented as cartoon and coloured green and orange and the RBD is represented as both cartoon and surface and coloured pink. The different epitope regions are labelled as A. B and C.
C. Sequence alignment of SARS-1 (SEQ ID NO:5) and SARS-2 (SEQ ID NO:11). Only the non-conserved amino acids are shown. The epitope C is boxed in black.

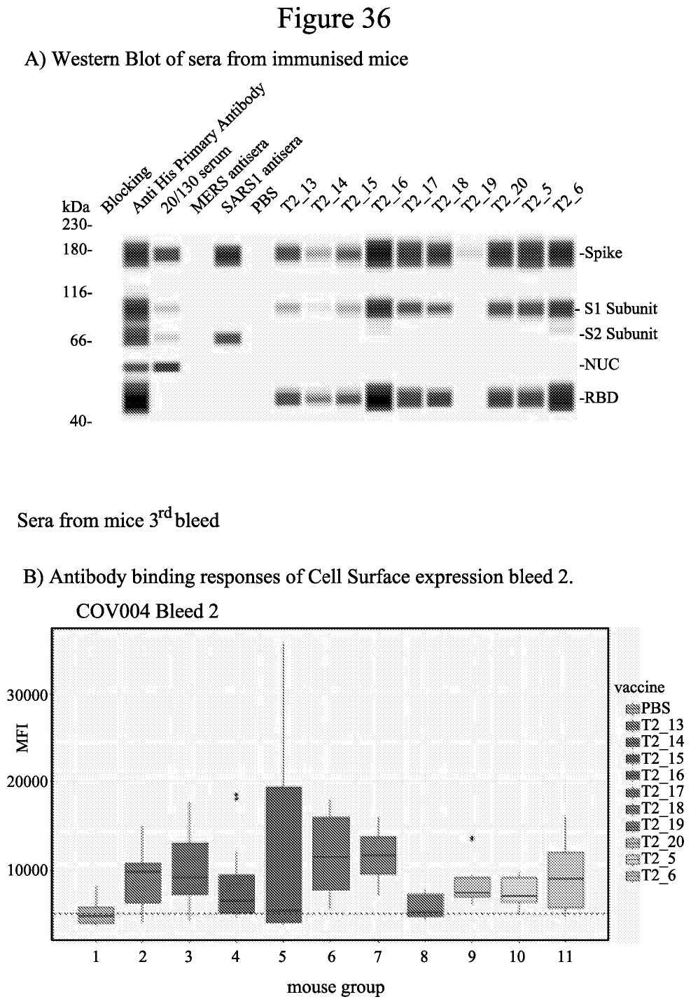

FIG. 36(A) shows a Western Blot of sera from mice immunised with the vaccine designs of Example 32 (COV_S_T2_13-20). FIG. 36 (B) shows antibody binding responses of Cell Surface expression bleed 2.

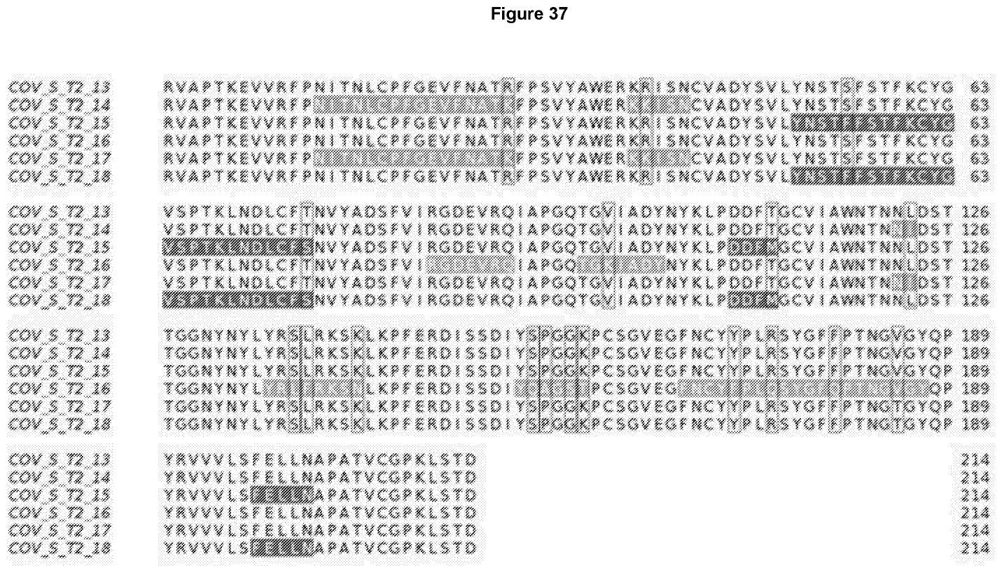

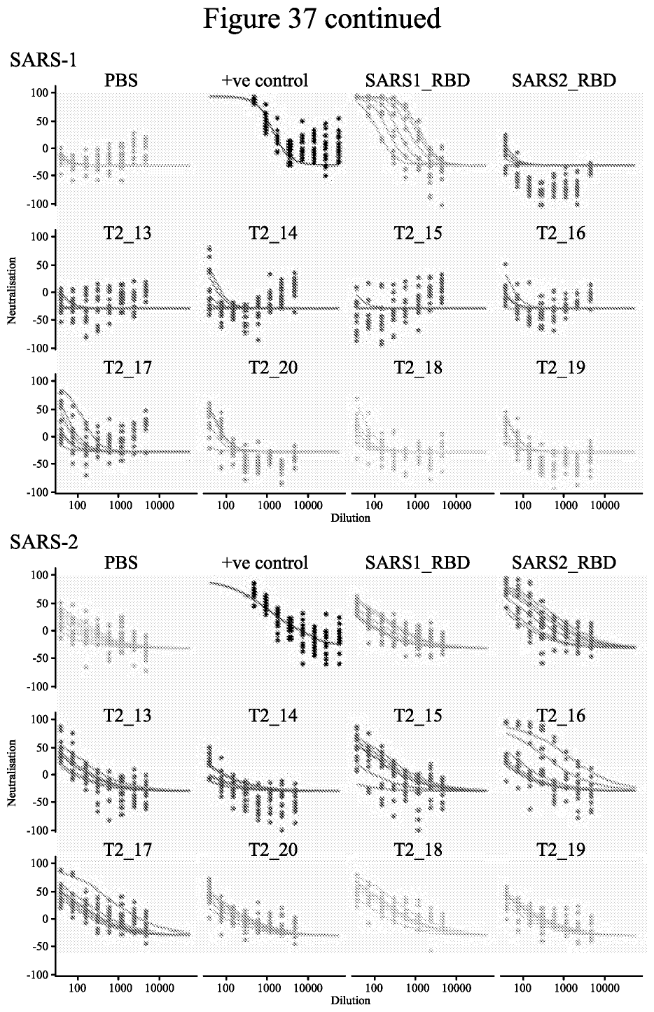

FIG. 37 Neutralisation data:
A. Sequence alignment of the vaccine designs (COV_S_T32_13-18) (SEQ ID NOs: 27-32, respectively). The epitopes are highlighted as coloured blocks. The amino acid residues differing between the designs are boxed in black.
B. Neutralisation curves of vaccine designs, SARS-1 RBD and SARS-2 RBD against SARS1 pseudotype (upper panel) and SARS2 pseudotype (lower panel). The X-axis represents the dilution of the sera and the Y-axis represent the percentage of neutralisation observed. Each curve in the plots represents an individual mouse.

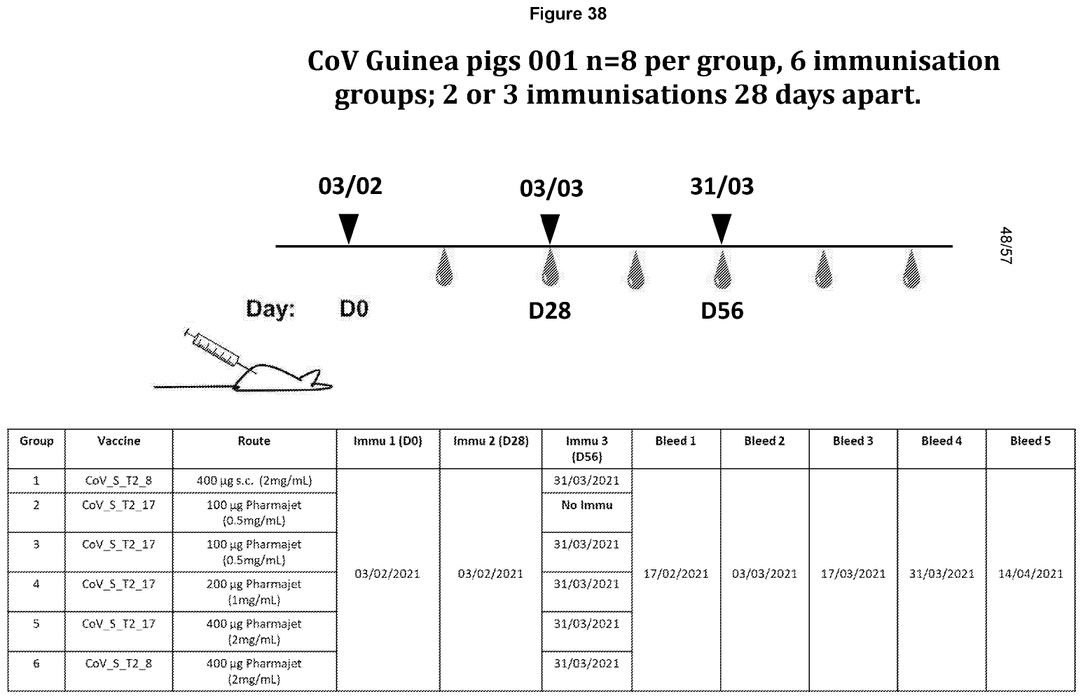

FIG. 38 represents the study protocol of a dose finding study of COV_S_T2_17 (SEQ ID NO:31).

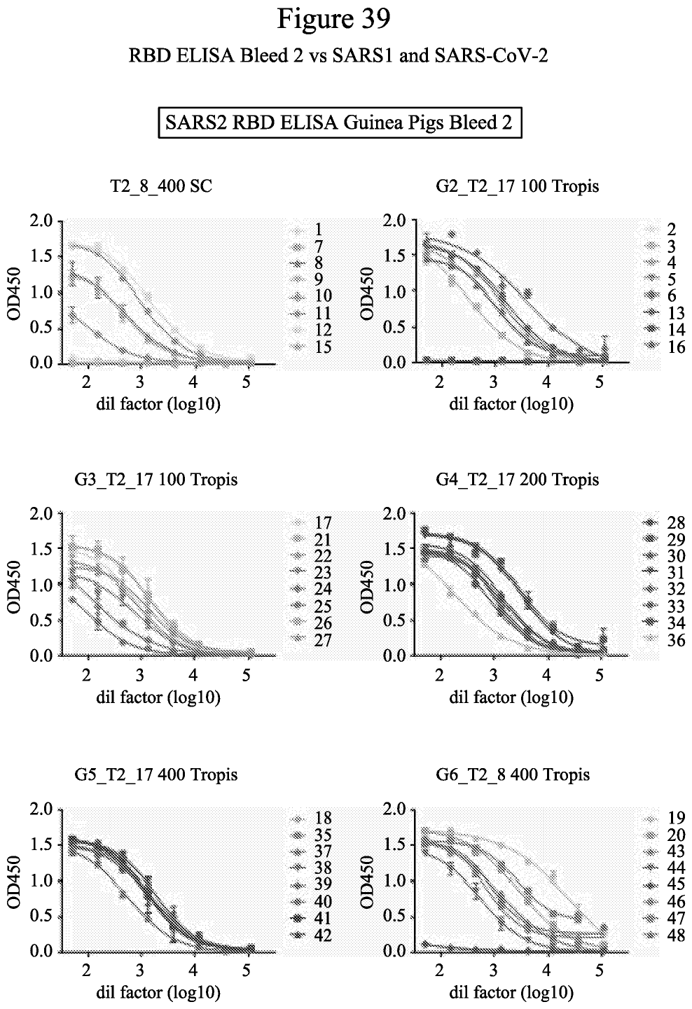

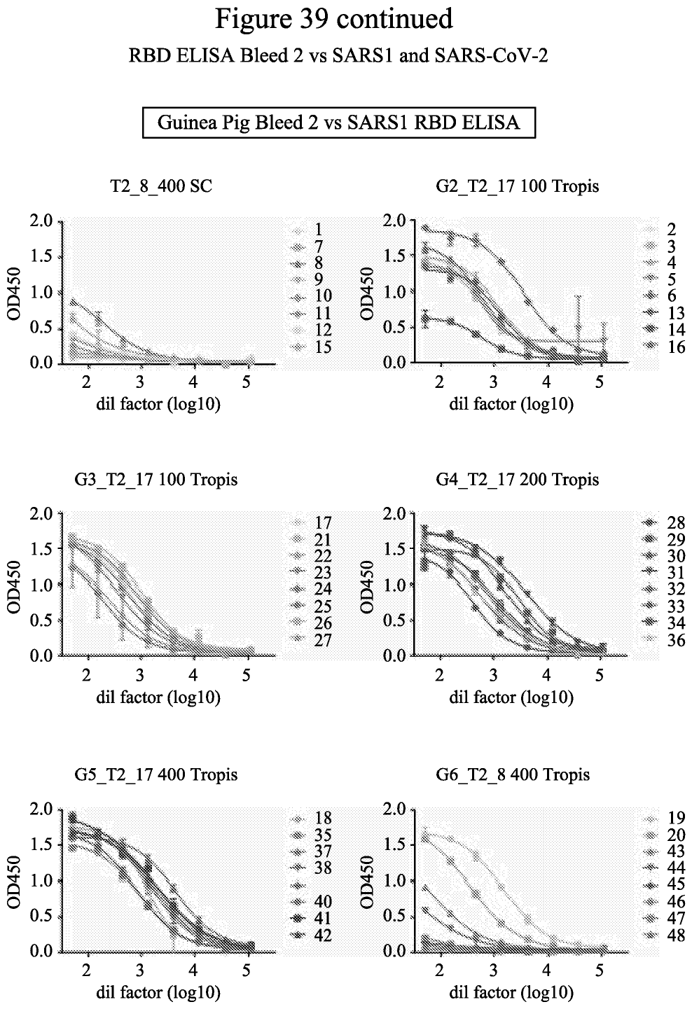

FIG. 39 shows the results of ELISA to determine the level of antibodies to the RBD of SARS-CoV-2, and SARS. Panel A (left) Plates coated with SARS-CoV-2 RBD. Panel B (right) Plates coated with SARS RBD;

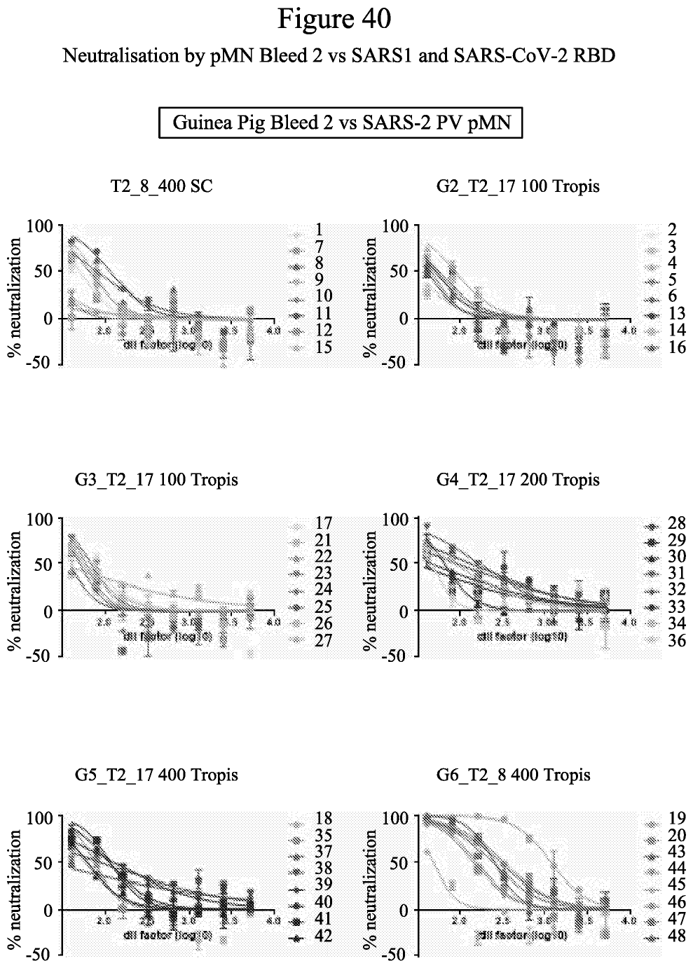

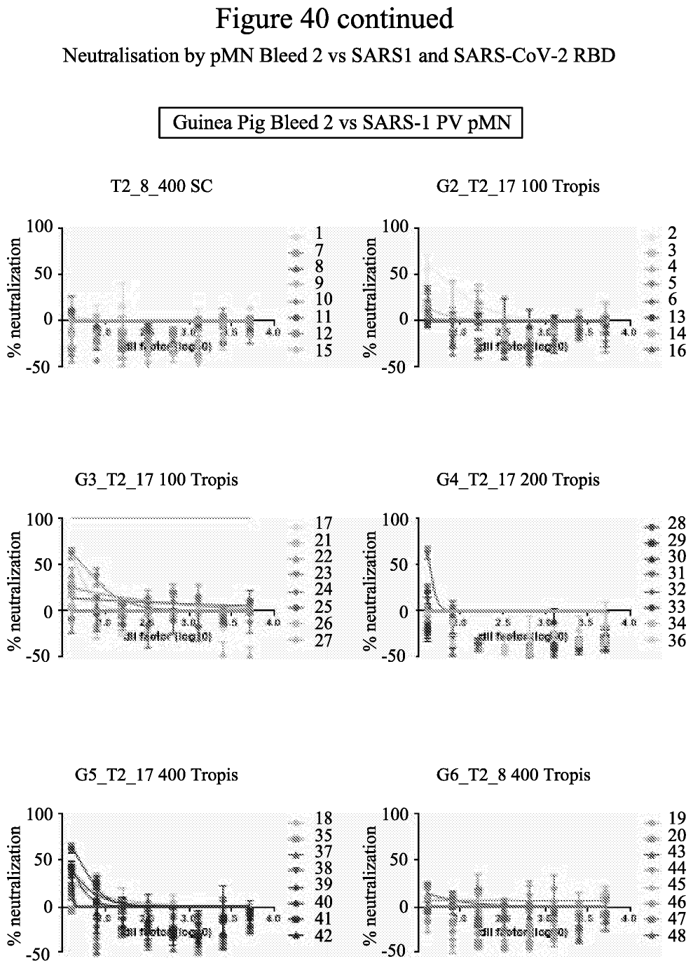

FIG. 40 shows virus neutralisation at day 28 after 1 immunisation (Pseudotype MicroNeutralisation or pMN assay). Panel A (left) Antibody neutralisation of SARS-CoV-2 28 days after 1 dose. Panel B (right) Antibody neutralisation of SARS 28 days after 1 dose.

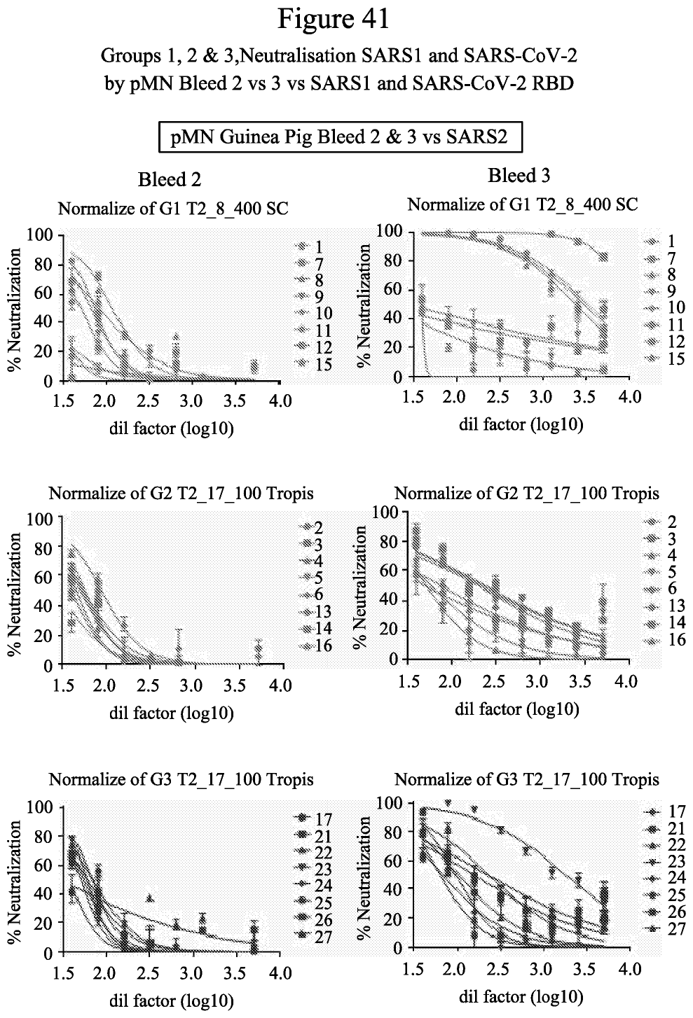

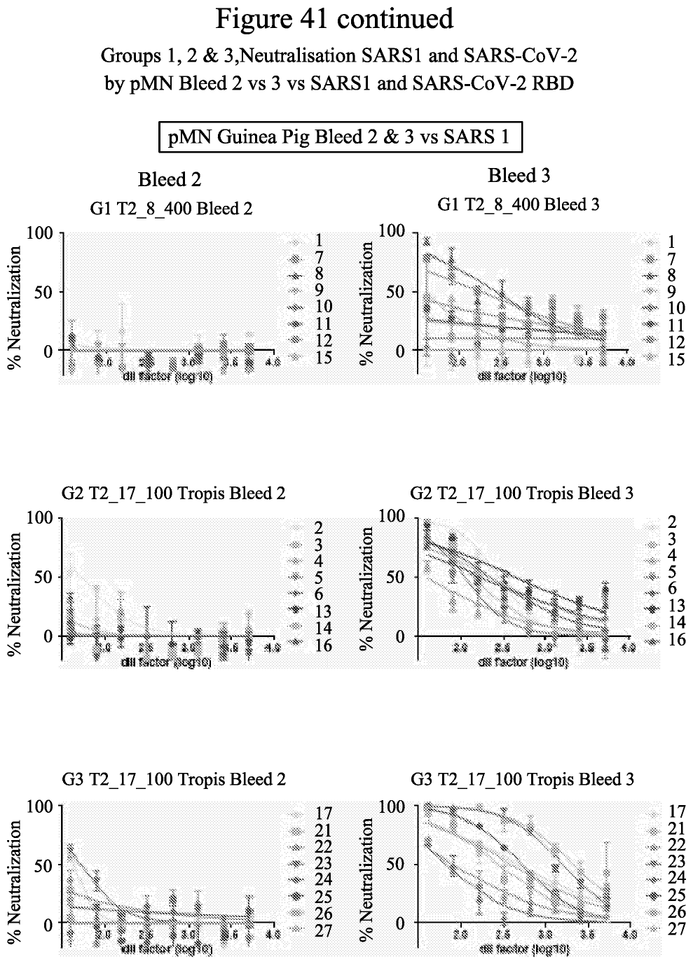

FIG. 41 shows (for Groups 1, 2, and 3) comparison of virus neutralisation responses after first to second immunisation. Panel A (left SARS-CoV-2) Comparing bleeds 2 (pre) and 3 (post) second immunisation (boost). Panel B (right SARS) Comparing bleeds 2 (pre) and 3 (post) second immunisation (boost).

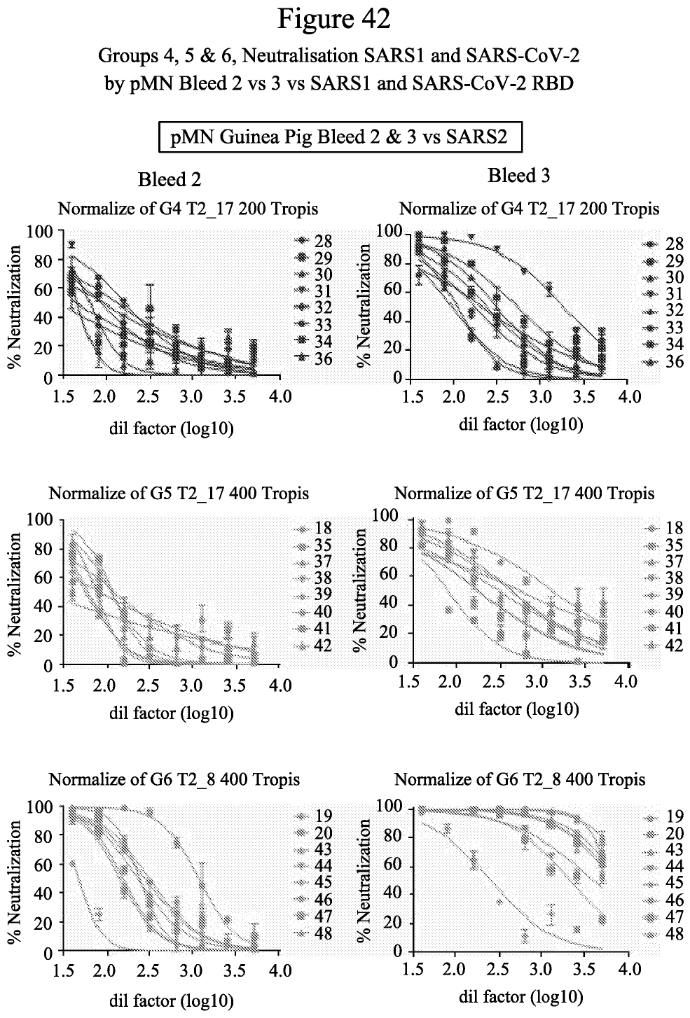

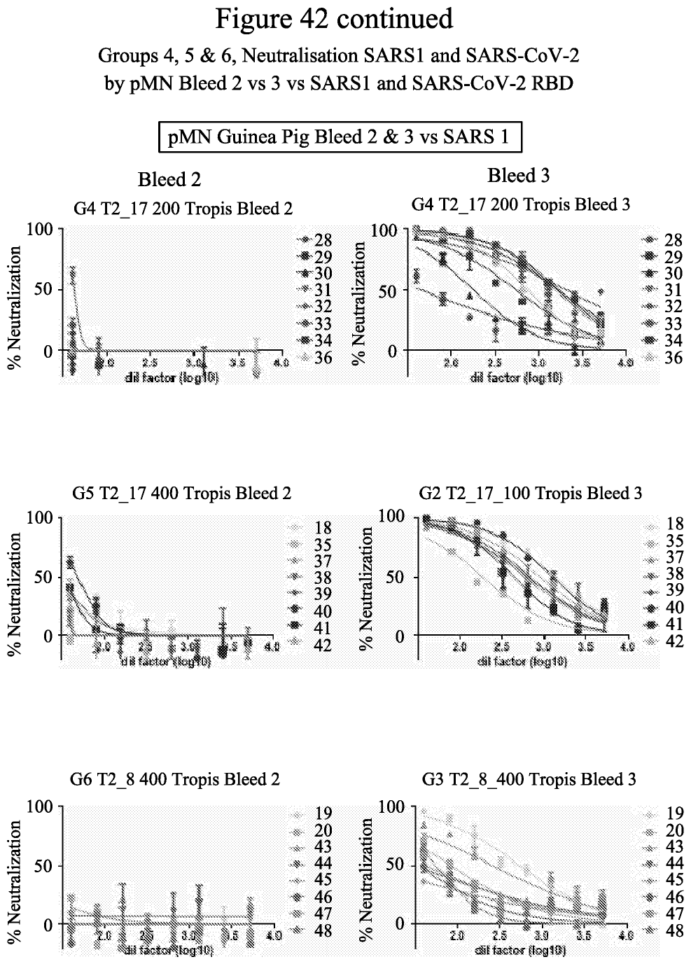

FIG. 42 shows (for groups 4, 5 and 6) comparison of virus neutralisation responses after first to second immunisation. Panel A (left SARS-CoV-2) Comparing bleeds 2 (pre) and 3 (post) second immunisation (boost). Panel B (right SARS) Comparing bleeds 2 (pre) and 3 (post) second immunisation (boost); and

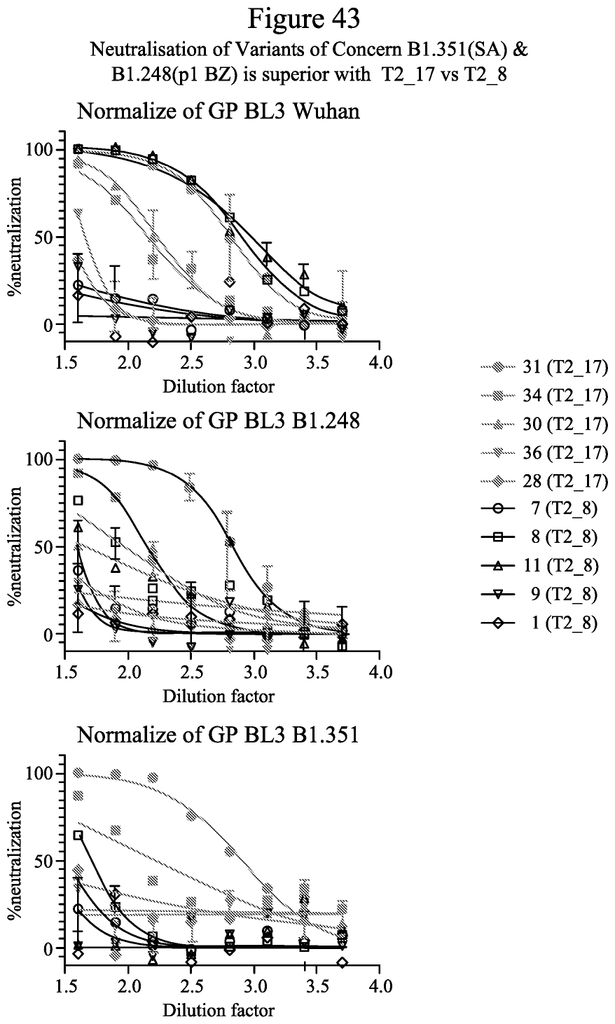

FIG. 43 shows neutralisation of variants of concern (B1.351(SA) & B1.248(P1_BZ) is superior with T2_17 vs T2_8).

We have developed vaccines that protect against Coronaviruses, such as SARS-CoV-2 and SARS-CoV-1, which have the potential to cause future outbreaks from zoonotic reservoirs. We have designed antigens to induce immune responses against the Sarbecoviruses (i.e. β-Coronavirus, Lineage B) in order to protect against the current pandemic and future outbreaks of related Coronaviruses.

A major concern for coronavirus vaccines is disease enhancement (Tseng et al. (2012) “Immunization with SARS Coronavirus Vaccines Leads to Pulmonary Immunopathology on Challenge with the SARS Virus”. PLoS ONE 7(4): e35421). We have modified our antigens to avoid antibody dependent enhancement (ADE) (or ADE-like pro-inflammatory responses) and hyper-activation of the complement pathway.

DNA sequences encoding the antigens are optimised for expression in mammalian cells before inserting into a DNA plasmid expression vector, such as pEVAC. The pEVAC vector is a flexible vaccine platform and any combination of antigens can be inserted to produce a different vaccine. A previous version was used in a SARS-1 clinical trial (Martin et al, Vaccine 2008 25:633). This platform is clinically proven and GMP compliant allowing rapid scale-up. The DNA vaccine may be administered using pain-free needless technology causing patients' cells to produce the antigens, which are recognised by the immune system to induce durable protection against SARS-CoV-2 and future outbreaks of related Coronaviruses.

While high affinity monoclonal antibodies are capable of protecting animals from SARS virus infection (Traggiai, et al. “An efficient method to make human monoclonal antibodies from memory B cells: potent neutralization of SARS coronavirus”. Nat Med 10, 871-875 (2004)), a robust antibody response in early infection in humans is associated with COVID-19 disease progression (Zhao et al, medRxiv: https://doi.org/10.1101/2020.03.02.20030189). Importantly, after recovery from infection and re-challenge of primates with SARS, lung pathology became more severe on secondary exposure, despite limited replication of the virus (Clay et al, “Primary Severe Acute Respiratory Syndrome Coronavirus Infection Limits Replication but Not Lung Inflammation upon Homologous Rechallenge”, J Virol. 2012 April; 86(8): 4234-4244). There is a growing body of evidence of adverse effects of vaccine induced Antibody Dependent Enhancement (ADE) due to post-vaccination infection (Peeples, Avoiding pitfalls in the pursuit of a COVID-19 vaccine, PNAS Apr. 14, 2020 117 (15) 8218-8221). Non-neutralizing antibodies to S-protein may enable an alternative infection pathway via Fc receptor-mediated uptake (Wan et al. Journal of Virology. 2020, 94(5):1-13). These and other reports underline the importance of discriminating between viral antigen structures that induce protective anti-viral effects and those which trigger pro-inflammatory responses. Thus, careful selection and modification of vaccine antigens and the type of vaccine vector that induce protective anti-viral effects, without enhancing lung pathology, is paramount.

Vaccine sequences described herein offer safety from ADE (or ADE-like pro-inflammatory responses), and also increase the breadth of the immune response that can be extended to SARS-CoV-2, SARS and related Bat Sarbecovirus Coronaviruses, which represent future pandemic threats.

Antigens encoded by vaccine sequences described herein have precision immunogenicity, are devoid of ADE sites, and are versatile and compatible with a great number of vaccine vector technologies. DNA molecules may be delivered by PharmaJet's needless-delivery device with demonstrated immunogenicity in advanced clinical trials for other viruses and cancer, or by other DNA delivery such as electroporation or direct injection. Alternatively, the vaccine inserts can be conveniently swapped out to other viral vector, or RNA delivery platforms, which may be easily scaled for greater capacity production or to induce immune responses with different characteristics.

We have designed Coronavirus antigens to induce a highly specific immune response that not only avoids deleterious immune responses induced by the virus, but will provide broader protection, for SARS-CoV-2, SARS-1 and other zoonotic Sarbeco-Coronaviruses. By using libraries of multiple antigens, we are able to down-select the optimal antigenic structures of each class (for instance RBD, E, and M proteins) and to combine the best in class to maximise the breadth of protection from Coronaviruses, by recruiting B- and T-cell responses against multiple targets.

Table of SEQ ID NOS:

| SEQ | |
|---|---|
| ID | |
| NO: | Description |
| 1 | AY274119 (CoV_T1_1): full length S-protein |
| 2 | Nucleic acid sequence encoding amino acid sequence of |
| | SEQ ID NO: 1 |
| 3 | AY274119_tr (CoV_T2_2): truncated S-protein |
| 4 | Nucleic acid sequence encoding amino acid sequence of |
| | SEQ ID NO: 3 |
| 5 | AY274119_RBD (COV_T2_5): RBD |
| 6 | Nucleic acid sequence encoding amino acid sequence of |
| | SEQ ID NO: 5 |
| 7 | EPI_ISL 402119 (CoV_T1_2): full length S-protein |
| 8 | Nucleic acid sequence encoding amino acid sequence of |
| | SEQ ID NO: 7 |
| 9 | EPI_ISL 402119_tr (CoV_T2_3): truncated S-protein |
| 10 | Nucleic acid sequence encoding amino acid sequence of |
| | SEQ ID NO: 9 |
| 11 | EPI_ISL_402119_RBD (CoV_T2_6): RBD |
| 12 | Nucleic acid sequence encoding amino acid sequence of |
| | SEQ ID NO: 11 |
| 13 | Wuhan_Node1 (CoV_T2_1): full length S-protein |
| 14 | Nucleic acid sequence encoding amino acid sequence of |
| | SEQ ID NO: 13 |
| 15 | Wuhan_Node1_tr (CoV_T2_4): truncated S-protein |
| 16 | Nucleic acid sequence encoding amino acid sequence of |
| | SEQ ID NO: 15 |
| 17 | Wuhan_Node1_RBD (CoV_T2_7): RBD |
| 18 | Nucleic acid sequence encoding amino acid sequence of |
| | SEQ ID NO: 17 |
| 19 | Sequence of pEVAC Multiple Cloning Site (MCS) |
| 20 | Entire Sequence of pEVAC |
| 21 | Amino acid sequence of the SARS envelope protein |
| 22 | COV_E_T2_1 (a designed Sarbecovirus sequence) |
| 23 | COV_E_T2_2 (a designed SARS2 sequence) |
| 24 | COV_M_T2_1/1-221 Sarbeco_M_root—Sarbecovirus root ancestor |
| 25 | COV_M_T2_2/1-222 Sarbeco_M_Node88b_epitope_optimised |
| 26 | COV_M_T1_1/1-222 NC_045512.2 SARS2 reference sequence |
| 27 | COV_S_T2_13 (designed S protein RBD sequence) |
| 28 | COV_S_T2_14 (designed S protein RBD sequence) |
| 29 | COV_S_T2_15 (designed S protein RBD sequence) |
| 30 | COV_S_T2_16 (designed S protein RBD sequence) |
| 31 | COV_S_T2_17 (designed S protein RBD sequence) |
| 32 | COV_S_T2_18 (designed S protein RBD sequence) |
| 33 | Designed S protein RBD sequence M7 |
| 34 | Designed S protein RBD sequence M8 |
| 35 | Designed S protein RBD sequence M9 |
| 36 | Designed S protein RBD sequence M10 |
| 37 | Nucleic acid sequence encoding designed S protein |
| | RBD sequence M7 |
| 38 | Nucleic acid sequence encoding designed S protein |
| | RBD sequence M8 |
| 39 | Nucleic acid sequence encoding designed S protein |
| | RBD sequence M9 |
| 40 | Nucleic acid sequence encoding designed S protein |
| | RBD sequence M10 |
| 41 | SARS2 reference E protein sequence |
| 42 | COV_E_T2_3 (SARS2_mutant) |
| 43 | COV_E_T2_4 (Env1_mutant) |
| 44 | COV_E_T2_5 (Env2_mutant) |
| 45 | YP_009724397.2/1-419 nucleocapsid phosphoprotein |
| | [SARS-COV-2] (reference sequence) |
| 46 | COV_N_T2 _1/1-418 Node1b 321-323 deleted |
| 47 | COV_N_T2_2/1-417 epitope optimised 321-323 deleted |
| 48 | COV_M_T2_3 |
| 49 | COV_M_T2_4 |
| 50 | COV_M_T2_5 |
| 51 | Amino acid sequence of “Ralf RBD protein” |
| | (Leader-RBD-Tag) |
| 52 | Amino acid sequence of full length S protein for strain |
| | EPI_ISL_402130_Wuhan |
| 53 | Amino acid sequence for designed full length S protein |
| | COV_S_T2_29 (“VOC Chimera” or “Super_spike”) |
| 54 | Amino acid sequence for designed full length S protein |
| | COV_S_T2_29, but with cysteine residues at positions 410 |
| | and 984 (i.e. G410C and P984C), which correspond to |
| | positions 413 and 987, respectively, of SEQ ID NO:52 |
| 55 | COV_S_T2_19 (designed S protein RBD sequence) |
| 56 | COV_S_T2_20 (designed S protein RBD sequence) |
| 57 | residues (i) of a discontinuous epitope present in |
| | COV_S_T2_14 and COV_S_T2_17: |
| | NITNLCPFGEVFNATK; |
| 58 | residues (ii) of a discontinuous epitope present in |
| | COV_S_T2_14 and COV_S_T2_17: KKISN; |
| 59 | residues (iii) of a discontinuous epitope present in |
| | COV_S_T2_14 and COV_S_T2_17: NI; |
| 60 | residues (i) of a discontinuous epitope present in |
| | COV_S_T2_15 and COV_S_T2_18: |
| | YNSTFFSTFKCYGVSPTKLNDLCFS; |
| 61 | residues (ii) of a discontinuous epitope present in |
| | COV_S_T2_15 and COV_S_T2_18: DDFM; |
| 62 | residues (iii) of a discontinuous epitope present in |
| | COV_S_T2_15 and COV_S_T2_18: FELLN; |
| 63 | residues (i) of a discontinuous epitope present in |
| | COV_S_T2_16: RGDEVRQ; |
| 64 | residues (ii) of a discontinuous epitope present in |
| | COV_S_T2_16: TGKIADY; |
| 65 | residues (iii) of a discontinuous epitope present in |
| | COV_S_T2_16: YRLFRKSN; |
| 66 | residues (iv) of a discontinuous epitope present in |
| | COV_S_T2_16: YQAGST; |
| 67 | residues (v) of a discontinuous epitope present in |
| | COV_S_T2_16: FNCYFPLQSYGFQPTNGVGY. |
| 68 | residues (i) of a discontinuous epitope present in |
| | COV_S_T2_13: NITNLCPFGEVFNATR |
| 69 | residues (ii) of a discontinuous epitope present in |
| | COV_S_T2_13: KRISN |
| 70 | residues (iii) of a discontinuous epitope present in |
| | COV_S_T2_13: NL |
| 71 | residues (i) of a discontinuous epitope present in |
| | COV_S_T2_13: YNSTSFSTFKCYGVSPTKLNDLCFT |
| 72 | residues (ii) of a discontinuous epitope present in |
| | COV_S_T2_13: DDFT |
| 73 | residues (ii) of a discontinuous epitope present in |
| | COV_S_T2_13: TGVIADY |
| 74 | residues (iii) of a discontinuous epitope present in |
| | COV_S_T2_13: YRSLRKSK |
| 75 | residues (iv) of a discontinuous epitope present in |
| | COV_S_T2_13: YSPGGK |
| 76 | residues (v) of a discontinuous epitope present in |
| | COV_S_T2_13: FNCYYPLRSYGFFPTNGVGY |
| 77 | residues (v) of a discontinuous epitope present in |
| | COV_S_T2_17, 18: FNCYYPLRSYGFFPTNGTGY |
| 78-85 | Nucleic acid encoding COV_S_T2_13-20 |
| 86 | SARS S protein—region around the S1 cleavage site |
| 87 | Beta CoV/bat/Yunnan/RaTG13/2013 S protein— |
| | region around the S1 cleavage site |
| 88 | Beta CoV/Wuhan/IVDCHB01/2019 S protein— |
| | region around the S1 cleavage site |
| 89 | Beta CoV/Wuhan/HBCDCHB01/2019 S protein— |
| | region around the S1 cleavage site |
| 90 | Beta CoV/Guangdong/20SF028/2020 S protein— |
| | region around the S1 cleavage site |
| 91 | Beta CoV/USA/IL1/2020\|EPL_ISL_404253 S protein— |
| | region around the S1 cleavage site |
| 92 | Consensus—region around the S1 cleavage site |
| 93 | NL63_Alpha E protein |
| 94 | 229E_Alpha E protein |
| 95 | HKU1_Beta E protein |
| 96 | HKU1_Beta E protein |
| 97 | KF600630_MERS_Beta E protein |
| 98 | Consensus E protein sequence (FIG. 7, upper) |
| 99 | Consensus E protein sequence (FIG. 7, middle) |
| 100 | Consensus E protein sequence (FIG. 7, lower) |
| 101 | M protein sequence |
| 102 | WIV16 S protein RBD |
| 103 | RaTG13 S protein RBD |
| 104 | SARS2 N1 protein |
| 105 | SARS1 N protein |
| 106 | RBD region amino acid sequence (FIG. 13) |
| 107 | SARS COV_2 isolate EPI_ISL 402130 (SEQ ID NO: 52) |
| | with residues substituted for cysteine residues to allow |
| | formation of a disulphide bridge to form a “closed |
| | S protein”. |

### EXAMPLE 1—VACCINE SEQUENCES

The CoV S-protein is a trimeric transmembrane glycoprotein essential for the entry of the virus particles into the host cell. The S-protein comprises two domains, the S1 domain responsible for ACE-2 receptor binding, and the S2 domain, responsible for fusion of the viral and cell membranes. The S-protein is the main target for immunisation. However, evidence has shown antibody dependent enhancement (ADE) of SARS-CoV infections, in particular of the S-protein, resulting in enhanced infection and immune evasion, and/or resulting proinflammatory responses. The S-protein contains non-neutralising epitopes which are bound by antibodies. This immune diversion results in enhanced disease progression due to the inability of the immune system to neutralise the pathogen. ADE can also increase infectivity of the pathogen into host cells. Neutralising antibodies produced after an initial infection of SARS-CoV may be non-neutralising to a second infection with a different SARS-CoV strain.

The high genetic similarity between SARS-CoV and SARS-CoV-2 means that it is possible to map boundaries of the S1 and S2 domains, as well as the RBD, onto a novel design scaffold. The applicant has generated a novel sequence for an S-protein, called CoV_T2_1 (also referred to as Wuhan-Node-1), which includes modifications to improve its immunogenicity, and to remove or mask epitopes that are responsible for ADE (or ADE-like pro-inflammatory responses).

This example provides amino acid and nucleic acid sequences of full length S-protein, truncated S-protein (tr, missing the C-terminal part of the S2 sequence), and the receptor binding domain (RBD) for:
SARS-TOR2 isolate AY274119;
SARS_CoV_2 isolate—hCov-19/Wuhan/LVDC-HB-01/2019 (EPI_ISL_402119); and
embodiments of the invention, termed “CoV_T2_1” (or “Wuhan_Node1”).

The CoV_T2_1 (Wuhan_Node1) sequences include modifications to provide effective vaccines that induce a broadly neutralising immune response to protect against diseases caused by CoVs, especially β-CoVs, such as SARS-CoV and SARS-CoV-2. The vaccines also lack non-neutralising epitopes that may result in virus immune evasion and disease progression by ADE (or ADE-like pro-inflammatory responses).

The following amino acid and nucleic acid sequences are provided in this example:

| SARS-TOR2 isolate AY274119: | |
|---|---|
| >AY274119 (CoV_T1_1): | |
| full length S-protein (SEQ ID NO: 1) and nucleic acid encoding full length S- | |
| protein (SEQ ID NO: 2) | |
| >AY274119_tr (CoV_T2_2): | |
| truncated S-protein (SEQ ID NO: 3) and nucleic acid encoding truncated S- | |
| protein (SEQ ID NO: 4) | |
| >AY274119_RBD (COV_T2_5): | |
| RBD (SEQ ID NO: 5) and nucleic acid encoding RBD (SEQ ID NO: 6) | |
| SARS_COV_2 isolate - hCov-19/Wuhan/LVDC-HB-01/2019 (EPI_ISL_402119): | |
| >EPI_ISL_402119 (CoV_T1_2): | |
| full length S-protein (SEQ ID NO: 7) and nucleic acid encoding full length S- | |
| protein (SEQ ID NO: 8) | |
| >EPI_ISL_402119_tr (CoV_T2_3): | |
| truncated S-protein (SEQ ID NO: 9) and nucleic acid encoding truncated S- | |
| protein (SEQ ID NO: 10) | |
| >EPI_ISL_402119_RBD (COV_T2_6): | |
| RBD (SEQ ID NO: 11) and nucleic acid encoding RBD (SEQ ID NO: 12) | |
| Sequences according to embodiments of the invention: CoV_T2_1 (Wuhan_Node1), | |
| CoV_T2_4 (Wuhan_Node1_tr), or CoV_T2_7 (Wuhan_Node1_RBD): | |
| >Wuhan_Node1 (CoV_T2_1): | |
| full length S-protein (SEQ ID NO: 13) and nucleic acid encoding full length S- | |
| protein (SEQ ID NO: 14) | |
| >Wuhan_Node1_tr (CoV_2_4): | |
| truncated S-protein (SEQ ID NO: 15) and nucleic acid encoding truncated S- | |
| protein (SEQ ID NO: 16) | |
| >Wuhan_Node1_RBD (CoV_T2_7): | |
| RBD (SEQ ID NO: 17) and nucleic acid encoding RBD (SEQ ID NO: 18) | |
| >AY274119 (CoV_T1_1) | |
| (SEQ ID NO: 1) | |
| Amino acid sequence: | |
| MFIFLLFLTLTSGSDLDRCTTFDDVQAPNYTQHTSSMRGVYYPDEIFRSDTLYLTQDLFLPFYSNVTG | |
| FHTINHTFGNPVIPFKDGIYFAATEKSNVVRGWVFGSTMNNKSQSVIIINNSTNVVIRACNFELCDNP | |
| FFAVSKPMGTQTHTMIFDNAFNCTFEYISDAFSLDVSEKSGNFKHLREFVFKNKDGFLYVYKGYQPID | |
| VVRDLPSGFNTLKPIFKLPLGINITNFRAILTAFSPAQDIWGTSAAAYFVGYLKPTTFMLKYDENGTI | |
| TDAVDCSQNPLAELKCSVKSFEIDKGIYQTSNFRVVPSGDVVRFPNITNLCPFGEVFNATKFPSVYAW | |
| ERKKISNCVADYSVLYNSTFFSTFKCYGVSATKLNDLCFSNVYADSFVVKGDDVRQIAPGQTGVIADY | |
| NYKLPDDFMGCVLAWNTRNIDATSTGNYNYKYRYLRHGKLRPFERDISNVPFSPDGKPCTPPALNCYW | |
| PLNDYGFYTTTGIGYQPYRVVVLSFELLNAPATVCGPKLSTDLIKNQCVNFNFNGLTGTGVLTPSSKR | |
| FQPFQQFGRDVSDFTDSVRDPKTSEILDISPCAFGGVSVITPGTNASSEVAVLYQDVNCTDVSTAIHA | |
| DQLTPAWRIYSTGNNVFQTQAGCLIGAEHVDTSYECDIPIGAGICASYHTVSLLRSTSQKSIVAYTMS | |
| LGADSSIAYSNNTIAIPTNFSISITTEVMPVSMAKTSVDCNMYICGDSTECANLLLQYGSFCTQLNRA | |
| LSGIAAEQDRNTREVFAQVKQMYKTPTLKYFGGFNFSQILPDPLKPTKRSFIEDLLFNKVTLADAGFM | |
| KQYGECLGDINARDLICAQKFNGLTVLPPLLTDDMIAAYTAALVSGTATAGWTFGAGAALQIPFAMQM | |
| AYRFNGIGVTQNVLYENQKQIANQFNKAISQIQESLTTTSTALGKLQDVVNQNAQALNTLVKQLSSNF | |
| GAISSVLNDILSRLDKVEAEVQIDRLITGRLQSLQTYVTQQLIRAAEIRASANLAATKMSECVIGQSK | |
| RVDFCGKGYHLMSFPQAAPHGVVFLHVTYVPSQERNFTTAPAICHEGKAYFPREGVFVFNGTSWFITQ | |
| RNFFSPQIITTDNTFVSGNCDVVIGIINNTVYDPLQPELDSFKEELDKYFKNHTSPDVDLGDISGINA | |
| SVVNIQKEIDRINEVAKNLNESLIDLQELGKYEQYIKWPWYVWLGFIAGLIAIVMVTILLCCMTSCCS | |
| CLKGACSCGSCCKFDEDDSEPVLKGVKLHYT | |
| >AY274119 (CoV_T1_1) | |
| (SEQ ID NO: 2) | |
| Nucleic acid sequence: | |
| atgtttatctttctgctgtttctgaccctgaccagcggcagcgacctggatagatgcacc | |
| accttcgacgatgtgcaggcccctaactacacccagcacaccagctctatgcggggcgtg | |
| tactaccccgacgagattttcagaagcgacaccctgtatctgacccaggacctgttcctg | |
| cctttctacagcaacgtgaccggcttccacaccatcaaccacaccttcggcaaccctgtg | |
| atccccttcaaggacggcatctactttgccgccaccgagaagtccaacgtcgtcagagga | |
| tgggtgttcggcagcaccatgaacaacaagagccagagcgtgatcatcatcaacaacagc | |
| accaacgtggtcatccgggcctgcaacttcgagctgtgcgacaacccattcttcgccgtg | |
| tccaagcctatgggcacccagacacacaccatgatcttcgacaacgccttcaactgcacc | |
| ttcgagtacatcagcgacgccttcagcctggacgtgtccgaaaagagcggcaacttcaag | |
| cacctgagggaattcgtgttcaagaacaaggatggcttcctgtacgtgtacaagggctac | |
| cagcctatcgacgtcgtgcgggatctgcccagcggcttcaataccctgaagcctatcttc | |
| aagctgcccctgggcatcaacatcaccaacttcagagccatcctgaccgctttcagcccc | |
| gctcaggatatctggggaacaagcgccgctgcctacttcgtgggctacctgaagccaacc | |
| accttcatgctgaagtacgacgagaacggcaccatcaccgacgccgtggactgtagccaa | |
| aatcctctggccgagctgaagtgcagcgtgaagtccttcgagatcgacaagggcatctac | |
| cagaccagcaatttcagagtggtgccctccggggatgtcgtgcggttccccaacatcaca | |
| aatctgtgccccttcggcgaggtgttcaacgccaccaagtttccctctgtgtacgcctgg | |
| gagcgcaaaaagatcagcaactgcgtggccgactacagcgtgctgtacaactccaccttc | |
| ttcagcaccttcaagtgctacggcgtgtccgccacaaagctgaacgacctgtgcttctcc | |
| aacgtgtacgccgacagcttcgtggtcaaaggcgacgacgttcggcagattgcccctgga | |
| caaacaggcgtgatcgccgattacaactacaagctgcctgacgacttcatgggctgcgtg | |
| ctggcctggaacaccagaaacatcgatgccacctccaccggcaactacaattacaagtac | |
| agatacctgcggcacggcaagctgcggcctttcgagagggatatcagcaatgtgcctttt | |
| agccccgacggcaagccctgcacacctcctgctctgaattgctactggcccctgaacgac | |
| tacggcttttacaccaccacaggcatcggctatcagccctatagagtggtggtcctgtcc | |
| tttgagctgctgaatgcccctgccacagtgtgcggacctaagctgtctaccgacctgatc | |
| aagaaccagtgcgtgaacttcaacttcaacggcctgaccggcaccggcgtgctgacacca | |
| agcagcaagagattccagcctttccagcagttcggccgggatgtgtccgacttcacagac | |
| agcgtcagagatcccaagaccagcgagatcctggacatcagcccttgtgcctttggcgga | |
| gtgtccgtgatcacccctggcacaaatgcctctagcgaagtggccgtgctgtatcaggac | |
| gtgaactgcaccgatgtgtccaccgccattcacgccgatcagctgactcccgcttggcgg | |
| atctatagcacaggcaacaacgtgttccagacacaagccggctgtctgatcggagccgag | |
| catgtggataccagctacgagtgcgacatccctatcggcgctggcatctgtgcctcttac | |
| cacaccgtgtctctgctgcggagcaccagccagaaatccatcgtggcctacaccatgagc | |
| ctgggcgccgattcttctatcgcctactccaacaacacaatcgctatccccaccaatttc | |
| agcatctccatcaccaccgaagtgatgcccgtgtccatggccaagacctccgtggattgc | |
| aacatgtacatctgcggcgacagcaccgagtgcgccaatctgctgctccagtacggcagc | |
| ttctgcacccagctgaatagagccctgtctggaattgccgccgagcaggacagaaacacc | |
| agagaagtgttcgcccaagtgaagcagatgtataagaccccgacactcaagtacttcggc | |
| gggttcaacttctcccagatcctgcctgatcctctgaagcccaccaagcggagcttcatc | |
| gaggacctgctgttcaacaaagtgaccctggccgacgccggctttatgaagcagtatggc | |
| gagtgcctgggcgacatcaacgccagggatctgatttgcgcccagaagtttaacggactg | |
| accgtgctgcctcctctgctgaccgatgatatgatcgccgcctacacagccgctctggtg | |
| tctggtacagctaccgccggatggacatttggagctggcgccgctctccagattccattc | |
| gctatgcagatggcctaccggttcaacggcatcggagtgacccagaatgtgctgtacgag | |
| aatcagaagcagatcgccaatcagttcaacaaggccatcagccagatccaagagagcctg | |
| accaccacaagcacagccctgggaaagctccaggacgtggtcaaccagaatgctcaggcc | |
| ctgaacaccctggtcaagcagctgagcagcaacttcggcgccatcagctccgtgctgaat | |
| gacatcctgagccggctggacaaggtggaagcagaggtqcagatcgaccggctgatcaca | |
| ggcagactccagagcctccagacctacgtgacacagcagctgatcagagccgccgagatt | |
| agagcctctgccaatctggccgccaccaaaatgagcgagtgtgtcctgggccagagcaag | |
| agagtggacttttgcggcaagggctatcacctgatgagcttcccacaggccgctcctcat | |
| ggcgtggtctttctgcacgtgacatacgtgcccagccaagagagaaacttcaccaccgct | |
| ccagccatctgccacgagggcaaagcctactttcccagagaaggcgtgttcgtgtttaac | |
| ggcacctcctggtttatcacccagcggaatttcttcagcccgcaaatcatcaccacagac | |
| aacaccttcgtgtccggcaactgtgacgtcgtgatcggcatcattaacaataccgtgtac | |
| gaccctctccagcctgagctggacagcttcaaagaggaactggataagtacttcaagaat | |
| cacacgagccccgatgtggacctgggcgatatctctggcatcaatgccagcgtcgtgaac | |
| atccagaaagagattgacaggctgaacgaggtggccaagaacctgaacgagtccctgatc | |
| gacctgcaagagctggggaagtacgagcagtacatcaagtggccttggtacgtgtggctg | |
| ggctttatcgccggactgatcgccatcgtgatggtcaccatcctgctgtgctgcatgacc | |
| agctgttgcagctgtctgaagggcgcctgtagctgtggctcctgctgcaagttcgatgag | |
| gacgactctgagccagtgctgaaaggcgtgaagctgcactacacc | |
| >AY274119_tr (CoV_T2_2) | |
| (SEQ ID NO: 3) | |
| Amino acid sequence: | |
| MFIFLLFLTLTSGSDLDRCTTFDDVQAPNYTQHTSSMRGVYYPDEIFRSDTLYLTQDLFLPFYSNVTG | |
| FHTINHTFGNPVIPFKDGIYFAATEKSNVVRGWVFGSTMNNKSQSVIIINNSTNVVIRACNFELCDNP | |
| FFAVSKPMGTQTHTMIFDNAFNCTFEYISDAFSLDVSEKSGNFKHLREFVFKNKDGFLYVYKGYQPID | |
| VVRDLPSGFNTLKPIFKLPLGINITNFRAILTAFSPAQDIWGTSAAAYFVGYLKPTTFMLKYDENGTI | |
| TDAVDCSQNPLAELKCSVKSFEIDKGIYQTSNFRVVPSGDVVRFPNITNLCPFGEVFNATKFPSVYAW | |
| ERKKISNCVADYSVLYNSTFFSTFKCYGVSATKLNDLCFSNVYADSFVVKGDDVRQIAPGQTGVIADY | |
| NYKLPDDFMGCVLAWNTRNIDATSTGNYNYKYRYLRHGKLRPFERDISNVPFSPDGKPCTPPALNCYW | |
| PLNDYGFYTTTGIGYQPYRVVVLSFELLNAPATVCGPKLSTDLIKNQCVNFNFNGLTGTGVLTPSSKR | |
| FQPFQQFGRDVSDFTDSVRDPKTSEILDISPCAFGGVSVITPGTNASSEVAVLYQDVNCTDVSTAIHA | |
| DQLTPAWRIYSTGNNVFQTQAGCLIGAEHVDTSYECDIPIGAGICASYHTVSLLRSTSQKSIVAYTMS | |
| LGADSSIAYSNNTIAIPTNFSISITTEVMPVSMAKTSVDCNMYICGDSTECANLLLQYGSFCTQLNRA | |
| LSGIAAEQDRNTREVFAQVKQMYKTPTLKYFGGFNFSQILPDPLKPTKRSFIEDLLFNKVTLADAGFM | |
| KQYGECLGDINARDLICAQKFNGLTVLPPLLTDDMIAAYTAALVSGTATAGWTFGAGAALQIPFAMQM | |
| AYRFNGIGVTQNVLYENQKQIANQFNKAISQIQESLTTTSTALGKLQDVVNQNAQALNTLVKQLSSNF | |
| GAISSVLNDILSRLDKVEAEVQIDRLITGRLQSLQTYVTQQLIRAAEIRASANLAATKMSECVLGQSK | |
| RVDFCGKGYHLMSFPQAAPHGVVFLHVTYVPSQERNFTTAPAICHEGKAYFPREGVFVFNGTSWFITQ | |
| RNFFSPQIITTDNTFVSGNCDVVIGIINNTVYDPLQPELDSFKEELDKYFKNHTSPDVDLGDIS | |
| >AY274119_tr(CoV_T2_2) | |
| (SEQ ID NO: 4) | |
| Nucleic acid sequence: | |
| atgtttatctttctgctgtttctgaccctgaccagcggcagcgacctggatagatgcacc | |
| accttcgacgatgtgcaggcccctaactacacccagcacaccagctctatgcggggcgtg | |
| tactaccccgacgagattttcagaagcgacaccctgtatctgacccaggacctgttcctg | |
| cctttctacagcaacgtgaccggcttccacaccatcaaccacaccttcggcaaccctgtg | |
| atccccttcaaggacggcatctactttgccgccaccgagaagtccaacgtcgtcagagga | |
| tgggtgttcggcagcaccatgaacaacaagagccagagcgtgatcatcatcaacaacagc | |
| accaacgtggtcatccgggcctgcaacttcgagctgtgcgacaacccattcttcgccgtg | |
| tccaagcctatgggcacccagacacacaccatgatcttcgacaacgccttcaactgcacc | |
| ttcgagtacatcagcgacgccttcagcctggacgtgtccgaaaagagcggcaacttcaag | |
| cacctgagggaattcgtgttcaagaacaaggatggcttcctgtacgtgtacaagggctac | |
| cagcctatcgacgtcgtgcgggatctgcccagcggcttcaataccctgaagcctatcttc | |
| aagctgcccctgggcatcaacatcaccaacttcagagccatcctgaccgctttcagcccc | |
| gctcaggatatctggggaacaagcgccgctgcctacttcgtgggctacctgaagccaacc | |
| accttcatgctgaagtacgacgagaacggcaccatcaccgacgccgtggactgtagccaa | |
| aatcctctggccgagctgaagtgcagcgtgaagtccttcgagatcgacaagggcatctac | |
| cagaccagcaatttcagagtggtgccctccggggatgtcgtgcggttccccaacatcaca | |
| aatctgtgccccttcggcgaggtgttcaacgccaccaagtttccctctgtgtacgcctgg | |
| gagcgcaaaaagatcagcaactgcgtggccgactacagcgtgctgtacaactccaccttc | |
| ttcagcaccttcaagtgctacggcgtgtccgccacaaagctgaacgacctgtgcttctcc | |
| aacgtgtacgccgacagcttcgtggtcaaaggcgacgacgttcggcagattgcccctgga | |
| caaacaggcgtgatcgccgattacaactacaagctgcctgacgacttcatgggctgcgtg | |
| ctggcctggaacaccagaaacatcgatgccacctccaccggcaactacaattacaagtac | |
| agatacctgcggcacggcaagctgcggcctttcgagagggatatcagcaatgtgcctttt | |
| agccccgacggcaagccctgcacacctcctgctctgaattgctactggcccctgaacgac | |
| tacggcttttacaccaccacaggcatcggctatcagccctatagagtggtggtcctgtcc | |
| tttgagctgctgaatgcccctgccacagtgtgcggacctaagctgtctaccgacctgatc | |
| aagaaccagtgcgtgaacttcaacttcaacggcctgaccggcaccggcgtgctgacacca | |
| agcagcaagagattccagcctttccagcagttcggccgggatgtgtccgacttcacagac | |
| agcgtcagagatcccaagaccagcgagatcctggacatcagcccttgtgcctttggcgga | |
| gtgtccgtgatcacccctggcacaaatgcctctagcgaagtggccgtgctgtatcaggac | |
| gtgaactgcaccgatgtgtccaccgccattcacgccgatcagctgactcccgcttggcgg | |
| atctatagcacaggcaacaacgtgttccagacacaagccggctgtctgatcggagccgag | |
| catgtggataccagctacgagtgcgacatccctatcggcgctggcatctgtgcctcttac | |
| cacaccgtgtctctgctgcggagcaccagccagaaatccatcgtggcctacaccatgagc | |
| ctgggcgccgattcttctatcgcctactccaacaacacaatcgctatccccaccaatttc | |
| agcatctccatcaccaccgaagtgatgcccgtgtccatggccaagacctccgtggattgc | |
| aacatgtacatctgcggcgacagcaccgagtgcgccaatctgctgctccagtacggcagc | |
| ttctgcacccagctgaatagagccctgtctggaattgccgccgagcaggacagaaacacc | |
| agagaagtgttcgcccaagtgaagcagatgtataagaccccgacactcaagtacticggc | |
| gggttcaacttctcccagatcctgcctgatcctctgaagcccaccaagcggagcttcatc | |
| gaggacctgctgttcaacaaagtgaccctggccgacgccggctttatgaagcagtatggc | |
| gagtgcctgggcgacatcaacgccagggatctgatttgcgcccagaagtttaacggactg | |
| accgtgctgcctcctctgctgaccgatgatatgatcgccgcctacacagccgctctggtg | |
| tctggtacagctaccgccggatggacatttggagctggcgccgctctccagattccattc | |
| gctatgcagatggcctaccggttcaacggcatcggagtgacccagaatgtgctgtacgag | |
| aatcagaagcagatcgccaatcagttcaacaaggccatcagccagatccaagagagcctg | |
| accaccacaagcacagccctgggaaagctccaggacgtggtcaaccagaatgctcaggcc | |
| ctgaacaccctggtcaagcagctgagcagcaacttcggcgccatcagctccgtgctgaat | |
| gacatcctgagccggctggacaaggtggaagcagaggtgcagatcgaccggctgatcaca | |
| ggcagactccagagcctccagacctacgtgacacagcagctgatcagagccgccgagatt | |
| agagcctctgccaatctggccgccaccaaaatgagcgagtgtgtcctgggccagagcaag | |
| agagtggacttttgcggcaagggctatcacctgatgagcttcccacaggccgctcctcat | |
| ggcgtggtctttctgcacgtgacatacgtgcccagccaagagagaaacttcaccaccgct | |
| ccagccatctgccacgagggcaaagcctactttcccagagaaggcgtgttcgtgtttaac | |
| ggcacctcctggtttatcacccagcggaatttcttcagcccgcaaatcatcaccacagac | |
| aacaccttcgtgtccggcaactgtgacgtcgtgatcggcatcattaacaataccgtgtac | |
| gaccctctccagcctgagctggacagcttcaaagaggaactggataagtacttcaagaat | |
| cacacgagccccgatgtggacctggggatatctct | |
| >AY274119_RBD (CoV_T2_5) | |
| (SEQ ID NO: 5) | |
| Amino acid sequence: | |
| RVVPSGDVVRFPNITNLCPFGEVFNATKFPSVYAWERKKISNCVADYSVLYNSTFFSTFKCYGVSATK | |
| LNDLCFSNVYADSFVVKGDDVRQIAPGQTGVIADYNYKLPDDFMGCVLAWNTRNIDATSTGNYNYKYR | |
| YLRHGKLRPFERDISNVPFSPDGKPCTPPALNCYWPLNDYGFYTTTGIGYQPYRVVVLSFELLNAPAT | |
| VCGPKLSTD | |
| >AY274119_RBD (CoV_T2_5) | |
| (SEQ ID NO: 6) | |
| Nucleic acid sequence: | |
| agagtggtgccctccggggatgtcgtgcggttccccaacatcacaaatctgtgccccttc | |
| ggcgaggtgttcaacgccaccaagtttccctctgtgtacgcctgggagcgcaaaaagatc | |
| agcaactgcgtggccgactacagcgtgctgtacaactccaccttcttcagcaccttcaag | |
| tgctacggcgtgtccgccacaaagctgaacgacctgtgcttctccaacgtgtacgccgac | |
| agcttcgtggtcaaaggcgacgacgttcggcagattgcccctggacaaacaggcgtgatc | |
| gccgattacaactacaagctgcctgacgacttcatgggctgcgtgctggcctggaacacc | |
| agaaacatcgatgccacctccaccggcaactacaattacaagtacagatacctgcggcac | |
| ggcaagctgcggcctttcgagagggatatcagcaatgtgccttttagccccgacggcaag | |
| ccctgcacacctcctgctctgaattgctactggcccctgaacgactacggcttttacacc | |
| accacaggcatcggctatcagccctatagagtggtggtcctgtcctttgagctgctgaat | |
| gcccctgccacagtgtgcggacctaagctgtctaccgac | |
| AY274119 (full length S protein amino acid sequence, with RBD | |
| residues shown in bold, and residues not present in truncated S | |
| protein shown underlined) | |
| (SEQ ID NO: 1) | |
| MFIFLLFLTL TSGSDLDRCT TFDDVQAPNY TQHTSSMRGV YYPDEIFRSD TLYLTQDLFL | 60 |
| PFYSNVTGFH TINHTFGNPV IPFKDGIYFA ATEKSNVVRG WVFGSTMNNK SQSVIIINNS | 120 |
| TNVVIRACNF ELCDNPFFAV SKPMGTQTHT MIFDNAFNCT FEYISDAFSL DVSEKSGNFK | 180 |
| HLREFVFKNK DGFLYVYKGY QPIDVVRDLP SGFNTLKPIF KLPLGINITN FRAILTAFSP | 240 |
| AQDIWGTSAA AYFVGYLKPT TFMLKYDENG TITDAVDCSQ NPLAELKCSV KSFEIDKGIY | 300 |
| QTSNFRVVPS GDVVRFPNIT NLCPFGEVFN ATKFPSVYAW ERKKISNCVA DYSVLYNSTF | 360 |
| FSTFKCYGVS ATKLNDLCFS NVYADSFVVK GDDVRQIAPG QTGVIADYNY KLPDDFMGCV | 420 |
| LAWNTRNIDA TSTGNYNYKY RYLRHGKLRP FERDISNVPF SPDGKPCTPP ALNCYWPLND | 480 |
| YGFYTTTGIG YQPYRVVVLS FELLNAPATV CGPKLSTDLI KNQCVNFNFN GLTGTGVLTP | 540 |
| SSKRFQPFQQ FGRDVSDFTD SVRDPKTSEI LDISPCAFGG VSVITPGTNA SSEVAVLYQD | 600 |
| VNCTDVSTAI HADQLTPAWR IYSTGNNVFQ TQAGCLIGAE HVDTSYECDI PIGAGICASY | 660 |
| HTVSLLRSTS QKSIVAYTMS LGADSSIAYS NNTIAIPTNF SISITTEVMP VSMAKTSVDC | 720 |
| NMYICGDSTE CANLLLQYGS FCTQLNRALS GIAAEQDRNT REVFAQVKQM YKTPTLKYFG | 780 |
| GFNFSQILPD PLKPTKRSFI EDLLFNKVTL ADAGFMKQYG ECLGDINARD LICAQKFNGL | 840 |
| TVLPPLLTDD MIAAYTAALV SGTATAGWTF GAGAALQIPF AMQMAYRFNG IGVTQNVLYE | 900 |
| NQKQIANQFN KAISQIQESL TTTSTALGKL QDVVNQNAQA LNTLVKQLSS NFGAISSVLN | 960 |
| DILSRLDKVE AEVQIDRLIT GRLQSLQTYV TQQLIRAAEI RASANLAATK MSECVLGQSK | 1020 |
| RVDFCGKGYH LMSFPQAAPH GVVFLHVTYV PSQERNFTTA PAICHEGKAY FPREGVFVFN | 1080 |
| GTSWFITQRN FFSPQIITTD NTFVSGNCDV VIGIINNTVY DPLQPELDSF KEELDKYFKN | 1140 |
| HTSPDVDLGD ISGINASVVN IQKEIDRLNE VAKNLNESLI DLQELGKYEQ YIKWPWYVWL | 1200 |
| GFIAGLIAIV MVTILLCCMT SCCSCLKGAC SCGSCCKFDE DDSEPVLKGV KLHYT | 1255 |
| >EPI_ISL_402119 (CoV_T1_2) | |
| (SEQ ID NO: 7) | |
| Amino acid sequence: | |
| MFVFLVLLPLVSSQCVNLTTRTQLPPAYTNSFTRGVYYPDKVFRSSVLHSTQDLFLPFFSNVTWFHAI | |
| HVSGTNGTKRFDNPVLPFNDGVYFASTEKSNIIRGWIFGTTLDSKTQSLLIVNNATNVVIKVCEFQFC | |
| NDPFLGVYYHKNNKSWMESEFRVYSSANNCTFEYVSQPFLMDLEGKQGNFKNLREFVFKNIDGYFKIY | |
| SKHTPINLVRDLPQGFSALEPLVDLPIGINITRFQTLLALHRSYLTPGDSSSGWTAGAAAYYVGYLQP | |
| RTFLLKYNENGTITDAVDCALDPLSETKCTLKSFTVEKGIYQTSNFRVQPTESIVRFPNITNLCPFGE | |
| VFNATRFASVYAWNRKRISNCVADYSVLYNSASFSTFKCYGVSPTKLNDLCFTNVYADSFVIRGDEVR | |
| QIAPGQTGKIADYNYKLPDDFTGCVIAWNSNNLDSKVGGNYNYLYRLFRKSNLKPFERDISTEIYQAG | |
| STPCNGVEGFNCYFPLQSYGFQPTNGVGYQPYRVVVLSFELLHAPATVCGPKKSTNLVKNKCVNFNFN | |
| GLTGTGVLTESNKKFLPFQQFGRDIADTTDAVRDPQTLEILDITPCSFGGVSVITPGTNTSNQVAVLY | |
| QDVNCTEVPVAIHADQLTPTWRVYSTGSNVFQTRAGCLIGAEHVNNSYECDIPIGAGICASYQTQTNS | |
| PRRARSVASQSIIAYTMSLGAENSVAYSNNSIAIPTNFTISVTTEILPVSMTKTSVDCTMYICGDSTE | |
| CSNLLLQYGSFCTQLNRALTGIAVEQDKNTQEVFAQVKQIYKTPPIKDFGGFNFSQILPDPSKPSKRS | |
| FIEDLLFNKVTLADAGFIKQYGDCLGDIAARDLICAQKFNGLTVLPPLLTDEMIAQYTSALLAGTITS | |
| GWTFGAGAALQIPFAMQMAYRFNGIGVTQNVLYENQKLIANQFNSAIGKIQDSLSSTASALGKLQDVV | |
| NQNAQALNTLVKQLSSNFGAISSVLNDILSRLDKVEAEVQIDRLITGRLQSLQTYVTQQLIRAAEIRA | |
| SANLAATKMSECVLGQSKRVDFCGKGYHLMSFPQSAPHGVVFLHVTYVPAQEKNFTTAPAICHDGKAH | |
| FPREGVFVSNGTHWFVTQRNFYEPQIITTDNTFVSGNCDVVIGIVNNTVYDPLQPELDSFKEELDKYF | |
| KNHTSPDVDLGDISGINASVVNIQKEIDRLNEVAKNLNESLIDLQELGKYEQYIKWPWYIWLGFIAGL | |
| IAIVMVTIMLCCMTSCCSCLKGCCSCGSCCKFDEDDSEPVLKGVKLHYT | |
| >EPI_ISL_402119 (CoV_T1_2) | |
| (SEQ ID NO : 8) | |
| Nucleic acid sequence: | |
| atgttcgtgtttctggtgctgctgcctctggtgtccagccagtgtgtgaacctgaccacc | |
| agaacacagctgcctccagcctacaccaacagctttaccagaggcgtgtactaccccgac | |
| aaggtgttcagatccagcgtgctgcactctacccaggacctgttcctgcctttcttcagc | |
| aacgtgacctggttccacgccatccacgtgtccggcaccaatggcaccaagagattcgac | |
| aaccccgtgctgcccttcaacgacggggtgtactttgccagcaccgagaagtccaacatc | |
| atcagaggctggatcttcggcaccacactggacagcaagacccagagcctgctgatcgtg | |
| aacaacgccaccaacgtggtcatcaaagtgtgcgagttccagttctgcaacgaccccttc | |
| ctgggcgtctactaccacaagaacaacaagagctggatggaaagcgagttccgggtgtac | |
| agcagcgccaacaactgcaccttcgagtacgtgtcccagcctttcctgatggacctggaa | |
| ggcaagcagggcaacttcaagaacctgcgcgagttcgtgttcaagaacatcgacggctac | |
| ttcaaaatctacagcaagcacacccctatcaacctcgtgcgggatctgcctcagggcttc | |
| tctgctctggaacccctggtggatctgcccatcggcatcaacatcacccggtttcagaca | |
| ctgctggccctgcacagaagctacctgacacctggcgatagcagcagcggatggacagct | |
| ggtgccgccgcttactacgtgggatacctccagccaagaaccttcctgctgaagtacaac | |
| gagaacggcaccatcaccgacgccgtggattgtgctctggaccctctgagcgagacaaag | |
| tgcaccctgaagtccttcaccgtggaaaagggcatctaccagaccagcaacttccgggtg | |
| cagcccaccgaatccatcgtgcggttccccaatatcaccaatctgtgccccttcggcgag | |
| gtgttcaatgccaccagattcgcctctgtgtacgcctggaaccggaagcggatcagcaat | |
| tgcgtggccgactactccgtgctgtacaactccgccagcttcagcaccttcaagtgctac | |
| ggcgtgtcccctaccaagctgaacgacctgtgcttcacaaacgtgtacgccgacagcttc | |
| gtgatccggggagatgaagtgcggcagattgcccctggacagacaggcaagatcgccgac | |
| tacaactacaagctgcccgacgacttcaccggctgtgtgattgcctggaacagcaacaac | |
| ctggactccaaagtcggcggcaactacaattacctgtaccggctgttccggaagtccaat | |
| ctgaagcccttcgagcgggacatcagcaccgaaatctatcaggccggcagcaccccttgc | |
| aacggcgtggaaggcttcaactgctacttcccactgcaaagctacggctttcagcccaca | |
| aatggcgtgggctaccagccttacagagtggtggtgctgagcttcgagctgctgcatgct | |
| cctgccacagtgtgcggccctaagaaatccaccaatctcgtgaagaacaaatgcgtgaac | |
| ttcaacttcaacggcctgaccggcaccggcgtgctgacagagagcaacaagaagttcctg | |
| ccattccagcagttcggccgggatatcgccgataccacagatgccgtcagagatccccag | |
| acactggaaatcctggacatcaccccatgcagcttcggcggagtgtctgtgatcacccct | |
| ggcaccaacaccagcaatcaggtggcagtgctgtaccaggacgtgaactgtaccgaagtg | |
| cccgtggccattcacgccgatcagctgacacctacatggcgggtgtactccaccggcagc | |
| aatgtgtttcagaccagagccggctgtctgatcggagccgagcacgtgaacaatagctac | |
| gagtgcgacatccccatcggcgctggcatctgcgcctcttaccagacacagacaaacagc | |
| cccagacgggctagaagcgtggccagccagagcatcattgcctacacaatgtctctgggc | |
| gccgagaacagcgtggcctactccaacaactctatcgctatccccaccaacttcaccatc | |
| agcgtgaccaccgagatcctgcctgtgtccatgaccaagaccagcgtggactgcaccatg | |
| tacatctgcggcgattccaccgagtgctccaacctgctgctccagtacggcagcttctgc | |
| acccagctgaatagagccctgacagggatcgccgtggaacaggacaagaacacccaagag | |
| gtgttcgcccaagtgaagcaaatctacaagacccctcctatcaaggacttcggcggcttc | |
| aatttcagccagattctgcccgatcctagcaagcccagcaagcggagcttcatcgaggac | |
| ctgctgttcaacaaagtgacactggccgacgccggcttcatcaagcagtacggcgattgt | |
| ctgggcgacattgccgccagggatctgatttgcgcccagaagtttaacggactgacagtg | |
| ctgcctcctctgctgaccgatgagatgatcgcccagtacacatctgccctgctggccggc | |
| acaatcacaagcggctggacatttggagctggcgccgctctccagattccattcgctatg | |
| cagatggcctaccggttcaacggcatcggagtgacccagaatgtgctgtacgagaaccag | |
| aagctgatcgccaaccagttcaacagcgccatcggcaagatccaggacagcctgagcagc | |
| acagcaagcgccctgggaaagctccaggacgtcgtgaaccagaatgcccaggcactgaac | |
| accctggtcaagcagctgtcctccaacttcggcgccatcagctctgtgctgaacgatatc | |
| ctgagcagactggacaaggtggaagccgaggtgcagatcgacagactgatcaccggcaga | |
| ctccagtctctccagacctacgtgacccagcagctgatcagagccgccgagattagagcc | |
| tctgccaatctggccgccaccaagatgtctgagtgtgtgctgggccagagcaagagagtg | |
| gacttttgcggcaagggctaccacctgatgagcttccctcagtctgcccctcacggcgtg | |
| gtgtttctgcacgtgacatacgtgcccgctcaagagaagaatttcaccaccgctccagcc | |
| atctgccacgacggcaaagcccactttcctagagaaggcgtgttcgtgtccaacggcacc | |
| cattggttcgtgacacagcggaacttctacgagccccagatcatcaccaccgacaacacc | |
| ttcgtgtctggcaactgcgacgttgtgatcggcattgtgaacaataccgtgtacgaccct | |
| ctccagcctgaactggactccttcaaagaggaactcgacaagtactttaagaaccacaca | |
| agccccgacgtggacctgggcgatatcagcggaatcaatgccagcgtggtcaacatccag | |
| aaagagatcgaccggctgaacgaggtggccaagaatctgaacgagagcctgatcgacctg | |
| caagaactggggaagtacgagcagtacatcaagtggccctggtacatctggctgggcttt | |
| atcgccggactgattgccatcgtgatggtcacaatcatgctgtgttgcatgaccagctgc | |
| tgtagctgcctgaagggctgttgtagctgtggctcctgctgcaagttcgacgaggacgat | |
| tctgagcccgtgctgaagggcgtgaaactgcactacacc | |
| >EPI_ISL_402119_tr (CoV_T2_3) | |
| (SEQ ID NO: 9) | |
| Amino acid sequence: | |
| MFVFLVLLPLVSSQCVNLTTRTQLPPAYTNSFTRGVYYPDKVFRSSVLHSTQDLFLPFFSNVTWFHAI | |
| HVSGTNGTKRFDNPVLPFNDGVYFASTEKSNIIRGWIFGTTLDSKTQSLLIVNNATNVVIKVCEFQFC | |
| NDPFLGVYYHKNNKSWMESEFRVYSSANNCTFEYVSQPFLMDLEGKQGNFKNLREFVFKNIDGYFKIY | |
| SKHTPINLVRDLPQGFSALEPLVDLPIGINITRFQTLLALHRSYLTPGDSSSGWTAGAAAYYVGYLQP | |
| RTFLLKYNENGTITDAVDCALDPLSETKCTLKSFTVEKGIYQTSNFRVQPTESIVRFPNITNLCPFGE | |
| VFNATRFASVYAWNRKRISNCVADYSVLYNSASFSTFKCYGVSPTKLNDLCFTNVYADSFVIRGDEVR | |
| QIAPGQTGKIADYNYKLPDDFTGCVIAWNSNNLDSKVGGNYNYLYRLFRKSNLKPFERDISTEIYQAG | |
| STPCNGVEGFNCYFPLQSYGFQPTNGVGYQPYRVVVLSFELLHAPATVCGPKKSTNLVKNKCVNFNFN | |
| GLTGTGVLTESNKKFLPFQQFGRDIADTTDAVRDPQTLEILDITPCSFGGVSVITPGTNTSNQVAVLY | |
| QDVNCTEVPVAIHADQLTPTWRVYSTGSNVFQTRAGCLIGAEHVNNSYECDIPIGAGICASYQTQTNS | |
| PRRARSVASQSIIAYTMSLGAENSVAYSNNSIAIPTNFTISVTTEILPVSMTKTSVDCTMYICGDSTE | |
| CSNLLLQYGSFCTQLNRALTGIAVEQDKNTQEVFAQVKQIYKTPPIKDFGGFNFSQILPDPSKPSKRS | |
| FIEDLLFNKVTLADAGFIKQYGDCLGDIAARDLICAQKFNGLTVLPPLLTDEMIAQYTSALLAGTITS | |
| GWTFGAGAALQIPFAMQMAYRFNGIGVTQNVLYENQKLIANQFNSAIGKIQDSLSSTASALGKLQDVV | |
| NQNAQALNTLVKQLSSNFGAISSVLNDILSRLDKVEAEVQIDRLITGRLQSLQTYVTQQLIRAAEIRA | |
| SANLAATKMSECVLGQSKRVDFCGKGYHLMSFPQSAPHGVVFLHVTYVPAQEKNFTTAPAICHDGKAH | |
| FPREGVFVSNGTHWFVTQRNFYEPQIITTDNTFVSGNCDVVIGIVNNTVYDPLQPELDSFKEELDKYF | |
| KNHTSPDVDLGDIS | |
| >EPI_ISL_402119_tr (CoV_T2_3) | |
| (SEQ ID NO: 10) | |
| Nucleic acid sequence: | |
| atgttcgtgtttctggtgctgctgcctctggtgtccagccagtgtgtgaacctgaccacc | |
| agaacacagctgcctccagcctacaccaacagctttaccagaggcgtgtactaccccgac | |
| aaggtgttcagatccagcgtgctgcactctacccaggacctgttcctgcctttcttcagc | |
| aacgtgacctggttccacgccatccacgtgtccggcaccaatggcaccaagagattcgac | |
| aaccccgtgctgcccttcaacgacggggtgtactttgccagcaccgagaagtccaacatc | |
| atcagaggctggatcttcggcaccacactggacagcaagacccagagcctgctgatcgtg | |
| aacaacgccaccaacgtggtcatcaaagtgtgcgagttccagttctgcaacgaccccttc | |
| ctgggcgtctactaccacaagaacaacaagagctggatggaaagcgagttccgggtgtac | |
| agcagcgccaacaactgcaccttcgagtacgtgtcccagcctttcctgatggacctggaa | |
| ggcaagcagggcaacttcaagaacctgcgcgagttcgtgttcaagaacatcgacggctac | |
| ttcaaaatctacagcaagcacacccctatcaacctcgtgcgggatctgcctcagggcttc | |
| tctgctctggaacccctggtggatctgcccatcggcatcaacatcacccggtttcagaca | |
| ctgctggccctgcacagaagctacctgacacctggcgatagcagcagcggatggacagct | |
| ggtgccgccgcttactacgtgggatacctccagccaagaaccttcctgctgaagtacaac | |
| gagaacggcaccatcaccgacgccgtggattgtgctctggaccctctgagcgagacaaag | |
| tgcaccctgaagtccttcaccgtggaaaagggcatctaccagaccagcaacttccgggtg | |
| cagcccaccgaatccatcgtgcggttccccaatatcaccaatctgtgccccttcggcgag | |
| gtgttcaatgccaccagattcgcctctgtgtacgcctggaaccggaagcggatcagcaat | |
| tgcgtggccgactactccgtgctgtacaactccgccagcttcagcaccttcaagtgctac | |
| ggcgtgtcccctaccaagctgaacgacctgtgcttcacaaacgtgtacgccgacagcttc | |
| gtgatccggggagatgaagtgcggcagattgcccctggacagacaggcaagatcgccgac | |
| tacaactacaagctgcccgacgacttcaccggctgtgtgattgcctggaacagcaacaac | |
| ctggactccaaagtcggcggcaactacaattacctgtaccggctgttccggaagtccaat | |
| ctgaagcccttcgagcgggacatcagcaccgaaatctatcaggccggcagcaccccttgc | |
| aacggcgtggaaggcttcaactgctacttcccactgcaaagctacggctttcagcccaca | |
| aatggcgtgggctaccagccttacagagtggtggtgctgagcttcgagctgctgcatgct | |
| cctgccacagtgtgcggccctaagaaatccaccaatctcgtgaagaacaaatgcgtgaac | |
| ttcaacttcaacggcctgaccggcaccggcgtgctgacagagagcaacaagaagttcctg | |
| ccattccagcagttcggccgggatatcgccgataccacagatgccgtcagagatccccag | |
| acactggaaatcctggacatcaccccatgcagcttcggcggagtgtctgtgatcacccct | |
| ggcaccaacaccagcaatcaggtggcagtgctgtaccaggacgtgaactgtaccgaagtg | |
| cccgtggccattcacgccgatcagctgacacctacatggcgggtgtactccaccggcagc | |
| aatgtgtttcagaccagagccggctgtctgatcggagccgagcacgtgaacaatagctac | |
| gagtgcgacatccccatcggcgctggcatctgcgcctcttaccagacacagacaaacagc | |
| cccagacgggctagaagcgtggccagccagagcatcattgcctacacaatgtctctgggc | |
| gccgagaacagcgtggcctactccaacaactctatcgctatccccaccaacttcaccatc | |
| agcgtgaccaccgagatcctgcctgtgtccatgaccaagaccagcgtggactgcaccatg | |
| tacatctgcggcgattccaccgagtgctccaacctgctgctccagtacggcagcttctgc | |
| acccagctgaatagagccctgacagggatcgccgtggaacaggacaagaacacccaagag | |
| gtgttcgcccaagtgaagcaaatctacaagacccctcctatcaaggacttcggcggcttc | |
| aatttcagccagattctgcccgatcctagcaagcccagcaagcggagcttcatcgaggac | |
| ctgctgttcaacaaagtgacactggccgacgccggcttcatcaagcagtacggcgattgt | |
| ctgggcgacattgccgccagggatctgatttgcgcccagaagtttaacggactgacagtg | |
| ctgcctcctctgctgaccgatgagatgatcgcccagtacacatctgccctgctggccggc | |
| acaatcacaagcggctggacatttggagctggcgccgctctccagattccattcgctatg | |
| cagatggcctaccggttcaacggcatcggagtgacccagaatgtgctgtacgagaaccag | |
| aagctgatcgccaaccagttcaacagcgccatcggcaagatccaggacagcctgagcagc | |
| acagcaagcgccctgggaaagctccaggacgtcgtgaaccagaatgcccaggcactgaac | |
| accctggtcaagcagctgtcctccaacttcggcgccatcagctctgtgctgaacgatatc | |
| ctgagcagactggacaaggtggaagccgaggtgcagatcgacagactgatcaccggcaga | |
| ctccagtctctccagacctacgtgacccagcagctgatcagagccgccgagattagagcc | |
| tctgccaatctggccgccaccaagatgtctgagtgtgtgctgggccagagcaagagagtg | |
| gacttttgcggcaagggctaccacctgatgagcttccctcagtctgcccctcacggcgtg | |
| gtgtttctgcacgtgacatacgtgcccgctcaagagaagaatttcaccaccgctccagcc | |
| atctgccacgacggcaaagcccactttcctagagaaggcgtgttcgtgtccaacggcacc | |
| cattggttcgtgacacagcggaacttctacgagccccagatcatcaccaccgacaacacc | |
| ttcgtgtctggcaactgcgacgttgtgatcggcattgtgaacaataccgtgtacgaccct | |
| ctccagcctgaactggactccttcaaagaggaactcgacaagtactttaagaaccacaca | |
| agccccgacgtggacctgggcgatatcagt | |
| >EPI_ISL_402119_RBD (CoV_T2_6) | |
| (SEQ ID NO: 11) | |
| Amino acid sequence: | |
| RVQPTESIVRFPNITNLCPFGEVFNATRFASVYAWNRKRISNCVADYSVLYNSASFSTFKCYGVSPTK | |
| LNDLCFTNVYADSFVIRGDEVRQIAPGQTGKIADYNYKLPDDFTGCVIAWNSNNLDSKVGGNYNYLYR | |
| LFRKSNLKPFERDISTEIYQAGSTPCNGVEGFNCYFPLQSYGFQPTNGVGYQPYRVVVLSFELLHAPA | |
| TVCGPKKSTN | |
| >EPI_ISL_402119_RBD (COV_T2_6) | |
| (SEQ ID NO: 12) | |
| Nucleic acid sequence: | |
| cgggtgcagcccaccgaatccatcgtgcggttccccaatatcaccaatctgtgccccttc | |
| ggcgaggtgttcaatgccaccagattcgcctctgtgtacgcctggaaccggaagcggatc | |
| agcaattgcgtggccgactactccgtgctgtacaactccgccagcttcagcaccttcaag | |
| tgctacggcgtgtcccctaccaagctgaacgacctgtgcttcacaaacgtgtacgccgac | |
| agcttcgtgatccggggagatgaagtgcggcagattgcccctggacagacaggcaagatc | |
| gccgactacaactacaagctgcccgacgacttcaccggctgtgtgattgcctggaacagc | |
| aacaacctggactccaaagtcggcggcaactacaattacctgtaccggctgttccggaag | |
| tccaatctgaagcccttcgagcgggacatcagcaccgaaatctatcaggccggcagcacc | |
| ccttgcaacggcgtggaaggcttcaactgctacttcccactgcaaagctacggctttcag | |
| cccacaaatggcgtgggctaccagccttacagagtggtggtgctgagcttcgagctgctg | |
| catgctcctgccacagtgtgcggccctaagaaatccaccaat | |
| EPI_ISL_402119 (full length S protein amino acid sequence, with RBD | |
| residues shown in bold, and residues not present in truncated S | |
| protein shown underlined) | |
| (SEQ ID NO: 7) | |
| MFVFLVLLPL VSSQCVNLTT RTQLPPAYTN SFTRGVYYPD KVFRSSVLHS TQDLFLPFFS | 60 |
| NVTWFHAIHV SGTNGTKRFD NPVLPFNDGV YFASTEKSNI IRGWIFGTTL DSKTQSLLIV | 120 |
| NNATNVVIKV CEFQFCNDPF LGVYYHKNNK SWMESEFRVY SSANNCTFEY VSQPFLMDLE | 180 |
| GKQGNFKNLR EFVFKNIDGY FKIYSKHTPI NLVRDLPQGF SALEPLVDLP IGINITRFQT | 240 |
| LLALHRSYLT PGDSSSGWTA GAAAYYVGYL QPRTFLLKYN ENGTITDAVD CALDPLSETK | 300 |
| CTLKSFTVEK GIYQTSNFRV QPTESIVRFP NITNLCPFGE VFNATRFASV YAWNRKRISN | 360 |
| CVADYSVLYN SASFSTFKCY GVSPTKLNDL CFTNVYADSF VIRGDEVRQI APGQTGKIAD | 420 |
| YNYKLPDDFT GCVIAWNSNN LDSKVGGNYN YLYRLFRKSN LKPFERDIST EIYQAGSTPC | 480 |
| NGVEGFNCYF PLQSYGFQPT NGVGYQPYRV VVLSFELLHA PATVCGPKKS TNLVKNKCVN | 540 |
| FNFNGLTGTG VLTESNKKFL PFQQFGRDIA DTTDAVRDPQ TLEILDITPC SFGGVSVITP | 600 |
| GTNTSNQVAV LYQDVNCTEV PVAIHADQLT PTWRVYSTGS NVFQTRAGCL IGAEHVNNSY | 660 |
| ECDIPIGAGI CASYQTQTNS PRRARSVASQ SIIAYTMSLG AENSVAYSNN SIAIPTNFTI | 720 |
| SVTTEILPVS MTKTSVDCTM YICGDSTECS NLLLQYGSFC TQLNRALTGI AVEQDKNTQE | 780 |
| VFAQVKQIYK TPPIKDFGGF NFSQILPDPS KPSKRSFIED LLFNKVTLAD AGFIKQYGDC | 840 |
| LGDIAARDLI CAQKFNGLTV LPPLLTDEMI AQYTSALLAG TITSGWTFGA GAALQIPFAM | 900 |
| QMAYRFNGIG VTQNVLYENQ KLIANQFNSA IGKIQDSLSS TASALGKLQD VVNQNAQALN | 960 |
| TLVKQLSSNF GAISSVLNDI LSRLDKVEAE VQIDRLITGR LQSLQTYVTQ QLIRAAEIRA | 1020 |
| SANLAATKMS ECVLGQSKRV DFCGKGYHLM SFPQSAPHGV VFLHVTYVPA QEKNFTTAPA | 1080 |
| ICHDGKAHFP REGVFVSNGT HWFVTQRNFY EPQIITTDNT FVSGNCDVVI GIVNNTVYDP | 1140 |
| LQPELDSFKE ELDKYFKNHT SPDVDLGDIS GINASVVNIQ KEIDRLNEVA KNLNESLIDL | 1200 |
| QELGKYEQYI KWPWYIWLGF IAGLIAIVMV TIMLCCMTSC CSCLKGCCSC GSCCKFDEDD | 1260 |
| SEPVLKGVKL HYT | 1273 |
| >Wuhan_Node1 (CoV_T2_1) | |
| (SEQ ID NO: 13) | |
| Amino acid sequence: | |
| MFLFLFIIIFAFFLLSAKANERCGIFTSKPQPKLAQVSSSRRGVYYPDDIFRSDVLHLTQDYFLPFDS | |
| NVTRYFSLNANGPDRIVYFDNPIIPFKDGVYFAATEKSNVIRGWIFGSTLDNTSQSVIIVNNSTNVII | |
| RVCNFDLCNDPFFTVSRPTDKHIKTWSIREFAVYQSAFNCTFEYVSKSFLLDVAEKPGNFKHLREFVF | |
| KNVDGFLNVYSTYKPINVVSGLPTGFSVLKPILKLPLGINITSFRVLLTMFRGDPTPGHTTANWLTAA | |
| AAYYVGYLKPTTFMLKYNENGTITDAVDCSQNPLAELKCTLKNFNVDKGIYQTSNFRVSPTQEVVRFP | |
| NITNLCPFDKVFNATRFPSVYAWERTKISDCVADYTVLYNSTSFSTFKCYGVSPSKLIDLCFTSVYAD | |
| TFLIRCSEVRQVAPGQTGVIADYNYKLPDDFTGCVIAWNTAKQDTGSSGNYNYYYRSHRKTKLKPFER | |
| DLSSDECSPDGKPCTPPAFNGVRGFNCYFTLSTYDFNPNVPVEYQATRVVVLSFELLNAPATVCGPKL | |
| STQLVKNQCVNFNFNGLKGTGVLTASSKRFQSFQQFGRDASDFTDSVRDPQTLEILDISPCSFGGVSV | |
| ITPGTNTSSEVAVLYQDVNCTDVPTAIHADQLTPAWRVYSTGVNVFQTQAGCLIGAEHVNASYECDIP | |
| IGAGICASYHTASNSPRILRSTGQKSIVAYTMSLGAENSIAYANNSIAIPTNFSISVTTEVMPVSMAK | |
| TSVDCTMYICGDSLECSNLLLQYGSFCTQLNRALTGIAIEQDKNTQEVFAQVKQMYKTPAIKDFGGFN | |
| FSQILPDPSKPTKRSFIEDLLFNKVTLADAGFMKQYGECLGDISARDLICAQKFNGLTVLPPLLTDEM | |
| IAAYTAALVSGTATAGWTFGAGAALQIPFAMQMAYRFNGIGVTQNVLYENQKQIANQFNKAISQIQES | |
| LTTTSTALGKLQDVVNQNAQALNTLVKQLSSNFGAISSVLNDILSRLDKVEAEVQIDRLITGRLQSLQ | |
| TYVTQQLIRAAEIRASANLAATKMSECVLGQSKRVDFCGKGYHLMSFPQAAPHGVVFLHVTYVPSQER | |
| NFTTAPAICHEGKAYFPREGVFVSNGTSWFITQRNFYSPQIITTDNTFVSGNCDVVIGIINNTVYDPL | |
| QPELDSFKEELDKYFKNHTSPDVDLGDISGINASVVNIQKEIDRLNEVAKNLNESLIDLQELGKYEQY | |
| IKWPWYVWLGFIAGLIAIVMATILLCCMTSCCSCLKGACSCGSCCKFDEDDSEPVLKGVKLHYT | |
| >Wuhan_Node1 (CoV_T2_1) | |
| (SEQ ID NO: 14) | |
| Nucleic acid sequence : | |
| atgtttctgttcctcttcattattatcttcgcattcttcctgctgagcgccaaggccaac | |
| gagagatgcggcatcttcaccagcaagccccagcctaagctggcccaggtgtccagttct | |
| agacggggcgtgtactaccccgacgacatcttcagatccgacgtgctgcatctgacccag | |
| gactacttcctgcctttcgacagcaacgtgacccggtacttcagcctgaacgccaacgga | |
| cccgaccggatcgtgtacttcgacaaccctatcatccccttcaaggacggggtgtacttt | |
| gccgccaccgagaagtccaacgtgatcagaggctggatcttcggcagcaccctggacaat | |
| accagccagagcgtgatcatcgtgaacaacagcaccaacgtcatcatccgcgtgtgcaac | |
| ttcgacctgtgcaacgacccattcttcaccgtgtccagaccaaccgacaagcacatcaag | |
| acctggtccatccgcgagttcgccgtgtaccagagcgccttcaattgcaccttcgagtac | |
| gtgtccaagagctttctgctggacgtggccgagaagcccggcaactttaagcacctgaga | |
| gaattcgtgttcaagaacgtggacggcttcctgaacgtgtacagcacctacaagcccatc | |
| aacgtggtgtccggcctgcctacaggattcagcgtgctgaagcccatcctgaagctgccc | |
| ctgggcatcaacatcaccagcttcagagtgctgctgaccatgttcagaggcgaccctaca | |
| cctggccacaccaccgctaattggctgacagccgccgctgcctactacgtgggatacctg | |
| aagcctaccaccttcatgctcaagtacaacgagaacggcaccatcaccgacgccgtggac | |
| tgtagccaaaatcctctggccgagctgaagtgcaccctgaagaacttcaacgtggacaag | |
| ggcatctaccagaccagcaacttccgggtgtcccctacacaagaggtcgtgcggttcccc | |
| aatatcaccaatctgtgccccttcgacaaggtgttcaacgccaccagatttcccagcgtg | |
| tacgcctgggagcgcaccaagatttccgattgcgtggccgactacaccgtgctgtataac | |
| tccacctccttcagcaccttcaagtgctacggcgtgtccccaagcaagctgatcgatctg | |
| tgcttcacctctgtgtacgccgacaccttcctgatccggtgtagcgaagtgcgacaggtg | |
| gcacctggacagacaggcgtgatcgccgattacaactacaagctgcccgacgacttcacc | |
| ggctgtgtgatcgcctggaataccgccaagcaggatacaggcagcagcggcaactacaac | |
| tactactacagaagccaccgcaagaccaagctgaagcctttcgagagggacctgagcagc | |
| gacgagtgtagccctgatggcaagccttgtacacctcctgccttcaatggcgtgcggggc | |
| ttcaactgctacttcaccctgagcacctacgacttcaaccccaacgtgcccgtggaatac | |
| caggccacaagagtggtggtgctgagcttcgagctgctgaatgcccctgccacagtgtgt | |
| ggccctaagctgtctacccagctggtcaagaaccagtgcgtgaacttcaatttcaacggc | |
| ctgaaaggcaccggcgtgctgaccgccagcagcaagagattccagagcttccagcagttc | |
| ggcagggacgccagcgatttcacagatagcgtcagagatccccagacactggaaatcctg | |
| gacatcagcccttgcagcttcggcggagtgtctgtgatcacccctggcaccaatacctct | |
| agcgaggtggcagtgctgtaccaggacgtgaactgcaccgatgtgcctacagccatccac | |
| gccgatcagctgacaccagcttggagagtgtactctaccggtgtcaacgtgttccagaca | |
| caagccggctgtctgattggagccgaacacgtgaacgccagctacgagtgcgacatccct | |
| atcggagccggcatctgtgcctcttaccacaccgcctctaacagccccagaatcctgaga | |
| agcaccggccagaaatccatcgtggcctacacaatgtctctgggcgccgagaactctatc | |
| gcctacgccaacaactccattgctatccccaccaacttcagcatctccgtgaccaccgaa | |
| gtgatgcctgtgtccatggccaagaccagcgtggactgcacaatgtacatctgcggcgac | |
| agcctggaatgcagcaacctgctgctccagtacggcagcttctgcacccagctgaataga | |
| gccctgaccggaatcgccatcgagcaggacaagaacacccaagaggtgttcgcccaagtg | |
| aagcagatgtataagacccctgccatcaaggacttcggcggctttaacttcagccagatc | |
| ctgcctgatcctagcaagcccaccaagcggagcttcatcgaggacctgctgttcaacaaa | |
| gtgaccctggccgacgccggctttatgaagcagtatggcgagtgcctgggcgacatctct | |
| gccagggatctgatttgcgcccagaagttcaacggactgaccgtgctgcctcctctgctg | |
| accgatgagatgatcgccgcctatacagccgctctggtgtctggcacagctaccgccgga | |
| tggacatttggagctggcgccgctctccagattccattcgctatgcagatggcctaccgc | |
| ttcaacggcatcggcgtgacccagaacgtgctgtacgagaaccagaagcagatcgccaac | |
| cagttcaacaaggccatcagtcagatccaagagagcctgaccacaaccagcacagccctg | |
| ggaaagctccaggacgtcgtgaaccagaatgcccaggctctgaacaccctggtcaagcag | |
| ctgagcagcaatttcggcgccatcagctccgtgctgaacgacatcctgagccggctggat | |
| aaggtggaagccgaggtgcagatcgaccggctgattacaggcagactccagtctctccag | |
| acctacgtgacacagcagctgatcagagccgccgagattagagcctctgccaatctggcc | |
| gccaccaagatgtctgagtgtgtgctgggccagtctaagagagtggacttctgcggcaag | |
| ggctaccacctgatgagcttccctcaggctgctcctcacggcgtggtgtttctgcacgtg | |
| acatacgtgcccagccaagagcggaacttcacaactgccccagccatctgccacgagggc | |
| aaagcctactttcccagagaaggcgtgttcgtgtccaacggcacctcctggttcatcacc | |
| cagagaaacttctacagccctcagatcatcaccaccgacaacaccttcgtgtccggcaac | |
| tgcgacgtggtcatcggcatcatcaacaataccgtgtacgaccctctccagccagaactg | |
| gatagcttcaaagaggaactcgacaagtacttcaagaatcacacaagccccgacgtggac | |
| ctgggcgatatcagcggaatcaatgccagcgtggtcaacatccagaaagagatcgacaga | |
| ctgaacgaggtggccaagaacctgaacgagtccctgatcgacctgcaagagctggggaag | |
| tacgagcagtacatcaagtggccttggtacgtgtggctgggctttatcgccggactgatc | |
| gccattgtgatggccaccatcctgctgtgctgcatgacaagctgctgtagctgcctgaag | |
| ggcgcctgtagctgtggcagctgctgcaagttcgacgaggacgattctgagcctgtgctg | |
| aaaggcgtgaagctgcactacacc | |
| >Wuhan_Node1_tr (CoV_T2_4) | |
| (SEQ ID NO: 15) | |
| Amino acid sequence: | |
| MFLFLFIIIFAFFLLSAKANERCGIFTSKPQPKLAQVSSSRRGVYYPDDIFRSDVLHLTQDYFLPFDS | |
| NVTRYFSLNANGPDRIVYFDNPIIPFKDGVYFAATEKSNVIRGWIFGSTLDNTSQSVIIVNNSTNVII | |
| RVCNFDLCNDPFFTVSRPTDKHIKTWSIREFAVYQSAFNCTFEYVSKSFLLDVAEKPGNFKHLREFVF | |
| KNVDGFLNVYSTYKPINVVSGLPTGFSVLKPILKLPLGINITSFRVLLTMFRGDPTPGHTTANWLTAA | |
| AAYYVGYLKPTTFMLKYNENGTITDAVDCSQNPLAELKCTLKNFNVDKGIYQTSNFRVSPTQEVVRFP | |
| NITNLCPFDKVFNATRFPSVYAWERTKISDCVADYTVLYNSTSFSTFKCYGVSPSKLIDLCFTSVYAD | |
| TFLIRCSEVRQVAPGQTGVIADYNYKLPDDFTGCVIAWNTAKQDTGSSGNYNYYYRSHRKTKLKPFER | |
| DLSSDECSPDGKPCTPPAFNGVRGFNCYFTLSTYDFNPNVPVEYQATRVVVLSFELLNAPATVCGPKL | |
| STQLVKNQCVNFNFNGLKGTGVLTASSKRFQSFQQFGRDASDFTDSVRDPQTLEILDISPCSFGGVSV | |
| ITPGTNTSSEVAVLYQDVNCTDVPTAIHADQLTPAWRVYSTGVNVFQTQAGCLIGAEHVNASYECDIP | |
| IGAGICASYHTASNSPRILRSTGQKSIVAYTMSLGAENSIAYANNSIAIPTNFSISVTTEVMPVSMAK | |
| TSVDCTMYICGDSLECSNLLLQYGSFCTQLNRALTGIAIEQDKNTQEVFAQVKQMYKTPAIKDFGGFN | |
| FSQILPDPSKPTKRSFIEDLLFNKVTLADAGFMKQYGECLGDISARDLICAQKFNGLTVLPPLLTDEM | |
| IAAYTAALVSGTATAGWTFGAGAALQIPFAMQMAYRFNGIGVTQNVLYENQKQIANQFNKAISQIQES | |
| LTTTSTALGKLQDVVNQNAQALNTLVKQLSSNFGAISSVLNDILSRLDKVEAEVQIDRLITGRLQSLQ | |
| TYVTQQLIRAAEIRASANLAATKMSECVLGQSKRVDFCGKGYHLMSFPQAAPHGVVFLHVTYVPSQER | |
| NFTTAPAICHEGKAYFPREGVFVSNGTSWFITQRNFYSPQIITTDNTFVSGNCDVVIGIINNTVYDPL | |
| QPELDSFKEELDKYFKNHTSPDVDLGDIS | |
| >Wuhan_Node1_tr (CoV_T2_4) | |
| (SEQ ID NO: 16) | |
| Nucleic acid sequence : | |
| atgtttctgttcctcttcattattatcttcgcattcttcctgctgagcgccaaggccaac | |
| gagagatgcggcatcttcaccagcaagccccagcctaagctggcccaggtgtccagttct | |
| agacggggcgtgtactaccccgacgacatcttcagatccgacgtgctgcatctgacccag | |
| gactacttcctgcctttcgacagcaacgtgacccggtacttcagcctgaacgccaacgga | |
| cccgaccggatcgtgtacttcgacaaccctatcatccccttcaaggacggggtgtacttt | |
| gccgccaccgagaagtccaacgtgatcagaggctggatcttcggcagcaccctggacaat | |
| accagccagagcgtgatcatcgtgaacaacagcaccaacgtcatcatccgcgtgtgcaac | |
| ttcgacctgtgcaacgacccattcttcaccgtgtccagaccaaccgacaagcacatcaag | |
| acctggtccatccgcgagttcgccgtgtaccagagcgccttcaattgcaccttcgagtac | |
| gtgtccaagagctttctgctggacgtggccgagaagcccggcaactttaagcacctgaga | |
| gaattcgtgttcaagaacgtggacggcttcctgaacgtgtacagcacctacaagcccatc | |
| aacgtggtgtccggcctgcctacaggattcagcgtgctgaagcccatcctgaagctgccc | |
| ctgggcatcaacatcaccagcttcagagtgctgctgaccatgttcagaggcgaccctaca | |
| cctggccacaccaccgctaattggctgacagccgccgctgcctactacgtgggatacctg | |
| aagcctaccaccttcatgctcaagtacaacgagaacggcaccatcaccgacgccgtggac | |
| tgtagccaaaatcctctggccgagctgaagtgcaccctgaagaacttcaacgtggacaag | |
| ggcatctaccagaccagcaacttccgggtgtcccctacacaagaggtcgtgcggttcccc | |
| aatatcaccaatctgtgccccttcgacaaggtgttcaacgccaccagatttcccagcgtg | |
| tacgcctgggagcgcaccaagatttccgattgcgtggccgactacaccgtgctgtataac | |
| tccacctccttcagcaccttcaagtgctacggcgtgtccccaagcaagctgatcgatctg | |
| tgcttcacctctgtgtacgccgacaccttcctgatccggtgtagcgaagtgcgacaggtg | |
| gcacctggacagacaggcgtgatcgccgattacaactacaagctgcccgacgacttcacc | |
| ggctgtgtgatcgcctggaataccgccaagcaggatacaggcagcagcggcaactacaac | |
| tactactacagaagccaccgcaagaccaagctgaagcctttcgagagggacctgagcagc | |
| gacgagtgtagccctgatggcaagccttgtacacctcctgccttcaatggcgtgcggggc | |
| ttcaactgctacttcaccctgagcacctacgacttcaaccccaacgtgcccgtggaatac | |
| caggccacaagagtggtggtgctgagcttcgagctgctgaatgcccctgccacagtgtgt | |
| ggccctaagctgtctacccagctggtcaagaaccagtgcgtgaacttcaatttcaacggc | |
| ctgaaaggcaccggcgtgctgaccgccagcagcaagagattccagagcttccagcagttc | |
| ggcagggacgccagcgatttcacagatagcgtcagagatccccagacactggaaatcctg | |
| gacatcagcccttgcagcttcggcggagtgtctgtgatcacccctggcaccaatacctct | |
| agcgaggtggcagtgctgtaccaggacgtgaactgcaccgatgtgcctacagccatccac | |
| gccgatcagctgacaccagcttggagagtgtactctaccggtgtcaacgtgttccagaca | |
| caagccggctgtctgattggagccgaacacgtgaacgccagctacgagtgcgacatccct | |
| atcggagccggcatctgtgcctcttaccacaccgcctctaacagccccagaatcctgaga | |
| agcaccggccagaaatccatcgtggcctacacaatgtctctgggcgccgagaactctatc | |
| gcctacgccaacaactccattgctatccccaccaacttcagcatctccgtgaccaccgaa | |
| gtgatgcctgtgtccatggccaagaccagcgtggactgcacaatgtacatctgcggcgac | |
| agcctggaatgcagcaacctgctgctccagtacggcagcttctgcacccagctgaataga | |
| gccctgaccggaatcgccatcgagcaggacaagaacacccaagaggtgttcgcccaagtg | |
| aagcagatgtataagacccctgccatcaaggacttcggcggctttaacttcagccagatc | |
| ctgcctgatcctagcaagcccaccaagcggagcttcatcgaggacctgctgttcaacaaa | |
| gtgaccctggccgacgccggctttatgaagcagtatggcgagtgcctgggcgacatctct | |
| gccagggatctgatttgcgcccagaagttcaacggactgaccgtgctgcctcctctgctg | |
| accgatgagatgatcgccgcctatacagccgctctggtgtctggcacagctaccgccgga | |
| tggacatttggagctggcgccgctctccagattccattcgctatgcagatggcctaccgc | |
| ttcaacggcatcggcgtgacccagaacgtgctgtacgagaaccagaagcagatcgccaac | |
| cagttcaacaaggccatcagtcagatccaagagagcctgaccacaaccagcacagccctg | |
| ggaaagctccaggacgtcgtgaaccagaatgcccaggctctgaacaccctggtcaagcag | |
| ctgagcagcaatttcggcgccatcagctccgtgctgaacgacatcctgagccggctggat | |
| aaggtggaagccgaggtgcagatcgaccggctgattacaggcagactccagtctctccag | |
| acctacgtgacacagcagctgatcagagccgccgagattagagcctctgccaatctggcc | |
| gccaccaagatgtctgagtgtgtgctgggccagtctaagagagtggacttctgcggcaag | |
| ggctaccacctgatgagcttccctcaggctgctcctcacggcgtggtgtttctgcacgtg | |
| acatacgtgcccagccaagagcggaacttcacaactgccccagccatctgccacgagggc | |
| aaagcctactttcccagagaaggcgtgttcgtgtccaacggcacctcctggttcatcacc | |
| cagagaaacttctacagccctcagatcatcaccaccgacaacaccttcgtgtccggcaac | |
| tgcgacgtggtcatcggcatcatcaacaataccgtgtacgaccctctccagccagaactg | |
| gatagcttcaaagaggaactcgacaagtacttcaagaatcacacaagccccgacgtggac | |
| ctgggcgatatcagt | |
| >Wuhan_Node1_RBD (CoV_T2_7) | |
| (SEQ ID NO: 17) | |
| Amino acid sequence: | |
| RVSPTQEVVRFPNITNLCPFDKVFNATRFPSVYAWERTKISDCVADYTVLYNSTSFSTFKCYGVSPSK | |
| LIDLCFTSVYADTFLIRCSEVRQVAPGQTGVIADYNYKLPDDFTGCVIAWNTAKQDTGSSGNYNYYYR | |
| SHRKTKLKPFERDLSSDECSPDGKPCTPPAFNGVRGFNCYFTLSTYDFNPNVPVEYQATRVVVLSFEL | |
| LNAPATVCGPKLSTQ | |
| >Wuhan_Node1_RBD (CoV_T2_7) | |
| (SEQ ID NO: 18) | |
| Nucleic acid sequence: | |
| cgggtgtcccctacacaagaggtcgtgcggttccccaatatcaccaatctgtgccccttc | |
| gacaaggtgttcaacgccaccagatttcccagcgtgtacgcctgggagcgcaccaagatt | |
| tccgattgcgtggccgactacaccgtgctgtataactccacctccttcagcaccttcaag | |
| tgctacggcgtgtccccaagcaagctgatcgatctgtgcttcacctctgtgtacgccgac | |
| accttcctgatccggtgtagcgaagtgcgacaggtggcacctggacagacaggcgtgatc | |
| gccgattacaactacaagctgcccgacgacttcaccggetgtgtgatcgcctggaatacc | |
| gccaagcaggatacaggcagcagcggcaactacaactactactacagaagccaccgcaag | |
| accaagctgaagcctttcgagagggacctgagcagcgacgagtgtagccctgatggcaag | |
| ccttgtacacctcctgccttcaatggcgtgcggggcttcaactgctacttcaccctgagc | |
| acctacgacttcaaccccaacgtgcccgtggaataccaggccacaagagtggtggtgctg | |
| agcttcgagctgctgaatgcccctgccacagtgtgtggccctaagctgtctacccag | |
| Wuhan_Node1 (CoV_T2_1) (full length S protein amino acid sequence, | |
| with RBD residues shown in bold, and residues not present in | |
| truncated S protein shown underlined) | |
| (SEQ ID NO: 13) | |
| MFLFLFIIIF AFFLLSAKAN ERCGIFTSKP QPKLAQVSSS RRGVYYPDDI FRSDVLHLTQ | 60 |
| DYFLPFDSNV TRYFSLNANG PDRIVYFDNP IIPFKDGVYF AATEKSNVIR GWIFGSTLDN | 120 |
| TSQSVIIVNN STNVIIRVCN FDLCNDPFFT VSRPTDKHIK TWSIREFAVY QSAFNCTFEY | 180 |
| VSKSFLLDVA EKPGNFKHLR EFVFKNVDGF LNVYSTYKPI NVVSGLPTGF SVLKPILKLP | 240 |
| LGINITSFRV LLTMFRGDPT PGHTTANWLT AAAAYYVGYL KPTTFMLKYN ENGTITDAVD | 300 |
| CSQNPLAELK CTLKNFNVDK GIYQTSNFRV SPTQEVVRFP NITNLCPFDK VFNATRFPSV | 360 |
| YAWERTKISD CVADYTVLYN STSFSTFKCY GVSPSKLIDL CFTSVYADTF LIRCSEVRQV | 420 |
| APGQTGVIAD YNYKLPDDFT GCVIAWNTAK QDTGSSGNYN YYYRSHRKTK LKPFERDLSS | 480 |
| DECSPDGKPC TPPAFNGVRG FNCYFTLSTY DFNPNVPVEY QATRVVVLSF ELLNAPATVC | 540 |
| GPKLSTQLVK NQCVNFNFNG LKGTGVLTAS SKRFQSFQQF GRDASDFTDS VRDPQTLEIL | 600 |
| DISPCSFGGV SVITPGTNTS SEVAVLYQDV NCTDVPTATH ADQLTPAWRV YSTGVNVFQT | 660 |
| QAGCLIGAEH VNASYECDIP IGAGICASYH TASNSPRILR STGQKSIVAY TMSLGAENSI | 720 |
| AYANNSIAIP TNFSISVTTE VMPVSMAKTS VDCTMYICGD SLECSNLLLQ YGSFCTQLNR | 780 |
| ALTGIAIEQD KNTQEVFAQV KQMYKTPAIK DFGGFNFSQI LPDPSKPTKR SFIEDLLFNK | 840 |
| VTLADAGFMK QYGECLGDIS ARDLICAQKF NGLTVLPPLL TDEMIAAYTA ALVSGTATAG | 900 |
| WTFGAGAALQ IPFAMQMAYR FNGIGVTQNV LYENQKQIAN QFNKAISQIQ ESLTTTSTAL | 960 |
| GKLQDVVNQN AQALNTLVKQ LSSNFGAISS VINDILSRLD KVEAEVQIDR LITGRLQSLQ | 1020 |
| TYVTQQLIRA AEIRASANLA ATKMSECVLG QSKRVDFCGK GYHLMSFPQA APHGVVFLHV | 1080 |
| TYVPSQERNF TTAPAICHEG KAYFPREGVF VSNGTSWFIT QRNFYSPQII TTDNTFVSGN | 1140 |
| CDVVIGIINN TVYDPLQPEL DSFKEELDKY FKNHTSPDVD LGDISGINAS VVNIQKEIDR | 1200 |
| LNEVAKNLNE SLIDLQELGK YEQYIKWPWY VWLGFIAGLI AIVMATILLC CMTSCCSCLK | 1260 |
| GACSCGSCCK FDEDDSEPVL KGVKLHYT | 1288 |

### EXAMPLE 2

| Alignment of full-length S-protein amino acid sequence of CoV_T2_1 (Wuhan_Node1) with | |
|---|---|
| AY274119 | |
| Score = 55060.0 | |
| Length of alignment = 1284 | |
| Sequence Wuhan_Node1/5-1288 (Sequence length = 1288) (SEQ ID NO: 13) | |
| Sequence AY274119/1-1255 (Sequence length = 1255) (SEQ ID NO:1) | |
| Wuhan_Node1/5-1288 | LFIIIFAFFLLSAKANERCGIFTSKPQPKLAQVSSSRRGVYYPDDIFRSDVLH |
| | .\|\|... . \| \|. .\|\| \| \|. .\| .\|\| \|\|\|\|\|\|\|.\|\|\|\|\| \| |
| AY274119/1-1255 | MFIFLLFLTLTSGSDLDRCTTFDDVQAPNYTQHTSSMRGVYYPDEIFRSDTLY |
| Wuhan_Node1/5-1288 | LTQDYFLPFDSNVTRYFSLNANGPDRIVYFDNPIIPFKDGVYFAATEKSNVIR |
| | \|\|\|\| \|\|\|\| \|\|\|\| . ..\| . \|.\|\|.\|\|\|\|\|\|.\|\|\|\|\|\|\|\|\|\|.\| |
| AY274119/1-1255 | LTQDLFLPFYSNVTGFHTIN-----HT--FGNPVIPFKDGIYFAATEKSNVVR |
| Wuhan_Node1/5-1288 | GWIFGSTLDNTSQSVIIVNNSINVIIRVCNFDLCNDPFFTVSRPTDKHIKTWS |
| | \|\|.\|\|\|\|..\| \|\|\|\|\|\|.\|\|\|\|\|\|.\|\| \|\|\|.\|\|..\|\|\|.\|\|.\| . .\| . |
| AY274119/1-1255 | GWVFGSTMNNKSQSVIIINNSTNVVIRACNFELCDNPFFAVSKPMG--TQTHT |
| Wuhan_Node1/5-1288 | IREFAVYQSAFNCTFEYVSKSFLLDVAEKPGNFKHLREFVFKNVDGFLNVYST |
| | ....\|\|\|\|\|\|\|\|.\| .\| \|\|\|.\|\|.\|\|\|\|\|\|\|\|\|\|\|\|\| \|\|\|\| \|\| |
| AY274119/1-1255 | ----MIFDNAFNCTFEYISDAFSLDVSEKSGNFKHLREFVFKNKDGFLYVYKG |
| Wuhan_Node1/5-1288 | YKPINVVSGLPTGFSVLKPILKLPLGINITSFRVLLTMFRGDPTPGHTTANWL |
| | \|.\|\|.\|\| .\|\|.\|\|. \|\|\|\|.\|\|\|\|\|\|\|\|\|.\|\| .\|\| \| .\|.. \| |
| AY274119/1-1255 | YQPIDVVRDLPSGFNTLKPIFKLPLGINITNFRAILTAF----SPAQDI--WG |
| Wuhan_Node1/5-1288 | TAAAAYYVGYLKPTTFMLKYNENGTITDAVDCSQNPLAELKCTLKNFNVDKGI |
| | \|.\|\|\|\|.\|\|\|\|\|\|\|\|\|\|\|\|\|.\|\|\|\|\|\|\|\|\|\|\|\|\|\|\|\|\|\|\|\|\|..\|.\|..\|\|\|\| |
| AY274119/1-1255 | TSAAAYFVGYLKPTTFMLKYDENGTITDAVDCSQNPLAELKCSVKSFEIDKGI |
| Wuhan_Node1/5-1288 | YQTSNFRVSPTQEVVRFPNITNLCPFDKVFNATRFPSVYAWERTKISDCVADY |
| | \|\|\|\|\|\|\|\| \|. .\|\|\|\|\|\|\|\|\|\|\|\|\|. \|\|\|\|\|.\|\|\|\|\|\|\|\|\| \|\|\|.\|\|\|\|\| |
| AY274119/1-1255 | YQTSNFRVVPSGDVVRFPNITNLCPFGEVFNATKFPSVYAWERKKISNCVADY |
| Wuhan_Node1/5-1288 | TVLYNSTSFSTFKCYGVSPSKLIDLCFTSVYADTFLIRCSEVRQVAPGQTGVI |
| | .\|\|\|\|\|\| \|\|\|\|\|\|\|\|\|\|..\|\| \|\|\|\|..\|\|\|\|.\|... .\|\|\|.\|\|\|\|\|\|\|\| |
| AY274119/1-1255 | SVLYNSTEFSTFKCYGVSATKLNDLCFSNVYADSFVVKGDDVRQIAPGQTGVI |
| Wuhan_Node1/5-1288 | ADYNYKLPDDFTGCVIAWNTAKQDTGSSGNYNYYYRSHRKTKLKPFERDLSSD |
| | \|\|\|\|\|\|\|\|\|\|\| \|\|\|.\|\|\|\| . \|. \|.\|\|\|\|\| \|\| \| \|\|.\|\|\|\|\|.\|. |
| AY274119/1-1255 | ADYNYKLPDDFMGCVLAWNTRNIDATSTGNYNYKYRYLRHGKLRPFERDISNV |
| Wuhan_Node1/5-1288 | ECSPDGKPCTPPAFNGVRGFNCYFTLSTYDFNPNVPVEYQATRVVVLSFELLN |
| | \|\|\|\|\|\|\|\|\|\|\| .\|\|\| \|. \|.\| . \|\|. \|\|\|\|\|\|\|\|\|\|\| |
| AY274119/1-1255 | PFSPDGKPCTPPA------LNCYWPLNDYGFYTTTGIGYQPYRVVVLSFELLN |
| Wuhan_Node1/5-1288 | APATVCGPKLSTQLVKNQCVNFNFNGLKGTGVLTASSKRFQSFQQFGRDASDF |
| | \|\|\|\|\|\|\|\|\|\|\|\|.\|.\|\|\|\|\|\|\|\|\|\|\|\| \|\|\|\|\|\|.\|\|\|\|\|\|.\|\|\|\|\|\|\| \|\|\| |
| AY274119/1-1255 | APATVCGPKLSTDLIKNQCVNFNFNGLTGTGVLTPSSKRFQPFQQFGRDVSDF |
| Wuhan_Node1/5-1288 | TDSVRDPQTLEILDISPCSFGGVSVITPGTNTSSEVAVLYQDVNCTDVPTAIH |
| | \|\|\|\|\|\|\|.\| \|\|\|\|\|\|\|\|.\|\|\|\|\|\|\|\|\|\|\|\|.\|\|\|\|\|\|\|\|\|\|\|\|\|\|\|\|.\|\|\|\| |
| AY274119/1-1255 | TDSVRDPKTSEILDISPCAFGGVSVITPGTNASSEVAVLYQDVNCTDVSTAIH |
| Wuhan_Node1/5-1288 | ADQLTPAWRVYSTGVNVFQTQAGCLIGAEHVNASYECDIPIGAGICASYHTAS |
| | \|\|\|\|\|\|\|\|\|.\|\|\|\| \|\|\|\|\|\|\|\|\|\|\|\|\|\|\|\|..\|\|\|\|\|\|\|\|\|\|\|\|\|\|\|\|\|\| \| |
| AY274119/1-1255 | ADQLTPAWRIYSTGNNVFQTQAGCLIGAEHVDTSYECDIPIGAGICASYHTVS |
| Wuhan_Node1/5-1288 | NSPRILRSTGQKSIVAYTMSLGAENSIAYANNSIAIPTNFSISVTTEVMPVSM |
| | .\|\|\|\|.\|\|\|\|\|\|\|\|\|\|\|\|\|..\|\|\|\|.\|\|.\|\|\|\|\|\|\|\|\|\|.\|\|\|\|\|\|\|\|\| |
| AY274119/1-1255 | ----LLRSTSQKSIVAYTMSLGADSSIAYSNNTIAIPTNFSISITTEVMPVSM |
| Wuhan_Node1/5-1288 | AKTSVDCTMYICGDSLECSNLLLQYGSFCTQLNRALTGIAIEQDKNTQEVFAQ |
| | \|\|\|\|\|\|\| \|\|\|\|\|\|\| \|\|.\|\|\|\|\|\|\|\|\|\|\|\|\|\|\|\|\|.\|\|\| \|\|\|.\|\|.\|\|\|\|\| |
| AY274119/1-1255 | AKTSVDCNMYICGDSTECANLLLQYGSFCTQLNRALSGIAAEQDRNTREVFAQ |
| Wuhan_Node1/5-1288 | VKQMYKTPAIKDFGGFNFSQILPDPSKPTKRSFIEDLLFNKVTLADAGFMKQY |
| | \|\|\|\|\|\|\|\|..\| \|\|\|\|\|\|\|\|\|\|\|\|\| \|\|\|\|\|\|\|\|\|\|\|\|\|\|\|\|\|\|\|\|\|\|\|\|\|\|\| |
| AY274119/1-1255 | VKQMYKTPTLKYFGGFNFSQILPDPLKPTKRSFIEDLLFNKVTLADAGFMKQY |
| Wuhan_Node1/5-1288 | GECLGDISARDLICAQKFNGLTVLPPLLTDEMIAAYTAALVSGTATAGWTFGA |
| | \|\|\|\|\|\|\|.\|\|\|\|\|\|\|\|\|\|\|\|\|\|\|\|\|\|\|\|\|\|.\|\|\|\|\|\|\|\|\|\|\|\|\|\|\|\|\|\|\|\|\|\| |
| AY274119/1-1255 | GECLGDINARDLICAQKFNGLTVLPPLLTDDMIAAYTAALVSGTATAGWTFGA |
| Wuhan_Node1/5-1288 | GAALQIPFAMQMAYRFNGIGVTQNVLYENQKQIANQFNKAISQIQESLTTTST |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119/1-1255 | GAALQIPFAMQMAYRFNGIGVTQNVLYENQKQIANQFNKAISQIQESLTTTST |
| Wuhan_Node1/5-1288 | ALGKLQDVVNQNAQALNTLVKQLSSNFGAISSVLNDILSRLDKVEAEVQIDRL |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119/1-1255 | ALGKLQDVVNQNAQALNTLVKQLSSNFGAISSVLNDILSRLDKVEAEVQIDRL |
| Wuhan_Node1/5-1288 | ITGRLQSLQTYVTQQLIRAAEIRASANLAATKMSECVLGQSKRVDFCGKGYHL |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119/1-1255 | ITGRLQSLQTYVTQQLIRAAEIRASANLAATKMSECVLGQSKRVDFCGKGYHL |
| Wuhan_Node1/5-1288 | MSFPQAAPHGVVFLHVTYVPSQERNFTTAPAICHEGKAYFPREGVFVSNGTSW |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119/1-1255 | MSFPQAAPHGVVFLHVTYVPSQERNFTTAPAICHEGKAYFPREGVFVFNGTSW |
| Wuhan_Node1/5-1288 | FITQRNFYSPQIITTDNTFVSGNCDVVIGIINNTVYDPLQPELDSFKEELDKY |
| | \|\|\|\|\|\|\|.\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119/1-1255 | FITQRNFFSPQIITTDNTFVSGNCDVVIGIINNTVYDPLQPELDSFKEELDKY |
| Wuhan_Node1/5-1288 | FKNHTSPDVDLGDISGINASVVNIQKEIDRLNEVAKNINESLIDLQELGKYEQ |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119/1-1255 | FKNHTSPDVDLGDISGINASVVNIQKEIDRLNEVAKNINESLIDLQELGKYEQ |
| Wuhan_Node1/5-1288 | YIKWPWYVWLGFIAGLIAIVMATILLCCMTSCCSCLKGACSCGSCCKFDEDDS |
| | \|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\| |
| AY274119/1-1255 | YIKWPWYVWLGFIAGLIAIVMVTILLCCMTSCCSCLKGACSCGSCCKFDEDDS |
| Wuhan_Node1/5-1288 | EPVLKGVKLHYT |
| | \|\|\|\|\|\|\|\|\|\|\|\| |
| AY274119/1-1255 | EPVLKGVKLHYT |

Percentage ID = 82.32

### EXAMPLE 3

| Alignment of full-length S-protein amino acid sequence of CoV_T2_1 (Wuhan_Node1) with | |
|---|---|
| EPI_ISL_402119 | |
| Score = 53960.0 | |
| Length of alignment = 1280 | |
| Sequence Wuhan_Node1/9-1288(Sequence length = 1288) (SEQ ID NO: 13) | |
| Sequence EPI_ISL_402119/1-1273(Sequence length = 1273) (SEQ ID NO: 7) | |
| Wuhan_Node1/9-1288 | IFAFFLLSAKANERCGIFTSKPQPKLAQVSSSRRGVYYPDDIFRSDVLHL |
| | .\| \|..\| . . .\| .\|.. \| \| .\| \|\|\|\|\|\|\| .\|\|\| \|\|\| |
| EPI_ISL_402119/1-1273 | MFVFLVLLPLVSSQCVNLTTRTQLPPAYTNSFTRGVYYPDKVFRSSVLHS |
| Wuhan_Node1/9-1288 | TQDYFLPFDSNVTRYFSLNANGPDRIVYFDNPIIPFKDGVYFAATEKSNV |
| | \|\|\| \|\|\|\| \|\|\|\|.. ... .\| . \|\|\|\|..\|\|.\|\|\|\|\|\|.\|\|\|\|\|. |
| EPI_ISL_402119/1-1273 | TQDLFLPFFSNVTWFHAIHVSGINGTKRFDNPVLPFNDGVYFASTEKSNI |
| Wuhan_Node1/9-1288 | IRGWIFGSTLDNTSQSVIIVNNSTNVIIRVCNFDLCNDPFFTVSRPTDKH |
| | \|\|\|\|\|\|\|.\|\|\|. .\|\|..\|\|\|\|.\|\|\|.\|.\|\|.\|..\|\|\|\|\|. \| .\|. |
| EPI_ISL_402119/1-1273 | IRGWIFGTTLDSKTQSLLIVNNATNVVIKVCEFQFCNDPFLGVY--YHKN |
| Wuhan_Node1/9-1288 | IKTWSIREFAVYQSAFNCTFEYVSKSFLLDVAEKPGNFKHLREFVFKNVD |
| | \|.\| \|\| \|\| \|\| \|\|\|\|\|\|\|\|..\|\|.\|. \| \|\|\|\|.\|\|\|\|\|\|\|\|.\| |
| EPI_ISL_402119/1-1273 | NKSWMESEFRVYSSANNCTFEYVSQPFLMDLEGKQGNFKNLREFVFKNID |
| Wuhan_Node1/9-1288 | GFLNVYSTYKPINVVSGLPTGFSVLKPILKLPLGINITSFRVLLTMFRGD |
| | \|....\|\| \|\|\|.\| .\|\| \|\|\| \| \|.. \|\|.\|\|\|\|\| \|. \|\|.. \|. |
| EPI_ISL_402119/1-1273 | GYFKIYSKATPINLVRDLPQGFSALEPLVDLPIGINITRFQTLLALHRSY |
| Wuhan_Node1/9-1288 | PTPGHTTANWLTAAAAYYVGYLKPTTFMLKYNENGTITDAVDCSQNPLAE |
| | \|\|\|.... \| ..\|\|\|\|\|\|\|\|\|.\| \|\|.\|\|\|\|\|\|\|\|\|\|\|\|\|\|\|. .\|\|.\| |
| EPI_ISL_402119/1-1273 | LTPGDSSSGWTAGAAAYYVGYLQPRTFLLKYNENGTITDAVDCALDPLSE |
| Wuhan_Node1/9-1288 | LKCTLKNFNVDKGIYQTSNFRVSPTQEVVRFPNITNLCPFDKVFNATRFP |
| | \|\|\|\|\|.\| \|.\|\|\|\|\|\|\|\|\|\|\| \|\|. .\|\|\|\|\|\|\|\|\|\|\|\|. \|\|\|\|\|\|\|. |
| EPI_ISL_402119/1-1273 | TKCTLKSFTVEKGIYQTSNFRVQPTESIVRFPNITNLCPFGEVFNATRFA |
| Wuhan_Node1/9-1288 | SVYAWERTKISDCVADYTVLYNSTSFSTFKCYGVSPSKLIDLCFTSVYAD |
| | \|\|\|\|\|.\| .\|\|.\|\|\|\|\|.\|\|\|\|\|.\|\|\|\|\|\|\|\|\|\|\|\|.\|\| \|\|\|\|\|.\|\|\|\| |
| EPI_ISL_402119/1-1273 | SVYAWNRKRISNCVADYSVLYNSASFSTFKCYGVSPTKLNDLCFTNVYAD |
| Wuhan_Node1/9-1288 | TFLIRCSEVRQVAPGQTGVIADYNYKLPDDFTGCVIAWNTAKQDTGSSGN |
| | .\|.\|\| \|\|\|\|.\|\|\|\|\|\| \|\|\|\|\|\|\|\|\|\|\|\|\|\|\|\|\|\|\|\|. . \|. .\|\| |
| EPI_ISL_402119/1-1273 | SFVIRGDEVRQIAPGQTGKIADYNYKLPDDFTGCVIAWNSNNLDSKVGGN |
| Wuhan_Node1/9-1288 | YNYYYRSHRKTKLKPFERDLSSDECSPDGKPCTPPAFNGVRGFNCYFTLS |
| | \|\|\| \|\| \|\|..\|\|\|\|\|\|\|.\|.. ... \|\| \|\|\| \|\|\|\|\|\| \| |
| EPI_ISL_402119/1-1273 | YNYLYRLFRKSNLKPFERDISTEIYQAGSTPC-----NGVEGFNCYFPLQ |
| Wuhan_Node1/9-1288 | TYDFNPNVPVEYQATRVVVLSFELLNAPATVCGPKLSTQLVKNQCVNFNF |
| | .\|.\|.\| \| \|\|. \|\|\|\|\|\|\|\|\|\|.\|\|\|\|\|\|\|\| \|\|.\|\|\|\|.\|\|\|\|\|\| |
| EPI_ISL_402119/1-1273 | SYGFQPTNGVGYQPYRVVVLSFELLHAPATVCGPKKSTNLVKNKCVNFNF |
| Wuhan_Node1/9-1288 | NGLKGTGVLTASSKRFQSFQQFGRDASDFTDSVRDPQTLEILDISPCSFG |
| | \|\|\| \|\|\|\|\|\| \|.\|.\| .\|\|\|\|\|\|\| .\| \|\|.\|\|\|\|\|\|\|\|\|\|\|\|.\|\|\|\|\| |
| EPI_ISL_402119/1-1273 | NGLTGTGVLTESNKKFLPFQQFGRDIADTTDAVRDPQTLEILDITPCSFG |
| Wuhan_Node1/9-1288 | GVSVITPGTNTSSEVAVLYQDVNCTDVPTAIHADQLTPAWRVYSTGVNVF |
| | \|\|\|\|\|\|\|\|\|\|\|\|..\|\|\|\|\|\|\|\|\|\|\|.\|\| \|\|\|\|\|\|\|\|\|.\|\|\|\|\|\|\| \|\|\| |
| EPI_ISL_402119/1-1273 | GVSVITPGTNTSNQVAVLYQDVNCTEVPVAIHADQLTPTWRVYSTGSNVF |
| Wuhan_Node1/9-1288 | QTQAGCLIGAEHVNASYECDIPIGAGICASYHTASNSPRILRSTGQKSIV |
| | \|\|.\|\|\|\|\|\|\|\|\|\|\| \|\|\|\|\|\|\|\|\|\|\|\|\|\|\|\|.\| .\|\|\|\| \|\| . .\|\|. |
| EPI_ISL_402119/1-1273 | QTRAGCLIGAEHVNNSYECDIPIGAGICASYQTQTNSPRRARSVASQSII |
| Wuhan_Node1/9-1288 | AYTMSLGAENSIAYANNSIAIPTNFSISVTTEVMPVSMAKTSVDCTMYIC |
| | \|\|\|\|\|\|\|\|\|\|\|.\|\|.\|\|\|\|\|\|\|\|\|\|.\|\|\|\|\|\|..\|\|\|\|.\|\|\|\|\|\|\|\|\|\|\| |
| EPI_ISL_402119/1-1273 | AYTMSLGAENSVAYSNNSIAIPTNFTISVTTEILPVSMTKTSVDCTMYIC |
| Wuhan_Node1/9-1288 | GDSLECSNLLLQYGSFCTQLNRALTGIAIEQDKNTQEVFAQVKQMYKTPA |
| | \|\|\| \|\|\|\|\|\|\|\|\|\|\|\|\|\|\|\|\|\|\|\|\|\|\|\|.\|\|\|\|\|\|\|\|\|\|\|\|\|\|\|.\|\|\|\|. |
| EPI_ISL_402119/1-1273 | GDSTECSNLLLQYGSFCTQLNRALTGIAVEQDKNTQEVFAQVKQIYKTPP |
| Wuhan_Node1/9-1288 | IKDFGGFNFSQILPDPSKPTKRSFIEDLLFNKVTLADAGFMKQYGECLGD |
| | \|\|\|\|\|\|\|\|\|\|\|\|\|\|\|\|\|\|\|.\|\|\|\|\|\|\|\|\|\|\|\|\|\|\|\|\|\|\|\|.\|\|\|\|.\|\|\|\| |
| EPI_ISL_402119/1-1273 | IKDFGGFNFSQILPDPSKPSKRSFIEDLLFNKVTLADAGFIKQYGDCLGD |
| Wuhan_Node1/9-1288 | ISARDLICAQKFNGLTVLPPLLTDEMIAAYTAALVSGTATAGWTFGAGAA |
| | \|.\|\|\|\|\|\|\|\|\|\|\|\|\|\|\|\|\|\|\|\|\|\|\|\|\|\| \|\|.\|\|..\|\| \|.\|\|\|\|\|\|\|\|\| |
| EPI_ISL_402119/1-1273 | IAARDLICAQKFNGLTVLPPLLTDEMIAQYTSALLAGTITSGWTFGAGAA |
| Wuhan_Node1/9-1288 | LQIPFAMQMAYRFNGIGVTQNVLYENQKQIANQFNKAISQIQESLITTST |
| | \|\|\|\|\|\|\|\|\|\|\|\|\|\|\|\|\|\|\|\|\|\|\|\|\|\|\|\| \|\|\|\|\|\| \|\|..\|\|.\|\|..\|.. |
| EPI_ISL_402119/1-1273 | LQIPFAMQMAYRFNGIGVTQNVLYENQKLIANQFNSAIGKIQDSLSSTAS |
| Wuhan_Node1/9-1288 | ALGKLQDVVNQNAQALNTLVKQLSSNFGAISSVLNDILSRLDKVEAEVQI |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| EPI_ISL_402119/1-1273 | ALGKLQDVVNQNAQALNTLVKQLSSNFGAISSVLNDILSRLDKVEAEVQI |
| Wuhan_Node1/9-1288 | DRLITGRLQSLQTYVTQQLIRAAEIRASANLAATKMSECVLGQSKRVDFC |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| EPI_ISL_402119/1-1273 | DRLITGRLQSLQTYVTQQLIRAAEIRASANLAATKMSECVLGQSKRVDFC |
| Wuhan_Node1/9-1288 | GKGYHLMSFPQAAPHGVVFLHVTYVPSQERNFTTAPAICHECKAYFPREG |
| | \|\|\|\|\|\|\|\|\|\|\|.\|\|\|\|\|\|\|\|\|\|\|\|\|\|.\|\|.\|\|\|\|\|\|\|\|\|\|.\|\|\| \|\|\|\|\| |
| EPI_ISL_402119/1-1273 | GKGYHLMSFPQSAPHGVVFLHVTYVPAQEKNFTTAPAICHDGKAHFPREG |
| Wuhan_Node1/9-1288 | VFVSNGTSWFITQRNFYSPQIITTDNTFVSGNCDVVIGIINNTVYDPLQP |
| | \|\|\|\|\|\|\| \|\|.\|\|\|\|\|\| \|\|\|\|\|\|\|\|\|\|\|\|\|\|\|\|\|\|\|\|\|.\|\|\|\|\|\|\|\|\|\| |
| EPI_ISL_402119/1-1273 | VFVSNGTHWFVTQRNFYEPQIITTDNTFVSGNCDVVIGIVNNTVYDPLQP |
| Wuhan_Node1/9-1288 | ELDSFKEELDKYFKNHTSPDVDLGDISGINASVVNIQKEIDRINEVAKNL |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| EPI_ISL_402119/1-1273 | ELDSFKEELDKYFKNHTSPDVDLGDISGINASVVNIQKEIDRLNEVAKNL |
| Wuhan_Node1/9-1288 | NESLIDLQELGKYEQYIKWPWYVWLGFIAGLIAIVMATILLCCMTSCCSC |
| | \|\|\|\|\|\|\|\|\|\|\|\|\|\|\|\|\|\|\|\|\|\|.\|\|\|\|\|\|\|\|\|\|\|\|\| \|\|.\|\|\|\|\|\|\|\|\|\| |
| EPI_ISL_402119/1-1273 | NESLIDLQELGKYEQYIKWPWYIWLGFIAGLIAIVMVTIMLCCMTSCCSC |
| Wuhan_Node1/9-1288 | LKGACSCGSCCKFDEDDSEPVLKGVKLHYT |
| | \|\|\| \|\|\|\|\|\|\|\|\|\|\|\|\|\|\|\|\|\|\|\|\|\|\|\|\|\| |
| EPI_ISL_402119/1-1273 | LKGCCSCGSCCKFDEDDSEPVLKGVKLHYT |

Percentage ID = 78.98

### EXAMPLE 4

| Alignment of_truncated S-protein amino acid sequence of CoV_T2_4 (Wuhan_Node1_tr) | |
|---|---|
| with AY274119 | |
| Score = 49480.0 | |
| Length of alignment = 1181 | |
| Sequence Wuhan_Node1_tr/5-1185 (Sequence length = 1185) (SEQ ID NO: 15) | |
| Sequence AY274119_tr (CoV_T2_2)/1-1152(Sequence length = 1152) (SEQ ID NO: 3) | |
| Wuhan_Node1_tr/5-1185 | LFIIIFAFFLLSAKANERCGIFTSKPQPKLAQVSSSRRGVYYP |
| | .\|\|... . \| \|. .\|\| \| \|. .\| .\|\| \|\|\|\|\|\| |
| AY274119_tr (CoV_T2_2)/1-1152 | MFIFLLFLTLTSGSDLDRCTTFDDVQAPNYTQHTSSMRGVYYP |
| Wuhan_Node1_tr/5-1185 | DDIFRSDVLALTQDYFLPFDSNVTRYFSLNANGPDRIVYFDNP |
| | \|.\|\|\|\|\| \| \|\|\|\| \|\|\|\| \|\|\|\| . ..\| . \|.\|\| |
| AY274119_tr (CoV_T2_2)/1-1152 | DEIFRSDTLYLTQDLFLPFYSNVTGFHTIN-----HT--FGNP |
| Wuhan_Node1_tr/5-1185 | IIPFKDGVYFAATEKSNVIRGWIFGSTLDNTSQSVIIVNNSTN |
| | .\|\|\|\|\|\|.\|\|\|\|\|\|\|\|\|\|.\|\|\|.\|\|\|\|..\| \|\|\|\|\|\|.\|\|\|\|\| |
| AY274119_tr(CoV_T2_2)/1-1152 | VIPFKDGIYFAATEKSNVVRGWVFGSTMNNKSQSVIIINNSTN |
| Wuhan_Node1_tr/5-1185 | VIIRVCNFDLCNDPFFTVSRPTDKHIKTWSIREFAVYQSAFNC |
| | \|.\|\| \|\|\|.\|\|..\|\|\|.\|\|.\| . .\| . ....\|\|\|\| |
| AY274119_tr (CoV_T2_2)/1-1152 | VVIRACNFELCDNPFFAVSKPMG--TQTHT----MIFDNAFNC |
| Wuhan_Node1_tr/5-1185 | TFEYVSKSFLLDVAEKPGNFKHLREFVFKNVDGFLNVYSTYKP |
| | \|\|\|\|.\| .\| \|\|\|.\|\|.\|\|\|\|\|\|\|\|\|\|\|\|\| \|\|\|\| \|\| \|.\| |
| AY274119_tr (CoV_T2_2)/1-1152 | TFEYISDAFSLDVSEKSGNFKHLREFVFKNKDGFLYVYKGYQP |
| Wuhan_Node1_tr/5-1185 | INVVSGLPTGFSVLKPILKLPLGINITSFRVLLTMFRGDPTPG |
| | \|.\|\| .\|\|.\|\|. \|\|\|\|.\|\|\|\|\|\|\|\|\|.\|\| .\|\| \| .\|. |
| AY274119_tr (CoV_T2_2)/1-1152 | IDVVRDLPSGFNTLKPIFKLPLGINITNFRAILTAF----SPA |
| Wuhan_Node1_tr/5-1185 | HTTANWLTAAAAYYVGYLKPTTFMLKYNENGTITDAVDCSQNP |
| | . \| \|.\|\|\|\|.\|\|\|\|\|\|\|\|\|\|\|\|\|.\|\|\|\|\|\|\|\|\|\|\|\|\|\|\| |
| AY274119_tr (CoV_T2_2)/1-1152 | QDI--WGTSAAAYFVGYLKPTTFMLKYDENGTITDAVDCSQNP |
| Wuhan_Node1_tr/5-1185 | LAELKCTLKNFNVDKGIYQTSNFRVSPTQEVVRFPNITNLCPF |
| | \|\|\|\|\|\|..\|.\|..\|\|\|\|\|\|\|\|\|\|\|\| \|. .\|\|\|\|\|\|\|\|\|\|\|\|\| |
| AY274119_tr(CoV_T2_2)/1-1152 | LAELKCSVKSFEIDKGIYQTSNFRVVPSGDVVRFPNITNLCPF |
| Wuhan_Node1_tr/5-1185 | DKVFNATRFPSVYAWERTKISDCVADYTVLYNSTSFSTFKCYG |
| | . \|\|\|\|\|.\|\|\|\|\|\|\|\|\| \|\|\|.\|\|\|\|\|.\|\|\|\|\|\| \|\|\|\|\|\|\|\| |
| AY274119_tr(CoV_T2_2)/1-1152 | GEVFNATKFPSVYAWERKKISNCVADYSVLYNSTFFSTFKCYG |
| Wuhan_Node1_tr/5-1185 | VSPSKLIDLCFTSVYADTFLIRCSEVRQVAPGQTGVIADYNYK |
| | \|\|..\|\| \|\|\|\|..\|\|\|\|.\|... .\|\|\|.\|\|\|\|\|\|\|\|\|\|\|\|\|\| |
| AY274119_tr(CoV_T2_2)/1-1152 | VSATKLNDLCFSNVYADSFVVKGDDVRQIAPGQTGVIADYNYK |
| Wuhan_Node1_tr/5-1185 | LPDDFTGCVIAWNTAKQDTGSSGNYNYYYRSHRKTKLKPFERD |
| | \|\|\|\|\| \|\|\|.\|\|\|\| . \|. \|.\|\|\|\|\| \|\| \| \|\|.\|\|\|\|\| |
| AY274119_tr (CoV_T2_2)/1-1152 | LPDDFMGCVLAWNTRNIDATSTGNYNYKYRYLRHGKLRPFERD |
| Wuhan_Node1_tr/5-1185 | LSSDECSPDGKPCTPPAFNGVRGFNCYFTLSTYDFNPNVPVEY |
| | .\|. \|\|\|\|\|\|\|\|\|\|\| .\|\|\| \|. \|.\| . \| |
| AY274119_tr (CoV_T2_2)/1-1152 | ISNVPFSPDGKPCTPPA------LNCYWPLNDYGFYTTTGIGY |
| Wuhan_Node1_tr/5-1185 | QATRVVVLSFELLNAPATVCGPKLSTQLVKNQCVNFNFNGLKG |
| | \|. \|\|\|\|\|\|\|\|\|\|\|\|\|\|\|\|\|\|\|\|\|\|\|.\|.\|\|\|\|\|\|\|\|\|\|\|\| \| |
| AY274119_tr (CoV_T2_2)/1-1152 | QPYRVVVLSFELLNAPATVCGPKLSTDLIKNQCVNFNFNGLTG |
| Wuhan_Node1_tr/5-1185 | TGVLTASSKRFQSFQQFGRDASDFTDSVRDPQTLEILDISPCS |
| | \|\|\|\|\|.\|\|\|\|\|\|.\|\|\|\|\|\|\| \|\|\|\|\|\|\|\|\|\|.\| \|\|\|\|\|\|\|\|. |
| AY274119_tr (CoV_T2_2)/1-1152 | TGVLTPSSKRFQPFQQFGRDVSDFTDSVRDPKTSEILDISPCA |
| Wuhan_Node1_tr/5-1185 | FGGVSVITPGTNTSSEVAVLYQDVNCTDVPTAIHADQLTPAWR |
| | \|\|\|\|\|\|\|\|\|\|\|\|.\|\|\|\|\|\|\|\|\|\|\|\|\|\|\|\|.\|\|\|\|\|\|\|\|\|\|\|\|\| |
| AY274119_tr(CoV_T2_2)/1-1152 | FGGVSVITPGTNASSEVAVLYQDVNCTDVSTAIHADQLTPAWR |
| Wuhan_Node1_tr/5-1185 | VYSTGVNVFQTQAGCLIGAEHVNASYECDIPIGAGICASYHTA |
| | .\|\|\|\| \|\|\|\|\|\|\|\|\|\|\|\|\|\|\|\|.\|\|\|\|\|\|\|\|\|\|\|\|\|\|\|\|\|\|\| |
| AY274119_tr(CoV_T2_2)/1-1152 | IYSTGNNVFQTQAGCLIGAEHVDTSYECDIPIGAGICASYHTV |
| Wuhan_Node1_tr/5-1185 | SNSPRILRSTGQKSIVAYTMSLGAENSIAYANNSIAIPTNFSI |
| | \| .\|\|\|\|.\|\|\|\|\|\|\|\|\|\|\|\|\|..\|\|\|\|.\|\|.\|\|\|\|\|\|\|\|\| |
| AY274119_tr (CoV_T2_2)/1-1152 | S----LLRSTSQKSIVAYTMSLGADSSIAYSNNTIAIPTNFSI |
| Wuhan_Node1_tr/5-1185 | SVTTEVMPVSMAKTSVDCTMYICGDSLECSNLLLQYGSFCTQL |
| | \|.\|\|\|\|\|\|\|\|\|\|\|\|\|\|\|\| \|\|\|\|\|\|\| \|\|.\|\|\|\|\|\|\|\|\|\|\|\|\| |
| AY274119_tr (CoV_T2_2)/1-1152 | SITTEVMPVSMAKTSVDCNMYICGDSTECANLLLQYGSFCTQL |
| Wuhan_Node1_tr/5-1185 | NRALTGIAIEQDKNTQEVFAQVKQMYKTPAIKDFGGFNFSQIL |
| | \|\|\|\|.\|\|\| \|\|\|.\|\|.\|\|\|\|\|\|\|\|\|\|\|\|\|..\| \|\|\|\|\|\|\|\|\|\| |
| AY274119_tr (CoV_T2_2)/1-1152 | NRALSGIAAEQDRNTREVFAQVKQMYKTPTLKYFGGFNFSQIL |
| Wuhan_Node1_tr/5-1185 | PDPSKPTKRSFIEDLLFNKVTLADAGFMKQYGECLGDISARDL |
| | \|\|\| \|\|\|\|\|\|\|\|\|\|\|\|\|\|\|\|\|\|\|\|\|\|\|\|\|\|\|\|\|\|\|\|\|\|.\|\|\|\| |
| AY274119_tr (CoV_T2_2)/1-1152 | PDPLKPTKRSFIEDLLFNKVTLADAGFMKQYGECLGDINARDL |
| Wuhan_Node1_tr/5-1185 | ICAQKFNGLTVLPPLLTDEMIAAYTAALVSGTATAGWTFGAGA |
| | \|\|\|\|\|\|\|\|\|\|\|\|\|\|\|\|\|\|.\|\|\|\|\|\|\|\|\|\|\|\|\|\|\|\|\|\|\|\|\|\|\|\| |
| AY274119_tr (CoV_T2_2)/1-1152 | ICAQKFNGLTVLPPLLTDDMIAAYTAALVSGTATAGWTFGAGA |
| Wuhan_Node1_tr/5-1185 | ALQIPFAMQMAYRFNGIGVTQNVLYENQKQIANQFNKAISQIQ |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119_tr (CoV_T2_2)/1-1152 | ALQIPFAMQMAYRFNGIGVTQNVLYENQKQIANQFNKAISQIQ |
| Wuhan_Node1_tr/5-1185 | ESLTTTSTALGKLQDVVNQNAQALNTLVKQLSSNFGAISSVLN |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119_tr (CoV_T2_2)/1-1152 | ESLTTTSTALGKLQDVVNQNAQALNTLVKQLSSNFGAISSVLN |
| Wuhan_Node1_tr/5-1185 | DILSRLDKVEAEVQIDRLITGRLQSLQTYVTQQLIRAAEIRAS |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119_tr (CoV_T2_2)/1-1152 | DILSRLDKVEAEVQIDRLITGRLQSLQTYVTQQLIRAAEIRAS |
| Wuhan_Node1_tr/5-1185 | ANLAATKMSECVLGQSKRVDFCGKGYHLMSFPQAAPHGVVFLH |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| |
| AY274119_tr (CoV_T2_2)/1-1152 | ANLAATKMSECVLGQSKRVDFCGKGYHIMSFPQAAPHGVVFLH |
| Wuhan_Node1_tr/5-1185 | VTYVPSQERNFTTAPAICHEGKAYFPREGVFVSNGTSWFITQR |
| | \|\|\|\|\|\|\|\|\|\|\|\|\|\|\|\|\|\|\|\|\|\|\|\|\|\|\|\|\|\|\|\| \|\|\|\|\|\|\|\|\|\| |
| AY274119_tr (CoV_T2_2)/1-1152 | VTYVPSQERNFTTAPAICHEGKAYFPREGVFVFNGTSWFITQR |
| Wuhan_Node1_tr/5-1185 | NFYSPQIITTDNTFVSGNCDVVIGIINNTVYDPLQPELDSFKE |
| | \|\|.\|\|\|\|\|\|\|\|\|\|\|\|\|\|\|\|\|\|\|\|\|\|\|\|\|\|\|\|\|\|\|\|\|\|\|\|\|\|\|\| |
| AY274119_tr (CoV_T2_2)/1-1152 | NFFSPQIITTDNTFVSGNCDVVIGIINNTVYDPLQPELDSFKE |
| Wuhan_Node1_tr/5-1185 | ELDKYFKNHTSPDVDLGDIS |
| | \|\|\|\|\|\|\|\|\|\|\|\|\|\|\|\|\|\|\|\| |
| AY274119_tr (CoV_T2_2)/1-1152 | ELDKYFKNHTSPDVDLGDIS |

Percentage ID = 80.86

### EXAMPLE 5

| Alignment of_truncated S-protein amino acid sequence of CoV_T2_4 (Wuhan_Node1_tr) | |
|---|---|
| with EPI_ISL_402119 | |
| Score = 48450.0 | |
| Length of alignment = 1177 | |
| Sequence Wuhan_Node1_tr/9-1185 (Sequence length = 1185) (SEQ ID NO: 15) | |
| Sequence EPI_ISL_402119_tr/1-1170 (Sequence length = 1170) (SEQ ID NO: 9) | |
| Wuhan_Node1_tr/9-1185 | IFAFFLLSAKANERCGIFTSKPQPKLAQVSSSRRGVYYPDDIFRSDV |
| | .\| \|..\| . . .\| .\|.. \| \| .\| \|\|\|\|\|\|\| .\|\|\| \| |
| EPI_ISL_402119_tr/1-1170 | MFVFLVLLPLVSSQCVNLTTRTQLPPAYTNSFTRGVYYPDKVFRSSV |
| Wuhan_Node1_tr/9-1185 | LHLTQDYFLPFDSNVTRYFSLNANGPDRIVYFDNPIIPFKDGVYFAA |
| | \|\| \|\|\| \|\|\|\| \|\|\|\|.. ... .\| . \|\|\|\|..\|\|.\|\|\|\|\|\|. |
| EPI_ISL_402119_tr/1-1170 | LHSTQDLFLPFFSNVTWFHAIHVSGTNGTKRFDNPVLPFNDGVYFAS |
| Wuhan_Node1_tr/9-1185 | TEKSNVIRGWIFGSTLDNTSQSVIIVNNSTNVIIRVCNFDLCNDPFF |
| | \|\|\|\|\|.\|\|\|\|\|\|\|.\|\|\|. .\|\|..\|\|\|\|.\|\|\|.\|.\|\|.\|..\|\|\|\|\|. |
| EPI_ISL_402119_tr/1-1170 | TEKSNIIRGWIFGTTLDSKTQSLLIVNNATNVVIKVCEFQFQNDPFL |
| Wuhan_Node1_tr/9-1185 | TVSRPTDKHIKTWSIREFAVYQSAFNCTFEYVSKSFLLDVAEKPGNF |
| | \| .\|. \|.\| \|\| \|\| \|\| \|\|\|\|\|\|\|\|..\|\|.\|. \| \|\|\| |
| EPI_ISL_402119_tr/1-1170 | GVY--YHKNNKSWMESEFRVYSSANNCTFEYVSQPFLMDLEGKQGNF |
| Wuhan_Node1_tr/9-1185 | KHLREFVFKNVDGFLNVYSTYKPINVVSGLPTGFSVLKPILKLPLGI |
| | \|.\|\|\|\|\|\|\|\|.\|\|....\|\| \|\|\|.\| .\|\| \|\|\| \| \|.. \|\|.\|\| |
| EPI_ISL_402119_tr/1-1170 | KNLREFVFKNIDGYFKIYSKHTPINLVRDLPQGFSALEPLVDLPIGI |
| Wuhan_Node1_tr/9-1185 | NITSFRVLLTMFRGDPTPGHTTANWLTAAAAYYVGYLKPTTFMLKYN |
| | \|\|\| \|. \|\|.. \|. \|\|\|.... \| ..\|\|\|\|\|\|\|\|\|.\| \|\|.\|\|\|\| |
| EPI_ISL_402119_tr/1-1170 | NITRFQTLLALHRSYLTPGDSSSGWTAGAAAYYVGYLQPRTFLLKYN |
| Wuhan_Node1_tr/9-1185 | ENGTITDAVDCSQNPLAELKCTLKNFNVDKGIYQTSNFRVSPTQEVV |
| | \|\|\|\|\|\|\|\|\|\|\|. .\|\|.\| \|\|\|\|\|.\| \|.\|\|\|\|\|\|\|\|\|\|\| \|\|. .\| |
| EPI_ISL_402119_tr/1-1170 | ENGTITDAVDCALDPLSETKCTLKSFTVEKGIYQTSNFRVQPTESIV |
| Wuhan_Node1_tr/9-1185 | RFPNITNLCPFDKVFNATRFPSVYAWERTKISDCVADYTVLYNSTSF |
| | \|\|\|\|\|\|\|\|\|\|\|. \|\|\|\|\|\|\|.\|\|\|\|\|.\| .\|\|.\|\|\|\|\|.\|\|\|\|\|.\|\| |
| EPI_ISL_402119_tr/1-1170 | RFPNITNLCPFGEVFNATRFASVYAWNRKRISNCVADYSVLYNSASF |
| Wuhan_Node1_tr/9-1185 | STFKCYGVSPSKLIDLCFTSVYADTFLIRCSEVRQVAPGQTGVIADY |
| | \|\|\|\|\|\|\|\|\|\|.\|\| \|\|\|\|\|.\|\|\|\|.\|.\|\| \|\|\|\|.\|\|\|\|\|\| \|\|\|\| |
| EPI_ISL_402119_tr/1-1170 | STFKCYGVSPTKLNDLCFTNVYADSFVIRGDEVRQIAPGQTGKIADY |
| Wuhan_Node1_tr/9-1185 | NYKLPDDFTGCVIAWNTAKQDTGSSGNYNYYYRSHRKTKLKPFERDL |
| | \|\|\|\|\|\|\|\|\|\|\|\|\|\|\|\|. . \|. .\|\|\|\|\| \|\| \|\|..\|\|\|\|\|\|\|. |
| EPI_ISL_402119_tr/1-1170 | NYKLPDDFTGCVIAWNSNNLDSKVGGNYNYLYRLFRKSNLKPFERDI |
| Wuhan_Node1_tr/9-1185 | SSDECSPDGKPCTPPAFNGVRGFNCYFTLSTYDFNPNVPVEYQATRV |
| | \|.. ... \|\| \|\|\| \|\|\|\|\|\| \| .\|.\|.\| \| \|\|. \|\| |
| EPI_ISL_402119_tr/1-1170 | STEIYQAGSTPC-----NGVEGFNCYFPLQSYGFQPTNGVGYQPYRV |
| Wuhan_Node1_tr/9-1185 | VVLSFELLNAPATVCGPKLSTQLVKNQCVNFNFNGLKGTGVLTASSK |
| | \|\|\|\|\|\|\|\|.\|\|\|\|\|\|\|\|\| \|\|.\|\|\|\|.\|\|\|\|\|\|\|\|\| \|\|\|\|\|\| \|.\| |
| EPI_ISL_402119_tr/1-1170 | VVLSFELLHAPATVCGPKKSTNLVKNKCVNFNFNGLTGTGVLTESNK |
| Wuhan_Node1_tr/9-1185 | RFQSFQQFGRDASDFTDSVRDPQTLEILDISPCSFGGVSVITPGTNT |
| | .\| .\|\|\|\|\|\|\| .\| \|\|.\|\|\|\|\|\|\|\|\|\|\|\|.\|\|\|\|\|\|\|\|\|\|\|\|\|\|\|\| |
| EPI_ISL_402119_tr/1-1170 | KFLPFQQFGRDIADTTDAVRDPQTLEILDITPCSFGGVSVITPGTNT |
| Wuhan_Node1_tr/9-1185 | SSEVAVLYQDVNCTDVPTAIHADQLTPAWRVYSTGVNVFQTQAGCLI |
| | \|..\|\|\|\|\|\|\|\|\|\|\|.\|\| \|\|\|\|\|\|\|\|\|.\|\|\|\|\|\|\| \|\|\|\|\|.\|\|\|\|\| |
| EPI_ISL_402119_tr/1-1170 | SNQVAVLYQDVNCTEVPVAIHADQLTPTWRVYSTGSNVFQTRAGCLI |
| Wuhan_Node1_tr/9-1185 | GAEHVNASYECDIPIGAGICASYHTASNSPRILRSTGQKSIVAYTMS |
| | \|\|\|\|\|\| \|\|\|\|\|\|\|\|\|\|\|\|\|\|\|\|.\| .\|\|\|\| \|\| . .\|\|.\|\|\|\|\| |
| EPI_ISL_402119_tr/1-1170 | GAEHVNNSYECDIPIGAGICASYQTQTNSPRRARSVASQSIIAYTMS |
| Wuhan_Node1_tr/9-1185 | LGAENSIAYANNSIAIPTNFSISVTTEVMPVSMAKTSVDCTMYICGD |
| | \|\|\|\|\|\|.\|\|.\|\|\|\|\|\|\|\|\|\|.\|\|\|\|\|\|..\|\|\|\|.\|\|\|\|\|\|\|\|\|\|\|\|\| |
| EPI_ISL_402119_tr/1-1170 | LGAENSVAYSNNSIAIPTNFTISVTTEILPVSMTKTSVDCTMYICGD |
| Wuhan_Node1_tr/9-1185 | SLECSNELLQYGSFCTQLNRALTGIAIEQDKNTQEVFAQVKQMYKTP |
| | \| \|\|\|\|\|\|\|\|\|\|\|\|\|\|\|\|\|\|\|\|\|\|\|\|.\|\|\|\|\|\|\|\|\|\|\|\|\|\|\|.\|\|\|\| |
| EPI_ISL_402119_tr/1-1170 | STECSNLLLQYGSFCTQLNRALTGIAVEQDKNTQEVFAQVKQIYKTP |
| Wuhan_Node1_tr/9-1185 | AIKDFGGFNFSQILPDPSKPTKRSFIEDLLFNKVTLADAGFMKQYGE |
| | .\|\|\|\|\|\|\|\|\|\|\|\|\|\|\|\|\|\|\|.\|\|\|\|\|\|\|\|\|\|\|\|\|\|\|\|\|\|\|\|.\|\|\|\|. |
| EPI_ISL_402119_tr/1-1170 | PIKDFGGFNFSQILPDPSKPSKRSFIEDLLFNKVTLADAGFIKQYGD |
| Wuhan_Node1_tr/9-1185 | CLGDISARDLICAQKFNGLTVLPPLLTDEMIAAYTAALVSGTATAGW |
| | \|\|\|\|\|.\|\|\|\|\|\|\|\|\|\|\|\|\|\|\|\|\|\|\|\|\|\|\|\|\|\| \|\|.\|\|..\|\| \|.\|\| |
| EPI_ISL_402119_tr/1-1170 | CLGDIAARDLICAQKFNGLTVLPPLLTDEMIAQYTSALLAGTITSGW |
| Wuhan_Node1_tr/9-1185 | TFGAGAALQIPFAMQMAYRFNGIGVTQNVLYENQKQIANQFNKAISQ |
| | \|\|\|\|\|\|\|\|\|\|\|\|\|\|\|\|\|\|\|\|\|\|\|\|\|\|\|\|\|\|\|\|\|\|\| \|\|\|\|\|\| \|\|.. |
| EPI_ISL_402119_tr/1-1170 | TFGAGAALQIPFAMQMAYRFNGIGVTQNVLYENQKLIANQFNSAIGK |
| Wuhan_Node1_tr/9-1185 | IQESLTTTSTALGKLQDVVNQNAQALNTLVKQLSSNFGAISSVLNDI |
| | \|\|.\|\|..\|..\|\|\|\|\|\|\|\|\|\|\|\|\|\|\|\|\|\|\|\|\|\|\|\|\|\|\|\|\|\|\|\|\|\|\|\|\| |
| EPI_ISL_402119_tr/1-1170 | IQDSLSSTASALGKLQDVVNQNAQALNTLVKQLSSNFGAISSVLNDI |
| Wuhan_Node1_tr/9-1185 | LSRLDKVEAEVQIDRLITGRLQSLQTYVTQQLIRAAEIRASANLAAT |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| EPI_ISL_402119_tr/1-1170 | LSRLDKVEAEVQIDRLITGRLQSLQTYVEQQLIRAAEIRASANLAAT |
| Wuhan_Node1_tr/9-1185 | KMSECVLGQSKRVDFCGKGYHLMSFPQAAPHGVVFLHVTYVPSQERN |
| | \|\|\|\|\|\|\|\|\|\|\|\|\|\|\|\|\|\|\|\|\|\|\|\|\|\|\|.\|\|\|\|\|\|\|\|\|\|\|\|\|\|.\|\|.\| |
| EPI_ISL_402119_tr/1-1170 | KMSECVLGQSKRVDFCGKGYHLMSFPQSAPHGVVFLHVTYVPAQEKN |
| Wuhan_Node1_tr/9-1185 | FTTAPAICHEGKAYFPREGVFVSNGTSWFITQRNFYSPQIITTDNTF |
| | \|\|\|\|\|\|\|\|\|.\|\|\| \|\|\|\|\|\|\|\|\|\|\|\| \|\|.\|\|\|\|\|\| \|\|\|\|\|\|\|\|\|\| |
| EPI_ISL_402119_tr/1-1170 | FTTAPAICHDGKAHFPREGVFVSNGTHWFVTQRNFYEPQIITTDNTF |
| Wuhan_Node1_tr/9-1185 | VSGNCDVVIGIINNTVYDPLQPELDSFKEELDKYFKNHTSPDVDLGD |
| | \|\|\|\|\|\|\|\|\|\|\|.\|\|\|\|\|\|\|\|\|\|\|\|\|\|\|\|\|\|\|\|\|\|\|\|\|\|\|\|\|\|\|\|\|\|\| |
| EPI_ISL_402119_tr/1-1170 | VSGNCDVVIGIVNNTVYDPLQPELDSFKEELDKYFKNHTSPDVDLGD |
| Wuhan_Node1_tr/9-1185 | IS |
| | \|\| |
| EPI_ISL_402119_tr/1-1170 | IS |

Percentage ID = 77.49

### EXAMPLE 6

| Alignment of S-protein RBD amino acid sequence | |
|---|---|
| of CoV_T2_7 (Wuhan_Node1_RBD) with | |
| Score = 8170.0 | |
| Length of alignment = 219 | |
| Sequence Wuhan Node1 RBD/1-219 (Sequence length = 219) (SEQ ID NO: 17) | |
| Sequence AY274119 RBD/1-213 (Sequence length = 213) (SEQ ID NO: 5) | |
| Wuhan_Model_RBD/1-219 | RVSPTQEVVREPNITNLCPEDKVENATREPSVYAWERTKISDCVADYTVL |
| | \|\| \|. .\|\|\|\|\|\|\|\|\|\|\|\|\|. \|\|\|\|\|.\|\|\|\|\|\|\|\|\| \|\|\|.\|\|\|\|\|.\|\| |
| AY274119_RBD/1-213 | RVVPSGDVVRFPNITNLCPFGEVENATKEPSVYAWERKKISNCVADYSVL |
| Wuhan_Model_RBD/1-219 | YNSTSFSTEKCYGVSPSKLIDLCFTSVYADTFLIRCSEVRQVAPGQTGVI |
| | \|\|\|\| \|\|\|\|\|\|\|\|\|\|..\|\| \|\|\|\|..\|\|\|\|.\|... .\|\|\|.\|\|\|\|\|\|\|\| |
| AY274119_RBD/1-213 | YNSTFFSTFKCYGVSATKLNDLCFSNVYADSFVVKGDDVRQIAPGQTGVI |
| Wuhan_Node1_RBD/1-219 | ADYNYKLPDDETGCVIAWNTAKQDTGSSGNYNYYYRSHRKTKLKPFERDL |
| | \|\|\|\|\|\|\|\|\|\|\| \|\|\|.\|\|\|\| . \|. \|.\|\|\|\|\| \|\| \| \|\|.\|\|\|\|\|. |
| AY274119_RBD/1-213 | ADYNYKLPDDFMGCVLAWNTRNIDATSTGNYNYKYRYLRHGKLRPFERDI |
| Wuhan_Node1_RBD/1-219 | SSDECSPDGKPCTPPAENGVRGENCYFTLSTYDENPNVPVEYQATRVVVL |
| | \|. \|\|\|\|\|\|\|\|\|\|\| .\|\|\| \|. \|.\| . \|\|. \|\|\|\|\| |
| AY274119_RBD/1-213 | SNVPFSPDGKPCTPPA------LNCYWPLNDYGFYTTTGIGYQPYRVVVL |
| Wuhan_Node1_RBD/1-219 | SFELLNAPATVCGPKLSTQ |
| | \|\|\|\|\|\|\|\|\|\|\|\|\|\|\|\|\|\|. |
| AY274119_RBD/1-213 | SFELLNAPATVCGPKLSTD |

Percentage ID = 70.32

### EXAMPLE 7

| Alignment of S-protein RBD amino acid sequence | |
|---|---|
| of CoV_T2_7 (Wuhan_Node1_RBD) with | |
| EPI_ISL_402119 | |
| Score = 8150.0 | |
| Length of alignment = 219 | |
| Sequence Wuhan Node1 RBD/1-219 (Sequence length = 219) (SEQ ID NO: 17) | |
| Sequence EPI_ISL_402119 RBD/1-214 (Sequence length = 214) (SEQ ID NO: 11) | |
| Wuhan_Node1_RBD/1-219 | RVSPTQEVVRFPNITNLCPFDKVFNATRFPSVYAWERTKISDCVADY |
| | \|\| \|\|. .\|\|\|\|\|\|\|\|\|\|\|\|. \|\|\|\|\|\|\|.\|\|\|\|\|.\| .\|\|.\|\|\|\|\| |
| EPI_ISL_402119_RBD/1-214 | RVQPTESIVRFPNITNLCPFGEVFNATRFASVYAWNRKRISNCVADY |
| Wuhan_Node1_RBD/1-219 | TVLYNSTSESTEKCYGVSPSKLIDLCFTSVYADTFLIRCSEVRQVAP |
| | .\|\|\|\|\|.\|\|\|\|\|\|\|\|\|\|\|\|.\|\| \|\|\|\|\|.\|\|\|\|.\|.\|\| \|\|\|\|.\|\| |
| EPI_ISL_402119_RBD/1-214 | SVLYNSASFSTEKCYGVSPTKLNDLCFTNVYADSFVIRGDEVRQIAP |
| Wuhan_Node1_RBD/1-219 | GQTGVIADYNYKLPDDETGCVIAWNTAKQDTGSSGNYNYYYRSHRKT |
| | \|\|\|\| \|\|\|\|\|\|\|\|\|\|\|\|\|\|\|\|\|\|\|\|. . \|. .\|\|\|\|\| \|\| \|\|. |
| EPI_ISL_402119_RBD/1-214 | GQTGKIADYNYKLPDDETGCVIAWNSNNLDSKVGGNYNYLYRLERKS |
| Wuhan_Node1_RBD/1-219 | KLKPFERDLSSDECSPDGKPCTPPAFNGVRGENCYFTLSTYDENPNV |
| | .\|\|\|\|\|\|\|.\|.. ... \|\| \|\|\| \|\|\|\|\|\| \| .\|.\|.\| |
| EPI_ISL_402119_RBD/1-214 | NLKPFERDISTEIYQAGSTPC-----NGVEGENCYFPLQSYGFQPTN |
| Wuhan_Node1_RBD/1-219 | PVEYQATRVVVLSFELLNAPATVCGPKLSTQ |
| | \| \|\|. \|\|\|\|\|\|\|\|\|\|.\|\|\|\|\|\|\|\| \|\|. |
| EPI_ISL_402119_RBD/1-214 | GVGYQPYRVVVLSFELLHAPATVCGPKKSTN |

Percentage ID = 70.32

### EXAMPLE 8

pEVAC Expression Vector

FIG. 3 shows a map of the pEVAC expression vector. The sequence of the multiple cloning site of the vector is given below, followed by its entire nucleotide sequence.

Sequence of pEVAC Multiple Cloning Site (MCS) (SEQ ID NO: 19):

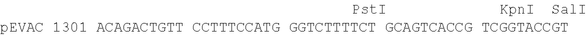

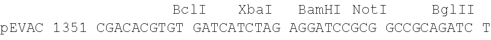

Entire Sequence of pEVAC (SEQ ID NO: 20):

| | | |
|---|---|---|
| CMV-IE-E/P: | 248-989 | CMV immediate early 1 enhancer/promoter |
| KanR: | 3445-4098 | Kanamycin resistance |
| SD: | 990-1220 | Splice donor |
| SA: | 1221-1343 | Splice acceptor |
| Tbgh: | 1392-1942 | Terminator signal from bovine growth hormone |
| pUC-ori: | 2096-2769 | pUC-plasmid origin of replication |

| | |
|---|---|
| 1 | TCGCGCGITT CGGTGATGAC GGTGAAAACC TCTGACACAT GCAGCTCCCG |
| 51 | GAGACGGTCA CAGCTTGTCT GTAAGCGGAT GCCGGGAGCA GACAAGCCCG |
| 101 | TCAGGGCGCG TCAGCGGGTG TTGGGGGGTG TCGGGGCTGG CTTAACTATG |
| 151 | CGGCATCAGA GCAGATTGTA CTGAGAGTGC ACCATATGCG GTGTGAAATA |
| 201 | CCGCACAGAT GCGTAAGGAG AAAATACCGC ATCAGATTGG CTATTGGCCA |
| 251 | TTGCATACGT TGTATCCATA TCATAATATG TACATTTATA TTGGCTCATG |
| 301 | TCCAACATTA CCGCCATGTT GACATTGATT ATTGACTAGT TATTAATAGT |
| 351 | AATCAATTAC GGGGTCATTA GTTCATAGCC CATATATGGA GTTCCGCGTT |
| 401 | ACATAACTTA CGGTAAATGG CCCGCCTGGC TGACCGCCCA ACGACCCCCG |
| 451 | CCCATTGACG TCAATAATGA CGTATGTTCC CATAGTAACG CCAATAGGGA |
| 501 | CTTTCCATTG ACGTCAATGG GTGGAGTATT TACGGTAAAC TGCCCACTTG |
| 551 | GCAGTACATC AAGTGTATCA TATGCCAAGT ACGCCCCCTA TTGACGTCAA |
| 601 | TGACGGTAAA TGGCCCGCCT GGCATTATGC CCAGTACATG ACCTTATGGG |
| 651 | ACTTTCCTAC TTGGCAGTAC ATCTACGTAT TAGTCATCGC TATTACCATG |
| 701 | GTGATGCGGT TTTGGCAGTA CATCAATGGG CGTGGATAGC GGTTTGACTC |
| 751 | ACGGGGATTT CCAAGTCTCC ACCCCATTGA CGTCAATGGG AGTTTGTTTT |
| 801 | GGCACCAAAA TCAACGGGAC TTTCCAAAAT GTCGTAACAA CTCCGCCCCA |
| 851 | TTGACGCAAA TGGGCGGTAG GCGTGTACGG TGGGAGGTCT ATATAAGCAG |
| 901 | AGCTCGTTTA GTGAACCGTC AGATCGCCTG GAGACGCCAT CCACGCTGTT |
| 951 | TTGACCTCCA TAGAAGACAC CGGGACCGAT CCAGCCTCCA TCGGCTCGCA |
| 1001 | TCTCTCCTTC ACGCGCCCGC CGCCCTACCT GAGGCCGCCA TCCACGCCGG |
| 1051 | TTGAGTCGCG TTCTGCCGCC TCCCGCCTGT GGTGCCTCCT GAACTGCGTC |
| 1101 | CGCCGTCTAG GTAAGTTTAA AGCTCAGGTC GAGACCGGGC CTTTGTCCGG |
| 1151 | CGCTCCCTTG GAGCCTACCT AGACTCAGCC GGCTCTCCAC GCTTTGCCTG |
| 1201 | ACCCTGCTTG CTCAACTCTA GTTAACGGTG GAGGGCAGTG TAGTCTGAGC |
| 1251 | AGTACTCGTT GCTGCCGCGC GCGCCACCAG ACATAATAGC TGACAGACTA |
| 1301 | ACAGACTGTT CCTTTCCATG GGTCTTTTCT GCAGTCACCG TCGGTACCGT |
| 1351 | CGACACGTGT GATCATCTAG AGGATCCGCG GCCGCAGATC TGCTGTGCCT |
| 1401 | TCTAGITGCC AGCCATCTGT TGTTTGCCCC TCCCCCGTGC CTTCCTTGAC |
| 1451 | CCTGGAAGGT GCCACTCCCA CTGTCCTTTC CTAATAAAAT GAGGAAATTG |
| 1501 | CATCGCATTG TCTGAGTAGG TGTCATTCTA TTCTGGGGGG TGGGGTGGGG |
| 1551 | CAGGACAGCA AGGGGGAGGA TTGGGAAGAC AATAGCAGGC ATGCTGGGGA |
| 1601 | TGCGGTGGGC TCTATGGCTA CCCAGGTGCT GAAGAATTGA CCCGGTTCCT |
| 1651 | CCTGGGCCAG AAAGAAGCAG GCACATCCCC TTCTCTGTGA CACACCCTGT |
| 1701 | CCACGCCCCT GGTTCTTAGT TCCAGCCCCA CTCATAGGAC ACTCATAGCT |
| 1751 | CAGGAGGGCT CCGCCTTCAA TCCCACCCGC TAAAGTACTT GGAGCGGTCT |
| 1801 | CTCCCTCCCT CATCAGCCCA CCAAACCAAA CCTAGCCTCC AAGAGTGGGA |
| 1851 | AGAAATTAAA GCAAGATAGG CTATTAAGTG CAGAGGGAGA GAAAATGCCT |
| 1901 | CCAACATGTG AGGAAGTAAT GAGAGAAATC ATAGAATTTT AAGGCCATGA |
| 1951 | TTTAAGGCCA TCATGGCCTT AATCTTCCGC TTCCTCGCTC ACTGACTCGC |
| 2001 | TGCGCTCGGT CGTTCGGCTG CGGCGAGCGG TATCAGCTCA CTCAAAGGCG |
| 2051 | GTAATACGGT TATCCACAGA ATCAGGGGAT AACGCAGGAA AGAACATGTG |
| 2101 | AGCAAAAGGC CAGCAAAAGG CCAGGAACCG TAAAAAGGCC GCGTTGCTGG |
| 2151 | CGTTTTTCCA TAGGCTCCGC CCCCCTGACG AGCATCACAA AAATCGACGC |
| 2201 | TCAAGTCAGA GGTGGCGAAA CCCGACAGGA CTATAAAGAT ACCAGGCGTT |
| 2251 | TCCCCCTGGA AGCTCCCTCG TGCGCTCTCC TGTTCCGACC CTGCCGCTTA |
| 2301 | CCGGATACCT GTCCGCCTTT CTCCCTTCGG GAAGCGTGGC GCTTTCTCAT |
| 2351 | AGCTCACGCT GTAGGTATCT CAGTTCGGTG TAGGTCGTTC GCTCCAAGCT |
| 2401 | GGGCTGTGTG CACGAACCCC CCGTTCAGCC CGACCGCTGC GCCTTATCCG |
| 2451 | GTAACTATCG TOTTGAGTCC AACCCGGTAA GACACGACTT ATCGCCACTG |
| 2501 | GCAGCAGCCA CTGGTAACAG GATTAGCAGA GCGAGGTATG TAGGCGGTGC |
| 2551 | TACAGAGTTC TTGAAGTGGT GGCCTAACTA CGGCTACACT AGAAGAACAG |
| 2601 | TATTTGGTAT CTGCGCTCTG CTGAAGCCAG TTACCTTCGG AAAAAGAGTT |
| 2651 | GGTAGCTCTT GATCCGGCAA ACAAACCACC GCTGGTAGCG GTGGTTTTTT |
| 2701 | TGTTTGCAAG CAGCAGATTA CGCGCAGAAA AAAAGGATCT CAAGAAGATC |
| 2751 | CTTTGATCTT TTCTACGGGG TCTGACGCTC AGTGGAACGA AAACTCACGT |
| 2801 | TAAGGGATTT TGGTCATGAG ATTATCAAAA AGGATCTICA CCTAGATCCT |
| 2851 | TTTAAATTAA AAATGAAGTT TTAAATCAAT CTAAAGTATA TATGAGTAAA |
| 2901 | CTTGGTCTGA CAGTTACCAA TGCTTAATCA GTGAGGCACC TATCTCAGCG |
| 2951 | ATCTGTCTAT TTCGTTCATC CATAGTTGCC TGACTCGGGG GGGGGGGGCG |
| 3001 | CTGAGGTCTG CCTCGTGAAG AAGGTGTTGC TGACTCATAC CAGGCCTGAA |
| 3051 | TCGCCCCATC ATCCAGCCAG AAAGTGAGGG AGCCACGGTT GATGAGAGCT |
| 3101 | TTGTTGTAGG TGGACCAGTT GGTGATTTTG AACTTTTGCT TTGCCACGGA |
| 3151 | ACGGTCTGCG TTGTCGGGAA GATGCGTGAT CTGATCCTTC AACTCAGCAA |
| 3201 | AAGTTCGATT TATTCAACAA AGCCGCCGTC CCGTCAAGTC AGCGTAATGC |
| 3251 | TCTGCCAGTG TTACAACCAA TTAACCAATT CTGATTAGAA AAACTCATCG |
| 3301 | AGCATCAAAT GAAACTGCAA TTTATTCATA TCAGGATTAT CAATACCATA |
| 3351 | TTTTTGAAAA AGCCGTTTCT GTAATGAAGG AGAAAACTCA CCGAGGCAGT |
| 3401 | TCCATAGGAT GGCAAGATCC TGGTATCGGT CTGCGATTCC GACTCGTCCA |
| 3451 | ACATCAATAC AACCTATTAA TTTCCCCTCG TCAAAAATAA GGTTATCAAG |
| 3501 | TGAGAAATCA CCATGAGTGA CGACTGAATC CGGTGAGAAT GGCAAAAGCT |
| 3551 | TATGCATTTC TTTCCAGACT TGTTCAACAG GCCAGCCATT ACGCTCGTCA |
| 3601 | TCAAAATCAC TCGCATCAAC CAAACCGTTA TTCATTCGTG ATTGCGCCTG |
| 3651 | AGCGAGACGA AATACGCGAT CGCTGTTAAA AGGACAATTA CAAACAGGAA |
| 3701 | TCGAATGCAA CCGGCGCAGG AACACTGCCA GCGCATCAAC AATATTTTCA |
| 3751 | CCTGAATCAG GATATTCTTC TAATACCTGG AATGCTGTTT TCCCGGGGAT |
| 3801 | CGCAGTGGTG AGTAACCATG CATCATCAGG AGTACGGATA AAATGCTTGA |
| 3851 | TGGTCGGAAG AGGCATAAAT TCCGTCAGCC AGTTTAGTCT GACCATCTCA |
| 3901 | TCTGTAACAT CATTGGCAAC GCTACCTTTG CCATGTTTCA GAAACAACTC |
| 3951 | TGGCGCATCG GGCTTCCCAT ACAATCGATA GATTGTCGCA CCTGATTGCC |
| 4001 | CGACATTATC GCGAGCCCAT TTATACCCAT ATAAATCAGC ATCCATGTTG |
| 4051 | GAATTTAATC GCGGCCTCGA GCAAGACGTT TCCCGTTGAA TATGGCTCAT |
| 4101 | AACACCCCTT GTATTACTGT TTATGTAAGC AGACAGTTTT ATTGTTCATG |
| 4151 | ATGATATATT TTTATCTTGT GCAATGTAAC ATCAGAGATT TTGAGACACA |
| 4201 | ACGTGGCTTT CCCCCCCCCC CCATTATTGA AGCATTTATC AGGGTTATTG |
| 4251 | TCTCATGAGC GGATACATAT TTGAATGTAT TTAGAAAAAT AAACAAATAG |
| 4301 | GGGTTCCGCG CACATTTCCC CGAAAAGTGC CACCTGACGT CTAAGAAACC |
| 4351 | ATTATTATCA TGACATTAAC CTATAAAAAT AGGCGTATCA CGAGGCCCTT |
| 4401 | TCGTC |

### EXAMPLE 9

Common Amino Acid Differences of Wuhan Node1_RBD (CoV_T2_7) Amino Acid Sequence (SEQ ID NO:17) with AY274119_RBD (CoV_T2_5) (SEQ ID NO:5) and EPI_ISL_402119_RBD (CoV_T2_6) (SEQ ID NO:11) Amino Acid Sequences

FIG. 4 shows Wuhan_Node1_RBD (CoV_T2_7) amino acid sequence (SEQ ID NO:17) with amino acid residue differences highlighted in bold and underline from the respective alignments with AY274119_RBD (CoV_T2_5) (SEQ ID NO:5) and EPI_ISL_402119_RBD (CoV_T2_6) (SEQ ID NO:11) amino acid sequences (Examples 6 and 7, respectively).

The amino acid residue differences from the two alignments are listed in the table below (the numbering of residue positions corresponds to positions of the Wuhan_Node1_RBD (CoV_T2_7) (SEQ ID NO:17) amino acid sequence. The common differences from the two alignments are at amino acid residues: 3, 6, 7, 21, 22, 38, 42, 48, 67, 70, 76, 81, 83, 86, 87, 92, 121, 122, 123, 125, 126, 128, 134, 137, 138, 141, 150, 152, 153, 154, 155, 167, 171, 178, 180, 181, 183, 185, 187, 188, 189, 191, 194, 195, 219 (shown with grey highlighting in FIG. 4, and in the table below):

Amino acid insertions are at positions 167-172 (compared to AY274119_RBD), and 163-167 (compared to EPI_ISL_402119_RBD) (shown boxed in FIG. 4).

### EXAMPLE 9

Immune Response Induced by DNA Vaccine Encoding “panS” Antigen

Mice (n=6) were immunised with DNA encoding a “panS” antigen according to an embodiment of the invention (Wuhan_Node1 (CoV_T2_1), nucleic acid of SEQ ID NO:13, encoding full length S-protein of amino acid SEQ ID NO:14), full-length S gene from SARS-Cov-1, or full-length S gene from SARS-CoV-2.

Antibodies in serum obtained from the mice were compared for their ability to bind wild-type antigens through FACS.

FIG. 5 shows dose response curves of antibody binding to SARS-CoV-1 (A) or SARS-CoV-2 (B) full length Spike protein expressed on HEK293T cells. Flow cytometry based cell display assay reported in MFI (Median Fluorescent Intensity).

Serum from mice immunised with either wildtype S gene show weak binding to heterologous protein. In contrast, serum from mice immunised with the “panS” antigen binds to both SARS-CoV-1 and SARS-CoV-2 Spike proteins.

It was concluded that the “panS” antigen induces an immune response that is more cross-reactive than wild-type antigens, indicating protection against future Sarbecovirus outbreaks not conferred by using naturally occurring antigens.

### EXAMPLE 10

### Envelope (E) Protein Vaccine Sequences

FIG. 6 shows an amino acid sequence of the SARS envelope protein (SEQ ID NO:21), and illustrates key features of the sequence:

(SEQ ID NO: 21)
MYSFVSEETG TLIVNSVLLF LAFVVFLLVT LAILTALRLC
AYCCNIVNVS LVKPTVYVYS RVKNINSSEG VPDLLV

FIG. 7 shows a multiple sequence alignment of coronavirus Envelope (E) protein sequences, comparing sequences for isolates of NL63 and 229E (alpha-coronaviruses), and HKU1, MERS, SARS, and SARS2 (beta-coronaviruses). The alignment shows that the C-terminal end of the E protein for the SARS2 and SARS sequences (beta-coronaviruses of subgenus Sarbeco) includes a deletion, compared with the other sequences, and that the SARS2 E protein sequence includes a deletion, and an Arginine (positively charged) amino acid residue, compared with the SARS sequence.

We have generated novel sequences for the Envelope (E) protein, called COV_E_T2_1 (a designed Sarbecovirus sequence) (SEQ ID NO:22) and COV_E_T2_2 (a designed SARS2 sequence) (SEQ ID NO:23):

>COV_E_T2_1
(SEQ ID NO: 22)
MYSFVSEETG TLIVNSVLLF LAFVVFLLVT LAILTALRLC
AYCCNIVNVS LVKPTFYVYS RVKNENSSQG VPDLLV
>COV_E_T2_2
(SEQ ID NO: 23)
MYSFVSEETG TLIVNSVLLF LAFVVFLLVT LAILTALRLC
AYCCNIVNVS LVKPTFYVYS RVKNENSSR- VPDLLV

Alignment of the SARS2 reference E protein sequence in FIG. 7 with these designed sequences highlights that there are four amino acid differences between the SARS2 reference E protein sequence and the COV_E_T2_1 designed sequence (SEQ ID NO:22), and two amino acid differences between the SARS2 reference E protein sequence and the COV_E_T2_2 designed sequence (SEQ ID NO:23) (see the boxed amino acid residues in the amino acid sequence alignment below):

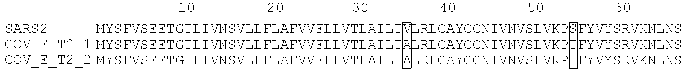

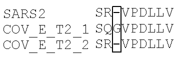

The C-terminal sequence of the COV_E_T2_2 sequence is identical to the SARS2 reference sequence. The C-terminal of the E protein is one of the identified epitopes for E-protein, so the amino acid deletion and the substitution with an Arginine residue present in the SARS2 reference sequence (compared with the SARS reference sequence in FIG. 6) have been retained in the COV_E_T2_2 designed sequence. The amino acid differences at the other positions are optimised to maximise induction of an immune response that recognises all Sarbeco viruses.

The amino acid differences are summarised in the table below:

| SARS2 E | SARS2 | COV_E_T2_1 | COV_E_T2_2 |
|---|---|---|---|
| protein | Reference | Amino | Amino |
| residue | Amino acid | acid | acid |
| position | residue | residue | residue |
| 36 | V | [symbol] | [symbol] |
| 55 | S | [symbol] | [symbol] |
| 69 | R | Q | R |
| 70 | — | G | — |

In the alignment above, residue 38 of the SARS2 reference sequence is shown as V, but is actually A (as correctly shown in FIG. 7 and SEQ ID NO:21). Alignment of SEQ ID NO:21 with the designed sequences highlights that there are three amino acid differences between the alternative SARS2 reference E protein sequence and the COV_E_T2_1 designed sequence (SEQ ID NO:22), and one amino acid difference between the SARS2 reference E protein sequence and the COV_E_T2_2 designed sequence (SEQ ID NO:23):

| | | |
|---|---|---|
| SARS2 COV_E_T2_1 COV_E_T2_2 | 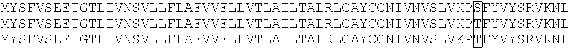 | 65 65 65 |
| SARS2 COV_E_T2_1 COV_E_T2_2 | 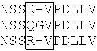 | 75 76 75 |

The amino acid differences are summarised in the table below:

| SARS2 E | SARS2 | COV_E_ | COV_E_ |
|---|---|---|---|
| protein | Reference | T2_1 | T2_2 |
| residue | Amino acid | Amino | Amino |
| position | residue | acid residue | acid residue |
| 55 | S | [symbol] | [symbol] |
| 69 | R | Q | R |
| 70 | — | G | — |

### EXAMPLE 11

### Membrane (M) Protein Vaccine Sequences

We have generated novel sequences for the coronavirus membrane (M) protein:
COV_M_T2_1 Sarbecovirus root ancestor (SEQ ID NO:24);
COV_M_T2_2 Epitope optimised version of SARS2 clade ancestor Node88b (D4 removed), SARS2 equivalent of B cell epitope from start and end added, and then T cell epitopes added whilst observing coevolving site constraints (SEQ ID NO:25).

The amino acid sequences of these designed sequences are:

>COV_M_T2_1/1-221 Sarbeco_M_root:
(SEQ ID NO: 24)
MADNGTITVE ELKQLLEQWN EVIGFLFLAW
IMLLQFAYSN RNRFLYIIKL VFLWLLWPVT
LACFVLAAVY RINWVIGGIA IAMACIVGLM
WLSYEVASER LFARTRSMWS FNPETNILLN
VPLRGTILTR PLMESELVIG AVIIRGHLRM
AGHSLGRCDI KDLPKEITVA TSRTLSYYKL
GASQRVGTDS GEAAYNRYRI GNYKINTDHA
GSNDNIALLV Q
>COV_M_T2_2/1-222 Sarbeco_M_Node88b_
epitope_optimised:
(SEQ ID NO: 25)
MADSNGTITV EELKKLLEQW NLVIGELFLT
WICLLQFAYS NRNRFLYIIK LIFLWLLWPV
TLACFVLAAV YRINWVIGGI AIAMACIVGL
MWLSYFVASF RLFARTRSMW SENPETNILL
NVPLRGSIIT RPLMESELVI GAVILRGHLR
MAGHSIGRCD IKDLPKEITV ATSRTLSYYK
LGASQRVASD SGFAVYNRYR IGNYKINTDH
SSSSDNIALL VQ

Alignment of the following SARS2 reference M protein sequence (SEQ ID NO:26) with the designed sequences is shown in FIG. 8. The reference M protein sequence is:
>COV_M_T1_1/1-222 NC_045512.2 SARS2 reference sequence:

(SEQ ID NO: 26)
MADSNGTITV EELKKLLEQW NIVIGFLFLT
WICLLQFAYA NRNRFLYIIK LIFLWLLWPV
TLACFVLAAV YRINWITGGI AIAMACIVGL
MWLSYFIASF RLFARTRSMW SENPETNILL
NVPLHGTILT RPLLESELVI GAVILRGHLR
IAGHHLGRCD IKDLPKEITV ATSRTLSYYK
LGASQRVAGD SGFAAYSRYR IGNYKINTDH
SSSSDNIALL VQ

The alignment shown in FIG. 8 highlights the amino acid differences between the SARS2 reference M protein sequence and the COV_M_T2_1 and COV_M_T2_2 designed sequences, as shown in the table below:

| SARS2 | SARS2 | COV_M_T2_1 | COV_M_ |
|---|---|---|---|
| M protein | Reference | Amino | T2_2 Amino |
| residue | Amino acid | acid | acid |
| position | residue | residue | residue |
| 4 | S | — | S |
| 15 | K | Q | K |
| 30 | T | A | T |
| 33 | C | M | C |
| 40 | A | [symbol] | [symbol] |
| 52 | I | V | I |
| 76 | I | [symbol] | [symbol] |
| 87 | L | [symbol] | [symbol] |
| 97 | I | [symbol] | [symbol] |
| 125 | H | [symbol] | [symbol] |
| 127 | T | T | S |
| 134 | L | [symbol] | [symbol] |
| 145 | L | I | L |
| 151 | I | [symbol] | [symbol] |
| 155 | H | [symbol] | [symbol] |
| 188 | A | G | A |
| 189 | G | T | S |
| 195 | A | A | V |
| 197 | S | [symbol] | [symbol] |
| 211 | S | A | S |
| 212 | S | G | S |
| 214 | S | N | S |

### EXAMPLE 12

### Clinical Trial Design

The study will consist of thirty SARS-CoV-2 PCR, antibody and T-cell negative healthy human volunteers enrolled for this trial, who agree to self-isolate and report back during the three immunisations, in order to demonstrate safety and immunogenicity.

The first of 3 study Groups will consist of:
Group 1; n=6 dose escalation;
Group 2; 12 healthy human volunteers with the needless PharmaJet delivery;
Group 3: 12 healthy human volunteers receiving direct intramuscular (IM) administration of DNA to benchmark the results by Martin et a (Vaccine, 2008).

The PharmaJet arm of the trial uses a dose-sparing needless delivery system, which minimises the barriers to people taking the vaccine. Power calculations are based on an estimated standard deviation of 0.27 log 10 units, using the ELISA data from the SARS clinical Trial (Martin et al, Vaccine, 2008).

Due to the pandemic emergency, primary and secondary endpoints will be analysed when the last patient has completed 3 months following primary immunisation (complete safety data for 28 days, and immunogenicity primary and key secondary endpoints to 3 months).

### Secondary Objective/Endpoints to Assess the Immunogenicity of the Vaccine:

Key immunogenicity endpoints to be analysed and reported at 3 months: Serology (t=0, 14 days, 28 days, 2 months, 3 months). In addition to antigen specific IgM and IgG ELISAs, ADE and ADCC assays will be performed at all time points. Standardised microneutralization assays to measure neutralizing capacity of vaccine antigen-specific antibodies in sera collected pre- and post-immunization at the defined time points.

Antigen-specific T cellular immune responses will be measured at t=0, 14 days, 28 days, 2 months, 3 months). Antigen-specific T cell immune responses will be evaluated in cryopreserved PBMC from vaccinees by proliferation assay (CFSE) and IFN gamma ELISPOT as a preliminary screening of positive responders. A detailed phenotypic analysis of the vaccine-induced T cell responses performed by flow cytometry will follow to determine subpopulations induced by the vaccine candidates [Central memory T-cells (TCM), Effector memory T-cells (TEM) and regulatory T-cells (Treg)] coupled to functional analysis of T cells by intracellular staining for different cytokines (IFN gamma, TNF-α, IL-17, IL-2 and IL-10). Ex vivo nCoV-specific CD8+ and CD4+ T cell subsets, tested for their expression of CD3, CD4, CD8, CD45RA/RO, CD62L, CCR7, CD127, CD25 and nuclear FoxP3, will be identified by multiparametric flow cytometry with fluorochrome-labelled dextramers. If necessary, dextramer analysis will be coupled to a 12-15 day in vitro re-stimulation with vaccine-specific synthetic peptides (20 amino acids overlapped by 12 amino-acids) spanning the Spike (S) protein. Moreover, supernatants of secondary cultures will be also assessed for a large panel of cytokines (IFN-gamma IL-4, IL-5, IL-2, IL-10, IL-13, IL-17, IL-21 and TNF-α) in order to precisely define T cell polarization allowing the identification of T helper subsets and poly-functionality by using the Bio-Plex Pro™ Human Cytokine plex Assay (Biorad).

### EXAMPLE 13

### Further Designed S Protein RBD Sequences.

We have generated further novel S protein RBD sequences by modifying the previous input alignment to our design algorithm: CoV_S_T2_13-CoV_S_T2_18. CoV_S_T2_13 is the direct output of the design algorithm, and CoV_S_T2_14-CoV_S_T2_18 are epitope-enriched versions of CoV_S_T2_13.

The amino acid sequences of these designed sequences are:

>COV_S_T2_13
(SEQ ID NO: 27)
RVAPTKEVVR FPNITNLCPF GEVENATRFP SVYAWERKRI
SNCVADYSVL YNSTSFSTEK CYGVSPTKLN DLCFINVYAD
SFVIRGDEVR QIAPGQTGVI ADYNYKLPDD FTGCVIAWNT
NNLDSTTGGN YNYLYRSLRK SKLKPFERDI SSDIYSPGGK
PCSGVEGENC YYPLRSYGFF PINGVGYQPY RVVVLSFELL
NAPATVCGPK LSTD
>COV_S T2_14
(SEQ ID NO: 28)
RVAPTKEVVR FPNITNLCPF GEVENATKEP SVYAWERKKI
SNCVADYSVL YNSTSESTEK CYGVSPTKLN DLCFTNVYAD
SFVIRGDEVR QIAPGQTGVI ADYNYKLPDD FTGCVIAWNT
NNIDSTTGGN YNYLYRSLRK SKLKPFERDI SSDIYSPGGK
PCSGVEGENC YYPLRSYGFF PINGVGYQPY RVVVLSFELL
NAPATVCGPK LSTD
>COV_S T2 15
(SEQ ID NO: 29)
RVAPTKEVVR FPNITNLCPF GEVENATREP SVYAWERKRI
SNCVADYSVL YNSTFFSTEK CYGVSPTKLN DLCFSNVYAD
SFVIRGDEVR QIAPGQTGVI ADYNYKLPDD FMGCVIAWNT
NNLDSTTGGN YNYLYRSLRK SKLKPFERDI SSDIYSPGGK
PCSGVEGENC YYPLRSYGFF PINGVGYQPY RVVVLSFELL
NAPATVCGPK LSTD
>COV_S_T2_16
(SEQ ID NO: 30)
RVAPTKEVVR FPNITNLCPF GEVENATREP SVYAWERKRI
SNCVADYSVL YNSTSFSTFK CYGVSPTKLN DLCFINVYAD
SFVIRGDEVR QIAPGQTGKI ADYNYKLPDD FTGCVIAWNT
NNLDSTTGGN YNYLYRLERK SNLKPFERDI SSDIYQAGST
PCSGVEGENC YFPLQSYGFQ PINGVGYQPY RVVVLSFELL
NAPATVCGPK LSTD
>COV_S T2 17
(SEQ ID NO: 31)
RVAPTKEVVR FPNITNLCPF GEVENATKEP SVYAWERKKI
SNCVADYSVL YNSTSFSTEK CYGVSPTKLN DLCFINVYAD
SFVIRGDEVR QIAPGQTGVI ADYNYKLPDD FTGCVIAWNT
NNIDSTTGGN YNYLYRSLRK SKLKPFERDI SSDIYSPGGK
PCSGVEGENC YYPLRSYGFF PINGTGYQPY RVVVLSFELL
NAPATVCGPK LSTD
>COV_S_T2_18
(SEQ ID NO: 32)
RVAPTKEVVR FPNITNLCPF GEVENATRFP SVYAWERKRI
SNCVADYSVL YNSTFFSTEK CYGVSPTKLN DLCFSNVYAD
SFVIRGDEVR QIAPGQTGVI ADYNYKLPDD EMGCVIAWNT
NNLDSTTGGN YNYLYRSLRK SKLKPFERDI SSDIYSPGGK
PCSGVEGENC YYPLRSYGFF PINGTGYQPY RVVVLSFELL
NAPATVCGPK LSTD

Alignment of these sequences (SEQ ID NO:27-32) with SARS2 Reference sequence (EPI_ISL_402119_RBD (CoV_T2_6) (SEQ ID NO:11)) is shown below (the boxed regions highlight sequence differences in the alignments):

| | | |
|---|---|---|
| SARS2 COV_S_T2_13 COV_S_T2_14 COV_S_T2_15 COV_S_T2_16 COV_S_T2_17 COV_S_T2_18 | 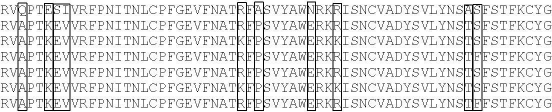 | 63 63 63 63 63 63 63 |
| SARS2 COV_S_T2_13 COV_S_T2_14 COV_S_T2_15 COV_S_T2_16 COV_S_T2_17 COV_S_T2_18 | 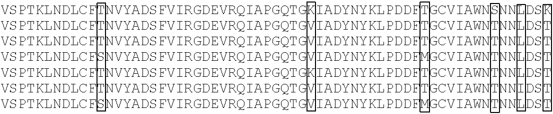 | 126 126 126 126 126 126 126 |
| SARS2 COV_S_T2_13 COV_S_T2_14 COV_S_T2_15 COV_S_T2_16 COV_S_T2_17 COV_S_T2_18 | 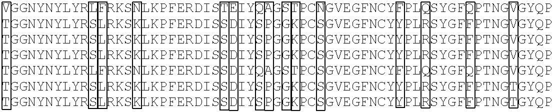 | 189 189 189 189 189 189 189 |
| SARS2 COV_S_T2_13 COV_S_T2_14 COV_S_T2_15 COV_S_T2_16 COV_S_T2_17 COV_S_T2_18 | 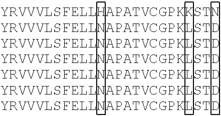 | 214 214 214 214 214 214 214 |

### EXAMPLE 14

Further Designed S Protein RBD Sequences (with Altered Glycosylation Sites)

Masking/de-masking of epitopes has been shown to alter the immune response by masking non-neutralising epitopes, or by de-masking important epitopes in MERS (Du L et. al., Nat. Comm, 2016).

We have prepared additional designed S protein RBD sequences in which we have deleted a glycosylation site of, or introduced a glycosylation site to, the SARS2 RBD sequence. The changes made are illustrated in FIG. 13. The figure shows amino acid sequence of the RBD region. The circled numbers show the positions at which a glycosylation site has been deleted or introduced. Numbers circled in light grey represent deletion of a glycosylation site. Numbers circled in dark grey represent introduction of a glycosylation site. At the position marked by circled number 3, a glycosylation site is present in the SARS wild-type sequence, but absent in the SARS-2 wild-type sequence. This may be important for non-neutralising epitope masking. The introduced glycosylation site is only present in the M8 design.

### Modifications in the RBD:

designs M7 and M9 include a glycosylation site introduced at the position indicated by circled number 4 (residue position 203);
designs M8 and M10 include a deleted glycosylation site at each of the positions indicated by circled numbers 1 and 2 (residue positions 13 and 25, respectively). The M8 design also includes an introduced glycosylation site at the position indicated by circled number 3 (residue position 54).

The amino acid sequences of SARS2 RBD designs M7, M8, M9, and M10 are shown below:

>M7
(SEQ ID NO: 33)
RVQPTESIVR FPNITNLCPF GEVENATREA SVYAWNRKRI
SNCVADYSVL YNSASESTEK CYGVSPTKLN DLCFINVYAD
SFVIRGDEVR QIAPGQTGKI ADYNYKLPDD ETGCVIAWNS
NNLDSKVGGN YNYLYRLERK SNLKPFERDI STEIYQAGST
PCNGVEGENC YFPLQSYGFQ PINGVGYQPY RVVVLSFELL
HANATVCGPK KSTN
>M8
(SEQ ID NO: 34)
RVQPTESIVR FPQITNLCPF GEVEQATREA SVYAWNRKRI
SNCVADYSVL YNSTSESTEK CYGVSPTKLN DLCFINVYAD
SFVIRGDEVR QIAPGQTGKI ADYNYKLPDD FTGCVIAWNS
NNLDSKVGGN YNYLYRLFRK SNLKPFERDI STEIYQAGST
PCNGVEGENC YFPLQSYGFQ PINGVGYQPY RVVVLSFELL
HAPATVCGPK KSTN
>M9
(SEQ ID NO: 35)
RVSPTQEVVR FPNITNLCPF DKVENATREP SVYAWERTKI
SDCVADYTVL YNSTSESTEK CYGVSPSKLI DLCFTSVYAD
TFLIRCSEVR QVAPGQTGVI ADYNYKLPDD FTGCVIAWNT
AKQDTGSSGN YNYYYRSHRK TKLKPFERDL SSDECSPDGK
PCTPPAENGV RGENCYFTLS TYDENPNVPV EYQATRVVVL
SFELLNANAT VCGPKLSTQ
>M10
(SEQ ID NO: 36)
RVSPTQEVVR FPQITNLCPF DKVFQATREP SVYAWERTKI
SDCVADYTVL YNSTSESTEK CYGVSPSKLI DLCFTSVYAD
TFLIRCSEVR QVAPGQTGVI ADYNYKLPDD FTGCVIAWNT
AKQDTGSSGN YNYYYRSHRK TKLKPFERDL SSDECSPDGK
PCTPPAENGV RGENCYFTLS TYDENPNVPV EYQATRVVVL
SFELLNAPAT VCGPKLSTQ

Alignment of these sequences (SEQ ID NOs:33-36) with the SARS2 Reference sequence (EPI_ISL_402119_RBD (CoV_T2_6) (SEQ ID NO:11)) is shown below (with the dots representing no difference in amino acid residue from the reference sequence, and the dashes representing positions where amino acid residues have been inserted in the M9 and M10 sequences):

| | | |
|---|---|---|
| SARS2_RBD | RVQPTESIVRFPNITNLCPFGEVENATRFASVYAWNRKRISNCVADYSVLYNSASFSTFKCYGV | 64 |
| M7 | ................................................................ | 64 |
| M8 | ............Q...........Q............................T.......... | 64 |
| M9 | ..S..QEV............DK.......P.....E.TK..D.....T.....T.......... | 64 |
| M10 | ..S..QEV....Q.......DK..Q....P.....E.TK..D.....T.....T.......... | 64 |
| SARS2_RBD | SPTELNQLCFINVYADSFVIRGDEVRQIAPGQTGKIADYNYELPQDFTGCVIAWNSNNLDSKVG | 128 |
| M7 | ................................................................ | 128 |
| M8 | ................................................................ | 128 |
| M9 | ..S..I.....S....T.L..CS....V......V....................TAKQ.TGSS | 128 |
| M10 | ..S..I.....S....T.L..CS....V......V....................TAKQ.TGSS | 128 |
| SARS2_RBD | GNYNYLYRLERKSNLKPFERDISTEIYQAGSTPC-----NGVEGENCYFPLQSYGFQPINGVGY | 187 |
| M7 | ..................................-----......................... | 187 |
| M8 | ..................................-----......................... | 187 |
| M9 | .....Y..SH..TK.......L.SDECSPDGK..TPPAF...R......T.ST.D.N.NVP.E. | 192 |
| M10 | .....Y..SH..TK.......L.SDECSPDGK..TPPAF...R......T.ST.D.N.NVP.E. | 192 |
| SARS2_RBD | QPYRVVVLSFELLHAPATVCGPKKSTN | 214 |
| M7 | ...............N........... | 214 |
| M8 | ........................... | 214 |
| M9 | .AT..........N.N.......L..Q | 219 |
| M10 | .AT..........N.........L..Q | 219 |

The amino acid differences of the designed sequences from the SARS2 reference sequence are summarised in the table below (with differences from the reference sequence highlighted in bold):

### EXAMPLE 15

### Nucleotide Sequences of Further Designed S Protein RBD Sequences

Nucleotide sequences encoding the M7, M8, M9, and M10 SARS2 RBD designs discussed in Example 14 are shown below:

>M7
(SEQ ID NO: 37)
cgggtgcagc ccaccgaatc catcgtgcgg ttccccaata tcaccaatct gtgccccttc 60
ggcgaggtgt tcaatgccac cagattcgcc tctgtgtacg cctggaaccg gaagcggatc 120
agcaattgcg tggccgacta ctccgtgctg tacaactccg ccagcttcag caccttcaag 180
tgctacggcg tgtcccctac caagctgaac gacctgtgct tcacaaacgt gtacgccgac 240
agcttcgtga tccggggaga tgaagtgcgg cagattgccc ctggacagac aggcaagatc 300
gccgactaca actacaagct gcccgacgac ttcaccggct gtgtgattgc ctggaacagc 360
aacaacctgg actccaaagt cggcggcaac tacaattacc tgtaccggct gttccggaag 420
tccaatctga agcccttcga gcgggacatc agcaccgaaa tctatcaggc cggcagcacc 480
ccttgcaacg gcgtggaagg cttcaactgc tacttcccac tgcaaagcta cggctttcag 540
cccacaaatg gcgtgggcta ccagccttac agagtggtgg tgctgagctt cgagctgctg 600
catgctaacg ccacagtgtg cggccctaag aaatccacca at 642
>M8
(SEQ ID NO: 38)
cgggtgcagc ccaccgaatc catcgtgcgg ttcccccaga tcaccaatct gtgccccttc 60
ggcgaggtgt tccaggccac cagattcgcc tctgtgtacg cctggaaccg gaagcggatc 120
agcaattgcg tggccgacta ctccgtgctg tacaactcca ccagcttcag caccttcaag 180
tgctacggcg tgtcccctac caagctgaac gacctgtgct tcacaaacgt gtacgccgac 240
agcttcgtga tccggggaga tgaagtgcgg cagattgccc ctggacagac aggcaagatc 300
gccgactaca actacaagct gcccgacgac ttcaccggct gtgtgattgc ctggaacagc 360
aacaacctgg actccaaagt cggcggcaac tacaattacc tgtaccggct gttccggaag 420
tccaatctga agcccttcga gcgggacatc agcaccgaaa tctatcaggc cggcagcacc 480
ccttgcaacg gcgtggaagg cttcaactgc tacttcccac tgcaaagcta cggctttcag 540
cccacaaatg gcgtgggcta ccagccttac agagtggtgg tgctgagctt cgagctgctg 600
catgctcctg ccacagtgtg cggccctaag aaatccacca at 642
>M9
(SEQ ID NO: 39)
cgggtgtccc ctacacaaga ggtcgtgcgg ttccccaata tcaccaatct gtgccccttc 60
gacaaggtgt tcaacgccac cagatttccc agcgtgtacg cctgggagcg caccaagatt 120
tccgattgcg tggccgacta caccgtgctg tataactcca cctccttcag caccttcaag 180
tgctacggcg tgtccccaag caagctgatc gatctgtgct tcacctctgt gtacgccgac 240
accttcctga tccggtgtag cgaagtgcga caggtggcac ctggacagac aggcgtgatc 300
gccgattaca actacaagct gcccgacgac ttcaccggct gtgtgatcgc ctggaatacc 360
gccaagcagg atacaggcag cagcggcaac tacaactact actacagaag ccaccgcaag 420
accaagctga agcctttcga gagggacctg agcagcgacg agtgtagccc tgatggcaag 480
ccttgtacac ctcctgcctt caatggcgtg cggggcttca actgctactt caccctgagc 540
acctacgact tcaaccccaa cgtgcccgtg gaataccagg ccacaagagt ggtggtgctg 600
agcttcgagc tgctgaatgc caacgccaca gtgtgtggcc ctaagctgtc tacccag 657
>M10
(SEQ ID NO: 40)
cgggtgtccc ctacacaaga ggtcgtgcgg ttcccccaga tcaccaatct gtgccccttc 60
gacaaggtgt tccaggccac cagatttccc agcgtgtacg cctgggagcg caccaagatt 120
tccgattgcg tggccgacta caccgtgctg tataactcca cctccttcag caccttcaag 180
tgctacggcg tgtccccaag caagctgatc gatctgtgct tcacctctgt gtacgccgac 240
accttcctga tccggtgtag cgaagtgcga caggtggcac ctggacagac aggcgtgatc 300
gccgattaca actacaagct gcccgacgac ttcaccggct gtgtgatcgc ctggaatacc 360
gccaagcagg atacaggcag cagcggcaac tacaactact actacagaag ccaccgcaag 420
accaagctga agcctttcga gagggacctg agcagcgacg agtgtagccc tgatggcaag 480
ccttgtacac ctcctgcctt caatggcgtg cggggcttca actgctactt caccctgagc 540
acctacgact tcaaccccaa cgtgcccgtg gaataccagg ccacaagagt ggtggtgctg 600
agcttcgagc tgctgaatgc ccctgccaca gtgtgtggcc ctaagctgtc tacccag 657

Differences between these sequences (SEQ ID NOs: 37-40) are highlighted in the alignment below (with the dots indicating that the nucleotide residue is the same as the corresponding M7 nucleotide residue):

| | | |
|---|---|---|
| M7 | CGGGTGCAGCCCACCGAATCCATCGTGCGGTTCCCCAATATCACCAATCTGTGCCCCTTCGGCGAGGT | 68 |
| M8 | ....................................C.G............................. | 68 |
| M9 | ......TCC..T..AC..GAGGA......................................A.A.... | 68 |
| M10 | ......TCC..T..AC..GAGGC.............C.G......................A.A.... | 68 |
| M7 | GTTCAATGCCACCAGATTCGCCTCTGTGTACGCETGGAACCGGAAGCGGATCAGCAATTGCGTGGCCG | 136 |
| M8 | ....C.G............................................................. | 136 |
| M9 | ......C...........TC..AGC............G.G..C.CCAA...TTC.G............ | 136 |
| M10 | ....C.G...........TC..AGC............G.G..C.CCAA...TTC.G............ | 136 |
| M7 | ACTACTCCGTGCTGTACAACTCCGCCAGCTTCAGCACCTTCAAGTGCTACGGCGTGTCCCCTACCAAG | 204 |
| M8 | .......................A............................................ | 204 |
| M9 | .....A........T........A..TC.................................A.G.... | 204 |
| M10 | .....A........T........A..TC.................................A.G.... | 204 |
| M7 | CTGAACGACCTGTGCTTCACAAACGTGTACGCCGACAGCTTCGTGATCCGGGGAGATGAAGTGCGGCA | 272 |
| M8 | .................................................................... | 272 |
| M9 | ....T...T...........CTCT.............C....C........T.TAG.........A.. | 272 |
| M10 | ....T...T...........CTCT.............C....C........T.TAG.........A.. | 272 |
| M7 | GATTGCCCCTGGACAGACAGGCAAGATCGCCGACTACAACTACAAGCTGCCCGACGACTTCACCGGCT | 340 |
| M8 | .................................................................... | 340 |
| M9 | .G.G..A...............GT.........T.................................. | 340 |
| M10 | .G.G..A...............GT.........T.................................. | 340 |
| M7 | GTGTGATTGCCTGGAACAGCAACAACCTGGACTCCAAAGTCGGCGGCAACTACAATTACCTGTACCGG | 408 |
| M8 | .................................................................... | 408 |
| M9 | .......C........T.C.GC...G.A.TA.AGGCAG.A...............C...TAC...A.A | 408 |
| M10 | .......C........T.C.GC...G.A.TA.AGGCAG.A...............C...TAC...A.A | 408 |
| M7 | CTGTTCCGGAAGTCCAATCTGAAGCCCTTCGAGCGGGACATCAGCACCGAAATCTATCAGGCCGGCAG | 476 |
| M8 | .................................................................... | 476 |
| M9 | AGCCA...C...A....G........T......A.....C.G....G...CGAG.G.AGCC.T.ATG. | 476 |
| M10 | AGCCA...C...A....G........T......A.....C.G....G...CGAG.G.AGCC.T.ATG. | 476 |
| M7 | CACCCCTTGC...............AACGGCGTGGAAGGCTTCAACTGCTACTTCCCACTGCAAAGCT | 529 |
| M8 | .................................................................... | 529 |
| M9 | ..AG.....TACACCTCCTGCCTTC..T......CGGA.................A.C...AGC.C.. | 544 |
| M10 | ..AG.....TACACCTCCTGCCTTC..T......CGGA.................A.C...AGC.C.. | 544 |
| M7 | ACGGCTTTCAGCCCACAAATGGCGTGGGCTACCAGCCTTACAGAGTGGTGGTGCTGAGCTTCGAGCTG | 597 |
| M8 | .................................................................... | 597 |
| M9 | ...A...CA.C....ACGTGCC.....AA......G.CACA........................... | 612 |
| M10 | ...A...CA.C....ACGTGCC.....AA......G.CACA........................... | 612 |
| M7 | CTGCATGCTAACGCCACAGTGTGCGGCCCTAAGAAATCCACCAAT | 642 |
| M8 | .........CCT................................. | 642 |
| M9 | ...A....C..............T.........CTG..T...C.G | 657 |
| M10 | ...A....CCCT...........T.........CTG..T...C.G | 657 |

### EXAMPLE 16

### Ability of Different Full-Length S Protein Genes to Induce Antibodies to SARS2 RBD

Mice were immunised with different full-length Coronavirus S protein genes (from SARS-1 and SARS-2), and the sera was collected and tested at different dilutions for binding (by ELISA) to SARS2 RBD. The sera were heat inactivated (HI) to check for non-specific interactions in the ELISA.

The results are shown in FIG. 9.

The binding of the sera to SARS-2 RBD was tested using ELISA. The ELISA protocol is as follows:

### Materials and Reagents:

F96 Nunc Maxisorp flat-bottom plates (Cat #: 44-2404-21, Thermo Scientific)
Plate sealers (Cat #: 676001, Greiner Bio-one)
Shaker (Cat #: 544-11200-00, Heidolph Instruments Titramax 100)
50 mL and 100 mL reservoirs (Cat #4870 Corning and #B3110-100 Argos)
U-bottom dilution plates (Cat #: 650201, Greiner bio-one)
1×PBS (—Ca/—Mg):
   Add 2 PBS tablets (Cat #: 18912-014, Gibco) to 1 L milliQ water
1×PBS (—Ca/—Mg)+0.1% Tween-20 (PBST):
   Add 4 PBS tablets (Cat #: 18912-014, Gibco) and 2 mL Tween-20 (Cat #: P1379-500ML, Sigma Aldrich) to 2 L milliQ water
3% (w/v) non-fat milk in 1×PBST (blocking solution):
   Add 1.5 g of semi-skimmed milk powder (Cat #: 70166-500G, Sigma Aldrich) in 50 mL of PBST
1% (w/v) non-fat milk in 1×PBST (serum dilution solution):
   0.5 g of milk powder (Cat #: 70166-500G, Sigma Aldrich) in 50 mL of PBST
HRP-conjugated secondary antibodies:
   Anti-mouse IgG-horseradish peroxidase (HRP) conjugated secondary antibody (Cat #:715-035-150, Jackson ImmunoResearch)
   Anti-human IgG/IgM/IgA-horseradish peroxidase (HRP) conjugated secondary antibody (Cat #: 109-035-064, Jackson ImmunoResearch)
1-Step™ Ultra TMB (Cat #34029, Thermo Scientific)
Stop solution of H₂SO₄ (add 28 mL of 1.84 kg/L H₂SO₄ to 472 mL milliQ water)
Serum samples (about 4 ul is needed to run a duplicate, starting at 1:50 dilution with 10-fold serial dilutions; about 5.5 ul is needed to run a duplicated, starting at 1:50 dilution with 2-fold serial dilutions)
Human positive control: strong antibody positive plasma from Covid-19 patient (Cat #20/130, NIBSC)
Human negative control: WHO Reference Anti-EBOV Negative human plasma (Cat #: 15/288, NIBSC)

### Method

### Day 0

1. Coat ninety-six well Nunc Maxisorp plates with 50 μl (per well) of 1 μg/mL of protein diluted in PBS−/−. Tap the plates gently against the counter to ensure that the liquid has fully coated the bottom of the plate.
2. Seal the plates tightly with plate sealer. Store plates in −4° C. fridge overnight, to a maximum of 4 days. Ensure that the liquid has not evaporated when using.
3. Prepare 3% and 1% non-fat milk, vortex and leave to dissolve on the shaker at 1350 rpm at room temperature. Leave to dissolve for at least one hour. Store in the −4° C. fridge overnight.

### Day 1

4. Prepare the negative and positive controls
   Mouse Negative control: Prepare a pool of all six mice from the PBS-immunized group (usually Group 1) from the corresponding bleed, at a final dilution of 1:50 in 1% non-fat milk in PBST
   Mouse Positive control: Prepare a 1:500 dilution of a known strong positive in 1% non-fat milk in PBST
   Human Negative control: Prepare a 1:50 dilution of the required amount of anti-EBOV plasma in 1% non-fat milk in PBST
   Human Positive control: Prepare a 1:500 dilution of the required amount of 20/130 in 1% non-fat milk in PBST
5. Decant the protein from the 96-well plate and add 100 μl of 3% non-fat milk per well. Incubate for 1 hour at room temperature on the shaker at 200-400 rpm.
6. During the blocking step, prepare serial dilutions of the serum in 1% non-fat milk in PBST using the U-bottom dilution plates.
   For a two-fold serial dilution starting at 1:50—Add 130 μl 1% non-fat milk to the first row with 2.6 μl of serum (in duplicates). Add 65 ul 1% non-fat milk to the remaining rows. Transfer 65 ul for the serial dilutions.
   For a ten-fold serial dilution starting at 1:50—Add 75 μl 1% non-fat milk to the first row with 1.5 μl of serum (in duplicates). Add 63 μl 1% non-fat milk to the remaining rows. Transfer 7 μl for the serial dilutions.
7. After the 1-hour blocking, decant the blocking solution and add 50 μl of the serial dilutions to the corresponding plates. Incubate on the shaker at 200-400 rpm for two hours at room temperature.
8. During the incubation, dilute the HRP-conjugated anti-mouse IgG secondary antibody 1:3000 in PBST. Make up 5 mL of diluted secondary per 96-well plate.
9. After the 2-hour primary antibody incubation, wash the plates three times with 200 μl (per well) of PBST. Tap dry after the last wash. Then add 50 μl (per well) of the diluted secondary antibody. Incubate on the shaker at 200-400 rpm at room temperature for 1 hour.
10. After adding the secondary antibody, take the appropriate volume of TMB and leave it on the counter to come to room temperature. Take 5 mL of TMB per 96-well plate.
11. After the 1-hour secondary antibody incubation, wash the plates three times with 200 μl (per well) of PBST. Tap dry after the last wash.
12. Add 50 μl (per well) of room temperature TMB. Agitate the plate gently. Leave for approximately 2-3 mins. Monitor the plate to ensure that the colour change does not become saturated. Add TMB to a maximum of 5 plates at a time.
13. Add 50 μl (per well) of room temperature stop solution. Agitate the plate gently. Read immediately.
14. Read endpoint optical density at 450 nm.

The following DNA vaccines were used:

### Heat Inactivation (HI)

SARS-1 (DNA encoding full length SARS-1 S protein)
SARS-2 (DNA encoding full length SARS-2 S protein)
DIOS-ancestor (Wuhan Node 1 full length)

### Not HI

SARS-1
SARS-2
DIOS-ancestor

Human sera against SARS-2 and anti-SARS1 spike monoclonal antibody were used as positive controls, and anti-MERS human sera was used as a negative control.

The figure shows that all the full-length S protein genes tested induced a relatively poor or negligible binding response to SARS2 RBD.

### EXAMPLE 17

### Ability of DNA Vaccines Encoding SARS1 and SARS2 Truncated Spike (S) Protein and RBD to Induce Antibodies to SARS1 and SARS2 S Protein

Mice were immunised with different DNA vaccines, and sera collected from the mice was used to test binding by FACS to SARS1 and SARS2 spike protein.

### 1—Reagents and Consumables

HEK293T/17 cells
DMEM with 10% FBS and 1% Pen/strep
OptiMEM
1×PBS
FuGENE-HD
   pEVAC expressing plasmid

### 2—Protocol

### Day 1—Seeding Cells

1. Seed 6-well plates with ˜150.000 cells per well for next day transfection (2 six well plates are enough for one 96 well plate)
2. Incubate overnight at 37° C., 5% CO₂.

### Day 2—Cell Transfection

1. Thaw producer cell plasmid DNA and pre-warm DMEM and OptiMEM to 37° C.
2. Prepare DNA mix in 600 μl OptiMEM (amount per plate; see table 1) in a labelled 1.5 ml tube
3. Incubate DNA mix for 5 minutes at room temperature
4. Add 9 μl of FuGENE-HD transfection reagent per 3 μg DNA in the transfection complex (see table below)
5. Incubate at room temperature for 20 minutes: mix by gently flicking the tube.
6. During incubation, remove depleted media from each well of the 6 well plate and replace with 2 ml DMEM per well.
7. After incubation, add the transfection complex to cells in a dropwise manner, and swirl to ensure even distribution.
8. Return cells to tissue culture incubator (37° C., 5% CO₂)

| Transfection | Glycoprotein | OptiMEM | FuGENE HD |
|---|---|---|---|
| complex | plasmid (ng) | (μl) | (μl) |
| pEVAC SARS1 | 3000 | 600 | 9.0 |
| pEVAC SARS2 | 3000 | 600 | 9.0 |

### Day 3—Antibody/Serum Dilution

1. Perform 1:2 serial dilution of serum or antibodies in cold PBS 1% FBS (e.g. 6 μl of serum in 300 μl of buffer, aliquot 150 μl for a duplicate. (6-well U-plate is preferred)
2. Human serum or IgG isotype controls must be included in the experimental plan

### Day 4—Flow Cytometry

3. Remove media and collect cells in a falcon
4. Centrifuge 5′ at 300×g
5. Resuspend cell pellet in 10 ml PBS (per plate)
6. Aliquot 100 μl of cell suspension per well in a 96 well plate V-bottom, using P100 multichannel and reservoir.
7. Centrifuge the plate 2′ at 300×g (R2 rotor in 227)
8. Flick out the plate in the sink
9. By using a multichannel, transfer 75 μl of diluted serum or antibodies from dilution plate to the FCAS plate and resuspend cells
10. Incubate RT 40′
11. Wash plate by adding 100 μl of PBS
12. Centrifuge the plate 2′ at 300×g
13. Flick out the plate in the sink
14. Wash plate by adding 180 μl of PBS and resuspend cell pellet
15. Flick out the plate
16. Add 60 μl/well of secondary antibody (20 μl/ml) and resuspend cells
17. Incubate RT 40′
18. Wash plate by adding 100 μl of PBS
19. Centrifuge the plate 2′ at 300×g
20. Flick out the plate in the sink
21. Wash plate by adding 180 μl of PBS and resuspend cells
22. Flick out the plate
23. Resuspend cells in 200 μl of PBS
   The DNA Vaccines Used were:

| | |
|---|---|
| COV_S_T2_2 | AY274119_tr (CoV_T2_2): nucleic acid encoding |
| | truncated S-protein (SEQ ID NO: 4) |
| COV_S_T2_3 | EPI_ISL_402119_tr (CoV_T2_3): nucleic acid |
| | encoding truncated S-protein (SEQ ID NO: 10) |
| COV_S_T2_5 | AY274119_RBD (CoV_T2_5): nucleic acid |
| | encoding RBD (SEQ ID NO: 6) |
| COV_S_T2_6 | EPI_ISL_402119_RBD (COV_T2_6): nucleic |
| | acid encoding RBD (SEQ ID NO: 12) |
| COV_S_T2_7 | Wuhan_Node1_RBD (CoV_T2_7): nucleic acid |
| | encoding RBD (SEQ ID NO: 18) |
| COV_S_T2_8 | “SARS_2 RBD_mut1” (the M7 construct, |
| | SEQ ID NO: 37) |
| COV_S_T2_10 | “SARS_an RBD_mut1” (the M9 construct, |
| | SEQ ID NO: 39) |

Binding of the sera obtained following the immunisations to SARS1 spike protein and SARS2 spike protein, at different dilutions, was assessed by FACS. The results are shown in FIG. 10.

The results show that the sera collected following immunisation with DNA encoding truncated spike protein and the RBD domains binds to the respective SARS protein. The M7 construct induced sera with better binding than the corresponding wild type SARS2 RBD.

### EXAMPLE 18

### Ability of DNA Vaccines Encoding Wild-Type SARS1 or SARS2 Spike Protein (Full-Length, Truncated, or RBD) to Induce a Neutralisation Response to SARS1 and SARS2 Pseudotypes

Mice were immunised with DNA vaccine encoding wild-type full-length SARS1 or SARS2 spike protein, DNA vaccine encoding wild-type truncated SARS1 or SARS2 spike protein, DNA vaccine encoding wild-type SARS1 or SARS2 spike RBD protein, or wild-type SARS1 or SARS2 RBD protein. Sera collected from the immunised mice were tested at different dilutions for their ability to neutralise SARS1 or SARS2 pseudotypes.

The vaccines used were:
DNA encoding full-length SARS1 or SARS2 spike protein;
DNA encoding truncated SARS1 or SARS2 spike protein;
DNA encoding SARS1 or SARS2 spike RBD; and
SARS1 or SARS2 RBD protein.

PBS was used as a negative control, and 20/130 (a National Institute for Biological Standards and Control (NIBSC) standard) and serum from patient 4 (a COVID-19 patient with strongly neutralising antibodies) were used as positive controls.

The results are shown in FIG. 11.

The results show that mice immunised with the SARS1 immunogens (DNA or protein) induce antibodies which neutralise SARS1 pseudotypes. However, the only SARS2 immunogen which induces SARS2 pseudotype neutralising antibodies is the DNA encoding SARS2 RBD.

### EXAMPLE 19

### Ability of SARS1 and SARS2 RBD Protein Vaccines to Induce Antibodies to SARS2 RBD

Mice were immunised with different protein vaccines. The sera were collected and tested for binding to SARS2 RBD at different dilutions.

The vaccines used were:
P-RBD-CoV1 (wild-type SARS1 RBD protein)
P-RBD-CoV2 (wild-type SARS2 RBD protein)
P-S_Stab_CoV2 (full-length spike protein stabilised by two proline mutations and removal of transmembrane region)

The results are shown in FIG. 12.

The results show that all of the protein vaccines tested induced SARS2 RBD-binding antibodies, including the SARS1 RBD (P-RBD-CoV1).

### EXAMPLE 20

### Ability of Different S Protein RBD DNA Vaccines to Induce Antibodies to SARS2 RBD

Mice were immunised with different S protein (truncated or RBD) DNA vaccines, then sera was collected and tested for binding to SARS2 RBD by ELISA (using the protocol described in Example 16).

The vaccines used were:
Ancestor RBD
Conv373 (positive control—sera from a Covid positive patient; data not shown)
Human_s (negative control, pre-Covid serum from Sigma)
SARS_1 RBD
SARS_1 trunc
SARS_2 RBD
SARS_2 RBD_mut1 (M7)
SARS_2 trunc
SARS_anc RBD_mut1 (M9)

The results are shown in FIG. 14.

The results show that the M7 SARS2 RBD DNA vaccine induced an immune response with stronger binding to SARS2 RBD than wild-type SARS2 RBD DNA in the early bleed.

### EXAMPLE 21

Inhibition of RBD-ACE2 Interaction by Sera Collected Following Immunisation with M7 and Wild-Type SARS2 RBD DNA Vaccines

A competition assay was used to show to what extent mouse sera, after immunisation of mice with M7 and wild-type RBD DNA vaccines, prevents binding of SARS2 pseudotypes to ACE2 receptors, using sera collected 2 and 8 weeks after immunisation.

The DNA vaccines used were:
D-RBD-CoV2 (DNA encoding wild-type SARS2 RBD);
D-RBD-M7_CoV2 (DNA encoding M7 SARS2 RBD)
D-RBD-TM_CoV2 (DNA encoding wild type RBD with a transmembrane domain, so that it remains tethered to the cell membrane rather than released as soluble protein like other RBD constructs)

The results are shown in FIG. 15.

The results presented in the left hand figure (a) (week 2) show that sera collected 2 weeks after immunisation with DNA encoding wild-type RBD and tethered wild-type RBD has no effect on binding of SARS2 pseudotypes to ACE2 receptors, but the sera collected 2 weeks after immunisation with DNA encoding M7 RBD does inhibit binding of SARS2 pseudotypes to ACE2 receptors.

The results presented in the right hand figure (b) (week 8) show that sera collected 8 weeks after immunisation with DNA encoding wild-type RBD and M7 RBD both show strong neutralisation.

It was concluded from these results that the DNA vaccine encoding wild-type RBD and M7 RBD elicit a neutralising immune response 8 weeks after immunisation, but that DNA vaccine encoding M7 SARS2 RBD elicits a neutralising immune response more rapidly than DNA vaccine encoding wild-type SARS2 RBD.

### Methods:

The competition assay was carried out using the GenScript SARS-CoV-2 Surrogate Virus Neutralization Test (sVNT) Kit, according to the manufacturer's protocol. The kit can detect circulating neutralizing antibodies against SARS-CoV-2 that block the interaction between the receptor binding domain of the viral spike glycoprotein (RBD) with the ACE2 cell surface receptor. The assay detects any antibodies in serum and plasma that neutralize the RBD-ACE2 interaction. The test is both species and isotype independent.

First, the samples and controls are pre-incubated with the HRP-RBD to allow the binding of the circulating neutralization antibodies to HRP-RBD. The mixture is then added to the capture plate which is pre-coated with the hACE2 protein. The unbound HRP-RBD as well as any HRP-RBD bound to non-neutralizing antibody will be captured on the plate, while the circulating neutralization antibodies-HRP-RBD complexes remain in the supernatant and get removed during washing. After washing steps, TMB solution is added, making the colour blue. By adding Stop Solution, the reaction is quenched and the colour turns yellow. This final solution can be read at 450 nm in a microtiter plate reader. The absorbance of the sample is inversely dependent on the titre of the anti-SARS-CoV-2 neutralizing antibodies.

### EXAMPLE 22

### Neutralisation of SARS2 Pseudotype Induced by M7 and Wild-Type SARS2 RBD DNA Vaccines

Mice were immunised with different RBD DNA vaccines listed below, then sera was collected and tested for SARS2 pseudotype neutralisation. Two studies were carried out (COV002.1 and COV002.2).

The DNA vaccines used were:
Ancestor RBD (DNA encoding ancestor RBD);
SARS_1 RBD (DNA encoding wild-type SARS1 RBD);
SARS_1 trunc (DNA encoding wild-type SARS1 truncated S protein);
SARS_2 RBD (DNA encoding wild-type SARS2 RBD)
SARS_2 RBD_mut1 (M7) (DNA encoding M7 SARS2 RBD)
SARS_2 trunc (DNA encoding wild-type SARS2 truncated S protein)
SARS_anc RBD_mut1 (M9) (DNA encoding M9 SARS ancestor RBD)

The results are shown in FIGS. 16 and 17.

The results from study COV002.1 and COV002.2 are shown in FIG. 16(a) (bleed at week 2 from the immunised mice), and the results from study COV002.1 and COV002.2 are shown in FIGS. 16(b) (bleed at week 3 from the immunised mice), and 16(c) (bleed at week 4 from the immunised mice).

FIG. 17 shows SARS2 pseudotype neutralisation IC₅₀ values for sera collected from the mice immunised with wild-type SARS2 RBD DNA vaccine, and M7 SARS2 RBD DNA vaccine. The dots in FIG. 17 show IC₅₀ values for individual mice, and the horizontal cross bars show the estimate based on all mice with 95% confidence intervals. The results shown in FIG. 17(a) are from study COV002.1 and COV002.2. The results shown in FIG. 17 (b) are from study COV002.2.

The results in FIGS. 16 and 17 show that the M7 SARS2 RBD DNA vaccine induces a more neutralising response than the wildtype SARS2 RBD DNA vaccine in sera collected from bleeds at weeks 1 and 2, but that by later bleeds there appears to be little difference between the two vaccines.

### EXAMPLE 23

Supernatant of Cells Expressing M7 SARS2 RBD Competes with Other ACE2 Binding Viruses for ACE2 Cell Entry

Supernatant of cells was used to compete with one of three coronavirus pseudotypes (NL63, SARS1, SARS2) for ACE2 receptors. The supernatant was either from cells expressing M7 or from cells transfected with the empty pEVAC. The results are shown in FIG. 18.

The results show that the M7 supernatant competes effectively with the three ACE2 binding viruses, although possibly to a lesser extent with SARS1.

### EXAMPLE 24

### M7 SARS2 RBD DNA Vaccine Induces T Cell Responses

An enzyme-linked immunospot (ELISPOT) assay against an RBD peptide pool was used to determine T cell responses induced by the M7 SARS2 RBD DNA vaccine (compared with PBS as a negative control). The results are shown in FIG. 19. The results show that T cell responses were induced by the M7 DNA vaccine that were reactive against peptides of the RBD peptide pool. The medium is used as the negative control.

The ELISPOT assay is a highly sensitive immunoassay that measures the frequency of cytokine-secreting cells (in this case, murine T cells secreting IFN-γ) at the single-cell level. In this assay, cells are cultured on a surface coated with a specific capture antibody in the presence or absence of stimuli. Proteins, such as cytokines, that are secreted by the cells will be captured by the specific antibodies on the surface. After an appropriate incubation time, cells are removed and the secreted molecule is detected using a detection antibody in a similar procedure to that employed by the enzyme-linked immunoassay (ELISA). The detection antibody is either biotinylated and followed by a streptavidin-enzyme conjugate or the antibody is directly conjugated to an enzyme. By using a substrate with a precipitating rather than a soluble product, the end result is visible spots on the surface. Each spot corresponds to an individual cytokine-secreting cell.

The ELISPOT assay was carried out according to the manufacturer's protocol (Cellular Technology Limited, CTL) repeated below:

### Murine IFN-γ Single-Color Enzymatic ELISPOT Assay:

PROCEDURE (If using precoated plates, start at Day 1)

### Day 0—Sterile Conditions

Prepare Murine IFN-γ Capture Solution (see Solutions).
Pipette 80 μl/well Murine IFN-γ Capture Solution. Seal plate with parafilm and incubate at 4° C. overnight. (Prewetting of plates with ethanol is not required but in some instances where a large response is expected, the assay can benefit from removing the underdrain, adding 15 μl of 70% ethanol/well for less than one minute, washing three times with 150 μl of PBS/well, replacing the underdrain, and immediately [before plate dries], add the Capture Solution. If using strip plates, there is no underdrain to remove before prewetting. As an alternative, one can purchase CTL precoated plates.) Note: Activation of the membrane with ethanol is instantaneous and can be seen visually as a graying of the membrane. Ethanol should be washed off as quickly as possible following activation.

### Day 1—Sterile Conditions

Prepare CTL-Test™ Medium (see Solutions).
Prepare antigen/mitogen solutions at two times final concentration in CTL-Test™ Medium.
Decant plate containing Capture Solution from Day 0 and wash one time with 150 μl PBS.
Plate antigen/mitogen solutions, 100 μl/well. Ensure the pH and temperature are ideal for cells by placing the plate containing antigens into a 37° C. incubator for 10-20 minutes before plating cells.
Adjust cells to desired concentration in CTL-Test™ Medium, e.g.: 3 million/ml corresponding to 300,000 cells/well (cell numbers can be adjusted according to expected spot counts since 100,000-800,000 cells/well will provide linear results). Keep cells at 37° C. in humidified incubator, 9% CO₂ while processing cells and until plating.
Plate cells 100 μl/well using large orifice tips. Once completed, gently tap the sides of the plate and immediately place into a 37° C. humidified incubator, 9% CO₂.
Incubate for 24 hours. Do not stack plates. Avoid shaking plates by carefully opening and dosing incubator door.

Do not touch plates during incubation.

### Day 2

Prepare Buffer Solutions: PBS, distilled water and Tween-PBS (see Wash Buffers).
Prepare Anti-murine IFN-γ Detection Solution (see Solutions).
Wash plate two times with PBS and then two times with 0.05% Tween-PBS, 200 μl/well each time.
Add 80 μl/well Anti-murine IFN-γ Detection Solution. Incubate at room temperature, two hours.
Prepare Tertiary Solution (see Solutions).
Wash plate three times with 0.05% Tween-PBS, 200 μl/well.
Add 80 μl/well of Tertiary Solution. Incubate at room temperature, 30 minutes.
During incubation, prepare Blue Developer Solution (see Solutions).
Wash plate two times with 0.05% Tween-PBS, and then two times with distilled water, 200 μl/well each time.
Add Blue Developer Solution, 80 μl/well. Incubate at room temperature, 15 minutes.
Stop reaction by gently rinsing membrane with tap water, decant, and repeat three times.
Remove protective underdrain from the plate and rinse back of plate with tap water.
Air-dry plate for two hours in running laminar flow hood or for 24 hours face down on paper towels on bench top.
Scan and count plate. (CTL has scanning and analysis services available and offers a trial version of ImmunoSpot® Software with the purchase of any kit. Email kitscanningservices@immunospot.com for more info.)

### Solutions

All solutions should be freshly-made prior to use. It is important to quick-spin the vials before use to ensure content volumes.
70% Ethanol (if prewetting—not included): Dilute 190-200 proof ethanol. For 10 ml, add 7 ml of ethanol to 3 ml of distilled water.
CTL-Test™ Medium: Prepare medium by adding 1% fresh L-glutamine. The amount of medium needed will depend on variables such as cell yield and number of samples tested but will be no less than 20 ml for one full plate.
Capture Solution: Dilute Murine IFN-γ Capture Antibody in Diluent A. For one plate, add 60 μl of Murine IFN-γ Capture Antibody to 10 ml of Diluent A.
Detection Solution: Dilute Anti-murine IFN-γ (Biotin) Detection Antibody in Diluent B. For one plate, add 10 μl of Anti-murine IFN-γ (Biotin) Detection Antibody to 10 ml of Diluent B.
Tertiary Solution: Dilute Strep-AP Solution in Diluent C, 1:1000. For one plate, add 10 μl of Strep-AP to 10 ml of Diluent C.
Blue Developer Solution: Add the Substrate Solutions in sequential steps to 10 ml of Diluent Blue.

For one plate:
Step 1—Add 160 μl of S1 to 10 ml of Diluent Blue. Mix well!
Step 2—Add 160 μl of S2. Mix well!
Step 3—Add 92 μl of S3. Mix well!

It is recommended to make the Blue Developer Solution within ten minutes of use and to keep it protected from direct light.

Wash Buffers (not included)

For each plate prepare:
0.05% Tween-PBS: 100 μl Tween-20 in 200 ml PBS
PBS, sterile, 100 ml
Distilled water, 100 ml

### Cryopreservation of Mouse Splenocytes

This was carried out according to the protocol of CELLULAR TECHNOLOGY LIMITED, repeated below:

Cell permeability, reagent toxicity, and cooling rates must be considered for each cell type when freezing. The osmotic pressure caused by DMSO (more than DMSO's intrinsic toxicity) is one of the primary factors that need to be controlled for successful freezing and thawing of splenocytes. To maintain the metabolic activity of the cells and their membrane lipid fluidity (so they can compensate for the osmotic pressure), all reagents should be at room temperature (preferably at 37° C.).

### Preparation:

1. Mix CTL-Cryo™ A with CTL-Cryo™ B in an 80% to 20% (v/v) ratio (4+1) by slowly adding CTL-Cryo™ B into CTL-Cryo™ A.
(CTL-Cryo™ B contains DMSO as a component. Please refer to MSDS, included.)
2. Warm the resulting CTL-Cryo™ A-B Mix and CTL-Cryo™ C in a 37° C. CO2 incubator. (It is advised to start with this step while counting cells).
3. Each cryotube should contain approximately 10×10⁸ cells (10-15 million). Freezing more cells per tube may lead to cell loss.

### After Washing:

1. After counting, centrifuge the cell suspension at room temperature at 330 g for 10 minutes with rapid acceleration and brake on high.
2. Decant supernatant and mix cells gently by tapping the tube with your finger. Do not use a pipette and avoid foam formation!
3. Slowly, over a time period of ˜2 minutes, add an equal volume of warm CTL-Cryo™ A-B Mix to the CTL-Cryo™ C containing the splenocytes. (Add CTL-Cryo™ A-B mix drop-by-drop while gently whirling the tube to ensure complete mixing of the two solutions.
4. Aliquot the resulting CTL-Cryo™ A-B-C suspension containing the splenocytes into pre-labeled 1.8 ml cryovials, 1 ml into each vial.

Pipette gently and slowly to minimize shear forces; do not attempt additional mixing with the pipette. The cells can remain in the completed CTL-Cryo™ A-B-C medium for 10-20 minutes without loss of viability or function.
5. Place cryovials into a room temperature Nalgene® cryofreezing container (Mr. Frosty™) filled with propanol and transfer into a −80° C. freezer for a minimum of 12 hours. Do not open the freezer during this time period. Use a dedicated −80° C. freezer in order to prevent shaking the samples and fluctuation of the freezer's temperature due to opening and closing of the freezer door.
6. After a minimum of 12 hours and no more than 48 hours, transfer the cryovials into vapor/liquid nitrogen tanks for storage.

### EXAMPLE 25

Further Designed E Protein Sequences (with Abrogated Ion Channel Activity)

SARS-CoV envelope (E) gene encodes a 76-amino acid transmembrane protein with ion channel (IC) activity, an important function in virus-host interaction. Infection of mice with viruses lacking or displaying E protein IC activity revealed that activation of the inflammasome pathway, and the exacerbated inflammatory response induced by SARS-CoV, was decreased in infections by ion channel-deficient viruses (Nieto-Torres et al., 2014, Severe Acute Respiratory Syndrome Coronavirus Envelope Protein Ion Channel Activity Promotes Virus Fitness and Pathogenesis. PLoS Pathog 10(5): e1004077).

We have made new E protein designs Cov_E_T2_3, CoV_E_T2_4 and CoV_E_T2_5, which correspond to SARS2, CoV_E_T2_1 and CoV_E_T2_2 (see Example 10), respectively. The new designs have a point mutation. N15A, which abrogates the ion channel activity, but does not influence the stability of the structure. Nieto-Torres et al., supra, discusses this mutation as well as the toxicity and inflammatory action of SARS E on the host cell.

The amino acid sequences of the new E protein designs are shown below:

>COV_E_T2_3 (SARS2_mutant)
(SEQ ID NO: 42)
MYSFVSEETG TLIVASVLLF LAFVVELLVT LAILTALRLC
AYCCNIVNVS LVKPSFYVYS RVKNLNSSR-VPDLLV
>COV_E_T2_4 (Env1_mutant)
(SEQ ID NO: 43)
MYSFVSEETG TLIVASVLLF LAFVVFLLVT LAILTALRLC
AYCCNIVNVS LVKPTFYVYS RVKNLNSSQGVPDLLV
>COV_E_T2_5 (Env2_mutant)
(SEQ ID NO: 44)
MYSFVSEETG TLIVASVLLF LAFVVELLVT LAILTALRLC
AYCCNIVNVS LVKPTFYVYS RVKNLNSSR-VPDLLV

Alignment of the E protein designs (SEQ ID NOs:22-23, and SEQ ID NOs:41-44) with SARS2 E protein reference sequence is shown below:

| | | | |
|---|---|---|---|
| SARA2 COV_E_T2_1 COV_E_T2_2 COV_E_T2_3 COV_E_T2_4 COV_E_T2_5 | 1 1 1 1 1 1 | 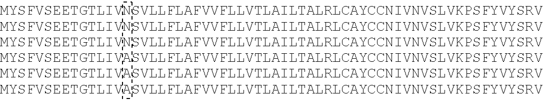 | 62 62 62 62 62 62 |
| SARA2 | 63 | KNLNSSR.VPDLLV | 75 |
| COV_E_T2_1 | 63 | KNLNSSQGVPDLLV | 76 |
| COV_E_T2_2 | 63 | KNLNSSR.VPDLLV | 75 |
| COV_E_T2_3 | 63 | KNLNSSR.VPDLLV | 75 |
| COV_E_T2_4 | 63 | KNLNSSQGVPDLLV | 76 |
| COV_E_T2_5 | 63 | KNLNSSR.VPDLLV | 75 |

The amino acid differences of the designed sequences from the SARS2 reference sequence are shown in the table below (with differences from the reference sequence highlighted in bold):

### EXAMPLE 26

### Nucleoprotein (N) Protein Vaccine Sequences

We have made new N protein designs, COV_N_T2_1 and COV_N_T2_2. The amino acid sequences of these designs is shown below. Sequence COV_N_T2_2 was designed using a methodology and algorithm which selected predicted epitopes to include based on their conservation across the sarbecoviruses (whilst minimising redundancy), the frequency and number of MHC alleles the epitope is restricted by the predicted epitope quality, and a handful of user specified weightings.

>YP 009724397.2/1-419 nucleocapsid phosphoprotein
(SARS-CoV-2) (reference sequence)
(SEQ ID NO: 45)
MSDNGPQ-NQ RNAPRITFGG PSDSTGSNQN GERSGARSKQ
RRPQGLPNNT ASWFTALTQH GKEDLKEPRG QGVPININSS
PDDQIGYYRR ATRRIRGGDG KMKDLSPRWY FYYLGTGPEA
GLPYGANKDG IIWVATEGAL NTPKDHIGTR NPANNAAIVL
QLPQGTTLPK GFYAEGSRGG SQASSRSSSR SRNSSRNSTP
GSSRGTSPAR MAGNGGDAAL ALLLLDRINQ LESKMSGKGQ
QQQGQTVTKK SAAEASKKPR QKRTATKAYN VTQAFGRRGP
EQTQGNFGDQ ELIRQGTDYK HWPQIAQFAP SASAFFGMSR
IGMEVTPSGT WLTYTGAIKL DDKDPNEKDQ VILLNKHIDA
YKTEPPTEPK KDKKKKADET QALPQRQKKQ QTVILLPAAD
LDDESKQLQQ SMSSA--DST
>CoV N T2 1/1-418 Node1b 321-323 deleted
(SEQ ID NO: 46)
MSDNGPQ-NQ RSAPRITEGG PSDSTDNNQN GERSGARPKQ
RRPQGLPNNT ASWFTALTQH GKEDLREPRG QGVPINTNSG
KDDQIGYYRR ATRRVRGGDG KMKELSPRWY FYYLGTGPEA
ALPYGANKEG IVWVATEGAL NTPKDHIGTR NPNNNAAIVL
QLPQGTTLPK GFYAEGSRGG SQASSRSSSR SRGNSRNSTP
GSSRGTSPAR MASGGGDTAL ALLLLDRLNQ LESKVSGKGQ
QQQGQTVTKK SAAEASKKPR QKRTATKQYN VTQAFGRRGP
EQTQGNEGDQ ELIRQGTDYK HWPQIAQFAP SASAFFGMSR
---EVTPSGT WLTYHGAIKL DDKDPQEKDN VILLNKHIDA
YKTFPPTEPK KDKKKKADEA QPLPQRQKKQ PTVTLLPAAD
LDDESKQLQN SMSGASADST QA
>CoV N T2 2/1-417 epitope optimised 321-323
deleted
(SEQ ID NO: 47)
MTDNGQQ-GP RNAPRITF-G VSDNEDNNQD GGRSGARPKQ
RRPQGLPNNT ASWFTALTQH GKEDLREPRG QGVPINTNSS
PDDQIGYYRR ATRRIRGGDG KMKDLSPRWY FYYLGTGPEA
ALPYGANKEG IVWVATEGAL NTPKDHIGTR NPNNNAAIVL
QLPQGTTLPK GFYAEGSRGG SQASSRSSSR SRNSSRNSTP
GSSRGTSPAR NLQAGGDTAL ALLLLDRINQ LESKMSGKGQ
QQQGQTVTKK SAAEASKKPR QKRTATKQYN VTQAFGRRGP
EQTQGNEGDQ ELIRQGTDYK QWPQIAQFAP SASAFFGMSR
---EVTPSGT WLTYTGAIKL DDKDPQEKDN VILLNKHIDA
YKTEPPTEPK KDKKKKADEA QPLPQRQKKQ QTVILLPAAD
LDDESRQLQN SMSGASADST QA

Alignment of the N protein designs with SARS2 N protein reference sequence is shown below:

| | |
|---|---|
| YP_009724397 | MSDNGPQNQRNAPRITFGGPSDSTGSNQNGERSGARSKQRRPQGLPNNTASWFTALTQHGKEDLKFPRGQGVPINTNSSPDDGIGYYRRATRRIRGGDGKMKDLSPPW 108 |
| COV_N_T2_1 | MSDNGPQNQRSAPRITFGGPSDSTQNNQNGERSGARPWARRPQGLPNNTASWFTALTQHGKEDLRFPRGQGVPINTNSGKDDGIGYYRRATRRVRGGDGKMKELSPRW 108 |
| COV_N_T2_2 | MTDNGQQGPRNAPRITF-GVSDNFDNNQDGGRSGARPKARRPQGLPNNTASWFTALTQHGKEDLRFPRGQGVPINTNSSPDDGIGYYRRATRRIRGGDGKMKDLSPPW 107 |
| YP_009724397 | YFYYLGTGPEAGLPYGANKDGIIWVATEGALNTPKDHIGTRNPANNAAIVLQLPQGTTLPK-GFYAEGSRGGGQASSRSPSRSRNSSRNSTPGSSRGTSPARMAGNGG 215 |
| COV_N_T2_1 | YFYYLGTGPEAALPYGANKEGIVWVATEGALNTPKDHIGTRNPNNNAAIVLQLPQGTTLPK-GFYAEGSRGGGQASSRSPSRSPGNSRNSTPGSSRGTSPARMASGGG 215 |
| COV_N_T2_2 | YFYYLGTGPEAALPYGANKEGIVWVATEGALNTPKDHIGTRNPNNNAAIVLQLPQGTTLPK-GFYAEGSRGGGQASSPSSSRSRNSSRNSTPGSSRGTSPARNLQAGG 214 |
| YP_009724397 | DAALALLLLQRLNGLESKMSGRGQQQQGQTYTRESAAEASKKPRQKRTATKAYNVTQAFGRRGPEQTQGNFGDQELIRQGTDYKHWPQIAQFAPSASAFFGMSRIGME 323 |
| COV_N_T2_1 | DTALALLLLQRLNGLESKYSGEGQQQQGQTYTRESAAEASKKPRAKRTATKQYNVTQAFGRRGPEQTQGNFGDQELIRQGTDYKHWPQIAQFAPSASAFFGMSR---E 320 |
| COV_N_T2_2 | DTALALLLLQRLNGLESKMSGRGQQQQGQTYTRESAAEASKKPRAKRTATKQYNVTQAFGRRGPEQTQGNFGDQELIRQGTDYKQWPQIAQFAPSASAFFGMSR---E 319 |
| YP_009724397 | VTPGGTWLTYTGAIKLDDKDPNFKDQVILLNKHIDAYKTFPPTEPKKDKKKKADETQALPQRQKKQQTVILLPAAD-LDDFSKQLQQSMSSA--DSTQA 419 |
| COV_N_T2_1 | VTPSGTWLTYHGAIKLDDKDPQFKDNVILLNKHIDAYKTFPPTEPKKDKKKKADEAQPLPQRQKKQPTVILLPAAD-LDDFSKQLQNSMSGASADSTQA 418 |
| COV_N_T2_2 | VTPSGTWLTYTGAIKLDDKDPQFKDNVILLNKHIDAYKTFPPTEPKKDKKKRADEAQPLPQRQKKQQTVILLPAAD-LDDFSRQLQNSMSGASADGTQA 417 |

The amino acid differences of the designed sequences from the SARS2 reference sequence are shown in the table below (with differences from the reference sequence highlighted in bold, and differences that are common to all the designed sequences underlined):

Positions 415 and 416 are italicised as they are not residues of the reference sequences, but include insertions in the N_T2_1 and N_T2-2 sequences.

### EXAMPLE 27

### Membrane (M) Protein Vaccine Sequences

We have made further new M protein designs. In these designs, we have deleted the 1st and the 2nd transmembrane region of the membrane protein to abrogate its interaction with the S protein:
The string construct with S, M and E was showing higher order aggregates.
Abrogation of interaction between S and M—can reduce aggregation.
M-del constructs (Cov_M_T2_(3-5)) designed to abrogate the interaction with S.

FIG. 20 shows an illustration of the M protein. Interaction between the M, E and N proteins is important for viral assembly. The M protein also binds to the nucleocapsid, and this interaction promotes the completion of virion assembly. These interactions have been mapped to the C-terminus of the endo-domain of the M protein, and the C-terminal domain of the N-protein. In FIG. 20, * denotes identification of immunodominant epitopes on the membrane protein of the Severe Acute Respiratory Syndrome-Associated Coronavirus, and ** denotes mapping of the Coronavirus membrane protein domains involved in interaction with the Spike protein.

The amino acid sequences of the new M protein designs are given below:

>COV_M_T2_3
(SEQ ID NO: 48)
MADSNGTITV EELKKLLEQI TGGIAIAMAC LVGLMWLSYF
IASFRLFART RSMWSENPET NILLNVPLHG TILTRPLLES
ELVIGAVILR GHLRIAGHHL GRCDIKDLPK EITVATSRTL
SYYKLGASQR VAGDSGFAAY SRYRIGNGKL NTDHSSSSDN
IALLVQ
>COV_M_T2_4
(SEQ ID NO: 49)
MADNGTITVE ELKQLLEQVT GGIAIAMACI VGLMWLSYFV
ASFRLFARTR SMWSENPETN ILLNVPLRGT ILTRPLMESE
LVIGAVIIRG HLRMAGHSLG RCDIKDLPKE ITVATSRTLS
YYKLGASQRV GTDSGFAAYN RYRIGNGKLN TDHAGSNDNI
ALLVQ
>COV_M_T2_5
(SEQ ID NO: 50)
MADSNGTITV EELKKLLEQV TGGIAIAMAC IVGLMWLSYF
VASFRLFART RSMWSENPET NILLNVPLRG SIITRPLMES
ELVIGAVILR GHLRMAGHSL GRCDIKDLPK EITVATSRTL
SYYKLGASQR VASDSGFAVY NRYRIGNGKL NTDHSSSSDN
IALLVQ

Sequence alignment of the new M protein designs (COV_M_T2_3, COV_M_T2_4, COV_M_T2_5) (SEQ ID NO:48-50) with the previous M protein designs (COV_M_T1_1, COV_M_T2_1, COV_M_T2_2) (SEQ ID NO:24-26) is shown below:

| | | |
|---|---|---|
| COV_M_T1_1 | MADSNGTITVEELKKELEQWNLVIGFLFLTWICLLQFAYANANRFLYIIKLIFLWLLWPVTLA | 63 |
| COV_M_T2_3 | MADSNGTITVEELKKELEQ-------------------------------------------- | 19 |
| COV_M_T2_1 | MAD-NGTITVEELKKELEQWNLVIGFLFLTWICLLQFAYANANRFLYIIKLIFLWLLWPVTLA | 62 |
| COV_M_T2_4 | MAD-NGTITVEELKKELEQ-------------------------------------------- | 18 |
| COV_M_T2_2 | MADSNGTITVEELKKELEQWNLVIGFLFLTWICLLQFAYANANRFLYIIKLIFLWLLWPVTLA | 63 |
| COV_M_T2_5 | MADSNGTITVEELKKELEQ-------------------------------------------- | 19 |
| COV_M_T1_1 | CFVLAAVYRINWITGGIAIAMACLVGLMWLSYFIASFRLFARTRSMWSFNPETNILLNVPLHG | 125 |
| COV_M_T2_3 | ------------ITGGIAIAMACLVGLMWLSYFIASFRLFARTRSMWSFNPETNILLNVPLHG | 70 |
| COV_M_T2_1 | CFVLAAVYRINWVTGGIAIAMACIVGLMWLSYFVASFRLFARTRSMWSFNPETNILLNVPLRG | 125 |
| COV_M_T2_4 | ------------VTGGIAIAMACIVGLMWLSYFVASFRLFARTRSMWSFNPETNILLNVPLRG | 69 |
| COV_M_T2_2 | CFVLAAVYRINWVTGGIAIAMACIVGLMWLSYFVASFRLFARTRSMWSENPETWILLNVPLRG | 126 |
| COV_M_T2_5 | ------------VTGGIATAMACIVGLMWLSYFVASFRLFARTRSMWSENPETWILLNVPLRG | 70 |
| COV_M_T1_1 | TILTRPLLESELVIGAVELRGHLRIAGHHLGRCDIKDLPKEITVATSRTLSYYKLGASQRVAG | 189 |
| COV_M_T2_3 | TILTRPLLESELVIGAVELRGHLRIAGHHLGRCDIKDLPKEITVATSRTLSYYKLGASQRVAG | 133 |
| COV_M_T2_1 | TILTRPLMESELVIGAVIIRGHERMAGHSLGRCDIKDLPKEITVATSRTLSYYKLGASQRVGT | 188 |
| COV_M_T2_4 | TILTRPLMESELVIGAVIIRGHERMAGHSLGRCDIKDLPKEITVATSRTLSYYKLGASQRVGT | 132 |
| COV_M_T2_2 | SHITRPLMESELVIGAVILRGHLRMAGHSLGRCDIKDLPKEITVATSRTLSYYKLGASQRVAS | 189 |
| COV_M_T2_5 | SITTRPLMESELVIGAVILRGHERMAGHSLGRCDIKDLPKEITVATSRTLSYYKLGASQRVAS | 133 |
| COV_M_T1_1 | DSGFAAYSRYRIGNYKENTDASSSSDNIALLVQ | 222 |
| COV_M_T2_3 | DSGFAAYSRYRIGNGKLNTDHSSSSDNIALLVQ | 166 |
| COV_M_T2_1 | DSGFAAYNRYRIGNYKLNTDRAGSNDNIALLVQ | 221 |
| COV_M_T2_4 | DSGFAAYNRYRIGNGKLNTDRAGSNDNIALLVQ | 165 |
| COV_M_T2_2 | DSGFAVYNRYRIGNYKLNTQNSSSSDNIALLVQ | 222 |
| COV_M_T2_5 | DSGFAVYNRYRIGNGKLNTDHSSSSDNIALLVQ | 166 |

The amino acid differences of the designed sequences from the SARS2 M protein reference sequence are shown in the table below (with differences from the reference sequence highlighted in bold):

### EXAMPLE 28

### Glycosylation of S Protein RBD Proteins

FIG. 21 shows the spectra overlap (MALDI MS) of supernatants derived from HEK cells transfected with pEVAC plasmid encoding the following S protein RBD sequences:
COV_S_T2_5 (wild-type SARS1 RBD)
COV_S_T2_6 (wild-type SARS2 RBD)
COV_S_T2_13
COV_S_T2_14
COV_S_T2_15
COV_S_T2_16
COV_S_T2_17
COV_S_T2_18
COV_S_T2_19
COV_S_T2_20
M7 RBD
TM RBD

The results show that the RBD is peaking at 25-26 KDa, and a second peak appears at 29 KDa.

FIG. 22 shows the spectra for the following examples of recombinant RBD proteins:
RBD (one sample labelled “LMB”);
His-tagged RBD;
Another RBD protein sample labelled “Ralph”.

The amino acid sequence of COV_S_T2_19 is below:

>COV_S_T2_19
(SEQ ID NO: 55)
RVAPTKEVVRFPNITNLCPFGEVFNATRFPSVYAWERKRISNCVA
DYSVLYNSTSFSTFKCYGVSPTKLNDLCFTNVYADSFVIRGDEVR
QIAPGQTGVIADYNYKLPDDFTGCVIAWNTNNLDSTTGGNYNYLY
RSLRKSKLKPFERDISSDIYSPGGKPCSGVEGFNCYYPLRSYGFF
PTNGVGYQPYRVVVLSFELLNAPATVCGPKLSTDGGGGSGGGGSG
GGGGGGGSKSSIASFFFIIGLIIGLFLVLRVGIHLCIKLKHTKKR
QIYTDIEMNRLGK

The amino acid sequence of COV_S_T2_20 is below:

>COV_S T2_20
(SEQ ID NO: 56)
RVAPTKEVVRFPNITNLCPFGEVFNATKFPSVYAWERKKISNCVA
DYSVLYNSTSFSTFKCYGVSPTKLNDLCFTNVYADSFVIRGDEVR
QIAPGQTGVIADYNYKLPDDFTGCVIAWNTNNIDSTTGGNYNYLY
RSLRKSKLKPFERDISSDIYSPGGKPCSGVEGFNCYYPLRSYGFF
PTNGTGYQPYRVVVLSFELLNAPATVCGPKLSTDGGGGSGGGGSG
GGGSGGGGSKSSIASFFFIIGLIIGLFLVLRVGIHLCIKLKHTKK
RQIYTDIEMNRLGK

COV_S_T2_19 is essentially COV_S_T2_13 with a transmembrane domain, and COV_S_T2_20 is COV_S_T2_17 with a transmembrane domain.

The amino acid sequence of RBD protein (Leader—RBD—Tag) is below:

(SEQ ID NO: 51)
MKRGLCCVLLLCGAVFVSPSAARVQPTESIVRFPNITNLCPFGEV
FNATRFASVYAWNRKRISNCVADYSVLYNSASFSTFKCYGVSPTK
LNDLCFTNVYADSFVIRGDEVRQIAPGQTGKIADYNYKLPDDFTG
CVIAWNSNNLDSKVGGNYNYLYRLFRKSNLKPFERDISTEIYQAG
STPCNGVEGFNCYFPLQSYGFQPTNGVGYQPYRVVVLSFELLHAP
ATVCGPKKSTNGGSGLNDIFEAQKIEWHEGSHHHHHH

FIG. 22 shows that the LMB and His-tagged RBD proteins peak at ˜26 KDa (LMB is the higher peak in the figure), and that the Ralph RBD sample peaks at ˜31-32 KDa. Peaks are also seen at ˜52 KDa for “LMB” and “his RBD” (LMB is the higher peak), and at ˜62-64 KDa for the Ralph RBD sample.

It was concluded from these results that there are two main glycosylated forms of the proteins obtained from the supernatant, in comparison to purified (recombinant) protein. The purified protein is non-glycosylated or sparsely glycosylated. This difference in glycosylation is believed to be important, as the glycosylation sites surround the epitope region and are conserved in most sarbecoviruses. These glycosylation sites are also important for interaction with some of the antibodies.

FIG. 23 provides a reference for glycosylation of the “S” Spike protein. As can be seen from the spectra, the glycosylation pattern of the spike protein is mixed. On average, the mass for each glycan is ˜2 kDa. There are three sites of glycosylation for four of the S protein RBD designs (COV_T2_13, COV_T2_14, COV_T2_15, and COV_T2_16) and wild-type SARS1 RBD, two for wild-type SARS2 RBD, and four for S protein RBD designs COV_T2_17, COV_T2_18.

The mass of “Ralf RBD protein” is 29.2 kDa. The mass of the designed RBD proteins, and wild-type RBD is ˜24 kDa.

### EXAMPLE 29

### Pan-Sarbecovirus Vaccine Coverage

Pan-Sarbecovirus protection: Beta-Coronaviruses including SARS-CoV-2 (SARS2), -1 (SARS1) & the many Bat SARSr-CoV (ACE2 receptor using) that threaten to spillover into humans.

FIG. 24 illustrates antigenic coverage achieved by universal Sarbecovirus B-cell and T-cell antigen targets. Part 1 shows Sarbecoviruses with the SARS1 and SARS2 clades highlighted along with human or bat host species. Part 2 shows machine learning predicted MHC class II binding (higher is stronger binding) of predicted epitopes within the insert. Lighter grey is for epitopes conserved within SARS2, darker grey are epitopes grafted in from other Sarbecoviruses such as SARS1.

### EXAMPLE 30

### Designed S Protein Sequence to Protect Against COVID-19 Variants

Multiple SARS-CoV-2 variants are circulating globally. Several new variants emerged in the fall of 2020, most notably:

In the United Kingdom (UK), a new variant of SARS-CoV-2 (known as 20I/501Y.V1, VOC 202012/01, or B.1.1.7) emerged with a large number of mutations. This variant has since been detected in numerous countries around the world, including the United States (US). In January 2021, scientists from UK reported evidence that suggests the B.1.1.7 variant may be associated with an increased risk of death compared with other variants, although more studies are needed to confirm this finding. This variant was reported in the US at the end of December 2020.

In South Africa, another variant of SARS-CoV-2 (known as 20H/501Y.V2 or B.1.351) emerged independently of B.1.1.7. This variant shares some mutations with B.1.1.7. Cases attributed to this variant have been detected in multiple countries outside of South Africa. This variant was reported in the US at the end of January 2021.

In Brazil, a variant of SARS-CoV-2 (known as P.1) emerged that was first was identified in four travelers from Brazil, who were tested during routine screening at Haneda airport outside Tokyo, Japan. This variant has 17 unique mutations, including three in the receptor binding domain of the spike protein. This variant was detected in the US at the end of January 2021.

Scientists are working to learn more about these variants to better understand how easily they might be transmitted and the effectiveness of currently authorized vaccines against them. New information about the virologic, epidemiologic, and clinical characteristics of these variants is rapidly emerging.

### B.1.1.7 Lineage (a.k.a. 20I/501Y.V1 Variant of Concern (VOC) 202012/01)

This variant has a mutation in the receptor binding domain (RBD) of the spike protein at position 501, where the amino acid asparagine (N) has been replaced with tyrosine (Y). The shorthand for this mutation is N501Y. This variant also has several other mutations, including:
69/70 deletion: occurred spontaneously many times and likely leads to a conformational change in the spike protein
P681H: near the S1/S2 furin cleavage site, a site with high variability in coronaviruses. This mutation has also emerged spontaneously multiple times.

This variant is estimated to have first emerged in the UK during September 2020.

Since Dec. 20, 2020, several countries have reported cases of the B.1.1.7 lineage, including the United States.

This variant is associated with increased transmissibility (i.e., more efficient and rapid transmission).

In January 2021, scientists from UK reported evidence (Horby P, Huntley C, Davies N, et al. NERVTAG note on B.1.1.7 severity. SAGE meeting report. Jan. 21, 2021) that suggests the B.1.1.7 variant may be associated with an increased risk of death compared with other variants.

Early reports found no evidence to suggest that the variant has any impact on the severity of disease or vaccine efficacy (Wu K, Werner A P, Moliva J I, et al. mRNA-1273 vaccine induces neutralizing antibodies against spike mutants from global SARS-CoV-2 variants. bioRxiv. Posted Jan. 25, 2021; Xie X, Zou J, Fontes-Garfias C R, et al. Neutralization of N501Y mutant SARS-CoV-2 by BNT162b2 vaccine-elicited sera. bioRxiv. Posted Jan. 7, 2021; Greaney A J, Loes A N, Crawford K H D, et al. Comprehensive mapping of mutations to the SARS-CoV-2 receptor-binding domain that affect recognition by polyclonal human serum antibodies. bioRxiv. [Preprint posted online Jan. 4, 2021]; Weisblum Y, Schmidt F, Zhang F, et al. Escape from neutralizing antibodies by SARS-CoV-2 spike protein variants. eLife 2020; 9:e61312.)

### B.1.351 Lineage (a.k.a. 20H/501Y.V2)

This variant has multiple mutations in the spike protein, including K417N, E484K, N501Y. Unlike the B.1.1.7 lineage detected in the UK, this variant does not contain the deletion at 69/70.

This variant was first identified in Nelson Mandela Bay, South Africa, in samples dating back to the beginning of October 2020, and cases have since been detected outside of South Africa, including the United States.

The variant also was identified in Zambia in late December 2020, at which time it appeared to be the predominant variant in the country.

Currently there is no evidence to suggest that this variant has any impact on disease severity.

There is some evidence to indicate that one of the spike protein mutations, E484K, may affect neutralization by some polyclonal and monoclonal antibodies (Weisblum Y, Schmidt F, Zhang F, et al. Escape from neutralizing antibodies by SARS-CoV-2 spike protein variants. eLife 2020; 9:e61312; Resende P C, Bezerra J F, de Vasconcelos R H T, at al. Spike E484K mutation in the first SARS-CoV-2 reinfection case confirmed in Brazil, 2020. [Posted on www.virological.org on Jan. 10, 2021])

### P.1 Lineage (a.k.a. 20J/501Y.V3)

The P.1 variant is a branch off the B.1.1.28 lineage that was first reported by the National Institute of Infectious Diseases (NIID) in Japan in four travelers from Brazil, sampled during routine screening at Haneda airport outside Tokyo.

The P.1 lineage contains three mutations in the spike protein receptor binding domain: K417T, E484K, and N501Y.

There is evidence to suggest that some of the mutations in the P.1 variant may affect its transmissibility and antigenic profile, which may affect the ability of antibodies generated through a previous natural infection or through vaccination to recognize and neutralize the virus. A recent study reported on a cluster of cases in Manaus, the largest city in the Amazon region, in which the P.1 variant was identified in 42% of the specimens sequenced from late December (Resende P C, Bezerra J F, de Vasconcelos R H T, at al. Spike E484K mutation in the first SARS-CoV-2 reinfection case confirmed in Brazil, 2020. [Posted on www.virological.org on Jan. 10, 2021]). In this region, it is estimated that approximately 75% of the population had been infected with SARS-CoV2 as of October 2020. However, since mid-December the region has observed a surge in cases. The emergence of this variant raises concerns of a potential increase in transmissibility or propensity for SARS-CoV-2 reinfection of individuals.

This variant was identified in the United States at the end of January 2021.

One specific mutation, called D614G, is shared by these three variants. It gives the variants the ability to spread more quickly than the predominant viruses, as described in a non-peer-reviewed preprint article (1Bin Zhou, Tran Thi Nhu Thao, Donata Hoffmann, et al. SARS-CoV-2 spike D614G variant confers enhanced replication and transmissibility bioRxiv 2020.10.27 doi: https://doi.org/10.1101/2020.10.27.357558; Volz E, Hill V, McCrone J, et al. Evaluating the Effects of SARS-CoV-2 Spike Mutation D614G on Transmissibility and Pathogenicity. Cell 2021; 184(64-75). doi: https://doi.org/10.1016/j.cell.2020.11.020). There also is epidemiologic evidence that variants with this specific mutation spread more quickly than viruses without the mutation (Korber B, Fischer W M, Gnanakaran S, et al. Tracking Changes in SARS-CoV-2 Spike: Evidence that D614G Increases Infectivity of the COVID-19 Virus. Cell 2021; 182(812-7). doi: https://doi.org/10.1016/j.cell.2020.06.043). This mutation was one of the first documented in the US in the initial stages of the pandemic, after having initially circulated in Europe (Yurkovetskiy L, Wang X, Pascal K E, et al. Structural and Functional Analysis of the D614G SARS-CoV-2 Spike Protein Variant. Cell 2020: 183(3): 739-1. doi: https://doi.org/10.1016/j.cell.2020.09.032).

The variants are summarised in the table below (https://www.cdc.gov/coronavirus/2019-ncov/cases-updates/variant-surveillance/variant-info.html):

| | | | Countries | | Transmis- |
|---|---|---|---|---|---|
| | Name | First | Reporting | Key | sibility |
| Name | (Nextstrain) | Detected | Cases | Mutations | Rate |
| B.1.1.7 | 20I/ | United | 70 | 69/70 | ~50% |
| | 501Y.V1 | Kingdom | | deletion | increase |
| | | | | 144Y | |
| | | | | deletion | |
| | | | | N501Y | |
| | | | | A570D | |
| | | | | D614G | |
| | | | | P681H | |
| P.1 | 20J/ | Japan/ | >4 | E484K | Not |
| | 501Y.V3 | Brazil | | K417N/T | determined |
| | | | | N501Y | |
| | | | | D614G | |
| B.1.351 | 20H/ | South | >30 | K417N | Not |
| | 501.V2 | Africa | | E484K | determined |
| | | | | N501Y | |
| | | | | D614G | |

We have designed a new full-length S protein sequence (referred to as “VOC Chimera”, or COV_S_T2_29) for use as a COVID-19 vaccine insert to protect against variants B.1.1.7, P.1, and B.1.351.

The full-length S protein amino acid sequence of SARS_CoV_2 isolate EPI_ISL_402130 (a reference sequence) is given below:

>EPI_ISL_402130 (Wuhan strain)
(SEQ ID NO: 52)
MFVELVLLPL VSSQCVNLTT RTQLPPAYEN SFTRGVYYPD KVERSSVLHS TQDLFLPEES 60
NVTWFHAIHV SGTNGTKRED NPVLPENDGV YFASTEKSNI IRGWIFGTTL DSKTQSLLIV 120
NNATNVVIKV CEFQFCNDPF LGVYYHKNNK SWMESEERVY SSANNCTFEY VSQPFLMDLE 180
GKQGNEKNLR EFVEKNIDGY FKIYSKHTPI NLVRDLPQGF SALEPLVDLP IGINITRFQT 240
LLALHRSYLT PGDSSSGWTA GAAAYYVGYL QPRTELLKYN ENGTITDAVD CALDPLSETK 300
CTLKSFTVEK GIYQTSNERV QPTESIVREP NITNLCPEGE VENATREASV YAWNRKRISN 360
CVADYSVLYN SASFSTEKCY GVSPTKLNDL CFTNVYADSF VIRGDEVRQI APGQTGKIAD 420
YNYKLPDDFT GCVIAWNSNN LDSKVGGNYN YLYRLERKSN LKPFERDIST EIYQAGSTPC 480
NGVEGENCYF PLQSYGFQPT NGVGYQPYRV VVLSFELLHA PATVCGPKKS TNLVKNKCVN 540
ENENGLIGTG VLTESNKKEL PFQQFGRDIA DTTDAVRDPQ TLEILDITPC SEGGVSVITP 600
GTNTSNQVAV LYQDVNCTEV PVAIHADQLT PTWRVYSTGS NVFQTRAGCL IGAEHVNNSY 660
ECDIPIGAGI CASYQTQTNS PRRARSVASQ SIIAYTMSLG AENSVAYSNN SIAIPTNFTI 720
SVTTEILPVS MTKTSVDCTM YICGDSTECS NLLLQYGSFC TQLNRALTGI AVEQDKNTQE 780
VEAQVKQIYK TPPIKDEGGF NESQILPDPS KPSKRSFIED LLENKVTLAD AGFIKQYGDC 840
LGDIAARDLI CAQKENGLTV LPPLLTDEMI AQYTSALLAG TITSGWTFGA GAALQIPFAM 900
QMAYRENGIG VTQNVLYENQ KLIANQFNSA IGKIQDSLSS TASALGKLQD VVNQNAQALN 960
TLVKQLSSNF GAISSVENDI LSRLDKVEAE VQIDRLITGR LQSLQTYVTQ QLIRAAEIRA 1020
SANLAATKMS ECVLGQSKRV DECGKGYHLM SFPQSAPHGV VELHVTYVPA QEKNETTAPA 1080
ICHDGKAHFP REGVFVSNGT HWFVTQRNFY EPQIITTDNT FVSGNCDVVI GIVNNTVYDP 1140
LQPELDSEKE ELDKYEKNHT SPDVDLGDIS GINASVVNIQ KEIDRLNEVA KNLNESLIDL 1200
QELGKYEQYI KWPWYIWLGF IAGLIAIVMV TIMLCCMTSC CSCLKGCCSC GSCCKEDEDD 1260
SEPVLKGVKL HYT 1273

The amino acid sequence of the designed full-length S protein sequence is given below:

>COV_S_T2_29 (VOC chimera)
(SEQ ID NO: 53)
MEVELVLLPL VSSQCVNETN RTQLPSAYIN SETRGVYYPD KVERSSVLHS TQDLFLPFES 60
NVTWFHAISG TNGTKREDNP VLPENDGVYF ASTEKSNIIR GWIEGTTLDS KTQSLLIVNN 120
ATNVVIKVCE FQFCNDPFLG VYHKNNKSWM ESEFRVYSSA NNCTFEYVSQ PELMDLEGKQ 180
GNFKNLREFV FKNIDGYFKI YSKHTPINLV RDLPQGESAL EPLVDLPIGI NITRFQTLLA 240
LHRSYLTPGD SSSGWTAGAA AYYVGYLQPR TELLKYNENG TITDAVDCAL DPLSETKCTL 300
KSFTVEKGIY QTSNERVQPT ESIVREPNIT NLCPFGEVEN ATREASVYAW NRKRISNCVA 360
DYSVLYNSAS ESTEKCYGVS PTKLNDLCFT NVYADSEVIR GDEVRQIAPG QTGNIADYNY 420
KLPDDFTGCV IAWNSNNLDS KVGGNYNYLY RLERKSNLKP FERDISTEIY QAGSTPCNGV 480
KGENCYFPLQ SYGFQPTYGV GYQPYRVVVL SFELLHAPAT VCGPKKSTNL VKNKCVNENE 540
NGLIGTGVLT ESNKKELPFQ QFGRDIADTT DAVRDPQTLE ILDITPCSFG GVSVITPGIN 600
TSNQVAVLYQ GVNCTEVPVA IHADQLTPTW RVYSTGSNVE QTRAGCLIGA EHVNNSYECD 660
IPIGAGICAS YQTQTNSHRR ARSVASQSII AYTMSLGAEN SVAYSNNSIA IPTNFTISVT 720
TEILPVSMTK TSVDCTMYIC GDSTECSNLL LQYGSFCTQL NRALTGIAVE QDKNTQEVEA 780
QVKQIYKTPP IKDEGGENFS QILPDPSKPS KRSFIEDLLE NKVTLADAGE IKQYGDCLGD 840
IAARDLICAQ KENGLTVLPP LLTDEMIAQY TSALLAGTIT SGWTFGAGAA LQIPFAMQMA 900
YRENGIGVTQ NVLYENQKLI ANQFNSAIGK IQDSLSSTAS ALGKLQDVVN QNAQALNTLV 960
KQLSSNFGAI SSVINDILSR LDPPEAEVQI DRLITGRLQS LQTYVTQQLI RAAEIRASAN 1020
LAATKMSECV LGQSKRVDFC GKGYHLMSFP QSAPHGVVFL HVTYVPAQEK NFTTAPAICH 1080
DGKAHFPREG VEVSNGTHWF VTQRNFYEPQ IITTDNTEVS GNCDVVIGIV NNTVYDPLQP 1140
ELDSFKEELD KYFKNHTSPD VDLGDISGIN ASVVNIQKEI DRINEVAKNL NESLIDLQEL 1200
GKYEQYIKWP WYIWLGFIAG LIAIVMVTIM LCCMTSCCSC LKGCCSCGSC CKEDEDDSEP 1260
VLKGVKLHYT 1270

Alignment of these two sequences is shown below. The amino acid differences between the sequences are shown boxed, with the two amino acid changes made to provide structure stability shown in the shaded box.

The amino acid differences of the designed sequence COV_S_T2_29 from the SARS2 S protein reference sequence (EPI_ISL_402130_Wuhan strain) are summarised in the table below:

| | SARS2 | |
|---|---|---|
| SARS2 S | Reference | COV_S_T2_29 |
| protein | amino | amino |
| residue | acid | acid |
| position | residue | residue |
| (SEQ ID | (SEQ ID | (SEQ ID |
| NO: 52) | NO: 52) | NO: 53) |
| 18 | L | F |
| 20 | T | N |
| 26 | P | S |
| 69 | H | Deletion |
| 70 | V | Deletion |
| 144 | Y | Deletion |
| 417 | K | N |
| 484 | E | K |
| 501 | N | Y |
| 614 | D | G |
| 681 | P | H |
| 986 | K | P |
| 987 | V | P |

| | |
|---|---|
| EPI_ISI_402130_Wuha COV_S_T2_29 | 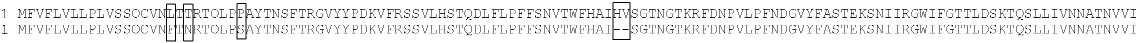 |
| EPI_ISI_402130_Wuha COV_S_T2_29 | 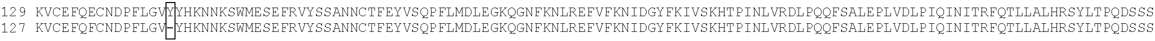 |
| EPI_ISI_402130_Wuha | 257 GWTAGAAAYYVGYLQPRTFLLKYNENGTITDAYDDALDPLSETKCTLKSFTVEKGIYQTSNFRVQPTESIVRFPNITNLCFFGEVFNATRFASVYAWNPXRISNCVADYSVLYNSASFSTFKCYGVSP |
| COV_S_T2_29 | 254 GWTAGAAAYYVGYLQPRTFLLKYNENGTITDAYDDALDPLSETKCTLKSFTVEKGIYQTSNFRVQPTESIVRFPNITNLCFFGEVFNATRFASVYAWNPXRISNCVADYSVLYNSASFSTFKCYGVSP |
| EPI_ISI_402130_Wuha COV_S_T2_29 | 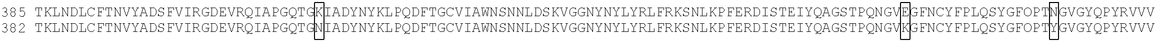 |
| EPI_ISI_402130_Wuha COV_S_T2_29 | 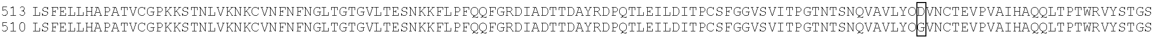 |
| EPI_ISI_402130_Wuha COV_S_T2_29 | 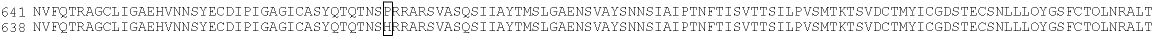 |
| EPI_ISI_402130_Wuha | 789 GIAVECDKNTQEVFAQYKQIYKTPPIKGFCGFNFSQILPQFSKPSKRSFIEDLLFNKVTLADAGFIKQYCDCLGDIAARDLICACKFNGLTVLFPLLTDEMIADYTSALLAGTITSGWTFGAGAALQI |
| COV_S_T2_29 | 786 GIAVECDKNTQEVFAQYKQIYKTPPIKGFCGFNFSQILPQFSKPSKRSFIEDLLFNKVTLADAGFIKQYCDCLGDIAARDLICACKFNGLTVLFPLLTDEMIADYTSALLAGTITSGWTFGAGAALQI |
| EPI_ISI_402130_Wuha COV_S_T2_29 | 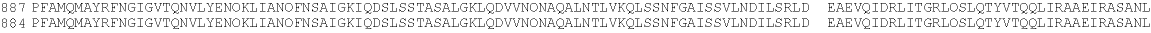 |
| EPI_ISI_402130_Wuha | 1025 AATKMSECVLGQSKRVDFCGKGYHLMSFPQSAFHQVVFLHVTVVPAQEKNFTTAPAICHDGKAHFPREGVFVSNGTHVFVTQRNFVEPQIITTDNTFVSGNCDVVIGIVNNTVYCPLQPELGSFKEEL |
| COV_S_T2_29 | 1022 AATKMSECVLGQSKRVDFCGKGYHLMSFPQSAFHQVVFLHVTVVPAQEKNFTTAPAICHDGKAHFPREGVFVSNGTHVFVTQRNFVEPQIITTDNTFVSGNCDVVIGIVNNTVYCPLQPELGSFKEEL |
| EPI_ISI_402130_Wuha | 1153 DKYFKNHTSPDVDLGDISGINASVVNIQKEIDRLNEVAKNLNEDLIDLQELGKYEQYIKWFWYIWLGFIAGLIAIVMVTIMLCCMTSCCSCLKGCCSCGSCCKFDEDDSEPVLKSYKLHYT |
| COV_S_T2_29 | 1150 DKYFKNHTSPDVDLGDISGINASVVNIQKEIDRLNEVAKNLNEDLIDLQELGKYEQYIKWFWYIWLGFIAGLIAIVMVTIMLCCMTSCCSCLKGCCSCGSCCKFDEDDSEPVLKSYKLHYT |

### EXAMPLE 31

### Designed S Protein Sequence in Closed State to Protect Against Known COVID-19 Variants, and Predicted Future Variants

The majority of SARS-CoV-2 vaccines in use or in advanced clinical development are based on the viral spike protein (S) as their immunogen. S is present on virions as pre-fusion trimers in which the receptor binding domain (RBD) is stochastically open or closed. Neutralizing antibodies have been described that act against both open and closed conformations. The long-term success of vaccination strategies will depend upon inducing antibodies that provide long-lasting broad immunity against evolving, circulating SARS-CoV-2 strains, while avoiding the risk of antibody dependent enhancement as observed with other Coronavirus vaccines.

Carnell et al. (“SARS-CoV-2 spike protein arrested in the closed state induces potent neutralizing responses”; https://doi.org/10.1101/2021.01.14.426695, posted 14 Jan. 2021) have assessed the results of immunization in a mouse model using an S protein trimer that is arrested in the closed state to prevent exposure of the receptor binding site and therefore interaction with the receptor. The authors compared this with a range of other modified S protein constructs, including representatives used in current vaccines. They found that all trimeric S proteins induce a long-lived, strongly neutralizing antibody response as well as T-cell responses. Notably, the protein binding properties of sera induced by the closed spike differed from those induced by standard S protein constructs. Closed S proteins induced more potent neutralising responses than expected based on the degree to which they inhibit interactions between the RBD and ACE2. The authors conclude that these observations suggest that closed spikes recruit different, but equally potent, virus-inhibiting immune responses than open spikes, and that this is likely to include neutralizing antibodies against conformational epitopes present in the closed conformation.

We have appreciated that the amino acid changes of the designed S protein sequences disclosed herein (and especially in Example 30 above) may optionally be present in a designed S protein that is arrested in the closed state, and thereby further improve the antibody response of the designed sequences. In particular, use of such structural constraints may reduce immunodominance to key regions, and spread the antibody response to focus on other, or less immunodominant sites.

SARS-CoV-2 is continually evolving, with more contagious mutations spreading rapidly. Zahradnik et al., 2021 (“SARS-CoV-2 RBD in vitro evolution follows contagious mutation spread, yet generates an able infection inhibitor”; doi: https://doi.org/10.1101/2021.01.06.425392, posted 29 Jan. 2021) recently reported using in vitro evolution to affinity maturate the receptor-binding domain (RBD) of the spike protein towards ACE2 resulting in the more contagious mutations, S477N, E484K, and N501Y, to be among the first selected, explaining the convergent evolution of the “European” (20E-EU1), “British” (501.V1), “South African” (501.V2), and “Brazilian” variants (501.V3). The authors report that further in vitro evolution enhancing binding by 600-fold provides guidelines towards potentially new evolving mutations with even higher infectivity. For example, Q498R epistatic to N501Y.

We have also appreciated that the designed S protein sequences (RBD, truncated, or full-length) disclosed herein (and especially in Example 30 above) may optionally also include amino acid substitutions at residue positions predicted to be mutated in future COVID-19 variants with a vaccine escape response.

The amino acid sequence alignment below shows the full-length S protein amino acid sequence of SARS_CoV_2 isolate EPI_ISL_402130 (a reference sequence: SEQ ID NO:52) with the amino acid changes made for the designed S protein sequence described in Example 30 above (“VOC Chimera”, or COV_S_T2_29; SEQ ID NO:53), shown underneath the isolate sequence (in the line referred to as “Super_spike”). This designed (“Super_spike”) S protein sequence may optionally also include one or more amino acid changes (a substitution or deletion) at one or more of the residue positions predicted to be mutated in future COVID-19 variants with a vaccine escape response.

The line underneath the “Super_spike” sequence alignment shows the residues that may be substituted for cysteine residues to allow formation of a disulphide bridge to form a “closed S protein” (SEQ ID NO:107). These cysteine substitutions may be combined with one or more (or all) of the amino acid changes made in the designed S protein sequence of the “Super_spike” sequence (COV_S_T2_29; SEQ ID NO:53), and optionally with one or more (or all) amino acid changes at the residue positions predicted to be mutated in future COVID-19 variants with a vaccine escape response (especially including, for example, Q498R).

The table below the alignment summarises the amino acid changes.

The shaded residues in the alignment (and table) are as follows:
Grey—amino acid residues that have been changed in the “Super_spike” design;
Dark grey—amino acid residues that may be substituted for a cysteine residue to allow formation of a “closed S protein”;
Light grey—amino acid residues that have been predicted to be mutated in future COVID-19 variants and potentially generate a vaccine escape response.

| EPI_ISL_402130 Super_spike X2 | 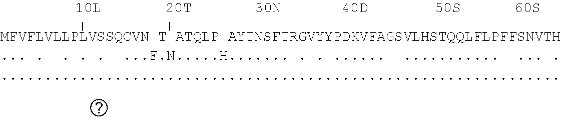 | | | |
|---|---|---|---|---|
| EPI_ISL_402130 Super_spike X2 | 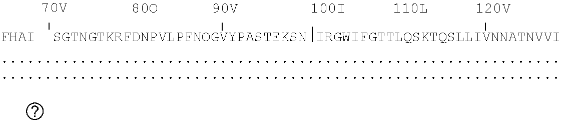 | | | |
| EPI_ISL_402130 Super_spike X2 | 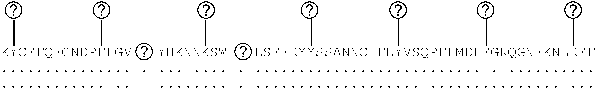 | | | |
| EPI_ISL_402130 Super_spike X2 | 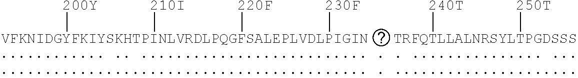 | | | |
| EPI_ISL_402130 Super_spike X2 | 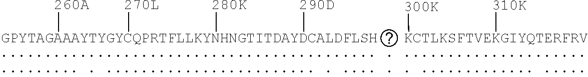 | | | |
| EPI_ISL_402130 Super_spike X2 | 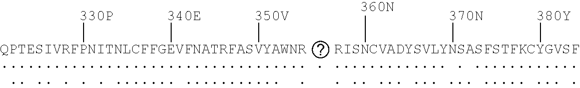 | | | |
| EPI_ISL_402130 Super_spike X2 | 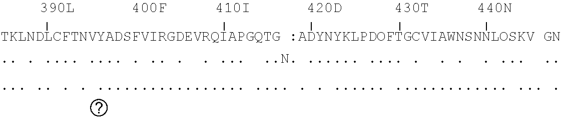 | | | |
| EPI_ISL_402130 Super_spike X2 | 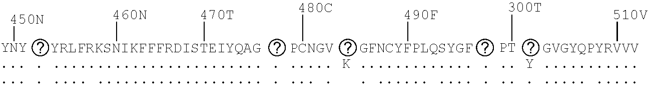 | | | |
| EPI_ISL_402130 Super_spike X2 | 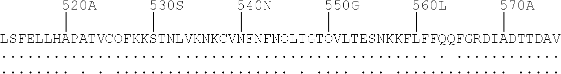 | | | |
| EPI_ISL_402130 Super_spike X2 | 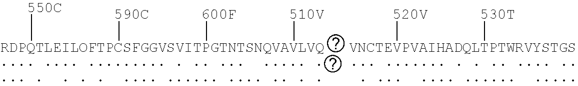 | | | |
| EPI_ISL_402130 Super_spike X2 | 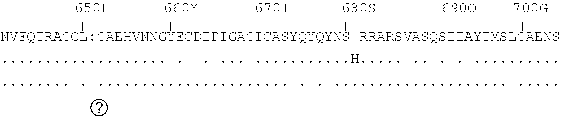 | | | |
| EPI_ISL_402130 Super_spike X2 | 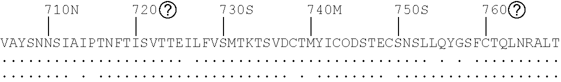 | | | |
| EPI_ISL_402130 Super_spike X2 | 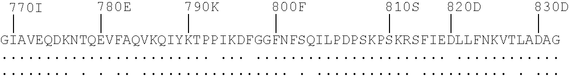 | | | |
| EPI_ISL_402130 Super_spike X2 | 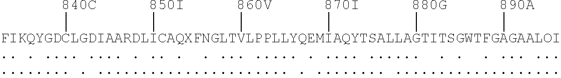 | | | |
| EPI_ISL_402130 Super_spike X2 | 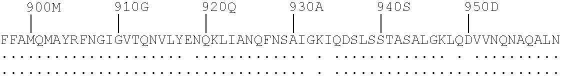 | | | |
| EPI_ISL_402130 Super_spike X2 | 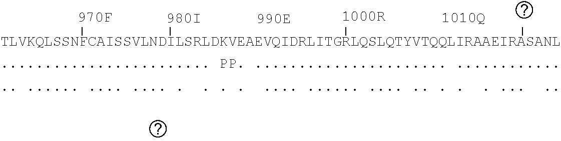 | | | |
| EPI_ISL_402130 Super_spike X2 | 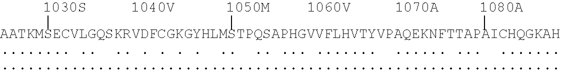 | | | |
| EPI_ISL_402130 Super_spike X2 | 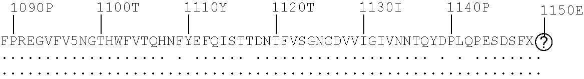 | | | |
| EPI_ISL_402130 Super_spike X2 | 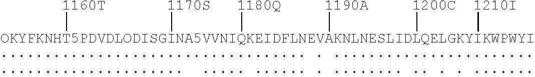 | | | |
| EPI_ISL_402130 Super_spike X2 | 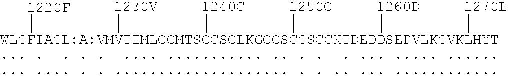 | | | |
| | | | Residues at | |
| | | | which mutations | |
| SARS2 S | | | have been | Residue at |
| protein | | | predicted to | which cysteine |
| residue | | COV_S_T2_29 | arise and | substitution can |
| position | SARS2 Reference | amino acid | generate | be made to form |
| (SEQ ID | amino acid residue | residue (SEQ ID | vaccine escape | closed spike |
| NO: 52) | (SEQ ID NO: 52) | NO: 53) | response | protein |
| 18 | L | F | | |
| 20 | T | N | | |
| 26 | P | S | | |
| 69 | H | Deletion | | |
| 70 | V | Deletion | | |
| 144 | Y | Deletion | | |
| 413 | 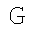 | | | 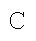 |
| 417 | K | N | | |
| 446 | 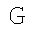 | | 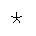 [SMILES: *] | |
| 452 | 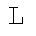 | | 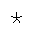 [SMILES: *] | |
| 477 | 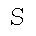 | | 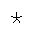 [SMILES: *] | |
| 484 | E | K | | |
| 498 | 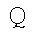 | | 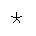 [SMILES: *] | |
| | | | (for example | |
| | | | Q498R) | |
| 501 | N | Y | | |
| 614 | D | G | | |
| 681 | P | H | | |
| 986 | K | P | | |
| 987 | 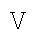 | P | | 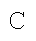 |

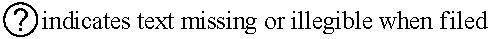

Optionally G413C and V987C is combined with one or more (or all) of the amino acid changes listed in the table below:

| SARS2 S | COV_S_T2_29 |
|---|---|
| protein | amino |
| residue | acid |
| position | residue |
| (SEQ ID | (SEQ ID |
| NO: 52 | NO: 53) |
| 18 | F |
| 20 | N |
| 26 | S |
| 69 | (deletion) |
| 70 | — (deletion) |
| 144 | — (deletion) |
| 417 | N |
| 484 | K |
| 501 | Y |
| 614 | G |
| 681 | H |

A further amino acid change that may optionally be included is K986P.

### EXAMPLE 32

### Epitope Optimised Broad Coverage Vaccine Designs for Sarbecoviruses

### Overview

To increase the coverage of our receptor binding domain (RBD) based vaccine designs to all the extant sarbecovirus sub-genus of Beta-coronaviruses, a phylogenetically optimised vaccine design is constructed. This design is further used as backbone for designing both epitope optimised and immune re-focused designs. The epitope information is derived largely from the known high-resolution structural data of spike protein-antibody complex. Few of these epitopes are reported to cross protect SARS-1 and SARS-2 and were included in the designs to increase the coverage of the vaccine designs. On further analysis of the sequence divergence of the epitopes, it was observed that one of the epitopes shows maximum divergence among sarbecovirus in comparison to other regions/epitopes of RBD. To enhance the immune response toward better conserved epitopes, post-translation modification—glycosylation was introduced at this epitope.

### Results

### Design of Broad Coverage Vaccine Antigens

To achieve broader response towards sarbecoviruses, we first generated a phylogenetically optimised design (COV_S_T2_13) (SEQ ID NO:27) where the amino acid sequence of RBD is optimised for all the extant sequences represented in FIG. 35A. Such a design is expected to generate broader antibody response compared to individual antigen from the extant species. To further understand the contribution of each epitope to antibody response, we modified the epitope sequences of COV_S_T2_13 to match the epitope sequences from SARS-1 and SARS-2. Three conformational epitopes (also referred to herein as “discontinuous epitopes”) are identified through structural analyses of RBD-antibody complex (FIG. 35B). Two of these epitopes (henceforth termed as A and B) are reported to bind antibodies that neutralise both SARS-1 and SARS-2. These epitopes on COV_S_T2_13 designs are modified to match the SARS-1 epitope sequence (COV_S_T2_14 (SEQ ID NO:28) and COV_S_T2_15 (SEQ ID NO:29)) to understand the contribution of these epitopes to generate neutralising antibody response against both SARS-1 and SARS-2. The third epitope (henceforth termed as C) is in and around the receptor binding region. This epitope shows maximum divergence (FIG. 35C) and is expected to generate a virus specific antibody response. To understand the importance of the amino acid composition of this epitope in generating neutralising antibody response, this epitope is modified to match the epitope from SARS-2 (COV_S_T2_16) (SEQ ID NO:30). Further to broaden the antibody response to both SARS-1 and SARS-2, a glycosylation site is introduced at the third epitope for both COV_S_T2_14 and COV_S_T2_15 (COV_S_T2_17 (SEQ ID NO:31) and COV_S_T2_18 (SEQ ID NO:32) respectively). To compare the efficacy in generating neutralising antibody response in soluble or membrane bound form, a membrane bound form for COV_S_T2_13 and COV_S_T2_17 (COV_S_T2_19 (SEQ ID NO:55) and COV_S_T2_20 (SEQ ID NO:56) respectively) is designed. All the designs are tabulated in the Table below. The sequence alignment of all the vaccine designs is shown in FIG. 37A. The residues that differ between the vaccine designs are boxed in black.

TABLE
Description of the vaccine designs used in the study.

| DESIGN | DESCRIPTION |
|---|---|
| COV_S_T2_13 | Phylogenetic optimised |
| COV_S_T2_14 | COV_S_T2_13 with epitope A and epitope |
| COV_S_T2_15 | B from SARS-1 |
| COV_S_T2_16 | COV_S_T2_13 with epitope C from SARS-2 |
| COV_S_T2_17 | COV_S_T2_13 with glycosylation site at |
| COV_S_T2_18 | epitope C |
| COV_S_T2_19 | Membrane bound version of COV_S_T2_13 |
| COV_S_T2_20 | Membrane bound version of COV_S_T2_17 |

FIG. 36(A) shows a Western Blot of sera from mice immunised with the vaccine designs.

FIG. 36 (B) shows antibody binding responses of Cell Surface expression bleed 2.

### Neutralisation Data

Sera from mice injected with the vaccine designs (COV_S_T2-13-20), SARS-1 RBD and SARS-2 RBD are checked for neutralisation of SARS-1 and SARS-2 pseudotypes. As a positive control, human sera from an infected individual are used. The neutralisation curves are shown in FIG. 37B. The phylogenetically optimised design (COV_S_T2_13) could generate neutralising antibody against SARS-2 but not for SARS-1. On comparing the sequence of the COV_S_T2_13 with SARS-1 and SARS-2, it is observed that the epitope C was enriched with amino acids from SARS-2 in comparison to other sarbecoviruses represented in phylogenetic tree (FIG. 35A). Sera from mice vaccinated with COV_S_T2_14, COV_S_T2_15, and COV_S_T2_16 showed data like COV_S_T2_13 for SARS-1, suggesting strongly that the epitope C is an immunodominant epitope and epitope A and B are immune sub-dominant epitope. Better neutralisation of SARS-2 by COV_S_T2_16 in comparison to COV_S_T2_13 suggests that the mutations at epitope C can lead to lower neutralisation of SARS-2. Substitution made in COV_S_T2_15 enhances the immunogenic response for SARS-2. The difference in immunogenic response could be due to the substitution of a small amino acid serine by bulky phenylalanine group.

Sera from COV_T2_S_17 and COV_T2_S_18 designs could neutralise both SARS-1 and SARS-2, suggesting that the introduction of glycosylation at epitope C successfully focused the immune response towards epitope A and epitope B. Thus, validating our design strategy. Comparison of neutralisation data of COV_T2_S_13 and COV_T2_S_17 with COV_S_T2_19 and COV_S_T2_20 respectively suggest that the membrane bound and soluble form similar immunogenic response in mice.

Neutralisation data for bat viruses (not shown) shows broader coverage. This rationalises the usage of phylogenetic optimised sequence as the template for further designs.

Competition data (not shown) shows that all the designs generate antibodies that block receptor binding.

### Discussion

A vaccine design which can generate antibody response against diverse sarbecovirus is desirable. To achieve this, we first generated a novel protein sequence (COV_S_T2_13) for the receptor binding domain of the spike protein by using sequence information for all the know extant sarbecoviruses. Each amino acid position in the sequence is chosen based on the phylogenetic relatedness of the input sequences. The novel sequence generated neutralising response against SARS-2 but not much against SARS-1. On comparison of the epitopes in the COV_S_T2_13 and SARS-1 and SARS-2, it was observed that the epitopes were more biased towards SARS-2 compared to SARS-1. To expand the reactivity towards SARS-1, two of the epitopes (which were also conserved between SARS-1 and SARS-2) were mutated to match the sequence from SARS-1 (COV_S_T2_14 and COV_S_T2_15) and the third epitope was mutated to match SARS-2 (COV_S_T2_16). Comparison of the neutralisation from these designs suggested that the two conserved epitopes are sub-dominant in nature compared to the third epitope. Also, comparison of COV_S_T2_16 with COV_S_T2_13 suggested that conservative mutations in the third epitope can cause immune escape. To focus the immune response towards the conserved epitopes, a glycosylation site was introduced at the more diverged third epitope (COV_S_T2_17 and COV_S_T2_18). The introduction of the glycosylation site indeed broadened the immune response to both SARS-1 and SARS-2, with cross-neutralisation observed for both the designs. The data presented here strongly supports the design strategy to broaden the coverage of vaccine designs by re-focusing the immune response to better conserved epitopes by introducing modifications in epitopes that more diverged.

### Methodology

### Phylogenetic Analysis

Protein sequences of spike proteins were downloaded from the NCBI virus database for all the known sarbecoviruses. Multiple sequence alignment (MSA) was generated using the MUSCLE algorithm. The resulting MSA was pruned to the RBD region and used as input for phylogenetic tree reconstruction. The phylogenetic tree was generated using IQTREE algorithm using protein model with best AIC score. The resultant tree was used for generation of phylogenetically optimised design using FASTML algorithm.

### Epitope Identification

Available structural data for Spike protein-antibody complexes for SARS-1 and SARS-2 were downloaded from the Protein Databank (PDB). These structural data were further pruned for antigen-antibody complexes where the epitope region is in the RBD. Amino acid residues of antigen that have at least one atom within SA radii of at least one atom of amino acid of antibody are defined as epitope residues. An epitope region is defined as contiguous stretch of at least 5 amino acids.

### Molecular Modelling

Structural models were generated for COV_S_T2_13 using MODELLER algorithm. The structural model with the highest DOPE score was chosen as the working model for the further molecular modelling. The side chains for the model were further optimised using SCWRL library and energy minimised using GROMACS package. Structural stability of the COV_S_T2_14-COV_S_T2_18 designs was checked for using POSSCAN and BUILD module of FOLDX algorithm using the optimised structural model of COV_S_T2_13.

### EXAMPLE 33

### Dose Finding Study of COV_S_T2_17 (SEQ ID NO:31), a Pan-Sarbeco Coronavirus Vaccine DNA Candidate, Delivered by Needleless Intradermal Administration

### Study Protocol in Brief (FIG. 38):

To determine the optimal dose of DNA, a pre-clinical vaccine study was undertaken in mature Hartley Guinea pigs. Animals were randomised into six groups of eight animals and pre-bled to determine the absence of anti-SARS-CoV-2 antibodies.

Group 1 (control) group received the high dose of 400 ug (2 mg/ml) of the modified SARS-CoV-2 RBD COV_S_T2_8 DNA subcutaneously, to compare to a second group the same control DNA of COV_S_T2_8 at 400 ug administered intradermally (ID) by the PharmaJet Tropis device. The remaining four groups received the pan-Sarbeco vaccine candidate, COV_S_T2_17 at 100 ug (0.5 mg/ml), 200 ug (1 mg/ml) (two groups, one receiving 2, the other 3 doses) or 400 ug (400 ug/ml) intradermally at day 0 and 28. Animals were bled at days 14, 28, 42, 56 and 70.

### ELISA to Determine the Level of Antibodies to the RBD of SARS-CoV-2, and SARS (FIG. 39):

Panel A (left) Plates coated with SARS-CoV-2 RBD.

28 days following the first immunisation an ELISA assay was performed to determine the titre of anti-SARS-CoV-2 RBD, or anti-SARS RBD antibodies induced 28 days after one DNA immunisation. The top left panel (T2_8 at 400 ug sc) demonstrates the antibody responses to SARS-CoV-2 in 5 out of 8 animals, compared to the bottom right hand panel (T2_8 at 400 ug DNA administered ID by the Tropis Pharmajet) where 7 of 8 animals respond strongly to SARS-CoV-2 RBD. The 4 remaining groups receiving COV_S_T2_17 ID by PharmaJet delivery, showed similar anti-SARS-CoV-2 responses to 400 ug of the SARS-CoV-2 RBD DNA administered at the maximal dose.

Panel B (right) Plates coated with SARS RBD.

The same 28 day serum samples at serial dilutions were tested for binding to the SARS RBD.

The top left panel (T2_8 at 400 ug sc) demonstrates low titre antibodies, with only 2 of 8 animals reaching an OD of 0.5. The same dose of the SARS-CoV-2 RBD vaccine given by the PharmaJet device (bottom right hand panel) demonstrates slightly improved but weak cross-reactive responses to the SARS RBD in contrast to its homotypic response to the SARS-CoV-2 RBD (panel A, left). In contrast all of the pan-Sarbeco T2_17 groups respond strongly to the SARS RBD in a dose-dependent manor, with all animals in the high (400 ug) (bottom row left in panel B) and medium doses (200 ug) groups (middle row panel B) responding strongly, and a more variable but distinct response in all 8 animals in the lowest (100 ug) T2_17 group (top right, panel B).

Virus Neutralisation at Day 28 after 1 Immunisation (Pseudotype MicroNeutralisation or pMN Assay) (FIG. 40):

Panel A (left) Antibody neutralisation of SARS-CoV-2 28 days after 1 dose.

Similar to RBD antibody responses, neutralising antibodies to SARS-CoV-2 were identified. In all groups 28 days following the first immunisation. The top left panel (T2_8 at 400 ug sc) had low level responses compared to the same vaccine candidate (T2_8 at 400 ug DNA) administered ID by the Tropis Pharmajet device, which was the strongest of all the groups. T2_17 ID by PharmaJet delivery, showed lower but significant responses to SARS-CoV-2.

Panel B (right) Antibody neutralisation of SARS 28 days after 1 dose.

The same 28 day serum samples at serial dilutions were tested for neutralising to SARS pseudotyped viruses. At this time point, after 1 administration, responses were absent in the T2_8 groups (top left and bottom right of panel B (right).

The pan-Sarbeco T2_17 groups respond at low and variable levels after 1 dose of vaccine, again with the best but weak response in the highest dose group (400 ug) (bottom row left in panel B)

Groups 1 to 3, Comparison of Virus Neutralisation Responses after First to Second Immunisation (FIG. 41):

Panel A (left SARS-CoV-2) Comparing bleeds 2 (pre) and 3 (post) second immunisation (boost)

There was significant boost effect with increased neutralising responses to SARS-CoV-2 in all groups, though not all animals in group 1 (T2_8 at 400 ug) administered subcutaneously. Groups 2 and 3, middle and lower rows of panel A, left, were more uniform and comparably boosted neutralising titres to SARS-CoV-2.

Panel B (right SARS) Comparing bleeds 2 (pre) and 3 (post) second immunisation (boost).

There was weak and variable boost effect in 5 of 8 animals to SARS in group 1 (T2_8 at 400 ug). Groups 2 and 3, middle and lower rows of panel A, left, were uniform and comparably strongly boosted with significant neutralising titres to SARS.

Groups 4, 5 and 6, Comparison of Virus Neutralisation Responses after First to Second Immunisation (FIG. 42):

Panel A (left SARS-CoV-2) Comparing bleeds 2 (pre) and 3 (post) second immunisation (boost).

Comparing the left hand column of groups 4, 5 and 6, there was significant boost effect with increased neutralising responses to SARS-CoV-2 in Group 4 200 ug T_17 Tropis, group 5 400 ug T_17 Tropis, and the SARS-CoV-2 specific 400 ug T2_8 also delivered by Tropis.

Panel B (right SARS) Comparing bleeds 2 (pre) and 3 (post) second immunisation (boost).

Comparing the left to the right hand column of groups 4, 5 and 6, there was clear boost effect with increased neutralising responses to SARS in all 3 groups, but most significantly in the two T2_17 immunised groups (4 and 5, upper right hand graphs) that received 200 ug (top row panel B), and 400 ug of T2_17 (middle row panel B), with a possible dose effect in the 400 ug dose. In contrast, the 400 ug T2_8 group was boosted to a much lower and variable effect.

### Neutralisation of Variants of Concern (FIG. 43):

Selected high, middle and low neutralising antibody responders from T2_8 and T2_17 guinea pig groups were tested for pseudotype based viral neutralisation of the original Wuhan strain (control), as well as variants of concern (VOC) lineages B1.248 (Brazil P1 lineage) and B1.351 (South Africa). Both these VOCs contain the E484K mutation that confers resistance to current vaccines in use (AstraZeneca, Pfizer, Moderna). High responding T2_8 guinea pig (8 and 11) antisera do not neutralise the VOCs, whereas high responders from the T2_17 group (31 and 34) still neutralise strongly.

### EXAMPLE 34

Nucleic Acid Sequences Encoding COV S T2 13-20
>COV_S_T2_13 encoding nucleic acid
(SEQ ID NO: 78)
AGAGTGGCCCCTACCAAAGAAGTCGTGCGGTTCCCCAACATCACC
AATCTGTGCCCTTTCGGCGAGGTGTTCAACGCCACCAGATTTCCC
TCTGTGTACGCCTGGGAGAGAAAGCGGATCAGCAACTGCGTGGCC
GACTACAGCGTGCTGTACAACAGCACCAGCTTCAGCACCTTCAAG
TGCTACGGCGTGTCACCCACCAAGCTGAACGACCTGTGCTTCACC
AACGTGTACGCCGACAGCTTCGTGATCAGAGGCGACGAAGTGCGG
CAGATTGCCCCTGGACAAACAGGCGTGATCGCCGATTACAACTAC
AAGCTGCCCGACGACTTCACCGGCTGTGTGATCGCCTGGAACACC
AACAACCTGGACAGCACCACCGGCGGCAACTACAACTACCTGTAC
AGAAGCCTGCGGAAGTCTAAGCTGAAGCCCTTCGAGCGGGACATC
AGCAGCGACATCTATAGCCCTGGCGGCAAGCCTTGTTCTGGCGTG
GAAGGCTTCAACTGCTACTACCCTCTGCGGAGCTACGGCTTCTTC
CCCACAAATGGCGTGGGCTACCAGCCTTACAGAGTGGTGGTCCTG
AGCTTCGAGCTGCTGAATGCCCCTGCCACAGTGTGTGGCCCTAAG
CTGTCTACCGAC
>COV_S_T2_14 encoding nucleic acid
(SEQ ID NO: 79)
AGAGTGGCCCCTACCAAAGAAGTCGTGCGGTTCCCCAACATCACC
AATCTGTGCCCTTTCGGCGAGGTGTTCAACGCCACCAAGTTTCCC
TCTGTGTACGCCTGGGAGCGCAAAAAGATCAGCAACTGCGTGGCC
GACTACAGCGTGCTGTACAACAGCACCAGOTTCAGCACCTTCAAG
TGCTACGGCGTGTCACCCACCAAGCTGAACGACCTGTGCTTCACC
AACGTGTACGCCGACAGCTTCGTGATCAGAGGCGACGAAGTGCGG
CAGATTGCCCCTGGACAAACAGGCGTGATCGCCGATTACAACTAC
AAGCTGCCCGACGACTTCACCGGCTGTGTGATCGCCTGGAACACC
AACAACATCGACAGCACCACCGGCGGCAACTACAACTACCTGTAC
AGAAGCCTGCGGAAGTCTAAGCTGAAGCCCTTCGAGCGGGACATC
AGCAGCGACATCTATAGCCCTGGCGGCAAGCCTTGTTCTGGCGTG
GAAGGCTTCAACTGCTACTACCCTCTGCGGAGCTACGGCTTCTTC
CCCACAAATGGCGTGGGCTACCAGCCTTACAGAGTGGTGGTCCTG
AGCTTCGAGCTGCTGAATGCCCCTGCCACAGTGTGTGGCCCTAAG
CTGTOTACCGAC
>COV_S_T2_15 encoding nucleic acid
(SEQ ID NO: 80)
AGAGTGGCCCCTACCAAAGAAGTCGTGCGGTTCCCCAACATCACC
AATCTGTGCCCTTTCGGCGAGGTGTTCAACGCCACCAGATTTCCC
TCTGTGTACGCCTGGGAGAGAAAGCGGATCAGCAACTGCGTGGCC
GACTACAGCGTGCTGTACAACAGCACCTTCTTCAGCACCTTTAAG
TGCTACGGCGTGTCACCCACCAAGCTGAACGACCTGTGOTTCAGC
AACGTGTACGCCGACAGCTTCGTGATCAGAGGCGACGAAGTGCGG
CAGATTGCCCCTGGACAAACAGGCGTGATCGCCGATTACAACTAC
AAGCTGCCCGACGACTTCATGGGCTGTGTGATCGCCTGGAACACC
AACAACCTGGACAGCACCACCGGCGGCAACTACAACTACCTGTAC
AGAAGCCTGCGGAAGTCTAAGCTGAAGCCCTTCGAGCGGGACATC
AGCAGCGACATCTATAGCCCTGGCGGCAAGCCTTGTTCTGGCGTG
GAAGGCTTCAACTGCTACTACCCTCTGCGGAGCTACGGCTTCTTC
CCCACAAATGGCGTGGGCTACCAGCCTTACAGAGTGGTGGTCCTG
AGCTTCGAGCTGCTGAATGCCCCTGCCACAGTGTGTGGCCCTAAG
CTGTCTACCGAC
>COV_S_T2_16 encoding nucleic acid
(SEQ ID NO: 81)
AGAGTGGCCCCTACCAAAGAAGTCGTGCGGTTCCCCAACATCACC
AATCTGTGCCCTTTCGGCGAGGTGTTCAACGCCACCAGATTTCCC
TCTGTGTACGCCTGGGAGAGAAAGCGGATOAGCAACTGCGTGGCC
GACTACAGCGTGCTGTACAACAGCACCAGOTTCAGCACCTTCAAG
TGCTACGGCGTGTCACCCACCAAGCTGAACGACCTGTGCTTCACC
AACGTGTACGCCGACAGCTTCGTGATCAGAGGCGACGAAGTGCGG
CAGATTGCCCCTGGACAGACAGGCAAGATCGCCGATTACAACTAC
AAGCTGCCCGACGACTTCACCGGCTGTGTGATCGCCTGGAACACC
AACAACCTGGACAGCACCACCGGCGGCAACTACAACTACCTGTAC
CGGCTGTTCCGGAAGTCCAACCTGAAGCCTTTCGAGCGGGACATC
AGCAGCGACATCTATCAGGCCGGCAGCACACCTTGTTCTGGCGTG
GAAGGCTTCAACTGCTACTTOCCACTGCAAAGCTACGGCTTCCAG
CCTACCAACGGCGTGGGCTACCAGCCTTATAGAGTGGTGGTCCTG
AGCTTCGAGCTGCTGAATGCCCCTGCCACAGTGTGTGGCCCTAAG
CTGTGTACCGAC
>COV_S_T2_17 encoding nucleic acid
(SEQ ID NO: 82)
AGAGTGGCCCCTACCAAAGAAGTCGTGCGGTTCCCCAACATCACC
AATCTGTGCCCTTTCGGCGAGGTGTTCAACGCCACCAAGTTTCCC
TCTGTGTACGCCTGGGAGCGCAAAAAGATCAGCAACTGCGTGGCC
GACTACAGCGTGCTGTACAACAGCACCAGCTTCAGCACCTTCAAG
TGCTACGGCGTGTCACCCACCAAGCTGAACGACCTGTGCTTCACC
AACGTGTACGCCGACAGCTTCGTGATCAGAGGCGACGAAGTGCGG
CAGATTGCCCCTGGACAAACAGGCGTGATCGCCGATTACAACTAC
AAGCTGCCCGACGACTTCACCGGCTGTGTGATCGCCTGGAACACC
AACAACATCGACAGCACCACCGGCGGCAACTACAACTACCTGTAC
AGAAGCCTGOGGAAGTCTAAGCTGAAGCCCTTCGAGCGGGACATC
AGCAGCGACATCTATAGCCCTGGCGGCAAGCCTTGTTCTGGCGTG
GAAGGCTTCAACTGCTACTACCCTCTGCGGAGCTACGGCTTCTTC
CCCACAAATGGCACAGGCTACCAGCCTTACAGAGTGGTGGTCCTG
AGCTTCGAGCTGCTGAATGCCCCTGCCACAGTGTGTGGCCCTAAG
CTGTCTACCGAC
>COV_S_T2_18 encoding nucleic acid
(SEQ ID NO: 83)
AGAGTGGCCCCTACCAAAGAAGTCGTGCGGTTCCCCAACATCACC
AATCTGTGCCCTTTCGGCGAGGTGTTCAACGCCACCAGATTTCCC
TCTGTGTACGCCTGGGAGAGAAAGCGGATCAGCAACTGCGTGGCC
GACTACAGCGTGCTGTACAACAGCACCTTCTTCAGCACCTTTAAG
TGCTACGGCGTGTCACCCACCAAGCTGAACGACCTGTGCTTCAGC
AACGTGTACGCCGACAGCTTCGTGATCAGAGGCGACGAAGTGCGG
CAGATTGCCCCTGGACAAACAGGCGTGATCGCCGATTACAACTAC
AAGCTGCCCGACGACTTCATGGGCTGTGTGATCGCCTGGAACACC
AACAACCTGGACAGCACCACCGGCGGCAACTACAACTACCTGTAC
AGAAGCCTGCGGAAGTCTAAGCTGAAGCCCTTCGAGCGGGACATC
AGCAGCGACATCTATAGCCCTGGCGGCAAGCCTTGTTCTGGCGTG
GAAGGCTTCAACTGCTACTACCCTCTGCGGAGCTACGGCTTCTTC
CCCACAAATGGCACAGGCTACCAGCCTTACAGAGTGGTGGTCCTG
AGCTTCGAGCTGCTGAATGCCCCTGCCACAGTGTGTGGCCCTAAG
CTGTCTACCGAC
>COV_S_T2 19 encoding nucleic acid
(SEQ ID NO: 84)
AGAGTGGCCCCTACCAAAGAAGTCGTGCGGTTCCCCAACATCACC
AATCTGTGCCCTTTCGGCGAGGTGTTCAACGCCACCAGATTTCCC
TCTGTGTACGCCTGGGAGAGAAAGOGGATCAGCAACTGCGTGGCC
GACTACAGCGTGCTGTACAACAGCACCAGCTTCAGCACCTTCAAG
TGCTACGGCGTGTCACCCACCAAGCTGAACGACCTGTGCTTCACC
AACGTGTACGCCGACAGCTTCGTGATCAGAGGCGACGAAGTGCGG
CAGATTGCCCCTGGACAAACAGGCGTGATCGCCGATTACAACTAC
AAGCTGCCCGACGACTTCACCGGCTGTGTGATCGCCTGGAACACC
AACAACCTGGACAGCACCACCGGCGGCAACTACAACTACCTGTAC
AGAAGCCTGCGGAAGTCTAAGCTGAAGCCCTTCGAGCGGGACATC
AGCAGCGACATCTATAGCCCTGGOGGCAAGCCTTGTTCTGGCGTG
GAAGGCTTCAACTGCTACTACCCTCTGCGGAGCTACGGCTTCTTC
CCCACAAATGGCGTGGGCTACCAGCCTTACAGAGTGGTGGTCCTG
AGCTTCGAGCTGCTGAATGCCCCTGCCACAGTGTGTGGCCCTAAG
CTGTCTACAGATGGCGGCGGAGGATCTGGCGGAGGTGGAAGCGGA
GGCGGAGGAÅGCGGTGGCGGOGGATCTAAATCTTCTATCGCCAGC
TTCTTCTTCATCATCGGCCTGATTATCGGCCTGTTCCTGGTGCTG
AGAGTGGGCATCCACCTGTGCATCAAGCTGAAACACACCAAGAAG
CGGCAAATCTACACCGACATCGAGATGAACCGGCTGGGCAAA
>COV_S_T2_20 encoding nucleic acid
(SEQ ID NO: 85)
AGAGTGGCCCCTACCAAAGAAGTCGTGCGGTTCCCCAACATCACC
AATCTGTGCCCTTTCGGCGAGGTGTTCAACGCCACCAAGTTTCCC
TCTGTGTACGCCTGGGAGCGCAAAAAGATCAGCAACTGCGTGGCC
GACTACAGCGTGCTGTACAACAGCACCAGCTTCAGCACCTTCAAG
TGCTACGGCGTGTCACCCACCAAGCTGAACGACCTGTGCTTCACC
AACGTGTACGCCGACAGCTTCGTGATCAGAGGCGACGAAGTGCGG
CAGATTGCCCCTGGACAAACAGGCGTGATCGCCGATTACAACTAC
AAGCTGCCCGACGACTTCACCGGCTGTGTGATCGCCTGGAACACC
AACAACATCGACAGCACCACCGGCGGCAACTACAACTACCTGTAC
AGAAGCCTGCGGAAGTCTAAGCTGAAGCCCTTCGAGCGGGACATC
AGCAGCGACATCTATAGCCCTGGCGGCAAGCCTTGTTCTGGCGTG
GAAGGCTTCAACTGCTACTACCCTCTGCGGAGCTACGGCTTCTTC
CCCACAAATGGCACAGGCTACCAGCCTTACAGAGTGGTGGTCCTG
AGCTTCGAGCTGCTGAATGCCCCTGCCACAGTGTGTGGCCCTAAG
CTGTCTACAGATGGGGGGGGAGGATCTGGCGGAGGTGGAAGCGGA
GGCGGAGGAAGCGGTGGGGGGGGATCTAAATCTTCTATCGCCAGC
TTCTTCTTCATCATCGGCCTGATTATCGGCCTGTTCCTGGTGCTG
AGAGTGGGCATCCACCTGTGCATCAAGCTGAAACACACCAAGAAG
CGGCAAATCTACACCGACATCGAGATGAACCGGCTGGGCAAA

1.-29. (canceled)

30. An isolated polypeptide, which comprises an amino acid sequence of SEQ ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEQ ID NO:31.

31. A polypeptide according to claim 30, which comprises at least one, or all of the amino acid residues, optionally at least five, at least ten, or at least fifteen of the amino acid residues, at positions corresponding to the amino acid residue positions of SEQ ID NO:11, as shown in the table below:

| SARS2 RBD | |
|---|---|
| residue | |
| position | |
| (CoV_T2_6; | |
| SEQ ID | |
| NO: 11) | Residue |
| 3 | A |
| 6 | K |
| 7 | E |
| 8 | V |
| 30 | P |
| 36 | E |
| 54 | T |
| 120 | T |
| 126 | T |
| 127 | T |
| 152 | S |
| 153 | D |
| 163 | S |
| 201 | N |
| 211 | L |
| 214 | D |

32. (canceled)

33. A polypeptide according to claim 30, which comprises at least one, or all of the amino acid residues at positions corresponding to the amino acid residue positions of SEQ ID NO:11, as shown in the table below:

| SARS2 RBD | |
|---|---|
| residue | |
| position | |
| (CoV_T2_6; | |
| SEQ ID | |
| NO: 11) | Residue |
| 99 | V |
| 137 | S |
| 138 | L |
| 142 | K |
| 156 | S |
| 157 | P |
| 159 | G |
| 160 | K |
| 172 | Y |
| 175 | R |
| 180 | F |

34. A polypeptide according to claim 30, which comprises at least one, or all of the amino acid residues at positions corresponding to the amino acid residue positions of SEQ ID NO:11, as shown in the table below:

| SARS2 RBD | |
|---|---|
| residue | |
| position | |
| (CoV_T2_6; | |
| SEQ ID | |
| NO: 11) | Residue |
| 28 | K |
| 39 | K |
| 123 | I |

35. A polypeptide according to claim 30, which comprises at least one, or all of the amino acid residues at positions corresponding to the amino acid residue positions of SE ID NO:11 as shown in the table below:

| SARS2 RBD | |
|---|---|
| residue | |
| position | |
| (CoV_T2_6; | |
| SEQ ID | Design |
| NO: 11) | residue |
| 185 | T |

36.-146. (canceled)

147. A polypeptide according to claim 30, which comprises the following discontinuous amino acid sequences:
a)
(i)
(SEQ ID NO: 57)
NITNLCPFGEVENATK;
(ii)
(SEQ ID NO: 58)
KKISN;
(iii)
(SEQ ID NO: 59)
NI;
b)
(i)
(SEQ ID NO: 71)
YNSTSFSTFKCYGVSPTKLNDLCFT;
(ii)
(SEQ ID NO: 72)
DDFT;
iii)
(SEQ ID NO: 62)
FELLN;
or
c)
(i)
(SEQ ID NO: 63)
RGDEVRQ;
(SEQ ID NO: 73
TGVIADY
iii)
(SEQ ID NO: 74)
YRSLRKSK;
(iv)
(SEQ ID NO: 75)
YSPGGK;
(v)
(SEQ ID NO: 77)
FNCYYPLRSYGFFPTNGTGY.

148.-181. (canceled)

182. An isolated nucleic acid molecule encoding a polypeptide according to claim 30, or the complement thereof.

183.-188. (canceled)

189. A vector comprising a nucleic acid molecule of claim 182, optionally which further comprises a promoter operably linked to the nucleic acid.

190.-195. (canceled)

196. A vector according to claim 189, wherein the promoter is for expression of a polypeptide encoded by the nucleic acid in mammalian cells.

197.-199. (canceled)

200. A vector according to claim 189, which is a vaccine vector, preferably a viral vaccine vector, a bacterial vaccine vector, an RNA vaccine vector, or a DNA vaccine vector.

201. (canceled)

202. An isolated cell comprising a vector of claim 189.

203. A fusion protein comprising a polypeptide according to claim 30.

204. A pharmaceutical composition comprising:
a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31;
a nucleic acid molecule encoding a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31, or the complement thereof; or
a vector comprising a nucleic acid molecule encoding a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31, or the complement thereof, optionally which further comprises a promoter operably linked to the nucleic acid;
and a pharmaceutically acceptable carrier, excipient, or diluent.

205.-214. (canceled)

215. A pharmaceutical composition according to claim 204, which further comprises an adjuvant for enhancing an immune response in a subject to the polypeptide, or to a polypeptide encoded by the nucleic acid, of the composition.

216. A pseudotyped virus comprising a polypeptide according to claim 30.

217. A method of inducing an immune response to a coronavirus in a subject, which comprises administering to the subject an effective amount of:
i) a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31;
ii) a nucleic acid molecule encoding a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31, or the complement thereof;
iii) a vector comprising a nucleic acid molecule encoding a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31, or the complement thereof; or
iv) a pharmaceutical composition comprising:
a) a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31, and a pharmaceutically acceptable carrier, excipient, or diluent;
b) a nucleic acid molecule encoding a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31, or the complement thereof, and a pharmaceutically acceptable carrier, excipient, or diluent; or
c) a vector comprising a nucleic acid molecule encoding a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31, or the complement thereof, and a pharmaceutically acceptable carrier, excipient, or diluent.

218. A method of immunising a subject against a coronavirus, which comprises administering to the subject an effective amount of;
i) a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31;
ii) a nucleic acid molecule encoding a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31, or the complement thereof;
iii) a vector comprising a nucleic acid molecule encoding a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31, or the complement thereof, optionally which further comprises a promoter operably linked to the nucleic acid; or
iv) a pharmaceutical composition comprising:
a) a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31, and a pharmaceutically acceptable carrier, excipient, or diluent;
b) a nucleic acid molecule encoding a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31, or the complement thereof, and a pharmaceutically acceptable carrier, excipient, or diluent; or
a vector comprising a nucleic acid molecule encoding a polypeptide which comprises an amino acid sequence of SEO ID NO: 31 (COV_S_T2_17), or an amino acid sequence which has at least 86%, 87%, 88%, 89%, 90%, 91%, 92%, 93%, 94%, 95%, 96%, 97%, 98%, or 99% amino acid identity over its entire length with the amino acid sequence of SEO ID NO:31, or the complement thereof, and a pharmaceutically acceptable carrier, excipient, or diluent.

219.-221. (canceled)

222. A method according to claim 217, wherein the coronavirus is a β-coronavirus, optionally wherein the β-coronavirus is a lineage B or C β-coronavirus.

223. (canceled)

224. A method according to claim 222, wherein the β-coronavirus is a lineage B β-coronavirus, preferably wherein the lineage B β-coronavirus is SARS-CoV or SARS-CoV-2.

225.-226. (canceled)

227. An isolated polypeptide comprising an amino acid sequence with the following discontinuous amino acid sequences:
a)
i)
(SEQ ID NO: 57)
NITNLCPFGEVENATK,
ii)
(SEQ ID NO: 58)
KKISN,
and
iii)
(SEQ ID NO: 59)
NI;
b)
i)
(SEQ ID NO: 71)
YNSTSFSTFKCYGVSPTKLNDLCFT,
ii)
(SEQ ID NO: 72)
DDFT,
and
iii)
(SEQ ID NO: 62)
FELLN;
or
c)
i)
(SEQ ID NO: 63)
RGDEVRQ,
ii)
(SEQ ID NO: 73)
TGVIADY,
iii)
(SEQ ID NO: 74)
YRSLRKSK,
iv)
(SEQ ID NO: 75)
YSPGGK,
and
v)
(SEQ ID NO: 77)
FNCYYPLRSYGFFPTNGTGY.


SEQUENCE LISTING (85 sequences)

SEQ ID NO: 1 - protein, 1255 aa, Artificial Sequence
  Coronavirus wild-type S-protein sequence
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - DNA, 3765 bp, Artificial Sequence
  Coronavirus wild-type S-protein sequence
atgtttatctttctgctgtttctgaccctgaccagcggcagcgacctggatagatgcaccaccttcgacgatgtgcaggcccctaactacacccagcacaccagctctatgcggggcgtgtactaccccgacgagattttcagaagcgacaccctgtatctgacccaggacctgttcctgcctttctacagcaacgtgaccggcttccacaccatcaaccacaccttcggcaaccctgtgatccccttcaaggacggcatctactttgccgccaccgagaagtccaacgtcgtcagaggatgggtgttcggcagcaccatgaacaacaagagccagagcgtgatcatcatcaacaacagcaccaacgtggtcatccgggcctgcaacttcgagctgtgcgacaacccattcttcgccgtgtccaagcctatgggcacccagacacacaccatgatcttcgacaacgccttcaactgcaccttcgagtacatcagcgacgccttcagcctggacgtgtccgaaaagagcggcaacttcaagcacctgagggaattcgtgttcaagaacaaggatggcttcctgtacgtgtacaagggctaccagcctatcgacgtcgtgcgggatctgcccagcggcttcaataccctgaagcctatcttcaagctgcccctgggcatcaacatcaccaacttcagagccatcctgaccgctttcagccccgctcaggatatctggggaacaagcgccgctgcctacttcgtgggctacctgaagccaaccaccttcatgctgaagtacgacgagaacggcaccatcaccgacgccgtggactgtagccaaaatcctctggccgagctgaagtgcagcgtgaagtccttcgagatcgacaagggcatctaccagaccagcaatttcagagtggtgccctccggggatgtcgtgcggttccccaacatcacaaatctgtgccccttcggcgaggtgttcaacgccaccaagtttccctctgtgtacgcctgggagcgcaaaaagatcagcaactgcgtggccgactacagcgtgctgtacaactccaccttcttcagcaccttcaagtgctacggcgtgtccgccacaaagctgaacgacctgtgcttctccaacgtgtacgccgacagcttcgtggtcaaaggcgacgacgttcggcagattgcccctggacaaacaggcgtgatcgccgattacaactacaagctgcctgacgacttcatgggctgcgtgctggcctggaacaccagaaacatcgatgccacctccaccggcaactacaattacaagtacagatacctgcggcacggcaagctgcggcctttcgagagggatatcagcaatgtgccttttagccccgacggcaagccctgcacacctcctgctctgaattgctactggcccctgaacgactacggcttttacaccaccacaggcatcggctatcagccctatagagtggtggtcctgtcctttgagctgctgaatgcccctgccacagtgtgcggacctaagctgtctaccgacctgatcaagaaccagtgcgtgaacttcaacttcaacggcctgaccggcaccggcgtgctgacaccaagcagcaagagattccagcctttccagcagttcggccgggatgtgtccgacttcacagacagcgtcagagatcccaagaccagcgagatcctggacatcagcccttgtgcctttggcggagtgtccgtgatcacccctggcacaaatgcctctagcgaagtggccgtgctgtatcaggacgtgaactgcaccgatgtgtccaccgccattcacgccgatcagctgactcccgcttggcggatctatagcacaggcaacaacgtgttccagacacaagccggctgtctgatcggagccgagcatgtggataccagctacgagtgcgacatccctatcggcgctggcatctgtgcctcttaccacaccgtgtctctgctgcggagcaccagccagaaatccatcgtggcctacaccatgagcctgggcgccgattcttctatcgcctactccaacaacacaatcgctatccccaccaatttcagcatctccatcaccaccgaagtgatgcccgtgtccatggccaagacctccgtggattgcaacatgtacatctgcggcgacagcaccgagtgcgccaatctgctgctccagtacggcagcttctgcacccagctgaatagagccctgtctggaattgccgccgagcaggacagaaacaccagagaagtgttcgcccaagtgaagcagatgtataagaccccgacactcaagtacttcggcgggttcaacttctcccagatcctgcctgatcctctgaagcccaccaagcggagcttcatcgaggacctgctgttcaacaaagtgaccctggccgacgccggctttatgaagcagtatggcgagtgcctgggcgacatcaacgccagggatctgatttgcgcccagaagtttaacggactgaccgtgctgcctcctctgctgaccgatgatatgatcgccgcctacacagccgctctggtgtctggtacagctaccgccggatggacatttggagctggcgccgctctccagattccattcgctatgcagatggcctaccggttcaacggcatcggagtgacccagaatgtgctgtacgagaatcagaagcagatcgccaatcagttcaacaaggccatcagccagatccaagagagcctgaccaccacaagcacagccctgggaaagctccaggacgtggtcaaccagaatgctcaggccctgaacaccctggtcaagcagctgagcagcaacttcggcgccatcagctccgtgctgaatgacatcctgagccggctggacaaggtggaagcagaggtgcagatcgaccggctgatcacaggcagactccagagcctccagacctacgtgacacagcagctgatcagagccgccgagattagagcctctgccaatctggccgccaccaaaatgagcgagtgtgtcctgggccagagcaagagagtggacttttgcggcaagggctatcacctgatgagcttcccacaggccgctcctcatggcgtggtctttctgcacgtgacatacgtgcccagccaagagagaaacttcaccaccgctccagccatctgccacgagggcaaagcctactttcccagagaaggcgtgttcgtgtttaacggcacctcctggtttatcacccagcggaatttcttcagcccgcaaatcatcaccacagacaacaccttcgtgtccggcaactgtgacgtcgtgatcggcatcattaacaataccgtgtacgaccctctccagcctgagctggacagcttcaaagaggaactggataagtacttcaagaatcacacgagccccgatgtggacctgggcgatatctctggcatcaatgccagcgtcgtgaacatccagaaagagattgacaggctgaacgaggtggccaagaacctgaacgagtccctgatcgacctgcaagagctggggaagtacgagcagtacatcaagtggccttggtacgtgtggctgggctttatcgccggactgatcgccatcgtgatggtcaccatcctgctgtgctgcatgaccagctgttgcagctgtctgaagggcgcctgtagctgtggctcctgctgcaagttcgatgaggacgactctgagccagtgctgaaaggcgtgaagctgcactacacc

SEQ ID NO: 3 - protein, 1152 aa, Artificial Sequence
  Coronavirus wild-type trS-protein sequence
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDIS

SEQ ID NO: 4 - DNA, 3456 bp, Artificial Sequence
  Coronavirus wild-type tr-S-protein sequence
atgtttatctttctgctgtttctgaccctgaccagcggcagcgacctggatagatgcaccaccttcgacgatgtgcaggcccctaactacacccagcacaccagctctatgcggggcgtgtactaccccgacgagattttcagaagcgacaccctgtatctgacccaggacctgttcctgcctttctacagcaacgtgaccggcttccacaccatcaaccacaccttcggcaaccctgtgatccccttcaaggacggcatctactttgccgccaccgagaagtccaacgtcgtcagaggatgggtgttcggcagcaccatgaacaacaagagccagagcgtgatcatcatcaacaacagcaccaacgtggtcatccgggcctgcaacttcgagctgtgcgacaacccattcttcgccgtgtccaagcctatgggcacccagacacacaccatgatcttcgacaacgccttcaactgcaccttcgagtacatcagcgacgccttcagcctggacgtgtccgaaaagagcggcaacttcaagcacctgagggaattcgtgttcaagaacaaggatggcttcctgtacgtgtacaagggctaccagcctatcgacgtcgtgcgggatctgcccagcggcttcaataccctgaagcctatcttcaagctgcccctgggcatcaacatcaccaacttcagagccatcctgaccgctttcagccccgctcaggatatctggggaacaagcgccgctgcctacttcgtgggctacctgaagccaaccaccttcatgctgaagtacgacgagaacggcaccatcaccgacgccgtggactgtagccaaaatcctctggccgagctgaagtgcagcgtgaagtccttcgagatcgacaagggcatctaccagaccagcaatttcagagtggtgccctccggggatgtcgtgcggttccccaacatcacaaatctgtgccccttcggcgaggtgttcaacgccaccaagtttccctctgtgtacgcctgggagcgcaaaaagatcagcaactgcgtggccgactacagcgtgctgtacaactccaccttcttcagcaccttcaagtgctacggcgtgtccgccacaaagctgaacgacctgtgcttctccaacgtgtacgccgacagcttcgtggtcaaaggcgacgacgttcggcagattgcccctggacaaacaggcgtgatcgccgattacaactacaagctgcctgacgacttcatgggctgcgtgctggcctggaacaccagaaacatcgatgccacctccaccggcaactacaattacaagtacagatacctgcggcacggcaagctgcggcctttcgagagggatatcagcaatgtgccttttagccccgacggcaagccctgcacacctcctgctctgaattgctactggcccctgaacgactacggcttttacaccaccacaggcatcggctatcagccctatagagtggtggtcctgtcctttgagctgctgaatgcccctgccacagtgtgcggacctaagctgtctaccgacctgatcaagaaccagtgcgtgaacttcaacttcaacggcctgaccggcaccggcgtgctgacaccaagcagcaagagattccagcctttccagcagttcggccgggatgtgtccgacttcacagacagcgtcagagatcccaagaccagcgagatcctggacatcagcccttgtgcctttggcggagtgtccgtgatcacccctggcacaaatgcctctagcgaagtggccgtgctgtatcaggacgtgaactgcaccgatgtgtccaccgccattcacgccgatcagctgactcccgcttggcggatctatagcacaggcaacaacgtgttccagacacaagccggctgtctgatcggagccgagcatgtggataccagctacgagtgcgacatccctatcggcgctggcatctgtgcctcttaccacaccgtgtctctgctgcggagcaccagccagaaatccatcgtggcctacaccatgagcctgggcgccgattcttctatcgcctactccaacaacacaatcgctatccccaccaatttcagcatctccatcaccaccgaagtgatgcccgtgtccatggccaagacctccgtggattgcaacatgtacatctgcggcgacagcaccgagtgcgccaatctgctgctccagtacggcagcttctgcacccagctgaatagagccctgtctggaattgccgccgagcaggacagaaacaccagagaagtgttcgcccaagtgaagcagatgtataagaccccgacactcaagtacttcggcgggttcaacttctcccagatcctgcctgatcctctgaagcccaccaagcggagcttcatcgaggacctgctgttcaacaaagtgaccctggccgacgccggctttatgaagcagtatggcgagtgcctgggcgacatcaacgccagggatctgatttgcgcccagaagtttaacggactgaccgtgctgcctcctctgctgaccgatgatatgatcgccgcctacacagccgctctggtgtctggtacagctaccgccggatggacatttggagctggcgccgctctccagattccattcgctatgcagatggcctaccggttcaacggcatcggagtgacccagaatgtgctgtacgagaatcagaagcagatcgccaatcagttcaacaaggccatcagccagatccaagagagcctgaccaccacaagcacagccctgggaaagctccaggacgtggtcaaccagaatgctcaggccctgaacaccctggtcaagcagctgagcagcaacttcggcgccatcagctccgtgctgaatgacatcctgagccggctggacaaggtggaagcagaggtgcagatcgaccggctgatcacaggcagactccagagcctccagacctacgtgacacagcagctgatcagagccgccgagattagagcctctgccaatctggccgccaccaaaatgagcgagtgtgtcctgggccagagcaagagagtggacttttgcggcaagggctatcacctgatgagcttcccacaggccgctcctcatggcgtggtctttctgcacgtgacatacgtgcccagccaagagagaaacttcaccaccgctccagccatctgccacgagggcaaagcctactttcccagagaaggcgtgttcgtgtttaacggcacctcctggtttatcacccagcggaatttcttcagcccgcaaatcatcaccacagacaacaccttcgtgtccggcaactgtgacgtcgtgatcggcatcattaacaataccgtgtacgaccctctccagcctgagctggacagcttcaaagaggaactggataagtacttcaagaatcacacgagccccgatgtggacctgggcgatatctct

SEQ ID NO: 5 - protein, 213 aa, Artificial Sequence
  Coronavirus wild-type S-protein RBD sequence
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTD

SEQ ID NO: 6 - DNA, 639 bp, Artificial Sequence
  Coronavirus wild-type S-protein RBD sequence
agagtggtgccctccggggatgtcgtgcggttccccaacatcacaaatctgtgccccttcggcgaggtgttcaacgccaccaagtttccctctgtgtacgcctgggagcgcaaaaagatcagcaactgcgtggccgactacagcgtgctgtacaactccaccttcttcagcaccttcaagtgctacggcgtgtccgccacaaagctgaacgacctgtgcttctccaacgtgtacgccgacagcttcgtggtcaaaggcgacgacgttcggcagattgcccctggacaaacaggcgtgatcgccgattacaactacaagctgcctgacgacttcatgggctgcgtgctggcctggaacaccagaaacatcgatgccacctccaccggcaactacaattacaagtacagatacctgcggcacggcaagctgcggcctttcgagagggatatcagcaatgtgccttttagccccgacggcaagccctgcacacctcctgctctgaattgctactggcccctgaacgactacggcttttacaccaccacaggcatcggctatcagccctatagagtggtggtcctgtcctttgagctgctgaatgcccctgccacagtgtgcggacctaagctgtctaccgac

SEQ ID NO: 7 - protein, 1273 aa, Artificial Sequence
  Coronavirus wild-type S-protein sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - DNA, 3819 bp, Artificial Sequence
  Coronavirus wild-type S-protein sequence
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaaaatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactacgtgggatacctccagccaagaaccttcctgctgaagtacaacgagaacggcaccatcaccgacgccgtggattgtgctctggaccctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaacggcgtggaaggcttcaactgctacttcccactgcaaagctacggctttcagcccacaaatggcgtgggctaccagccttacagagtggtggtgctgagcttcgagctgctgcatgctcctgccacagtgtgcggccctaagaaatccaccaatctcgtgaagaacaaatgcgtgaacttcaacttcaacggcctgaccggcaccggcgtgctgacagagagcaacaagaagttcctgccattccagcagttcggccgggatatcgccgataccacagatgccgtcagagatccccagacactggaaatcctggacatcaccccatgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgcgcctcttaccagacacagacaaacagccccagacgggctagaagcgtggccagccagagcatcattgcctacacaatgtctctgggcgccgagaacagcgtggcctactccaacaactctatcgctatccccaccaacttcaccatcagcgtgaccaccgagatcctgcctgtgtccatgaccaagaccagcgtggactgcaccatgtacatctgcggcgattccaccgagtgctccaacctgctgctccagtacggcagcttctgcacccagctgaatagagccctgacagggatcgccgtggaacaggacaagaacacccaagaggtgttcgcccaagtgaagcaaatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtacggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctccagattccattcgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctccaggacgtcgtgaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggcagactccagtctctccagacctacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgttgtgatcggcattgtgaacaataccgtgtacgaccctctccagcctgaactggactccttcaaagaggaactcgacaagtactttaagaaccacacaagccccgacgtggacctgggcgatatcagcggaatcaatgccagcgtggtcaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggctcctgctgcaagttcgacgaggacgattctgagcccgtgctgaagggcgtgaaactgcactacacc

SEQ ID NO: 9 - protein, 1170 aa, Artificial Sequence
  Coronavirus wild-type trS-protein sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDIS

SEQ ID NO: 10 - DNA, 3510 bp, Artificial Sequence
  Coronavirus wild-type trS-protein sequence
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaaaatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactacgtgggatacctccagccaagaaccttcctgctgaagtacaacgagaacggcaccatcaccgacgccgtggattgtgctctggaccctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaacggcgtggaaggcttcaactgctacttcccactgcaaagctacggctttcagcccacaaatggcgtgggctaccagccttacagagtggtggtgctgagcttcgagctgctgcatgctcctgccacagtgtgcggccctaagaaatccaccaatctcgtgaagaacaaatgcgtgaacttcaacttcaacggcctgaccggcaccggcgtgctgacagagagcaacaagaagttcctgccattccagcagttcggccgggatatcgccgataccacagatgccgtcagagatccccagacactggaaatcctggacatcaccccatgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgcgcctcttaccagacacagacaaacagccccagacgggctagaagcgtggccagccagagcatcattgcctacacaatgtctctgggcgccgagaacagcgtggcctactccaacaactctatcgctatccccaccaacttcaccatcagcgtgaccaccgagatcctgcctgtgtccatgaccaagaccagcgtggactgcaccatgtacatctgcggcgattccaccgagtgctccaacctgctgctccagtacggcagcttctgcacccagctgaatagagccctgacagggatcgccgtggaacaggacaagaacacccaagaggtgttcgcccaagtgaagcaaatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtacggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctccagattccattcgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctccaggacgtcgtgaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggcagactccagtctctccagacctacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgttgtgatcggcattgtgaacaataccgtgtacgaccctctccagcctgaactggactccttcaaagaggaactcgacaagtactttaagaaccacacaagccccgacgtggacctgggcgatatcagt

SEQ ID NO: 11 - protein, 214 aa, Artificial Sequence
  Coronavirus wild-type S-protein RBD sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 12 - DNA, 642 bp, Artificial Sequence
  Coronavirus S-protein RBD sequence
cgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaacggcgtggaaggcttcaactgctacttcccactgcaaagctacggctttcagcccacaaatggcgtgggctaccagccttacagagtggtggtgctgagcttcgagctgctgcatgctcctgccacagtgtgcggccctaagaaatccaccaat

SEQ ID NO: 13 - protein, 1288 aa, Artificial Sequence
  Coronavirus S-protein designed sequence
MFLFLFIIIFAFFLLSAKANERCGIFTSKPQPKLAQVSSSRRGVYYPDDIFRSDVLHLTQDYFLPFDSNVTRYFSLNANGPDRIVYFDNPIIPFKDGVYFAATEKSNVIRGWIFGSTLDNTSQSVIIVNNSTNVIIRVCNFDLCNDPFFTVSRPTDKHIKTWSIREFAVYQSAFNCTFEYVSKSFLLDVAEKPGNFKHLREFVFKNVDGFLNVYSTYKPINVVSGLPTGFSVLKPILKLPLGINITSFRVLLTMFRGDPTPGHTTANWLTAAAAYYVGYLKPTTFMLKYNENGTITDAVDCSQNPLAELKCTLKNFNVDKGIYQTSNFRVSPTQEVVRFPNITNLCPFDKVFNATRFPSVYAWERTKISDCVADYTVLYNSTSFSTFKCYGVSPSKLIDLCFTSVYADTFLIRCSEVRQVAPGQTGVIADYNYKLPDDFTGCVIAWNTAKQDTGSSGNYNYYYRSHRKTKLKPFERDLSSDECSPDGKPCTPPAFNGVRGFNCYFTLSTYDFNPNVPVEYQATRVVVLSFELLNAPATVCGPKLSTQLVKNQCVNFNFNGLKGTGVLTASSKRFQSFQQFGRDASDFTDSVRDPQTLEILDISPCSFGGVSVITPGTNTSSEVAVLYQDVNCTDVPTAIHADQLTPAWRVYSTGVNVFQTQAGCLIGAEHVNASYECDIPIGAGICASYHTASNSPRILRSTGQKSIVAYTMSLGAENSIAYANNSIAIPTNFSISVTTEVMPVSMAKTSVDCTMYICGDSLECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMATILLCCMTSCCSCLKGACSCGSCCKFDEDDSEPVLKGVKLHYT

SEQ ID NO: 14 - DNA, 3864 bp, Artificial Sequence
  Coronavirus S-protein designed sequence
atgtttctgttcctcttcattattatcttcgcattcttcctgctgagcgccaaggccaacgagagatgcggcatcttcaccagcaagccccagcctaagctggcccaggtgtccagttctagacggggcgtgtactaccccgacgacatcttcagatccgacgtgctgcatctgacccaggactacttcctgcctttcgacagcaacgtgacccggtacttcagcctgaacgccaacggacccgaccggatcgtgtacttcgacaaccctatcatccccttcaaggacggggtgtactttgccgccaccgagaagtccaacgtgatcagaggctggatcttcggcagcaccctggacaataccagccagagcgtgatcatcgtgaacaacagcaccaacgtcatcatccgcgtgtgcaacttcgacctgtgcaacgacccattcttcaccgtgtccagaccaaccgacaagcacatcaagacctggtccatccgcgagttcgccgtgtaccagagcgccttcaattgcaccttcgagtacgtgtccaagagctttctgctggacgtggccgagaagcccggcaactttaagcacctgagagaattcgtgttcaagaacgtggacggcttcctgaacgtgtacagcacctacaagcccatcaacgtggtgtccggcctgcctacaggattcagcgtgctgaagcccatcctgaagctgcccctgggcatcaacatcaccagcttcagagtgctgctgaccatgttcagaggcgaccctacacctggccacaccaccgctaattggctgacagccgccgctgcctactacgtgggatacctgaagcctaccaccttcatgctcaagtacaacgagaacggcaccatcaccgacgccgtggactgtagccaaaatcctctggccgagctgaagtgcaccctgaagaacttcaacgtggacaagggcatctaccagaccagcaacttccgggtgtcccctacacaagaggtcgtgcggttccccaatatcaccaatctgtgccccttcgacaaggtgttcaacgccaccagatttcccagcgtgtacgcctgggagcgcaccaagatttccgattgcgtggccgactacaccgtgctgtataactccacctccttcagcaccttcaagtgctacggcgtgtccccaagcaagctgatcgatctgtgcttcacctctgtgtacgccgacaccttcctgatccggtgtagcgaagtgcgacaggtggcacctggacagacaggcgtgatcgccgattacaactacaagctgcccgacgacttcaccggctgtgtgatcgcctggaataccgccaagcaggatacaggcagcagcggcaactacaactactactacagaagccaccgcaagaccaagctgaagcctttcgagagggacctgagcagcgacgagtgtagccctgatggcaagccttgtacacctcctgccttcaatggcgtgcggggcttcaactgctacttcaccctgagcacctacgacttcaaccccaacgtgcccgtggaataccaggccacaagagtggtggtgctgagcttcgagctgctgaatgcccctgccacagtgtgtggccctaagctgtctacccagctggtcaagaaccagtgcgtgaacttcaatttcaacggcctgaaaggcaccggcgtgctgaccgccagcagcaagagattccagagcttccagcagttcggcagggacgccagcgatttcacagatagcgtcagagatccccagacactggaaatcctggacatcagcccttgcagcttcggcggagtgtctgtgatcacccctggcaccaatacctctagcgaggtggcagtgctgtaccaggacgtgaactgcaccgatgtgcctacagccatccacgccgatcagctgacaccagcttggagagtgtactctaccggtgtcaacgtgttccagacacaagccggctgtctgattggagccgaacacgtgaacgccagctacgagtgcgacatccctatcggagccggcatctgtgcctcttaccacaccgcctctaacagccccagaatcctgagaagcaccggccagaaatccatcgtggcctacacaatgtctctgggcgccgagaactctatcgcctacgccaacaactccattgctatccccaccaacttcagcatctccgtgaccaccgaagtgatgcctgtgtccatggccaagaccagcgtggactgcacaatgtacatctgcggcgacagcctggaatgcagcaacctgctgctccagtacggcagcttctgcacccagctgaatagagccctgaccggaatcgccatcgagcaggacaagaacacccaagaggtgttcgcccaagtgaagcagatgtataagacccctgccatcaaggacttcggcggctttaacttcagccagatcctgcctgatcctagcaagcccaccaagcggagcttcatcgaggacctgctgttcaacaaagtgaccctggccgacgccggctttatgaagcagtatggcgagtgcctgggcgacatctctgccagggatctgatttgcgcccagaagttcaacggactgaccgtgctgcctcctctgctgaccgatgagatgatcgccgcctatacagccgctctggtgtctggcacagctaccgccggatggacatttggagctggcgccgctctccagattccattcgctatgcagatggcctaccgcttcaacggcatcggcgtgacccagaacgtgctgtacgagaaccagaagcagatcgccaaccagttcaacaaggccatcagtcagatccaagagagcctgaccacaaccagcacagccctgggaaagctccaggacgtcgtgaaccagaatgcccaggctctgaacaccctggtcaagcagctgagcagcaatttcggcgccatcagctccgtgctgaacgacatcctgagccggctggataaggtggaagccgaggtgcagatcgaccggctgattacaggcagactccagtctctccagacctacgtgacacagcagctgatcagagccgccgagattagagcctctgccaatctggccgccaccaagatgtctgagtgtgtgctgggccagtctaagagagtggacttctgcggcaagggctaccacctgatgagcttccctcaggctgctcctcacggcgtggtgtttctgcacgtgacatacgtgcccagccaagagcggaacttcacaactgccccagccatctgccacgagggcaaagcctactttcccagagaaggcgtgttcgtgtccaacggcacctcctggttcatcacccagagaaacttctacagccctcagatcatcaccaccgacaacaccttcgtgtccggcaactgcgacgtggtcatcggcatcatcaacaataccgtgtacgaccctctccagccagaactggatagcttcaaagaggaactcgacaagtacttcaagaatcacacaagccccgacgtggacctgggcgatatcagcggaatcaatgccagcgtggtcaacatccagaaagagatcgacagactgaacgaggtggccaagaacctgaacgagtccctgatcgacctgcaagagctggggaagtacgagcagtacatcaagtggccttggtacgtgtggctgggctttatcgccggactgatcgccattgtgatggccaccatcctgctgtgctgcatgacaagctgctgtagctgcctgaagggcgcctgtagctgtggcagctgctgcaagttcgacgaggacgattctgagcctgtgctgaaaggcgtgaagctgcactacacc

SEQ ID NO: 15 - protein, 1185 aa, Artificial Sequence
  Coronavirus trS-protein designed sequence
MFLFLFIIIFAFFLLSAKANERCGIFTSKPQPKLAQVSSSRRGVYYPDDIFRSDVLHLTQDYFLPFDSNVTRYFSLNANGPDRIVYFDNPIIPFKDGVYFAATEKSNVIRGWIFGSTLDNTSQSVIIVNNSTNVIIRVCNFDLCNDPFFTVSRPTDKHIKTWSIREFAVYQSAFNCTFEYVSKSFLLDVAEKPGNFKHLREFVFKNVDGFLNVYSTYKPINVVSGLPTGFSVLKPILKLPLGINITSFRVLLTMFRGDPTPGHTTANWLTAAAAYYVGYLKPTTFMLKYNENGTITDAVDCSQNPLAELKCTLKNFNVDKGIYQTSNFRVSPTQEVVRFPNITNLCPFDKVFNATRFPSVYAWERTKISDCVADYTVLYNSTSFSTFKCYGVSPSKLIDLCFTSVYADTFLIRCSEVRQVAPGQTGVIADYNYKLPDDFTGCVIAWNTAKQDTGSSGNYNYYYRSHRKTKLKPFERDLSSDECSPDGKPCTPPAFNGVRGFNCYFTLSTYDFNPNVPVEYQATRVVVLSFELLNAPATVCGPKLSTQLVKNQCVNFNFNGLKGTGVLTASSKRFQSFQQFGRDASDFTDSVRDPQTLEILDISPCSFGGVSVITPGTNTSSEVAVLYQDVNCTDVPTAIHADQLTPAWRVYSTGVNVFQTQAGCLIGAEHVNASYECDIPIGAGICASYHTASNSPRILRSTGQKSIVAYTMSLGAENSIAYANNSIAIPTNFSISVTTEVMPVSMAKTSVDCTMYICGDSLECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVSGNCDVVIGIINNTVYDPLQPELDSFKEELDKYFKNHTSPDVDLGDIS

SEQ ID NO: 16 - DNA, 3555 bp, Artificial Sequence
  Coronavirus trS-protein designed sequence
atgtttctgttcctcttcattattatcttcgcattcttcctgctgagcgccaaggccaacgagagatgcggcatcttcaccagcaagccccagcctaagctggcccaggtgtccagttctagacggggcgtgtactaccccgacgacatcttcagatccgacgtgctgcatctgacccaggactacttcctgcctttcgacagcaacgtgacccggtacttcagcctgaacgccaacggacccgaccggatcgtgtacttcgacaaccctatcatccccttcaaggacggggtgtactttgccgccaccgagaagtccaacgtgatcagaggctggatcttcggcagcaccctggacaataccagccagagcgtgatcatcgtgaacaacagcaccaacgtcatcatccgcgtgtgcaacttcgacctgtgcaacgacccattcttcaccgtgtccagaccaaccgacaagcacatcaagacctggtccatccgcgagttcgccgtgtaccagagcgccttcaattgcaccttcgagtacgtgtccaagagctttctgctggacgtggccgagaagcccggcaactttaagcacctgagagaattcgtgttcaagaacgtggacggcttcctgaacgtgtacagcacctacaagcccatcaacgtggtgtccggcctgcctacaggattcagcgtgctgaagcccatcctgaagctgcccctgggcatcaacatcaccagcttcagagtgctgctgaccatgttcagaggcgaccctacacctggccacaccaccgctaattggctgacagccgccgctgcctactacgtgggatacctgaagcctaccaccttcatgctcaagtacaacgagaacggcaccatcaccgacgccgtggactgtagccaaaatcctctggccgagctgaagtgcaccctgaagaacttcaacgtggacaagggcatctaccagaccagcaacttccgggtgtcccctacacaagaggtcgtgcggttccccaatatcaccaatctgtgccccttcgacaaggtgttcaacgccaccagatttcccagcgtgtacgcctgggagcgcaccaagatttccgattgcgtggccgactacaccgtgctgtataactccacctccttcagcaccttcaagtgctacggcgtgtccccaagcaagctgatcgatctgtgcttcacctctgtgtacgccgacaccttcctgatccggtgtagcgaagtgcgacaggtggcacctggacagacaggcgtgatcgccgattacaactacaagctgcccgacgacttcaccggctgtgtgatcgcctggaataccgccaagcaggatacaggcagcagcggcaactacaactactactacagaagccaccgcaagaccaagctgaagcctttcgagagggacctgagcagcgacgagtgtagccctgatggcaagccttgtacacctcctgccttcaatggcgtgcggggcttcaactgctacttcaccctgagcacctacgacttcaaccccaacgtgcccgtggaataccaggccacaagagtggtggtgctgagcttcgagctgctgaatgcccctgccacagtgtgtggccctaagctgtctacccagctggtcaagaaccagtgcgtgaacttcaatttcaacggcctgaaaggcaccggcgtgctgaccgccagcagcaagagattccagagcttccagcagttcggcagggacgccagcgatttcacagatagcgtcagagatccccagacactggaaatcctggacatcagcccttgcagcttcggcggagtgtctgtgatcacccctggcaccaatacctctagcgaggtggcagtgctgtaccaggacgtgaactgcaccgatgtgcctacagccatccacgccgatcagctgacaccagcttggagagtgtactctaccggtgtcaacgtgttccagacacaagccggctgtctgattggagccgaacacgtgaacgccagctacgagtgcgacatccctatcggagccggcatctgtgcctcttaccacaccgcctctaacagccccagaatcctgagaagcaccggccagaaatccatcgtggcctacacaatgtctctgggcgccgagaactctatcgcctacgccaacaactccattgctatccccaccaacttcagcatctccgtgaccaccgaagtgatgcctgtgtccatggccaagaccagcgtggactgcacaatgtacatctgcggcgacagcctggaatgcagcaacctgctgctccagtacggcagcttctgcacccagctgaatagagccctgaccggaatcgccatcgagcaggacaagaacacccaagaggtgttcgcccaagtgaagcagatgtataagacccctgccatcaaggacttcggcggctttaacttcagccagatcctgcctgatcctagcaagcccaccaagcggagcttcatcgaggacctgctgttcaacaaagtgaccctggccgacgccggctttatgaagcagtatggcgagtgcctgggcgacatctctgccagggatctgatttgcgcccagaagttcaacggactgaccgtgctgcctcctctgctgaccgatgagatgatcgccgcctatacagccgctctggtgtctggcacagctaccgccggatggacatttggagctggcgccgctctccagattccattcgctatgcagatggcctaccgcttcaacggcatcggcgtgacccagaacgtgctgtacgagaaccagaagcagatcgccaaccagttcaacaaggccatcagtcagatccaagagagcctgaccacaaccagcacagccctgggaaagctccaggacgtcgtgaaccagaatgcccaggctctgaacaccctggtcaagcagctgagcagcaatttcggcgccatcagctccgtgctgaacgacatcctgagccggctggataaggtggaagccgaggtgcagatcgaccggctgattacaggcagactccagtctctccagacctacgtgacacagcagctgatcagagccgccgagattagagcctctgccaatctggccgccaccaagatgtctgagtgtgtgctgggccagtctaagagagtggacttctgcggcaagggctaccacctgatgagcttccctcaggctgctcctcacggcgtggtgtttctgcacgtgacatacgtgcccagccaagagcggaacttcacaactgccccagccatctgccacgagggcaaagcctactttcccagagaaggcgtgttcgtgtccaacggcacctcctggttcatcacccagagaaacttctacagccctcagatcatcaccaccgacaacaccttcgtgtccggcaactgcgacgtggtcatcggcatcatcaacaataccgtgtacgaccctctccagccagaactggatagcttcaaagaggaactcgacaagtacttcaagaatcacacaagccccgacgtggacctgggcgatatcagt

SEQ ID NO: 17 - protein, 219 aa, Artificial Sequence
  Coronavirus S-protein RBD designed sequence
RVSPTQEVVRFPNITNLCPFDKVFNATRFPSVYAWERTKISDCVADYTVLYNSTSFSTFKCYGVSPSKLIDLCFTSVYADTFLIRCSEVRQVAPGQTGVIADYNYKLPDDFTGCVIAWNTAKQDTGSSGNYNYYYRSHRKTKLKPFERDLSSDECSPDGKPCTPPAFNGVRGFNCYFTLSTYDFNPNVPVEYQATRVVVLSFELLNAPATVCGPKLSTQ

SEQ ID NO: 18 - DNA, 657 bp, Artificial Sequence
  Coronavirus S-protein RBD designed sequence
cgggtgtcccctacacaagaggtcgtgcggttccccaatatcaccaatctgtgccccttcgacaaggtgttcaacgccaccagatttcccagcgtgtacgcctgggagcgcaccaagatttccgattgcgtggccgactacaccgtgctgtataactccacctccttcagcaccttcaagtgctacggcgtgtccccaagcaagctgatcgatctgtgcttcacctctgtgtacgccgacaccttcctgatccggtgtagcgaagtgcgacaggtggcacctggacagacaggcgtgatcgccgattacaactacaagctgcccgacgacttcaccggctgtgtgatcgcctggaataccgccaagcaggatacaggcagcagcggcaactacaactactactacagaagccaccgcaagaccaagctgaagcctttcgagagggacctgagcagcgacgagtgtagccctgatggcaagccttgtacacctcctgccttcaatggcgtgcggggcttcaactgctacttcaccctgagcacctacgacttcaaccccaacgtgcccgtggaataccaggccacaagagtggtggtgctgagcttcgagctgctgaatgcccctgccacagtgtgtggccctaagctgtctacccag

SEQ ID NO: 19 - DNA, 91 bp, Artificial Sequence
  pEVAC Multiple Cloning Site sequence
acagactgttcctttccatgggtcttttctgcagtcaccgtcggtaccgtcgacacgtgtgatcatctagaggatccgcggccgcagatct

SEQ ID NO: 20 - DNA, 4405 bp, Artificial Sequence
  pEVAC sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgtcgacacgtgtgatcatctagaggatccgcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 21 - protein, 76 aa, Artificial Sequence
  SARS envelope protein sequence
MYSFVSEETGTLIVNSVLLFLAFVVFLLVTLAILTALRLCAYCCNIVNVSLVKPTVYVYSRVKNLNSSEGVPDLLV

SEQ ID NO: 22 - protein, 76 aa, Artificial Sequence
  Coronavirus envelope protein designed sequence
MYSFVSEETGTLIVNSVLLFLAFVVFLLVTLAILTALRLCAYCCNIVNVSLVKPTFYVYSRVKNLNSSQGVPDLLV

SEQ ID NO: 23 - protein, 75 aa, Artificial Sequence
  Coronavirus envelope protein designed sequence
MYSFVSEETGTLIVNSVLLFLAFVVFLLVTLAILTALRLCAYCCNIVNVSLVKPTFYVYSRVKNLNSSRVPDLLV

SEQ ID NO: 24 - protein, 221 aa, Artificial Sequence
  Coronavirus M protein designed sequence
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTILTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 25 - protein, 222 aa, Artificial Sequence
  Coronavirus M protein designed sequence
MADSNGTITVEELKKLLEQWNLVIGFLFLTWICLLQFAYSNRNRFLYIIKLIFLWLLWPVTLACFVLAAVYRINWVTGGIAIAMACIVGLMWLSYFVASFRLFARTRSMWSFNPETNILLNVPLRGSIITRPLMESELVIGAVILRGHLRMAGHSLGRCDIKDLPKEITVATSRTLSYYKLGASQRVASDSGFAVYNRYRIGNYKLNTDHSSSSDNIALLVQ

SEQ ID NO: 26 - protein, 222 aa, Artificial Sequence
  Coronavirus M protein reference sequence
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 27 - protein, 214 aa, Artificial Sequence
  Coronavirus S-protein RBD designed sequence
RVAPTKEVVRFPNITNLCPFGEVFNATRFPSVYAWERKRISNCVADYSVLYNSTSFSTFKCYGVSPTKLNDLCFTNVYADSFVIRGDEVRQIAPGQTGVIADYNYKLPDDFTGCVIAWNTNNLDSTTGGNYNYLYRSLRKSKLKPFERDISSDIYSPGGKPCSGVEGFNCYYPLRSYGFFPTNGVGYQPYRVVVLSFELLNAPATVCGPKLSTD

SEQ ID NO: 28 - protein, 214 aa, Artificial Sequence
  Coronavirus S-protein RBD designed sequence
RVAPTKEVVRFPNITNLCPFGEVFNATKFPSVYAWERKKISNCVADYSVLYNSTSFSTFKCYGVSPTKLNDLCFTNVYADSFVIRGDEVRQIAPGQTGVIADYNYKLPDDFTGCVIAWNTNNIDSTTGGNYNYLYRSLRKSKLKPFERDISSDIYSPGGKPCSGVEGFNCYYPLRSYGFFPTNGVGYQPYRVVVLSFELLNAPATVCGPKLSTD

SEQ ID NO: 29 - protein, 214 aa, Artificial Sequence
  Coronavirus S-protein RBD designed sequence
RVAPTKEVVRFPNITNLCPFGEVFNATRFPSVYAWERKRISNCVADYSVLYNSTFFSTFKCYGVSPTKLNDLCFSNVYADSFVIRGDEVRQIAPGQTGVIADYNYKLPDDFMGCVIAWNTNNLDSTTGGNYNYLYRSLRKSKLKPFERDISSDIYSPGGKPCSGVEGFNCYYPLRSYGFFPTNGVGYQPYRVVVLSFELLNAPATVCGPKLSTD

SEQ ID NO: 30 - protein, 214 aa, Artificial Sequence
  Coronavirus S-protein RBD designed sequence
RVAPTKEVVRFPNITNLCPFGEVFNATRFPSVYAWERKRISNCVADYSVLYNSTSFSTFKCYGVSPTKLNDLCFTNVYADSFVIRGDEVRQIAPGQTGKIADYNYKLPDDFTGCVIAWNTNNLDSTTGGNYNYLYRLFRKSNLKPFERDISSDIYQAGSTPCSGVEGFNCYFPLQSYGFQPTNGVGYQPYRVVVLSFELLNAPATVCGPKLSTD

SEQ ID NO: 31 - protein, 214 aa, Artificial Sequence
  Coronavirus S-protein RBD designed sequence
RVAPTKEVVRFPNITNLCPFGEVFNATKFPSVYAWERKKISNCVADYSVLYNSTSFSTFKCYGVSPTKLNDLCFTNVYADSFVIRGDEVRQIAPGQTGVIADYNYKLPDDFTGCVIAWNTNNIDSTTGGNYNYLYRSLRKSKLKPFERDISSDIYSPGGKPCSGVEGFNCYYPLRSYGFFPTNGTGYQPYRVVVLSFELLNAPATVCGPKLSTD

SEQ ID NO: 32 - protein, 214 aa, Artificial Sequence
  Coronavirus S-protein RBD designed sequence
RVAPTKEVVRFPNITNLCPFGEVFNATRFPSVYAWERKRISNCVADYSVLYNSTFFSTFKCYGVSPTKLNDLCFSNVYADSFVIRGDEVRQIAPGQTGVIADYNYKLPDDFMGCVIAWNTNNLDSTTGGNYNYLYRSLRKSKLKPFERDISSDIYSPGGKPCSGVEGFNCYYPLRSYGFFPTNGTGYQPYRVVVLSFELLNAPATVCGPKLSTD

SEQ ID NO: 33 - protein, 214 aa, Artificial Sequence
  Coronavirus S-protein RBD designed sequence
  MISC_FEATURE (203)..(203): Glycosylation site introduced at this residue
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHANATVCGPKKSTN

SEQ ID NO: 34 - protein, 214 aa, Artificial Sequence
  Coronavirus S-protein RBD designed sequence
  MISC_FEATURE (13)..(13): Glycosylation site deleted at this residue
  MISC_FEATURE (25)..(25): Glycosylation site deleted at this residue
  MISC_FEATURE (54)..(54): Glycosylation site introduced at this residue
RVQPTESIVRFPQITNLCPFGEVFQATRFASVYAWNRKRISNCVADYSVLYNSTSFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 35 - protein, 219 aa, Artificial Sequence
  Coronavirus S-protein RBD designed sequence
  MISC_FEATURE (203)..(203): Glycosylation site introduced at this residue
RVSPTQEVVRFPNITNLCPFDKVFNATRFPSVYAWERTKISDCVADYTVLYNSTSFSTFKCYGVSPSKLIDLCFTSVYADTFLIRCSEVRQVAPGQTGVIADYNYKLPDDFTGCVIAWNTAKQDTGSSGNYNYYYRSHRKTKLKPFERDLSSDECSPDGKPCTPPAFNGVRGFNCYFTLSTYDFNPNVPVEYQATRVVVLSFELLNANATVCGPKLSTQ

SEQ ID NO: 36 - protein, 219 aa, Artificial Sequence
  Coronavirus S-protein RBD designed sequence
  MISC_FEATURE (13)..(13): Glycosylation site deleted at this residue
  MISC_FEATURE (25)..(25): Glycosylation site deleted at this residue
RVSPTQEVVRFPQITNLCPFDKVFQATRFPSVYAWERTKISDCVADYTVLYNSTSFSTFKCYGVSPSKLIDLCFTSVYADTFLIRCSEVRQVAPGQTGVIADYNYKLPDDFTGCVIAWNTAKQDTGSSGNYNYYYRSHRKTKLKPFERDLSSDECSPDGKPCTPPAFNGVRGFNCYFTLSTYDFNPNVPVEYQATRVVVLSFELLNAPATVCGPKLSTQ

SEQ ID NO: 37 - DNA, 642 bp, Artificial Sequence
  Coronavirus S-protein RBD designed sequence
cgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaacggcgtggaaggcttcaactgctacttcccactgcaaagctacggctttcagcccacaaatggcgtgggctaccagccttacagagtggtggtgctgagcttcgagctgctgcatgctaacgccacagtgtgcggccctaagaaatccaccaat

SEQ ID NO: 38 - DNA, 642 bp, Artificial Sequence
  Coronavirus S-protein RBD designed sequence
cgggtgcagcccaccgaatccatcgtgcggttcccccagatcaccaatctgtgccccttcggcgaggtgttccaggccaccagattcgcctctgtgtacgcctggaaccggaagcggatcagcaattgcgtggccgactactccgtgctgtacaactccaccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatcagcaccgaaatctatcaggccggcagcaccccttgcaacggcgtggaaggcttcaactgctacttcccactgcaaagctacggctttcagcccacaaatggcgtgggctaccagccttacagagtggtggtgctgagcttcgagctgctgcatgctcctgccacagtgtgcggccctaagaaatccaccaat

SEQ ID NO: 39 - DNA, 657 bp, Artificial Sequence
  Coronavirus S-protein RBD designed sequence
cgggtgtcccctacacaagaggtcgtgcggttccccaatatcaccaatctgtgccccttcgacaaggtgttcaacgccaccagatttcccagcgtgtacgcctgggagcgcaccaagatttccgattgcgtggccgactacaccgtgctgtataactccacctccttcagcaccttcaagtgctacggcgtgtccccaagcaagctgatcgatctgtgcttcacctctgtgtacgccgacaccttcctgatccggtgtagcgaagtgcgacaggtggcacctggacagacaggcgtgatcgccgattacaactacaagctgcccgacgacttcaccggctgtgtgatcgcctggaataccgccaagcaggatacaggcagcagcggcaactacaactactactacagaagccaccgcaagaccaagctgaagcctttcgagagggacctgagcagcgacgagtgtagccctgatggcaagccttgtacacctcctgccttcaatggcgtgcggggcttcaactgctacttcaccctgagcacctacgacttcaaccccaacgtgcccgtggaataccaggccacaagagtggtggtgctgagcttcgagctgctgaatgccaacgccacagtgtgtggccctaagctgtctacccag

SEQ ID NO: 40 - DNA, 657 bp, Artificial Sequence
  Coronavirus S-protein RBD designed sequence
cgggtgtcccctacacaagaggtcgtgcggttcccccagatcaccaatctgtgccccttcgacaaggtgttccaggccaccagatttcccagcgtgtacgcctgggagcgcaccaagatttccgattgcgtggccgactacaccgtgctgtataactccacctccttcagcaccttcaagtgctacggcgtgtccccaagcaagctgatcgatctgtgcttcacctctgtgtacgccgacaccttcctgatccggtgtagcgaagtgcgacaggtggcacctggacagacaggcgtgatcgccgattacaactacaagctgcccgacgacttcaccggctgtgtgatcgcctggaataccgccaagcaggatacaggcagcagcggcaactacaactactactacagaagccaccgcaagaccaagctgaagcctttcgagagggacctgagcagcgacgagtgtagccctgatggcaagccttgtacacctcctgccttcaatggcgtgcggggcttcaactgctacttcaccctgagcacctacgacttcaaccccaacgtgcccgtggaataccaggccacaagagtggtggtgctgagcttcgagctgctgaatgcccctgccacagtgtgtggccctaagctgtctacccag

SEQ ID NO: 41 - protein, 75 aa, Artificial Sequence
  SARS2 envelope protein reference sequence
MYSFVSEETGTLIVNSVLLFLAFVVFLLVTLAILTALRLCAYCCNIVNVSLVKPSFYVYSRVKNLNSSRVPDLLV

SEQ ID NO: 42 - protein, 75 aa, Artificial Sequence
  Coronavirus envelope protein designed sequence
MYSFVSEETGTLIVASVLLFLAFVVFLLVTLAILTALRLCAYCCNIVNVSLVKPSFYVYSRVKNLNSSRVPDLLV

SEQ ID NO: 43 - protein, 76 aa, Artificial Sequence
  Coronavirus envelope protein designed sequence
MYSFVSEETGTLIVASVLLFLAFVVFLLVTLAILTALRLCAYCCNIVNVSLVKPTFYVYSRVKNLNSSQGVPDLLV

SEQ ID NO: 44 - protein, 75 aa, Artificial Sequence
  Coronavirus envelope protein designed sequence
MYSFVSEETGTLIVASVLLFLAFVVFLLVTLAILTALRLCAYCCNIVNVSLVKPTFYVYSRVKNLNSSRVPDLLV

SEQ ID NO: 45 - protein, 419 aa, Artificial Sequence
  Coronavirus nucleoprotein reference sequence
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 46 - protein, 418 aa, Artificial Sequence
  Coronavirus nucleoprotein designed sequence
MSDNGPQNQRSAPRITFGGPSDSTDNNQNGERSGARPKQRRPQGLPNNTASWFTALTQHGKEDLRFPRGQGVPINTNSGKDDQIGYYRRATRRVRGGDGKMKELSPRWYFYYLGTGPEAALPYGANKEGIVWVATEGALNTPKDHIGTRNPNNNAAIVLQLPQGTTLPKGFYAEGSRGGSQASSRSSSRSRGNSRNSTPGSSRGTSPARMASGGGDTALALLLLDRLNQLESKVSGKGQQQQGQTVTKKSAAEASKKPRQKRTATKQYNVTQAFGRRGPEQTQGNFGDQELIRQGTDYKHWPQIAQFAPSASAFFGMSREVTPSGTWLTYHGAIKLDDKDPQFKDNVILLNKHIDAYKTFPPTEPKKDKKKKADEAQPLPQRQKKQPTVTLLPAADLDDFSKQLQNSMSGASADSTQA

SEQ ID NO: 47 - protein, 417 aa, Artificial Sequence
  Coronavirus nucleoprotein designed sequence
MTDNGQQGPRNAPRITFGVSDNFDNNQDGGRSGARPKQRRPQGLPNNTASWFTALTQHGKEDLRFPRGQGVPINTNSSPDDQIGYYRRATRRIRGGDGKMKDLSPRWYFYYLGTGPEAALPYGANKEGIVWVATEGALNTPKDHIGTRNPNNNAAIVLQLPQGTTLPKGFYAEGSRGGSQASSRSSSRSRNSSRNSTPGSSRGTSPARNLQAGGDTALALLLLDRLNQLESKMSGKGQQQQGQTVTKKSAAEASKKPRQKRTATKQYNVTQAFGRRGPEQTQGNFGDQELIRQGTDYKQWPQIAQFAPSASAFFGMSREVTPSGTWLTYTGAIKLDDKDPQFKDNVILLNKHIDAYKTFPPTEPKKDKKKKADEAQPLPQRQKKQQTVTLLPAADLDDFSRQLQNSMSGASADSTQA

SEQ ID NO: 48 - protein, 166 aa, Artificial Sequence
  Coronavirus membrane protein designed sequence
MADSNGTITVEELKKLLEQITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNGKLNTDHSSSSDNIALLVQ

SEQ ID NO: 49 - protein, 165 aa, Artificial Sequence
  Coronavirus membrane protein designed sequence
MADNGTITVEELKQLLEQVTGGIAIAMACIVGLMWLSYFVASFRLFARTRSMWSFNPETNILLNVPLRGTILTRPLMESELVIGAVIIRGHLRMAGHSLGRCDIKDLPKEITVATSRTLSYYKLGASQRVGTDSGFAAYNRYRIGNGKLNTDHAGSNDNIALLVQ

SEQ ID NO: 50 - protein, 166 aa, Artificial Sequence
  Coronavirus membrane protein designed sequence
MADSNGTITVEELKKLLEQVTGGIAIAMACIVGLMWLSYFVASFRLFARTRSMWSFNPETNILLNVPLRGSIITRPLMESELVIGAVILRGHLRMAGHSLGRCDIKDLPKEITVATSRTLSYYKLGASQRVASDSGFAVYNRYRIGNGKLNTDHSSSSDNIALLVQ

SEQ ID NO: 51 - protein, 262 aa, Artificial Sequence
  Coronavirus S-protein RBD designed protein
MKRGLCCVLLLCGAVFVSPSAARVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNGGSGLNDIFEAQKIEWHEGSHHHHHH

SEQ ID NO: 52 - protein, 1273 aa, Artificial Sequence; identical to SEQ ID NO: 7
  Coronavirus S-protein mutant sequence

SEQ ID NO: 53 - protein, 1270 aa, Artificial Sequence
  Coronavirus S-protein designed sequence
MFVFLVLLPLVSSQCVNFTNRTQLPSAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 54 - protein, 1270 aa, Artificial Sequence
  Coronavirus S-protein designed sequence
MFVFLVLLPLVSSQCVNFTNRTQLPSAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPCQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPCEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 55 - protein, 284 aa, Artificial Sequence
  Designed S protein RBD sequence COV_S_T2_19
RVAPTKEVVRFPNITNLCPFGEVFNATRFPSVYAWERKRISNCVADYSVLYNSTSFSTFKCYGVSPTKLNDLCFTNVYADSFVIRGDEVRQIAPGQTGVIADYNYKLPDDFTGCVIAWNTNNLDSTTGGNYNYLYRSLRKSKLKPFERDISSDIYSPGGKPCSGVEGFNCYYPLRSYGFFPTNGVGYQPYRVVVLSFELLNAPATVCGPKLSTDGGGGSGGGGSGGGGSGGGGSKSSIASFFFIIGLIIGLFLVLRVGIHLCIKLKHTKKRQIYTDIEMNRLGK

SEQ ID NO: 56 - protein, 284 aa, Artificial Sequence
  Designed S protein RBD sequence COV_S_T2_20
RVAPTKEVVRFPNITNLCPFGEVFNATKFPSVYAWERKKISNCVADYSVLYNSTSFSTFKCYGVSPTKLNDLCFTNVYADSFVIRGDEVRQIAPGQTGVIADYNYKLPDDFTGCVIAWNTNNIDSTTGGNYNYLYRSLRKSKLKPFERDISSDIYSPGGKPCSGVEGFNCYYPLRSYGFFPTNGTGYQPYRVVVLSFELLNAPATVCGPKLSTDGGGGSGGGGSGGGGSGGGGSKSSIASFFFIIGLIIGLFLVLRVGIHLCIKLKHTKKRQIYTDIEMNRLGK

SEQ ID NO: 57 - protein, 16 aa, Artificial Sequence
  Part of discontinuous epitope of COV_S_T2_14 and Part of discontinuous epitope of COV_S_T2_17
NITNLCPFGEVFNATK

SEQ ID NO: 58 - protein, 5 aa, Artificial Sequence
  Part of discontinuous epitope of COV_S_T2_14 and COV_S_T2_17
KKISN

SEQ ID NO: 59 - protein, 2 aa, Artificial Sequence
  Part of discontinuous epitope of COV_S_T2_14 and COV_S_T2_17
NI

SEQ ID NO: 60 - protein, 25 aa, Artificial Sequence
  Part of discontinuous epitope of COV_S_T2_15 and COV_S_T2_18
YNSTFFSTFKCYGVSPTKLNDLCFS

SEQ ID NO: 61 - protein, 4 aa, Artificial Sequence
  Part of discontinuous epitope of COV_S_T2_15 and COV_S_T2_18
DDFM

SEQ ID NO: 62 - protein, 5 aa, Artificial Sequence
  Part of discontinuous epitope of COV_S_T2_15 and COV_S_T2_18
FELLN

SEQ ID NO: 63 - protein, 7 aa, Artificial Sequence
  Part of discontinuous epitope of COV_S_T2_16
RGDEVRQ

SEQ ID NO: 64 - protein, 7 aa, Artificial Sequence
  Part of discontinuous epitope of COV_S_T2_16
TGKIADY

SEQ ID NO: 65 - protein, 8 aa, Artificial Sequence
  Part of discontinuous epitope of COV_S_T2_16
YRLFRKSN

SEQ ID NO: 66 - protein, 6 aa, Artificial Sequence
  Part of discontinuous epitope of COV_S_T2_16
YQAGST

SEQ ID NO: 67 - protein, 20 aa, Artificial Sequence
  Part of discontinuous epitope of COV_S_T2_16
FNCYFPLQSYGFQPTNGVGY

SEQ ID NO: 68 - protein, 16 aa, Artificial Sequence
  Part of discontinuous epitope present in COV_S_T2_13
NITNLCPFGEVFNATR

SEQ ID NO: 69 - protein, 5 aa, Artificial Sequence
  Part of discontinuous epitope present in COV_S_T2_13
KRISN

SEQ ID NO: 70 - protein, 2 aa, Artificial Sequence
  Part of discontinuous epitope present in COV_S_T2_13
NL

SEQ ID NO: 71 - protein, 25 aa, Artificial Sequence
  Part of discontinuous epitope present in COV_S_T2_13
YNSTSFSTFKCYGVSPTKLNDLCFT

SEQ ID NO: 72 - protein, 4 aa, Artificial Sequence
  Part of discontinuous epitope present in COV_S_T2_13
DDFT

SEQ ID NO: 73 - protein, 7 aa, Artificial Sequence
  Part of discontinuous epitope present in COV_S_T2_13
TGVIADY

SEQ ID NO: 74 - protein, 8 aa, Artificial Sequence
  Part of discontinuous epitope present in COV_S_T2_13
YRSLRKSK

SEQ ID NO: 75 - protein, 6 aa, Artificial Sequence
  Part of discontinuous epitope present in COV_S_T2_13
YSPGGK

SEQ ID NO: 76 - protein, 20 aa, Artificial Sequence
  Part of discontinuous epitope present in COV_S_T2_13
FNCYYPLRSYGFFPTNGVGY

SEQ ID NO: 77 - protein, 20 aa, Artificial Sequence
  Part of discontinuous epitope present in COV_S_T2_13
FNCYYPLRSYGFFPTNGTGY

SEQ ID NO: 78 - DNA, 642 bp, Artificial Sequence
  COV_S_T2_13 encoding nucleic acid
agagtggcccctaccaaagaagtcgtgcggttccccaacatcaccaatctgtgccctttcggcgaggtgttcaacgccaccagatttccctctgtgtacgcctgggagagaaagcggatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacctggacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcgtgggctaccagccttacagagtggtggtcctgagcttcgagctgctgaatgcccctgccacagtgtgtggccctaagctgtctaccgac

SEQ ID NO: 79 - DNA, 642 bp, Artificial Sequence
  COV_S_T2_14 encoding nucleic acid
agagtggcccctaccaaagaagtcgtgcggttccccaacatcaccaatctgtgccctttcggcgaggtgttcaacgccaccaagtttccctctgtgtacgcctgggagcgcaaaaagatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacatcgacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcgtgggctaccagccttacagagtggtggtcctgagcttcgagctgctgaatgcccctgccacagtgtgtggccctaagctgtctaccgac

SEQ ID NO: 80 - DNA, 642 bp, Artificial Sequence
  COV_S_T2_15 encoding nucleic acid
agagtggcccctaccaaagaagtcgtgcggttccccaacatcaccaatctgtgccctttcggcgaggtgttcaacgccaccagatttccctctgtgtacgcctgggagagaaagcggatcagcaactgcgtggccgactacagcgtgctgtacaacagcaccttcttcagcacctttaagtgctacggcgtgtcacccaccaagctgaacgacctgtgcttcagcaacgtgtacgccgacagcttcgtgatcagaggcgacgaagtgcggcagattgcccctggacaaacaggcgtgatcgccgattacaactacaagctgcccgacgacttcatgggctgtgtgatcgcctggaacaccaacaacctggacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcgtgggctaccagccttacagagtggtggtcctgagcttcgagctgctgaatgcccctgccacagtgtgtggccctaagctgtctaccgac

SEQ ID NO: 81 - DNA, 642 bp, Artificial Sequence
  COV_S_T2_16 encoding nucleic acid
agagtggcccctaccaaagaagtcgtgcggttccccaacatcaccaatctgtgccctttcggcgaggtgttcaacgccaccagatttccctctgtgtacgcctgggagagaaagcggatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacagacaggcaagatcgccgattacaactacaagctgcccgacgacttcaccggctgtgtgatcgcctggaacaccaacaacctggacagcaccaccggcggcaactacaactacctgtaccggctgttccggaagtccaacctgaagcctttcgagcgggacatcagcagcgacatctatcaggccggcagcacaccttgttctggcgtggaaggcttcaactgctacttcccactgcaaagctacggcttccagcctaccaacggcgtgggctaccagccttatagagtggtggtcctgagcttcgagctgctgaatgcccctgccacagtgtgtggccctaagctgtctaccgac

SEQ ID NO: 82 - DNA, 642 bp, Artificial Sequence
  COV_S_T2_17 encoding nucleic acid
agagtggcccctaccaaagaagtcgtgcggttccccaacatcaccaatctgtgccctttcggcgaggtgttcaacgccaccaagtttccctctgtgtacgcctgggagcgcaaaaagatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacatcgacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcacaggctaccagccttacagagtggtggtcctgagcttcgagctgctgaatgcccctgccacagtgtgtggccctaagctgtctaccgac

SEQ ID NO: 83 - DNA, 642 bp, Artificial Sequence
  COV_S_T2_18 encoding nucleic acid
agagtggcccctaccaaagaagtcgtgcggttccccaacatcaccaatctgtgccctttcggcgaggtgttcaacgccaccagatttccctctgtgtacgcctgggagagaaagcggatcagcaactgcgtggccgactacagcgtgctgtacaacagcaccttcttcagcacctttaagtgctacggcgtgtcacccaccaagctgaacgacctgtgcttcagcaacgtgtacgccgacagcttcgtgatcagaggcgacgaagtgcggcagattgcccctggacaaacaggcgtgatcgccgattacaactacaagctgcccgacgacttcatgggctgtgtgatcgcctggaacaccaacaacctggacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcacaggctaccagccttacagagtggtggtcctgagcttcgagctgctgaatgcccctgccacagtgtgtggccctaagctgtctaccgac

SEQ ID NO: 84 - DNA, 852 bp, Artificial Sequence
  COV_S_T2_19 encoding nucleic acid
agagtggcccctaccaaagaagtcgtgcggttccccaacatcaccaatctgtgccctttcggcgaggtgttcaacgccaccagatttccctctgtgtacgcctgggagagaaagcggatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacctggacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcgtgggctaccagccttacagagtggtggtcctgagcttcgagctgctgaatgcccctgccacagtgtgtggccctaagctgtctacagatggcggcggaggatctggcggaggtggaagcggaggcggaggaagcggtggcggcggatctaaatcttctatcgccagcttcttcttcatcatcggcctgattatcggcctgttcctggtgctgagagtgggcatccacctgtgcatcaagctgaaacacaccaagaagcggcaaatctacaccgacatcgagatgaaccggctgggcaaa

SEQ ID NO: 85 - DNA, 852 bp, Artificial Sequence
  COV_S_T2_20 encoding nucleic acid
agagtggcccctaccaaagaagtcgtgcggttccccaacatcaccaatctgtgccctttcggcgaggtgttcaacgccaccaagtttccctctgtgtacgcctgggagcgcaaaaagatcagcaactgcgtggccgactacagcgtgctgtacaacagcaccagcttcagcaccttcaagtgctacggcgtgtcacccaccaagctgaacgacctgtgcttcaccaacgtgtacgccgacagcttcgtgatcagaggcgacgaagtgcggcagattgcccctggacaaacaggcgtgatcgccgattacaactacaagctgcccgacgacttcaccggctgtgtgatcgcctggaacaccaacaacatcgacagcaccaccggcggcaactacaactacctgtacagaagcctgcggaagtctaagctgaagcccttcgagcgggacatcagcagcgacatctatagccctggcggcaagccttgttctggcgtggaaggcttcaactgctactaccctctgcggagctacggcttcttccccacaaatggcacaggctaccagccttacagagtggtggtcctgagcttcgagctgctgaatgcccctgccacagtgtgtggccctaagctgtctacagatggcggcggaggatctggcggaggtggaagcggaggcggaggaagcggtggcggcggatctaaatcttctatcgccagcttcttcttcatcatcggcctgattatcggcctgttcctggtgctgagagtgggcatccacctgtgcatcaagctgaaacacaccaagaagcggcaaatctacaccgacatcgagatgaaccggctgggcaaa